SEQUENCE LISTING (44 sequences)

SEQ ID NO: 1 - protein, 290 aa, Methanosaeta sp.
  source 1..290: note = Methanosaeta sp. PtaU1.Bin055
MLQNTKNEYKPRDGGGHRPIIGFMSDLGTEDDSVGICKGVMLGLCPDAVIVDISHSMTPWDIDQGSRLIVDLPKFFPNWTTFATTSYRETGTSARSVAIKLPSGHVYVAPNNGLLTRVIEDHGYVEAYEVTTVGAIPAEPEPTWFSRDMVAYPAAAIAAGFPLENVGRPLNDSEIVRADLPRYTQAEDGIIQGIVTTIDRPFGNIWTNIPRRIIENELRIAYGTNIRVVLDNLLVLEVPFMRTFGDVGLKKPMCYINSRGYFSLAYYGGNLADPYNIRRGMPVKIESITR

SEQ ID NO: 2 - DNA, 873 bp, Methanosaeta sp.
  source 1..873: mol_type = genomic DNA; note = Methanosaeta sp. PtaU1.Bin055
atgttgcagaataccaagaacgaatacaaaccgcgcgatgggggcggccaccgacctataatcggattcatgagcgatcttggtactgaagatgattctgtaggaatatgcaaaggagtgatgcttggcctatgcccggatgcagtaattgtggatataagccattctatgacgccttgggatatcgatcaaggatcgagactaattgttgatttgcccaaattcttcccaaattggactacttttgcgactacaagctatcgtgaaacaggaacatcggcaagatctgtggcgattaagcttccaagtggccatgtatacgttgcacctaataatgggctattaactcgcgtcattgaggaccacggatatgtagaagcatatgaggttactacagtaggagcaatccctgcagaacctgagcccacttggtttagcagggatatggttgcttaccccgcagcggctatagcggcaggtttcccattagaaaatgttgggcggcctctaaatgattccgaaattgtaagagcagatttacctcgatatactcaggctgaggacggcattattcagggaatcgtgaccactatcgataggcctttcgggaacatttggacaaacatcccaagacgaataatcgaaaacgagcttagaatcgcctatggaacaaatataagggtcgttctcgataatttactggtccttgaagtcccattcatgcgtacatttggggatgttggtctcaaaaaacccatgtgttacatcaacagtcgtgggtatttttctcttgcttattatgggggaaatttagcagatccttataatatccgtaggggaatgcctgttaaaatcgaatctatcacgagataa

SEQ ID NO: 3 - DNA, 873 bp, Methanosaeta sp.
  source 1..873: mol_type = other DNA; note = Methanosaeta sp. PtaU1.Bin055
atgttgcaaaataccaaaaatgagtacaaaccacgcgatggcggagggcaccgcccaattattggattcatgtcagatttgggcacagaggacgattcagtgggaatttgtaaaggggtgatgttggggttatgtccggatgcggttatcgtggacatcagccatagtatgacgccgtgggacatcgaccagggatcgcgtctgattgtggacttgccgaagttcttccccaactggaccactttcgcaaccacttcctatcgcgaaaccggtacgtcggcacgttctgtggctatcaaattaccttctggccacgtctacgttgcgccaaataatggacttttgacacgtgttattgaggaccatggctatgtagaggcgtatgaagtgactacagtcggagctattcccgcagagcccgaaccgacgtggttttcacgtgatatggtcgcttaccctgctgccgcgattgcagccggctttccgttagagaacgtaggtcgtccgttaaacgattccgaaattgtacgtgcagacttaccccgttatactcaggctgaggacggcatcattcagggcatcgtcaccactattgatcgcccgttcggtaatatttggactaatatcccacgccgtatcatcgaaaatgagcttcgtattgcctacgggacgaacatccgcgttgtactggataatctgttagttcttgaagttccgttcatgcgtacctttggagacgttggtctgaagaaacccatgtgctatatcaacagccgtggatattttagcttagcatattatggtggaaacttagcagacccctacaatattcgccgtggaatgccggtgaagatcgaatccatcacgcgttaa

SEQ ID NO: 4 - DNA, 248 bp, synthetic construct
  source 1..248: mol_type = other DNA
tttggccctctttcgtaagcggtgactcgaagacgccttcgcttgcctcgtactccgaaacacgattttacattttggacagacctagctaagatcggcgcattggagatggcattcctccattaacaaaccgctgcgcccgtagcagctgatgatgcctacagaaacctgatcaaaccaggtctgtaggcgttcgcgcttagaatcccttctttggtcaggcccacttattttttgtggctggccaa

SEQ ID NO: 5 - DNA, 276 bp, synthetic construct
  source 1..276: mol_type = other DNA
atcctcttttggccctctttcgtaagcggtgactcgaagacgccttcgcttgcctcgtactccgaaacacgattttacattttggacagacctagctaagatcggcgcattggagatggcattcctccattaacaaaccgctgcgcccgtagcagctgatgatgcctacagaaacctgatcaaaccaggtctgtaggcgttcgcgcttagaatcccttctttggtcaggcccacttattttttgtggctggccaaatgtctaaatttcgacgacct

SEQ ID NO: 6 - DNA, 1307 bp, synthetic construct
  source 1..1307: mol_type = other DNA
tctagggcggcggatttgtcctactcaggagagcgttcaccgacaaacaacagataaaacgaaaggcccagtctttcgactgagcctttcgttttatttgatgcctttaattaaagcggataacaatttcacacaggaggccgcctaggccgcggccgcgcgaattcgagctcggtacccggggatcctcttttggccctctttcgtaagcggtgactcgaagacgccttcgcttgcctcgtactccgaaacacgattttacattttggacagacctagctaagatcggcgcattggagatggcattcctccattaacaaaccgctgcgcccgtagcagctgatgatgcctacagaaacctgatcaaaccaggtctgtaggcgttcgcgcttagaatcccttctttggtcaggcccacttattttttgtggctggccaaatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttgacttatggtgttcaatgcttttcaagatacccagatcatatgaaacggcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaatagatgcaagcttgcggccgcgtcgtgactgggaaaaccctggcgactagtcttggactcctgttgatagatccagtaatgacctcagaactccatctggatttgttcagaacgctcggttgccgccgggcgttttttattggtgagaatccag

SEQ ID NO: 7 - DNA, 1313 bp, synthetic construct
  source 1..1313: mol_type = other DNA
tctagggcggcggatttgtcctactcaggagagcgttcaccgacaaacaacagataaaacgaaaggcccagtctttcgactgagcctttcgttttatttgatgcctttaattaaagcggataacaatttcacacaggaggccgcctaggccgcggccgcgcgaattcgagctcggtacccggggatcctctttgttgacaattaatcatcggcatagtatatcggcatagtataatacgacaaggtgaggaactaaaccagacctagctaagatcggcgcattggagatggcattcctccattaacaaaccgctgcgcccgtagcagctgatgatgcctacagaaacctgatcaaaccaggtctgtaggcgttcgcgcttagaatcccttctttggtcaggcccacttattttttgtggctggccaaatgtctaaatttcgacgacctagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttgacttatggtgttcaatgcttttcaagatacccagatcatatgaaacggcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaatagatgcaagcttgcggccgcgtcgtgactgggaaaaccctggcgactagtcttggactcctgttgatagatccagtaatgacctcagaactccatctggatttgttcagaacgctcggttgccgccgggcgttttttattggtgagaatccag

SEQ ID NO: 8 - DNA, 1295 bp, synthetic construct
  source 1..1295: mol_type = other DNA
tctagggcggcggatttgtcctactcaggagagcgttcaccgacaaacaacagataaaacgaaaggcccagtctttcgactgagcctttcgttttatttgatgcctttaattaaagcggataacaatttcacacaggaggccgcctaggccgcggccgcgcgaattcgagctcggtacccggggatcctctttgttgacaattaatcatcggcatagtatatcggcatagtataatacgacaaggtgaggaactaaaccagacctagctaagatcggcgcattggagatggcattcctccattaacaaaccgctgcgcccgtagcagctgatgatgcctacagaaacctgatcaaaccaggtctgtaggcgttcgcgcttagaatcccttctttggtcaggcccacttattttttgtggctggccaaatgagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccatggccaacacttgtcactactttgacttatggtgttcaatgcttttcaagatacccagatcatatgaaacggcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaatagatgcaagcttgcggccgcgtcgtgactgggaaaaccctggcgactagtcttggactcctgttgatagatccagtaatgacctcagaactccatctggatttgttcagaacgctcggttgccgccgggcgttttttattggtgagaatccag

SEQ ID NO: 9 - DNA, 375 bp, Pseudomonas putida
  source 1..375: mol_type = genomic DNA
atgattgcactcatcgccgcggtcagcgccggtggtattgctggtaccttgttgcgctttgctaccaccaactgggtggcggctcactggccacggcacttctatgccggtacgctggcggtcaacctggttggctgcctgctgatcggcttgctctatggcttgttcttgcacaagccgctggcgccggtcgaattgcgcgccggcctgattgtcggctttctcggtggcctgacgaccttttcctccttctcgctcgataccgtgcgcctgatggaaagcgggcaagtgccccttgccttgggctataccagtatcagcgtggtgggcgggctgctcgcaacctgggctggcctgtccctgacccgattctga

SEQ ID NO: 10 - protein, 124 aa, Pseudomonas putida
MIALIAAVSAGGIAGTLLRFATTNWVAAHWPRHFYAGTLAVNLVGCLLIGLLYGLFLHKPLAPVELRAGLIVGFLGGLTTFSSFSLDTVRLMESGQVPLALGYTSISVVGGLLATWAGLSLTRF

SEQ ID NO: 11 - protein, 296 aa, Streptomyces sp.
  source 1..296: note = Streptomyces sp. MA37
MRESGGAAFYSADLGIIGGSGLYAFDGLQDVTEAELRTPYGDPSGPLVLGTAGNRRIAFLRHGRGHRIAPSQVPARANVYALKSLGVREVISVSAVGSLRGDYAPGDMVVLDQLFDRTAGLRPASFGDGMVAHVSMADPYCGRLRPFLEQVAAEEHPTVHGSGTYCCIEGPQFSTRAESEVYRSWGMDVIGMTALPEAKLAREAELCYTGLALVTDYDCWHPGHASVDAQGVAEIMEANVAAARRVVMRFAESSKPAPCVCQRALDKAIMTDSQIIQGGPGNTVPTLLRDRIAPVV

SEQ ID NO: 12 - DNA, 897 bp, Streptomyces sp.
  source 1..897: mol_type = genomic DNA; note = Streptomyces sp. MA37
atgcgagaatcgggcggcgcggccttctactcggccgacctcggaatcatcggcggcagcgggctctacgccttcgacggcctgcaggacgtgacggaggccgaactgcggaccccgtacggcgaccccagcgggccgctggtcctgggcacggccgggaaccgccgcatcgccttcctgccgcgccacgggcgcggccaccgcatagccccgtcgcaggtcccggcgcgggccaacgtctacgcgctgaagtccctcggcgtccgcgaagtgatcagcgtcagcgccgtgggcagcctgcgcggcgactatgccccgggcgacatggtcgtcctcgaccagctcttcgaccgcaccgccgggctgcggcccgcctcgttcttcggggacgggatggtggcccacgtgtcgatggcggacccgtactgcggccggctgcgccccttcctcgaacaggtggcggccgaggagcatcccacggtccacgggtccgggacgtactgctgcatcgaggggccccagttctccacccgcgccgagtccgaggtgtaccgctcatgggggatggacgtcatcggcatgacggcgctgccggaggcgaaactcgcccgtgaggctgaactctgctacaccgggctcgccctcgtgacggactacgactgctggcacccgggccacgcgtcggtggacgcccagggggtcgcggagatcatggaggccaacgtggccgccgcccggagggtcgtcatgcggttcgccgagtcgtcgaagcccgcgccctgcgtgtgtcagcgggcgctggacaaagccattatgacggacagtcagatcattcagggaggtccggggaacacggtgcccacgctcctgcgcgaccggatcgcgccggtggtgtga

SEQ ID NO: 13 - DNA, 897 bp, Streptomyces sp.
  source 1..897: mol_type = other DNA; note = Streptomyces sp. MA37
atgcgcgagagcggcggcgccgccttctacagcgccgatctgggcatcatcggcggcagcggcctgtacgccttcgacggcctgcaagacgtgaccgaagccgaactgcgcaccccgtacggcgacccgagcggcccgctggtgctgggcaccgccggcaaccgtcgtatcgcgttcctgccgcgccatggccgcggccaccgtatcgccccgagccaggtgccggcccgtgccaacgtgtatgccctgaagagcctgggcgtgcgtgaggtgatcagcgtgtccgccgtgggctccctgcgcggcgactatgcccccggtgacatggtggtgctggatcagctgttcgaccgcaccgcgggcctgcgtccggccagcttcttcggcgatggcatggtggcccatgtgtcgatggccgacccgtattgcggccgtctgcgcccgttcctggaacaggtcgccgccgaggaacacccgaccgtgcatggcagcggcacctactgctgcatcgagggcccgcagttcagcacccgcgccgaaagcgaggtgtaccgcagctggggcatggacgtcatcggcatgaccgccctgcccgaggccaaactggcgcgtgaagccgagctgtgctataccggcctggccctggtgaccgattacgactgctggcaccccggccacgccagcgtggatgcccagggcgtggccgaaatcatggaggcgaacgtggccgccgcccgccgtgtggtgatgcgtttcgcggaaagcagcaagcccgccccgtgcgtctgccagcgtgccctggataaggccatcatgaccgatagccagatcatccagggcggccccggtaacaccgtgccgaccctgctgcgcgatcgcatcgccccggtcgtgtga

SEQ ID NO: 14 - DNA, 738 bp, Escherichia coli
  source 1..738: mol_type = genomic DNA
atggctaccccacacattaatgcagaaatgggcgatttcgctgacgtagttttgatgccaggcgacccgctgcgtgcgaagtatattgctgaaactttccttgaagatgcccgtgaagtgaacaacgttcgcggtatgctgggcttcaccggtacttacaaaggccgcaaaatttccgtaatgggtcacggtatgggtatcccgtcctgctccatctacaccaaagaactgatcaccgatttcggcgtgaagaaaattatccgcgtgggttcctgtggcgcagttctgccgcacgtaaaactgcgcgacgtcgttatcggtatgggtgcctgcaccgattccaaagttaaccgcatccgttttaaagaccatgactttgccgctatcgctgacttcgacatggtgcgtaacgcagtagatgcagctaaagcactgggtattgatgctcgcgtgggtaacctgttctccgctgacctgttctactctccggacggcgaaatgttcgacgtgatggaaaaatacggcattctcggcgtggaaatggaagcggctggtatctacggcgtcgctgcagaatttggcgcgaaagccctgaccatctgcaccgtatctgaccacatccgcactcacgagcagaccactgccgctgagcgtcagactaccttcaacgacatgatcaaaatcgcactggaatccgttctgctgggcgataaagagcatcatcaccaccaccattga

SEQ ID NO: 15 - protein, 239 aa, Escherichia coli
MATPHINAEMGDFADVVLMPGDPLRAKYIAETFLEDAREVNNVRGMLGFTGTYKGRKISVMGHGMGIPSCSIYTKELITDFGVKKIIRVGSCGAVLPHVKLRDVVIGMGACTDSKVNRIRFKDHDFAAIADFDMVRNAVDAAKALGIDARVGNLFSADLFYSPDGEMFDVMEKYGILGVEMEAAGIYGVAAEFGAKALTICTVSDHIRTHEQTTAAERQTTFNDMIKIALESVLLGDKE

SEQ ID NO: 16 - DNA, 699 bp, Escherichia coli
  source 1..699: mol_type = genomic DNA
atgaaaatcggcatcattggtgcaatggaagaagaagttacgctgctgcgtgacaaaatcgaaaaccgtcaaactatcagtctcggcggttgcgaaatctataccggccaactgaatggaaccgaggttgcgcttctgaaatcgggcatcggtaaagtcgctgcggcgctgggtgccactttgctgttggaacactgcaagccagatgtgattattaacaccggttctgccggtggcctggcaccaacgttgaaagtgggcgatatcgttgtctcggacgaagcacgttatcacgacgcggatgtcacggcatttggttatgaatacggtcagttaccaggctgtccggcaggctttaaagctgacgataaactgatcgctgccgctgaggcctgcattgccgaactgaatcttaacgctgtacgtggcctgattgttagcggcgacgctttcatcaacggttctgttggtctggcgaaaatccgccacaacttcccacaggccattgctgtagagatggaagcgacggcaatcgcccatgtctgccacaatttcaacgtcccgtttgttgtcgtacgcgccatctccgacgtggccgatcaacagtctcatcttagcttcgatgagttcctggctgttgccgctaaacagtccagcctgatggttgagtcactggtgcagaaacttgcacatggctaa

SEQ ID NO: 17 - protein, 232 aa, Escherichia coli
MKIGIIGAMEEEVTLLRDKIENRQTISLGGCEIYTGQLNGTEVALLKSGIGKVAAALGATLLLEHCKPDVIINTGSAGGLAPTLKVGDIVVSDEARYHDADVTAFGYEYGQLPGCPAGFKADDKLIAAAEACIAELNLNAVRGLIVSGDAFINGSVGLAKIRHNFPQAIAVEMEATAIAHVCHNFNVPFVVVRAISDVADQQSHLSFDEFLAVAAKQSSLMVESLVQKLAHG

SEQ ID NO: 18 - DNA, 669 bp, Bacillus thuringiensis
  source 1..669: mol_type = genomic DNA
atgcatcatcaccaccaccatttattacaaaaagaaagagaagaaattgtagcgtatggaaagaaaatgatttctagtggtttaacaaaggggacgggtggtaatattagtatctttaatcgtgaacaaggtcttgttgcgattagcccaagtggtttagagtactatgaaacaaagcctgaagatgtagttatattaaacctagatggtgaagtaatagaaggagagagaaaaccatcaagtgaattagatatgcaccttatttactataggaagcgtgaagatataaatgcgcttgtgcatacacattctccttacgcgaagacgattgcgtcattaggatgggagcttccggctgtgtcatatttaattgcttttgcaggtccgaatgtccgctgtgcgccgtatgaaacatttggtacgaagcaattagcggatgctgcttttgaaggcatgattgatcgtcgtgctgtgctacttgcaaatcacggtttaattgctggtgcaaataatataaaaatggcatttactgttgcagaagagattgagttttgtgctcaaatttattatcaaacgaaaagcattggggaaccaaagctattgccagaagatgagatggagaatttggcgaagaagtttgaagggtatgggcaacagtatctcgagtga

SEQ ID NO: 19 - protein, 246 aa, Streptomyces coelicolor
MHHHHHHTYSRAGGAEGAGEPDREAAGDPTAEPAREAAWAELVATARRTVADGLVVGTSGNVSVRVGDTVLVTPSGVPYDRLTPDDATGVDLDGRQVLGTLVPTSELPMHLAVYRADPGARAVVHTHAVHATAVSLLVPELPPVHYMTAALGGPVRVAPYAAYGTAELARGMLDALAGRTGCLLRNHGTITYGTSLDQAYDRTAQLEWMCRLWLTASSVPGHSPSLLTPAQLAEVTERLRGYGQRS

SEQ ID NO: 20 - DNA, 741 bp, Streptomyces coelicolor
  source 1..741: mol_type = other DNA
atgcatcatcaccaccaccatacctacagccgcgccggcggcgccgaaggcgccggcgagccggaccgtgaggccgccggcgacccgaccgccgagccggcccgtgaggcggcctgggccgagctggtggccaccgcccgtcgtaccgtggccgatggcctggtggtgggcaccagcggcaacgtgagcgtgcgtgtgggcgacaccgtgctggtgaccccgagcggcgtgccgtatgatcgcctgaccccggacgatgccaccggcgtggacctggacggccgccaagtgctgggcaccctggtgccgacctcggagctgccgatgcatctggccgtgtaccgcgcggaccccggtgcccgtgccgtggtgcacacccatgcggtgcacgccaccgccgtgagcctgctggtgccggaactgccgcccgtgcactacatgaccgccgccctgggcggcccggtgcgtgtggccccgtacgccgcctatggcaccgccgaactggcccgtggcatgttggacgccctggcgggccgtaccggctgcctgctgcgtaaccacggcaccatcacctacggcacctccctggaccaggcctacgatcgcaccgcccagctggaatggatgtgccgcctgtggctgaccgccagcagcgtgccgggccatagcccgagcctgctgaccccggcccagctggcggaagtgaccgagcgtctgcgcggctacggccagcgtagctga

SEQ ID NO: 21 - protein, 506 aa, Pseudomonas aeruginosa
MIYAAPGTPGAVVTFKPRYGNYIGGEFVPPVKGQYFTNTSPVNGQPIAEFPRSTAEDIDKALDAAHAAAEAWGRTSVQERSNILLKIADRIEQNLELLAVTETWDNGKAVRETLNADIPLAADHFRYFAGCIRAQEGSAAEINDSTVAYHIHEPLGVVGQIIPWNFPLLMAAWKLAPALAAGNCVVLKPAEQTPLGICVLLELIGDLLPPGVLNVVQGFGREAGEALATSKRIAKIAFTGSTPVGSHILKCAAENIIPSTVELGGKSPNIYFEDIMQAEPAFIEKAAEGLVLAFFNQGEVCTCPSRALVQESIYPAFMEEVLKKVRAIKRGDPLDTETMVGAQASQQQYEKILSYLDIAQQEGAELLAGGSVEKLEGNLASGYYIQPTLLKGHNGMRVFQEEIFGPVVGVTTFKDEAEALAIANDTEYGLGAGLWTRDINRAYRMGRGIKAGRVWTNCYHLYPAHAAFGGYKKSGVGRETHKMMLDHYQQTKNLLVSYDINPLGFF

SEQ ID NO: 22 - DNA, 1521 bp, Pseudomonas aeruginosa
  source 1..1521: mol_type = genomic DNA
atgatctacgcagcgcccggaacccccggtgccgtcgtcaccttcaagcctcgctacggcaactacatcggcggcgagttcgtgcctccggtcaagggccagtacttcaccaacacctcgccggtgaacggccagccgatcgccgagttcccccgttccaccgccgaggacatcgacaaggccctcgacgccgcccacgccgccgccgaggcctggggccgcacctcggtgcaggagcgttcgaacatcctgctgaagatcgccgaccgcatcgagcagaacctggaactgctcgccgtcaccgagacctgggacaacggcaaggccgtgcgcgagaccctcaacgccgacattcccctggccgccgaccacttccgctacttcgccggctgcatccgcgcccaggaaggctccgccgccgagatcaacgacagcaccgtggcctaccacatccatgagccgctgggcgtggtcgggcagatcatcccgtggaacttcccgctgctgatggccgcctggaagctggccccggcgctggccgccggcaactgcgtggtgctcaagccggccgagcagaccccgctgggcatctgcgtgctgctggaactcatcggcgacctgctgccgccgggcgtgctcaacgtcgtccagggcttcggcagggaggccggcgaggccctggccaccagcaagcgcatcgccaagatcgccttcaccggttccaccccggtcggctcgcacatcctcaagtgcgcggcggagaacatcattccctccaccgtcgagctgggcggcaagagcccgaacatctatttcgaagacatcatgcaggccgagccggcgttcatcgagaaggccgccgagggcctggtgctggctttcttcaaccagggcgaggtgtgcacctgcccgtcccgcgcgctggtccaggagtcgatctacccggcgttcatggaagaggtgctgaaaaaggtcagggcgatcaagcgcggcgacccgctggacaccgagaccatggtcggcgcccaggcttcccagcagcagtacgagaagatcctctcctacctcgacatcgcccagcaggaaggcgccgagctgctcgccggcggcagcgtcgagaagctcgaaggcaacctggccagcggctactacatccagccgaccctgctcaaggggcacaacggcatgcgcgtgttccaggaggaaatcttcggcccggtggtcggcgtcaccaccttcaaggacgaagccgaggccctggccatcgccaacgacaccgagtacggcctcggcgccggcctctggacccgcgacatcaaccgcgcctaccgcatgggccgcgggatcaaggccggtcgcgtgtggaccaactgctaccacctgtacccggcccacgccgcgttcggcggctacaagaagtccggcgtcggtcgcgagacgcacaagatgatgctcgaccactaccagcagaccaagaacctgctggtgagctacgacatcaacccgctgggcttcttctga

SEQ ID NO: 23 - protein, 506 aa, Pseudomonas aeruginosa
MRYAHPGSEGAIVSFKARYGNYIGGEFVPPVKGQYFTNTSPVNGQPIAEFPRSTAEDIDKALDAAHAAADAWGRTSVQERSNILLKIADRIEQNLELLAVTETWDNGKAVRETLNADIPLAADHFRYFAGCIRAQEGSAAEINDSTVAYHIHEPLGVVGQIIPWNFPLLMAAWKLAPALAAGNCVVLKPAEQTPLGICVLLELIGDLLPPGVLNVVQGFGREAGEALATSKRIAKIAFTGSTPVGSHILKCAAENIIPSTVELGGKSPNIYFEDIMQAEPAFIEKAAEGLVLAFFNQGEVCTCPSRALVQESIYPAFMEEVLKKVRAIKRGDPLDTETMVGAQASQQQYEKILSYLDIAQQEGAELLAGGSVEKLEGNLASGYYIQPTLLKGHNGMRVFQEEIFGPVVGVTTFKDEAEALAIANDTEYGLGAGLWTRDINRAYRMGRGIKAGRVWTNCYHLYPAHAAFGGYKKSGVGRETHKMMLDHYQQTKNLLVSYDINPLGFF

SEQ ID NO: 24 - DNA, 1521 bp, Pseudomonas aeruginosa
  source 1..1521: mol_type = genomic DNA
atgcgttacgcccatcccggtagcgaaggcgccatcgtttccttcaaggcccgctacggcaactacatcggcggcgagttcgtgcctccggtcaagggccagtacttcaccaacacctcgccggtgaacggccagccgatcgccgagttcccccgttccaccgccgaggacatcgacaaggccctcgacgccgcccacgccgccgccgacgcctggggccgcacctcggtgcaggagcgctcgaacatcctgctgaagatcgccgaccgcatcgagcagaacctggaactgctcgccgtcaccgagacctgggacaacggcaaggccgtgcgcgagaccctcaacgccgacatccccctggccgccgaccacttccgctacttcgctggctgcattcgcgcccaggaaggctccgccgccgagatcaacgacagcaccgtggcctaccacatccatgagccgctgggcgtggtcgggcagatcatcccgtggaacttcccgctgctgatggccgcctggaagctggccccggcgctggccgccggcaactgcgtggtgctcaagccggccgagcagaccccgctgggcatctgcgtgctgctggaactcatcggcgacctgctgccgccgggcgtgctcaatgtcgtccagggcttcggcagggaggccggcgaggccctggccaccagcaagcgcatcgccaagatcgccttcaccggttccaccccggtcggctcgcacatcctcaagtgcgcggcggagaacatcattccctccaccgtcgagttgggcggcaagagcccgaacatctatttcgaagacatcatgcaggccgagccggcgttcatcgagaaggccgccgagggcctggtgctggccttcttcaaccagggcgaggtgtgcacctgcccgtcccgcgcgctggtccaggagtcgatctacccggcgttcatggaagaggtgctgaaaaaggtcagggcgatcaagcgcggcgacccgctggacaccgagaccatggtcggcgcccaggcttcccagcagcagtacgagaagatcctctcctacctcgacatcgcccagcaggaaggcgccgagctgctcgccggcggcagcgtcgagaagctcgaaggcaacctggccagcggctactacatccagccgaccctgctcaagggacacaacggcatgcgcgtgttccaggaggaaatcttcggcccggtggtcggcgtcaccaccttcaaggacgaagccgaggccctggccatcgccaacgacaccgagtacggcctcggcgccggcctctggacccgcgacatcaaccgcgcctaccgcatgggccgcgggatcaaggccggtcgcgtgtggaccaactgctaccacctgtacccggcccacgccgcgttcggcggctacaagaagtccggcgtcggtcgcgagacgcacaagatgatgctcgaccactaccagcagaccaagaacctgctggtgagctacgacatcaacccgctgggcttcttctag

SEQ ID NO: 25 - protein, 467 aa, Escherichia coli
MNQQDIEQVVKAVLLKMQSSDTPSAAVHEMGVFASLDDAVAAAKVAQQGLKSVAMRQLAIAAIREAGEKHARDLAELAVSETGMGRVEDKFAKNVAQARGTPGVECLSPQVLTGDNGLTLIENAPWGVVASVTPSTNPAATVINNAISLIAAGNSVIFAPHPAAKKVSQRAITLLNQAIVAAGGPENLLVTVANPDIETAQRLFKFPGIGLLVVTGGEAVVEAARKHTNKRLIAAGAGNPPVVVDETADLARAAQSIVKGASFDNNIICADEKVLIVVDSVADELMRLMEGQHAVKLTAEQAQQLQPVLLKNIDERGKGTVSRDWVGRDAGKIAAAIGLKVPQETRLLFVETTAEHPFAVTELMMPVLPVVRVANVADAIALAVKLEGGCHHTAAMHSRNIENMNQMANAIDTSIFVKNGPCIAGLGLGGEGWTTMTITTPTGEGVTSARTFVRLRRCVLVDAFRIV

SEQ ID NO: 26 - DNA, 1404 bp, Escherichia coli
  source 1..1404: mol_type = genomic DNA
atgaatcaacaggatattgaacaggtggtgaaagcggtactgctgaaaatgcaaagcagtgacacgccgtccgccgccgttcatgagatgggcgttttcgcgtccctggatgacgccgttgcggcagccaaagtcgcccagcaagggttaaaaagcgtggcaatgcgccagttagccattgctgccattcgtgaagcaggcgaaaaacacgccagagatttagcggaacttgccgtcagtgaaaccggcatggggcgcgttgaagataaatttgcaaaaaacgtcgctcaggcgcgcggcacaccaggcgttgagtgcctctctccgcaagtgctgactggcgacaacggcctgaccctaattgaaaacgcaccctggggcgtggtggcttcggtgacgccttccactaacccggcggcaaccgtaattaacaacgccatcagcctgattgccgcgggcaacagcgtcatttttgccccgcatccggcggcgaaaaaagtctcccagcgggcgattacgctgctcaaccaggcgattgttgccgcaggtgggccggaaaacttactggttactgtggcaaatccggatatcgaaaccgcgcaacgcttgttcaagtttccgggtatcggcctgctggtggtaaccggcggcgaagcggtagtagaagcggcgcgtaaacacaccaataaacgtctgattgccgcaggcgctggcaacccgccggtagtggtggatgaaaccgccgacctcgcccgtgccgctcagtccatcgtcaaaggcgcttctttcgataacaacatcatttgtgccgacgaaaaggtactgattgttgttgatagcgtagccgatgaactgatgcgtctgatggaaggccagcacgcggtgaaactgaccgcagaacaggcgcagcagctgcaaccggtgttgctgaaaaatatcgacgagcgcggaaaaggcaccgtcagccgtgactgggttggtcgcgacgcaggcaaaatcgcggcggcaatcggccttaaagttccgcaagaaacgcgcctgctgtttgtggaaaccaccgcagaacatccgtttgccgtgactgaactgatgatgccggtgttgcccgtcgtgcgcgtcgccaacgtggcggatgccattgcgctagcggtgaaactggaaggcggttgccaccacacggcggcaatgcactcgcgcaacatcgaaaacatgaaccagatggcgaatgctattgataccagcattttcgttaagaacggaccgtgcattgccgggctggggctgggcggggaaggctggaccaccatgaccatcaccacgccaaccggtgaaggggtaaccagcgcgcgtacgtttgtccgtctgcgtcgctgtgtattagtcgatgcgtttcgcattgtttaa

SEQ ID NO: 27 - protein, 291 aa, Moorella thermoacetica
MDKVKVAVIGPGNIGSDLMYKILRSRHLEMALMTGIIESEGIKRARKLGIKTSIEGVKAVLAEDDIKIVFDATGAKPHLQHAPLLKEAGKIAIDLTPAAVGPYVVPCVNLDQVKAEPNLNMVTCGGQATVPIVYAINRVAGARYAEIVACIASKSAGPGTRQNIDEFTQTTAKALEVVGGAKKGKAIIILNPAEPPIMMHNTIYVEVEKPDIEAIRASVEAMVKEIQSYVPGYRLVVPPILDGNKVTAAVEVEGAGDFLPKYSGNLDIITSAAVAVAEKLAQELQVKEGVA

SEQ ID NO: 28 - DNA, 876 bp, Moorella thermoacetica
  source 1..876: mol_type = genomic DNA
atggacaaggtaaaagtggctgttatcggcccgggcaatatcgggtccgacctgatgtacaagatcctgcgcagccggcatctagagatggccctgatgaccggtattatcgaatccgagggcatcaagcgggccaggaagctgggtatcaagacatccatcgaaggtgtcaaggccgtcctggcggaagacgatatcaagattgtctttgacgccaccggggccaaaccccacttacagcatgcgccgcttttaaaagaagcgggcaagatcgccatcgacttgacaccggcggcagtaggtccctacgtggtaccctgtgtcaacctggaccaggttaaggccgaaccaaaccttaacatggtcacctgcggtggccaggctacagtacccatcgtctatgccatcaaccgggtggccggggccagatacgctgagattgtagcctgcatcgcttccaagagcgccgggccggggacccgccagaacattgacgagttcacccagactacggccaaggccctggaggttgtcggcggggcgaagaagggcaaagccatcattattctgaatcctgccgagccacccattatgatgcataacaccatttatgtagaggtcgaaaagcccgacatcgaagccattcgcgcttcggttgaagccatggtcaaggaaatccagagctatgttcccgggtaccggctcgtcgtgccgcccattcttgacggcaacaaagtaacggcagctgttgaagtggagggtgctggtgacttcctgcccaaatactccggtaatctggatattatcacttcagcagccgtggcggtagctgaaaagctggcccaggaactgcaggtaaaggaaggggtagcatga

SEQ ID NO: 29 - protein, 222 aa, Bacillus thuringiensis
MHHHHHHLLQKEREEIVAYGKKMISSGLTKGTGGNISIFNREQGLVAISPSGLEYYETKPEDVVILNLDGEVIEGERKPSSELDMHLIYYRKREDINALVHTHSPYAKTIASLGWELPAVSYLIAFAGPNVRCAPYETFGTKQLADAAFEGMIDRRAVLLANHGLIAGANNIKMAFTVAEEIEFCAQIYYQTKSIGEPKLLPEDEMENLAKKFEGYGQQYLE

SEQ ID NO: 30 - protein, 299 aa, Streptomyces sp.
  source 1..299: note = Streptomyces sp. MA37
MAANGSQRPIIAFMSDLGTTDDSVAQCKGLMHSICPGVTVVDVCHSMTPWDVEEGARYIVDLPRFFPEGTVFATTTYPATGTTTRSVAVRIRQAAKGGARGQWAGSGDGFERADGSYIYIAPNNGLLTTVLEEHGYIEAYEVTSTKVIPANPEPTFYSREMVAIPSAHLAAGFPLAEVGRRLDDSEIVRFHRPAVEISGEALSGVVTAIDHPFGNIWTNIHRTDLEKAGIGQGKHLKIILDDVLPFEAPLTPTFADAGAIGNIAFYLNSRGYLSLARNAASLAYPYNLKAGLKVRVEAR

SEQ ID NO: 31 - DNA, 902 bp, Streptomyces sp.
  source 1..902: mol_type = genomic DNA; note = Streptomyces sp. MA37
tggtggctgcaaacggctctcagcgccccatcatcgccttcatgagcgacctcggcaccacggacgactcggtggcccagtgcaagggcctcatgcacagcatctgccccggcgtcacggtggtggacgtctgccactccatgacgccgtgggacgtcgaggagggtgcgcgctacatcgtcgacctcccccgcttcttccccgagggcacggtgttcgccaccaccacctatcccgcgaccggcaccacgacccgctccgtggccgtgcggatccggcaggcggccaagggcggcgcccgcggccagtgggccggctccggcgacggcttcgagcgggccgacggctcgtacatctacatcgcgcccaacaacggcctgctgaccaccgtgctcgaggagcacggatacatcgaggcctacgaggtcacgtccaccaaggtcatccccgcgaaccccgaaccgaccttctacagccgcgagatggtggccatcccctccgcccacctcgccgccggcttcccgctggccgaggtcggccgccggctcgacgacagcgagatcgtccgcttccaccggcccgcggtcgagatctccggggaggcgctctccggcgtcgtcaccgcgatcgaccaccccttcggcaacatctggaccaacatccaccgcacggacctggagaaggccggcatcggccaggggaagcacctgaagatcatcctcgacgacgtcctgccgttcgaggccccgctcaccccgaccttcgcggacgccggggcaatcggcaacatcgccttctacctcaacagccgcggctacctgtccctcgcccgcaacgccgccagcctcgcctacccctacaacctcaaggccggtctgaaggtccgcgtggaagcgcgctga

SEQ ID NO: 32 - DNA, 900 bp, Streptomyces sp.
  source 1..900: mol_type = other DNA; note = Streptomyces sp. MA37
atggccgccaacggcagccagcgcccgatcatcgcgttcatgtccgacctgggcaccaccgacgacagcgtggcgcagtgcaagggcctgatgcacagcatctgccccggtgtgaccgtggtggatgtgtgccacagcatgaccccgtgggacgtcgaggagggcgcccgttacatcgtggacctgccgcgtttcttcccggagggcaccgtcttcgccaccaccacctatcccgccaccggcaccaccacccgcagcgtggccgtgcgtatccgccaggccgccaaaggcggcgcccgtggccagtgggccggcagcggcgacggcttcgaacgtgccgacggcagctacatctacatcgccccgaacaacggcctgctgaccaccgtgctggaggaacacggctatatcgaggcctacgaggtgaccagcaccaaggtgatcccggcgaacccggagccgaccttctacagccgcgaaatggtggccatcccgtccgcccacctggccgccggcttcccgctggccgaggtgggccgtcgtctggatgacagcgagatcgtccgtttccaccgccccgccgtcgagatctccggcgaagccctgagcggcgtggtgaccgccatcgaccacccgttcggcaacatctggaccaacatccaccgtaccgacctggaaaaggccggcatcggccagggcaaacacctgaagatcatcctggacgacgtgctgccgttcgaagccccgctgacccccaccttcgccgacgccggcgccatcggcaacatcgccttctacctgaacagccgcggctatctgagcctggcccgcaacgccgccagcctggcctatccgtacaacctgaaagccggcctgaaggtgcgcgtggaggcccgttga

SEQ ID NO: 33 - protein, 409 aa, Bacillus thuringiensis
MSKFTKYFLMEASDVIAYVKEKLSKFEHAKGLKCKEIGDGNLNYVFRVWDKKENMSVIVKQAGDTARISDEFKLSTNRIRIESDVLQLENELAPGLVPKVYLFDSVMNCCVMEDLSNHTILRTALINHQIFPQLADDLTTFMVNTLLLTSDVVMNHKEKKELVKNYINPELCEITEDLVYSEPFTNHNKRNELFPLNEGWIREHIYSDKELRMEVAKRKFSFMTNAQALLHGDLHTGSVFVRDDSTKVIDPEFAFYGPMGYDVGNVMANLMFAWVNADATMPPGAEKDTYMDWLQTTMVEVIDLFKKKFLDAWDIHVTEIMAKEEGFNEVYLQSVLEDTAAVTGLELIRRIVGLAKVKDITCIENEEARARAEQICLKVAKSFILRANQYRTGRSFVETLKEQSMHYAE

SEQ ID NO: 34 - DNA, 1230 bp, Bacillus thuringiensis
  source 1..1230: mol_type = genomic DNA
atgtctaagttcacaaagtattttttaatggaagctagcgatgtgattgcatatgtgaaagagaaattatctaagtttgaacatgcaaaggggctaaagtgtaaagaaataggtgatggcaatttaaattatgtgttccgcgtttgggataaaaaagagaacatgtctgtcatcgtaaagcaagctggggatacagctcgcatttcagatgagtttaagttatcgacgaatcgtattcgtatagaatcagatgttttgcagttagagaatgagttagcacctggacttgttcctaaggtgtatttatttgatagtgtgatgaactgttgcgtaatggaagatttatcgaatcacacaatattacgtacagcacttataaatcatcaaatatttccgcagcttgctgacgacttaactacttttatggtaaatacacttttattaacatctgatgttgtaatgaatcataaagagaagaaggaacttgtgaagaattatataaatcctgaattatgtgagattacagaggacctcgtatattcagaaccatttacaaatcataataagcgtaatgagttatttcctttaaatgagggatggattagggagcatatttatagtgataaagagcttcgtatggaagtagcgaaacgtaaattttcttttatgacgaatgcacaggcattgcttcatggtgatttacatactggttctgtttttgtaagagatgattctacaaaggttattgatcctgagtttgctttttatggacctatgggatatgacgttgggaatgtaatggcgaatttaatgtttgcttgggtgaatgcagatgcaacgatgccacctggagctgagaaagatacgtatatggattggttacaaactacgatggtagaggtaattgatttatttaagaagaagtttttagatgcttgggatattcatgtgaccgagattatggcgaaggaagaaggctttaacgaagtatatttacaatctgtattagaggatacagctgcagtgacaggtcttgaattaatccgccgtattgttggattagcgaaagtaaaagatattacttgtattgagaatgaggaagcacgtgctagagcggagcaaatttgtcttaaagtagcgaagagttttattttacgagcgaatcaatatagaacaggtagaagttttgtagaaacgttaaaagaacagtcaatgcactacgcggagtaa

SEQ ID NO: 35 - protein, 353 aa, Bacillus thuringiensis
MHHHHHHEEQLIPIQWKDDALVLLDQTLLPNEIVYESFKTAESVWDAIQVMKVRGAPAIGVSAAYGVYLGVREFAESTEEGFMDEVRKVCTYLATSRPTAVNLFWALERMESVAADNIHLSILQLKDRLLEEAKEIHREDEEINRQIGEHALTLFHDGMGVLTHCNAGALATTKYGTATAPMYLAKEKGWDLKIFSDETRPRLQGSTLTALELQRAGIDVTVITDNMAAMVMSQGKIDAVIVGCDRVAANGDIANKIGTLGVSILAKYYNIPFYVAAPTPTIDLKTPTGKEIPIEERDASEVINRFGQYSAPKESKVYNPAFDVTPHENVTAIITEKGIVKAPFTENLKKIFQ

SEQ ID NO: 36 - DNA, 1062 bp, Bacillus thuringiensis
  source 1..1062: mol_type = genomic DNA
atgcatcatcaccaccaccatgaagagcaattaataccaattcagtggaaagatgatgctttagttttgttagatcaaacgttattaccgaatgaaattgtctatgaatcatttaaaacagctgaaagtgtgtgggatgccattcaagtaatgaaagtacgaggtgcaccagcgattggtgtctcagcagcttatggagtgtatttaggggtaagggaatttgctgaaagtacggaagaaggatttatggatgaagtaagaaaggtatgtacatacttggcgacatcaagaccgactgcagtaaatttattttgggcacttgaaagaatggaaagtgtagcggcagataatattcatttatcaatcttacagttgaaagatagattactagaagaggcaaaagaaattcatagagaagatgaagaaattaaccgtcaaattggagaacatgcattaacattattccatgatggaatgggagtattaacacattgtaatgctggtgcgttagcgacgactaagtatggtactgcgacggctccaatgtacttagcaaaagaaaaaggatgggacttaaaaatcttttcggatgaaacacgtccaagattacaagggtcaacgttaacggcattagaactacagcgagcgggaattgatgttactgtcattacggataatatggcggctatggtcatgtcacaagggaagattgacgctgtaattgttggatgcgaccgtgtggcagcgaacggtgatatagcaaataaaattggaacattaggcgtatctattttagcgaaatactacaacattccgttttatgtagcagcgccaacgccgacaatcgacttaaaaacaccgacaggaaaagaaattcccatcgaggaaagagatgcttctgaagtgattaatcgcttcggacaatattctgctccgaaagaaagtaaagtatataatccagcatttgatgttactccacatgaaaatgtaacagcgattattacagaaaaagggattgtaaaggcaccgtttacggagaatttaaaaaagatatttcaataa

SEQ ID NO: 37 - protein, 411 aa, Streptomyces cattleya
MSYYHHHHHHDYDIPTTENLYFQGAMGDQSVQPLAKGTGSGTPEPKPALRWEEPPEGPVLVLLDQTRLPVEEVELFCTDVPALVQAIRTLAVRGAPLLGLAGAYGVALAAARGYDVGQAADELAGARPTAVNLSYGVRRALAAYRTAVTGGADDTGAAAATLAEARALHAEDARASERMARNGLALLDELVPGGGYRVLTHCNTGALVSGGEGTALAVVLAAHRGGLLRRLWVDETRPLLQGARLTAYEAARAGVAHTLLPDGAAGSLFAAGEVDAVLIGADRIAADGSTANKVGSYPLAVLARYHNVPFVVVAPTTTIDLATPDGTAIEVEQRPAQEVTELTGPRPGPDREGATGIPVAPLGTPAYNPAFDVTPPELITAVVTETGVASPVTGSSIAALAARPGPVRAQP

SEQ ID NO: 38 - DNA, 1236 bp, Streptomyces cattleya
  source 1..1236: mol_type = genomic DNA
atgtcgtactaccatcaccatcaccatcacgattacgatatcccaacgaccgaaaacctgtattttcagggcgccatgggtgatcagtccgtacagcctttggccaagggcacggggtccgggaccccggagccgaaacccgctctccgctgggaagagcctcccgaagggcccgtgctggtcctcctcgaccagacccggctccccgtcgaggaggtcgaactgttctgtacggacgtgcccgcgctcgtccaggccatccgtaccctcgccgtccgcggcgcgccgctgctcgggctcgccggagcgtacggcgtcgccctggccgccgcccgtggctacgacgtcgggcaggccgccgacgaactcgccggcgcccggcccaccgccgtcaacctctcctacggggtgcgccgcgcgctggccgcgtaccgtaccgcggtcaccggcggcgccgacgacacgggcgcggcggcggccaccctcgccgaggcccgcgcgctgcacgccgaggacgccagggccagcgaacgcatggcccgcaacggcctggcgctgctggacgaactcgtccccggcggcggctaccgggtgctgacccactgcaacaccggcgccctggtctccggcggcgagggcaccgccctggccgtcgtcctcgccgcccaccgcggcggactcctgcgccggctgtgggtggacgagacccggccgctgctccagggcgcccggctgaccgcctacgaggccgcccgggccggcgtcgcccacacgttgctgccggacggcgcggccgggtcgctcttcgcggccggcgaggtcgacgcggtgctgatcggcgccgaccggatcgccgcggacggctcgaccgccaacaaggtcggcagctacccgctggccgtcctcgcccggtaccacaacgtccccttcgtcgtggtcgcccccaccaccacgatcgacctggccacccccgacggcaccgcgatcgaggtggagcagcgccccgcgcaggaggtgaccgagctgaccggaccgcgccccggcccggaccgcgagggcgccaccggcatccccgtcgcgcccctgggcacgccggcgtacaacccggcgttcgacgtcaccccgcccgaactgatcaccgccgtggtcaccgagaccggcgtggcctccccggtcaccggctcctccatagccgccctggccgcccgccccggccccgtccgcgcccagccgtga

SEQ ID NO: 39 - protein, 572 aa, Bacillus subtilis
MNLKALPAIEGDHNLKNYEETYRHFDWAEAEKHFSWHETGKLNAAYEAIDRHAESFRKNKVALYYKDAKRDEKYTFKEMKEESNRAGNVLRRYGNVEKGDRVFIFMPRSPELYFIMLGAIKIGAIAGPLFEAFMEGAVKDRLENSEAKVVVTTPELLERIPVDKLPHLQHVFVVGGEAESGTNIINYDEAAKQESTRLDIEWMDKKDGFLLHYTSGSTGTPKGVLHVHEAMIQQYQTGKWVLDLKEEDIYWCTADPGWVTGTVYGIFAPWLNGATNVIVGGRFSPESWYGTIEQLGVNVWYSAPTAFRMLMGAGDEMAAKYDLTSLRHVLSVGEPLNPEVIRWGHKVFNKRIHDTWWMTETGSQLICNYPCMDIKPGSMGKPIPGVEAAIVDNQGNELPPYRMGNLAIKKGWPSMMHTIWNNPEKYESYFMPGGWYVSGDSAYMDEEGYFWFQGRVDDVIMTSGERVGPFEVESKLVEHPAIAEAGVIGKPDPVRGEIIKAFIALREGFEPSDKLKEEIRLFVKQGLAAHAAPREIEFKDKLPKTRSGKIMRRVLKAWELNLPAGDLSTMED

SEQ ID NO: 40 - DNA, 1719 bp, Bacillus subtilis
  source 1..1719: mol_type = genomic DNA
atgaacttgaaagcgttaccagcaatagagggggatcataacttaaaaaactatgaagaaacgtaccggcattttgattgggccgaggcagagaaacatttctcttggcatgagacagggaaactgaatgcggcgtatgaagcgattgaccgccatgccgaatcgtttcgaaaaaacaaagtagcgctttattataaagacgcaaaaagggatgaaaaatacacatttaaagaaatgaaggaagaatcaaacagagccgggaatgtgctgagacggtatggaaatgtggaaaaaggggaccgcgtttttatttttatgccgagatcacccgagctttattttattatgcttggcgcaatcaaaattggcgccatcgccgggccgctgttcgaagcatttatggagggagcggtgaaagaccggcttgaaaacagtgaggcaaaggttgttgtcacaacgcctgagctgctggagagaataccggtagacaaactgcctcacttgcagcatgtcttcgtagtcgggggagaggctgagagcggcacgaatatcatcaattatgatgaagcagcgaaacaggaaagcacaagattggatatcgaatggatggataaaaaagacggctttctgcttcactatacatcaggttccactggtacgccaaagggcgtgttgcatgtccatgaagcgatgattcagcaatatcaaacaggaaagtgggtccttgatttaaaggaagaagacatttattggtgcacggctgatccaggctgggtgacaggtacggtatacggcatttttgcaccgtggctgaacggagcgacaaatgtcatcgtcggcggacgtttcagcccggaaagctggtatggaacgattgaacagcttggcgtcaatgtctggtacagcgcgccgacagcttttcggatgctgatgggagcgggagatgaaatggctgcgaaatatgatctaacttcactccggcatgtgctcagtgtcggtgagccgctaaatccggaagtcatcagatggggacataaagtttttaacaaacgaatccatgatacctggtggatgaccgaaacgggcagtcagctcatctgcaactatccttgcatggatattaaaccgggttcaatgggtaagccgattccaggagtggaggcagcgatcgttgacaatcaaggcaacgagctaccgccgtaccgaatgggcaatctcgccatcaaaaagggctggccttccatgatgcataccatttggaataaccctgaaaagtatgaatcgtatttcatgccgggcggctggtatgtgtctggggattctgcttacatggatgaagagggatacttttggttccaaggcagagttgatgacgtcatcatgacctccggtgagcgcgtcggcccatttgaagtggaaagcaagcttgtcgaacatccggctattgcagaagcaggcgttatcggaaagcctgacccggtgcgtggagaaatcattaaagcctttattgcactcagggaaggatttgagccgtctgataaactgaaagaagagatccgcctatttgtaaagcagggtcttgcagcccatgcggctccgcgtgagatcgaatttaaagataagcttccgaaaaccagaagcggaaagatcatgaggcgcgtgctgaaggcatgggagcttaatctgccggctggagatctgtcaacaatggaggattaa

SEQ ID NO: 41 - protein, 550 aa, Cupriavidus necator
MRDYAQAFDGFSYDDAVARQLHGSLEAMNACVECCDRHALPGRIALFWEGRDGNSRSWTFTELQALSAQFAGFLKAQGVQPGDRVAGLLPRNAELLVTILGTWRAGAVYQPLFTAFGPKAIEHRLNASGAKIVVTDGANRPKLDDVDGCPAIVTVAGDKGRGLVRGDFSFWAELERQPASFEPVPRRGDDPFLMMFTSGTTGPAKPLLVPLKAIAAFAGYMRDAVDLRAEDAFWNLADPGWAYGLYYAVTGPLALGHPTTFYDGPFTVESTCRVIRKYGITNLAGSPTAYRLLIAAGEAVSGPLRGRLRAVSSAGEPLNPEVIRWFASELGVTIHDHYGQTELGMVLCNHHALAHPVRMGAAGFASPGHRVVVVDDEQRELPPGQPGTLALDLKRSPMCWFGGYHGTSTSGFAGGYYLTGDSAELNDDGSISFIGRADDVITTSGYRVGPFDVESALIEHPAVVEAAVIGKPDPERTELIKAFVVLDPQYRAAPELAEALRQHVRKRLAAHAYPREIEFVAELPKTPSGKVQRFILRNQEVARAREAAAA

SEQ ID NO: 42 - DNA, 1653 bp, Cupriavidus necator
  source 1..1653: mol_type = genomic DNA
atgcgcgactacgcccaagccttcgacggattttcctatgacgacgccgtggcacggcagctgcacggcagcctcgaggcaatgaacgcctgcgtcgaatgctgcgaccggcacgcgctgcccggccgtatcgcgctgttctgggaagggcgcgacggcaattcgcgcagctggacctttaccgagctgcaggcactgtccgcgcagtttgccggtttcctgaaggcgcagggcgtgcagccgggcgaccgcgtggcgggcctgctgccgcgcaatgcggaactgctggtgacgattctcggcacctggcgcgccggcgcggtgtaccagccgctgttcacggccttcggccccaaggccatcgagcaccggctcaatgcgtccggcgcgaagatagtggtcaccgatggcgccaaccgccccaagctggatgacgtggatggctgtcccgccatcgtcaccgtggccggcgacaagggccgcggcctggtgcgcggcgacttcagcttctgggccgagctggaacgccagccggcgtcgttcgagccggtgccgcgccggggcgacgaccccttcctgatgatgttcacctccggcaccaccggcccggccaagccgctgctggtgccgctcaaggccatcgccgcgtttgccggctatatgcgcgacgcggtcgacctgcgcgcggaagatgctttctggaacctggccgatccgggctgggcctacggcctgtattacgcggtcacgggcccgctggcgctgggccatcccaccaccttttatgatggcccgttcaccgtggagagcacatgccgtgtgatccgcaagtacggcatcaccaacctggccggctcgcccacggcataccggctgctgatcgccgcgggcgaggccgtgtcaggcccgctgcgcgggcggctgcgcgccgtcagcagcgcgggcgagccgctcaacccggaagtgatccgctggttcgccagcgaactgggcgtgaccatccacgaccactacggccagaccgagctgggcatggtgctgtgcaaccaccatgcgctggcgcatccggtgcgcatgggcgcggccggctttgccagccctgggcaccgcgtggtggtggtggacgatgaacagcgtgaactgccgccgggccagccgggcacgctggcgctggacctgaagcgctcgccgatgtgctggttcggcggctatcacggcacgtccaccagcgggtttgccggcggctactacctgaccggcgattccgccgagctgaacgacgacggcagcatcagcttcatcggccgggccgacgacgtcatcaccacctcgggctaccgcgtgggcccgttcgacgtggaaagcgcgctgatcgaacacccggccgtggtcgaggcagcggtgatcggcaagcccgatccggagcgcaccgagctgatcaaggcctttgtcgtgctggacccgcaatatcgcgccgcgccggaactggccgaggcgctgcgccagcacgtgcgcaagcgcctggccgcccatgcctacccgcgcgagatcgagttcgtcgccgaactgcccaagacccccagcggcaaggtccagcgctttatcctgcgcaaccaggaagtggcccgtgcgcgcgaggcagccgctgcctga

SEQ ID NO: 43 - protein, 651 aa, Streptomyces coelicolor
MSNESLANLLKEERRFAPPADLAANANVTAEAYEQAKADRLGFWAEQARRLTWAKEPTETLDWSNPPFAKWFKDGTLNVAYNCVDRHVEAGNGDRVAIHFEGESGDSRALTYAQLKDEVSKAANALLELGVQKGDRVAIYMPMIPETAIAMLACARIGAAHSVVFGGFSSDALATRIQDADARVVITADGGYRRGKPSALKPAVDEAVERAGIVEHVLVVRRTGQDVAWDDSRDKWWHETVDGQSAEHTPEAFDAEHPLFILYTSGTTGKPKGILHTSGGYLTQTAYTHWAVFDLKPETDVFWCTADVGWVTGHSYIVYGPLANGATQVMYEGTPDTPHQGRFWEIVQKYGVTILYTAPTAIRTFMKWGDDIPAKFDLSSLRVLGSVGEPINPEAWIWYRKNIGADATPVVDTWWQTETGAMMITPLPGVTHAKPGSAQRPLPGISATVVDDEANEVPNGGGGYLVLTEPWPSMLRTIWGDDQRFIDTYWSRFEGKYFAGDGAKKDDDGDIWLLGRVDDVMLVSGHNISTTEVESALVSHPSVAEAAVVGATDETTGQAIVAFVILRGTTAESEDLVAELRNHVGATLGPIAKPKRILPVSELPKTRSGKIMRRLLRDVAENRQVGDVTTLADSTVMDLIQTKLPAAPSED

SEQ ID NO: 44 - DNA, 1956 bp, Streptomyces coelicolor
  source 1..1956: mol_type = genomic DNA
gtgagcaacgaatccttggccaacctgctcaaggaagaacgcaggttcgcaccccccgccgacctggccgcgaacgccaacgtcacggcggaggcgtacgaacaggccaaggctgacaggctcggcttctgggccgagcaggcccgccggctgacctgggcgaaggagccgaccgagacgctcgactggtcgaacccgccgttcgccaagtggttcaaggacggcacgctgaacgtcgcctacaactgcgtggaccgccacgtggaggccggcaacggcgaccgcgtcgccatccacttcgagggcgagtccggcgacagccgcgccctcacctacgcccagctcaaggacgaggtctccaaggccgccaacgccctgctggagctgggcgtgcagaagggcgaccgggtcgccatctacatgccgatgatccccgagaccgcgatcgcgatgctggcctgcgcccgcatcggcgccgcgcactcggtggtcttcggcggcttctcctcggacgcgctggccacccgcatccaggacgccgacgcccgcgtcgtcatcaccgccgacggcggctaccggcgcggcaagccgtccgcgctcaagccggccgtcgacgaggccgtcgagcgcgcggggatcgtcgagcacgtgctcgtcgtccgccgcaccggccaggacgtcgcctgggacgactcccgggacaagtggtggcacgagaccgtcgacgggcagtccgccgagcacacgcccgaggcgttcgacgccgagcacccgctgttcatcctgtacacgtccggcacgacggggaagcccaagggcatcctgcacacctccggcggctacctcacacagacggcgtacacccactgggcggtcttcgacctcaagccggagaccgacgtcttctggtgcaccgccgacgtcggctgggtcaccggacactcgtacatcgtctacggcccgctcgccaacggcgccacccaggtcatgtacgagggcaccccggacaccccgcaccagggccgcttctgggagatcgtgcagaagtacggcgtgacgatcctctacacggcgcccaccgcgatccggacgttcatgaagtggggcgacgacatccccgcgaagttcgacctgtcctccctgcgcgtcctcggctcggtcggcgagccgatcaaccccgaggcgtggatctggtaccgcaagaacatcggcgcggacgccaccccggtggtcgacacctggtggcagaccgagaccggcgcgatgatgatcaccccgctgccgggcgtcacccacgccaagccgggctccgcccagcgcccgctgcccggcatctccgccaccgtcgtcgacgacgaggccaacgaggtgccgaacggtggaggcggctacctggtgctcaccgagccgtggccgtcgatgctgcgcaccatctggggcgacgaccagcggttcatcgacacctactggtcgcgcttcgagggcaagtacttcgcgggcgacggcgccaagaaggacgacgacggcgacatctggctgctcggccgcgtcgacgacgtgatgctcgtctccggccacaacatctccaccaccgaggtggagtcggccctcgtctcccacccctcggtcgccgaggcggccgtggtcggcgcgacggacgagacgaccgggcaggccatcgtggccttcgtgatcctgcgcggcaccaccgcggagagcgaggacctggtcgccgagctgcgcaaccacgtcggtgccacgctcggcccgatcgccaagcccaagcggatcctgccggtctcggagctgccgaagacccgctccggcaagatcatgcggcgcctgctgcgagacgtcgcggagaaccggcaggtcggcgacgtcacgacgctggcggactcgacggtcatggacctcatccagacgaagctcccggccgcccccagcgaggactga

(19) United States
(12) Patent Application Publication
(10) Pub. No.: US 2025/0388936 A1
(43) Pub. Date: Dec. 25, 2025

(54) METHODS AND CELLS FOR THE PRODUCTION OF FLUORINATED COMPOUNDS

(71) Applicant: Danmarks Tekniske Universitet, Kongens Lyngby (DK)

(72) Inventors: Pablo Iván Nikel, Kongens Lyngby (DK); Patricia Maria Calero Valdayo, Kongens Lyngby (DK); Antonin Cros, Kongens Lyngby (DK); Robert Haas, Kongens Lyngby (DK); Isabel Pardo Mendoza, Kongens Lyngby (DK)

(21) Appl. No.: 18/844,072

(22) PCT Filed: Mar. 7, 2023
(86) PCT No.: PCT/EP2023/055733

§ 371 Date: Sep. 5, 2024

(30) Foreign Application Priority Data
Mar. 7, 2022 (EP) 22160541.3
Jul. 21, 2022 (EP) 22186275.8

(51) Int. Cl. C12P 7/06; C12N 9/00; C12N 9/02; C12N 9/04; C12N 9/06; C12N 9/24; C12N 15/52

(52) U.S. Cl. CPC C12P 7/06; C12N 9/0006; C12N 9/0008; C12N 9/0016; C12N 9/2497; C12N 9/93; C12N 15/52; C12Y 101/01008; C12Y 102/01003; C12Y 104/01001; C12Y 602/01001

(57) ABSTRACT

The present invention relates to a cell capable of producing a fluorinated compound, in particular F-acetaldehyde and optionally F-acetyl-CoA and F-acetate, methods for producing fluorinated compounds in a cell and expression systems thereof.

### TECHNICAL FIELD

The present invention relates to a cell capable of producing one or more fluorinated compounds, in particular F-acetaldehyde and optionally F-acetyl-CoA and F-acetate, methods for producing fluorinated compounds in a cell, expression systems thereof.

### BACKGROUND

The chemistry of halogens is essential for key aspects of our current lifestyle. Fluorine (F), in particular, is indispensable for industrial applications in the pharmaceutical, agriculture and material sectors. Almost 25% of all pharmaceutical molecules contain F atoms. The latest figures indicate that the market of organic fluorinated compounds will continue expanding. However, F is the most electronegative element in the periodic table, and its reactive chemistry is beyond the catalytic scope of the vast majority of the conventional enzymes. Only 12 naturally-occurring organofluorines have been identified to date. As such, the design of enzymatic routes for the site-selective introduction of the F atom into structurally diverse molecules under mild-operating conditions remains a challenge. The discovery of the fluorinase [5′-fluoro-5′-deoxyadenosine (5′-FDA) synthase] in Streptomyces and related Gram-positive species offered a unique opportunity to address this challenge. To date, this is the only enzyme known to incorporate inorganic fluoride (F⁻) into organic compounds by catalyzing the S_{N}2 addition of F to the universal C1 donor S-adenosyl-L-methionine (SAM), thereby generating 5′-FDA.

5′-FDA can be phosphorylated by a purine nucleoside phosphorylase (PNP) to 5′-fluoro-5′-deoxy-D-ribose 1-phosphate (5′-FDRP), and the resulting fluorosugar can be converted into fluoroacetaldehyde (FAld) by the sequential activities of an isomerase and an aldolase. In S. cattleya, FAld is subsequently transformed into either fluoroacetate (FAc) or 4-fluorothreonine (4-FThr). Whereas this pathway has been characterized in vitro (Deng et al., 2008), its biotechnological exploitation in vivo has been limited mostly because of the high toxicity of the resulting fluorinated molecules. The only successful in vivo fluorometabolite production was performed in Salinospora tropice, where the chlorinase from this organism was replaced with a fluorinase to produce fluorosalinosporamide (Eustaquio et al., 2010).

Despite the recent achievements on biofluorination in vivo biosynthesis of key fluorinated building blocks such as FAld, FAc, F-ethanol (FEtOH), and fluoro-acetyl-CoA (FAcCoA) remain to be achieved.

### SUMMARY

The invention is as defined in the claims.

The invention concerns efficient biosynthesis pathways enabling bioproduction of fluorinated compounds from glucose and F—, in particular 5′-fluoro-5′-deoxyadenosine (5′-FDA), 5′-fluorodeoxyribose (5′-FDR), 5′-fluoro-5′-deoxy-D-ribose 1-phosphate (5′-FDRP), 5′-(3R,4S)-5′-fluoro-5′-deoxy-D-ribulose-1-phosphate (FDRuIP), fluoroacetaldehyde (FAld), fluoroacetate (FAc), fluoroethanol (FEtOH), and/or fluoro-acetyl-CoA (FAcCoA) using a cell. Examples of such pathways are depicted in FIGS. 1, 3 and 12. With the development of biosynthesis of fluorinated compounds, the inventors have broadened the repertoire of fluorinated molecules that can be efficiently synthesised biologically, facilitating a sustainable fluorine biochemistry industry. Surprisingly, the inventors have found that the polypeptide (EnsemblBacteria: OPY51785.1, Uniprot: A0A1V5AZT2) with SEQ ID NO: 1 from the archaea Methanosaeta sp. PtaU1.Bin055, that was predicted to be a chlorinase, encodes a non-conventional fluorinase with turnover rates far superior to those of all fluorinases reported to date.

Herein disclosed is a cell capable of producing F-acetaldehyde (FAld) and optionally F-acetate (FAc), F-ethanol (FEtOH) and/or F-acetyl-CoA (FAcCoA) and/or one or more derivatives thereof from a fluorinated compound selected from 5′-fluoro-5′-deoxy-D-ribose 1-phosphate (5′-FDRP) and/or (3R,4S)-5′-fluoro-5′-deoxy-D-ribulose-1-phosphate (5′-FDRuIP), said cell expressing:
i. an isomerase (EC 5.3.1.23); and/or
ii. an aldolase (EC 4.1.2.62);
whereby said cell is capable of catalysing formation of FAld and optionally FEtOH; and
iii. optionally, said cell further expressing:
   a. an acetylating acetaldehyde dehydrogenase (EC 1.2.1.10); and/or
   b. a fluoroacetaldehyde dehydrogenase (EC 1.2.1.69), and/or an acetyl-CoA synthetase (EC 6.2.1.1);
whereby said cell is capable of catalysing formation of FAcCoA, FEtOH, and/or FAc, and/or one or more derivatives of FAld, FAc, FEtOH, and/or FAcCoA, optionally said cell further expressing:
iv. a fluorinase (EC 2.5.1.63), such as a fluorinase selected from the group consisting of FlA_{PtaU1} as set forth in SEQ ID NO: 1 and FlA1_{MA37} as set forth in SEQ ID NO: 30, or a functional variant thereof having at least 70% homology, identity or similarity to SEQ ID NO: 1 or SEQ ID NO: 30, respectively,
v. a purine nucleoside phosphorylase (PNP) (EC 2.4.2.1 and/or EC 2.4.2.28); and/or
vi. a nucleosidase (EC 3.2.2.9) and/or a kinase (EC 2.7.1.100),
   whereby said cell is further capable of fluorinating a substrate in the presence of fluoride, thereby producing the fluorinated compound, such as 5′-FDRP.

Herein disclosed is also a method for production of FAld and optionally FAcCoA, FEtOH, and/or FAc and/or one or more derivatives thereof from a fluorinated compound selected from 5′-fluoro-5′-deoxy-D-ribose 1-phosphate (5′-FDRP) and/or (3R,4S)-5′-fluoro-5′-deoxy-D-ribulose-1-phosphate (5′-FDRuIP), said method comprising the steps of:
i. providing a cell, said cell expressing:
   a) an isomerase (EC 5.3.1.23); and/or
   b) an aldolase (EC 4.1.2.62);
whereby said cell is capable of catalysing formation of FAld and optionally FEtOH; and
   c) optionally, said cell further expressing:
      I. an AcAldh (EC 1.2.1.10); and/or
      II. an F-Aldh (EC 1.2.1.69), and/or
         an Acs (EC 6.2.1.1); and
ii. propagating said cell in a medium, optionally wherein the medium comprises the fluorinated compound or a compound such as a substrate which can be converted to the fluorinated compound by said cell;
   whereby FAld and optionally FAcCoA, FEtOH, and/or FAc and/or one or more derivatives thereof is produced.

Herein disclosed is also a method for manufacturing a fluorinated compound of interest, said method comprising the steps of:
i. providing a fluorinated compound by a method disclosed herein; and
ii. optionally converting said fluorinated compound to the fluorinated compound of interest.

Also provided is a method for manufacturing a fluorinated compound of interest, said method comprising the steps of:
i) providing a fluorinated compound by the methods described herein; and
ii) optionally converting said fluorinated compound to the fluorinated compound of interest.

### DESCRIPTION OF THE DRAWINGS

FIG. 1: Canonical fluorination pathway depicting the enzymatic steps used to convert SAM and F⁻ into F-acetyl-CoA.

FIG. 2: Illustration of a synthetic circuit for fluoride-triggered biofluorination of SAM and F⁻ into F-acetyl-CoA. The T7 RNA polymerase gene is integrated in the genome; upon exposure to F⁻, the plasmid modules containing the different fluorination pathway genes regulated by the promoter PT7 (in arrows) are expressed and the enzymes of the pathway (in circles) are produced, catalyzing the conversion of SAM and F⁻ into F-acetyl-CoA.

FIG. 3: Pathway for production of 5′-FDRP using SAM and F⁻ as substrates in a first fluorination step using a fluorinase, followed by a two-step phosphorylation bypass, in which adenine is first removed by a nucleosidase and then phosphorylated using a kinase and ATP to form the final product.

FIG. 4: Procedure to prepare cell-free extracts from cultures of P. putida KT2440 containing the synthetic circuit for fluoride-triggered biofluorination. F⁻ is used as inducer of the fluoride-triggered biofluorination in exponentially growing cells. After an overnight incubation, cells are harvested, washed, and opened using a physical method to obtain a cell-free extract. This extract, containing the biofluorination enzymes, is then mixed with the substrates of the reaction, SAM and F⁻, and incubated for 20 h at 30° C. prior to detection of fluorometabolites by LC-MS.

FIG. 5: Plasmid construction used in this assay, containing the PT7→flA1·pfs·kin module, formed by the fluorinase (flA1) from Streptomyces sp. MA37, nucleosidase (pfs) from E. coli, and kinase (kin) from B. thuringiensis.

FIG. 6: Peak intensities of the three fluorometabolites of the pathway, 5′-FDA, 5′-FDR and 5′-FDRP, obtained by detection by LC-MS using cell-extracts of cultures of P. putida expressing the PT7→flA1·pfs·kin module. Error bars represent 3 biological replicates.

FIG. 7: The enzymatic reactions performed by an isomerase, converting 5′-FDRP into 5′-FDRuIP, and an aldolase, converting 5′-FDRuIP into F-acetaldehyde, are represented. In these reactions, a molecule of DHAP is released. Underlined in light grey, the coupled enzyme GPDH consumes a molecule of NADH when catalyzing the conversion of DHAP into G3P.

FIG. 8: Absorbance of NADH during the course of the enzymatic assay of isomerase, aldolase and GPDH. Two combinations of the isomerase of B. thuringiensis and S. cattleya (isoBt and isoSc) together with aldolases of B. thuringiensis and S. coelicolor (aldBt and aldSc) were tested. As negative controls, only NADH, only aldolase with substrate, and isomerase with aldolase without substrate.

FIG. 9: Same as in FIG. 8, but using the best combination of isomerase and aldolase (isomerase of S. cattleya, isoSc, and aldolase of S. coelicolor, aldSc) mixed together with nucleosidase and kinase (pfs-kinBt-isoSc-aldSc) or the PNP DeoD (deoD-isoSc-aldSc).

FIG. 10: Table with the specific activities of the different reactions calculated from the slopes obtained in the enzymatic assays. The enzymes correspond to the isomerase of B. thuringiensis and S. cattleya (isoBt and isoSc), aldolases of B. thuringiensis and S. coelicolor (aldBt and aldSc), nucleosidase and kinase (pfs-kin), and PNP DeoD (deoD).

FIG. 11: LC-MS peak of DHAP in the enzymatic reaction of isomerase of S. cattleya (isoSc) and aldolase of B. thuringiensis (aldBt) at the beginning (t0) or after 16 h (16 h). As negative controls, an isomerase reaction, only containing the isomerase of S. cattleya (isoSc) at the beginning (t0) or after 16 h (16 h).

FIG. 12: Representation of the enzymatic reactions implanted into P. putida KT2440 to transform the fluorometabolite 5′-FDA into F-acetaldehyde.

FIG. 13: Maps of the plasmids used in the experiment, containing the aldolase of B. thuringiensis (aldBt), isomerase of S. cattleya (isoSc), and either a combination of nucleosidase and kinase (pfs+kin), PT7→ald·iso·pfs·kin, or a PNP from Streptomyces MA37 (flB1) with a N-terminal His-tag), PT7→ald·iso·flB1.

FIG. 14: F¹⁹-NMR profiles of the cell-free extract reactions, expressing pFB·PT7AldIsoPfsKin, which contains the aldolase of B. thuringiensis (aldBt), the isomerase of S. cattleya (isoSc), and the combination of nucleosidase and kinase (pfs+kin), incubated with 5′-FDA for 20 hours.

FIG. 15: Same as in FIG. 14, but using cell-free extracts from cells expressing pFB·PT7AldIsoFlB1, which contains the aldolase of B. thuringiensis (aldBt), the isomerase of S. cattleya (isoSc), and the PNP from Streptomyces MA37 (flB1) with a N-terminal His-tag.

FIG. 16: Reactions of conversion of F-acetaldehyde.

FIG. 17: F-acetyl-CoA production in vitro using purified enzymes from different organisms, including ExaC (P.aer 1984) and HdhA (P.aer 4022) from P. aeruginosa, EutE from E. coli, and an aldehyde dehydrogenase, Moth_1776, from Moorella thermoacetica (M.therm 1776), using F-acetaldehyde as substrate.

FIG. 18: Kinetics of the four best enzymes, ExaC (P.aer 1984) and HdhA (P.aer 4022) from P. aeruginosa, EutE from E. coli, and the aldehyde dehydrogenase from Moorella thermoacetica (M.therm 1776), that can catalyse the reaction using F-acetaldehyde as substrate to produce F-acetyl-CoA.

FIG. 19: Kinetic parameters, Kcat and Km, of acetyl-CoA syntethases from Bacillus subtilis (AcsBs), Cupriavidus necator (AcsCn) and Streptomyces coelicolor (AcsSc).

FIG. 20: Reactions catalyzed by the fluorinase/chlorinase enzyme: (1) forward fluorination reaction; (II) forward chlorination reaction; and (Ill) reverse chlorination reaction. Step IV shows the common step in fluorometabolite biosynthetic pathways. V, VI, VII, and VIII are the steps in the canonical fluoroacetate and 4-fluoro-L-threonine biosynthetic pathway. Steps IX and X are the 5′-fluoro-5′-deoxy-D-ribose biosynthetic pathway. Compound abbreviations: 5′-ClDA, 5′-chloro-5′-deoxyadenosine; SAM, S-adenosyl-L-methionine; 5′-FDA, 5′-fluoro-5′-deoxyadenosine; 5-FDRP, 5-fluoro-5-deoxy-D-ribose-1-phosphate; 5-FDRuIP, 5-fluoro-5-deoxy-D-ribulose-1-phosphate; FAld, fluoroacetaldehyde; FAc, fluoroacetate; 4-FT, 4-fluoro-L-threonine; 5-FDR, 5-fluoro-5-deoxy-D-ribose; 5-FHPA, 5-fluoro-2,3,4-trihidroxypentanoic acid. Enzyme abbreviations (in bold): FlA, fluorinase; FlB, 5′-fluoro-5′-deoxyadenosine phosphorylase; Fllso, 5-fluoro-5-deoxy-D-ribose-1-phosphate isomerase; FlFT, 4-fluoro-L-threonine transaldolase; FdrA, 5-fluoro-5-deoxy-D-ribose-1-phosphate phosphoesterase; FdrC, 5-fluoro-5-deoxy-D-ribose dehydrogenas FIG. 21: Residues specified as essential for the EnzymeMiner search, based on the crystal structure of FlA^{MA37} (PDB ID 5B6I). The SAM substrate is shown in ball-and-stick representation.

FIG. 22: Phylogenetic tree of retrieved fluorinase sequences obtained using MEGA-X software, inferred using the Neighbor-Joining method with a bootstrap of 10,000 iterations. The percentage of replicate trees in which the associated taxa clustered together in the bootstrap test are shown next to the branches. The tree is drawn to scale, with branch lengths in the same units as those of the evolutionary distances used to infer the phylogenetic tree. Sequences sourced from Actinomycetes are highlighted as blue squares.

FIG. 23: 3D structures for FlA^{MA37} (left), •SalL^{Stro} (center, PDB ID 6RYZ), and FlA^{PtaU1} (right, modelled from the FlA^{Scat} crystal structure PDB ID 2V7V). Two chains from the homo-trimer for each structure are shown as cartoon and surface representations, respective.

FIG. 24: Phylogenetic tree of 16S rRNA sequences from organisms encoding fluorinases. Searches were performed using the NCBI BLASTn tool against the selected organisms, using the 16S rRNA sequence from Streptomyces sp. MA37 as query. Sequence accession numbers are given in Table 3. For Nocardia brasiliensis, only sequences from strain ATCC 700358 could be retrieved. Sequences specific to Methanosaeta sp. PtaU1.Bin055 could not be retrieved, so the 16S rRNA sequence from another Methanosaeta sp. was used. When more than one sequences were obtained from the whole-genome sequence of the same organism, these were all included for multiple sequence alignment and phylogenetic tree construction using the MEGA software (sub-trees collapsed below for clarity). The phylogenetic tree was inferred using the Neighbor-Joining method with a bootstrap of 10,000 iterations. The percentage of replicate trees in which the associated taxa clustered together in the boot-strap test are shown next to the branches. The tree is drawn to scale, with branch lengths in the same units as those of the evolutionary distances used to infer the phylogenetic tree. Sequences sourced from Actinomycetes are highlighted in a grey square.

FIG. 25: Steady-state fluorination assay using FlA^{PtaU1} using increasing SAM concentrations. Reactions were carried out at 37° C. in 50 mM HEPES buffer, pH 7.8, with 75 mM KF. Dotted lines show fits to the Michaelis-Menten equation (R²>0.95).

FIG. 26: Steady-state fluorination assay using FlA^{SAJ15} using increasing SAM concentrations. Reactions were carried out at 37° C. in 50 mM HEPES buffer, pH 7.8, with 75 mM KF. Dotted lines show fits to the Michaelis-Menten equation (R²>0.95).

FIG. 27: Steady-state fluorination assay using FlA^{Sxin} using increasing SAM concentrations. Reactions were carried out at 37° C. in 50 mM HEPES buffer, pH 7.8, with 75 mM KF. Dotted lines show fits to the Michaelis-Menten equation (R²>0.95).

FIG. 28: Steady-state fluorination assay using FlA^{MA37} using increasing SAM concentrations. Reactions were carried out at 37° C. in 50 mM HEPES buffer, pH 7.8, with 75 mM KF. Dotted lines show fits to the Michaelis-Menten equation (R²>0.95).

FIG. 29: End-point (1 h) transhalogenation assays with increasing 5′-CIDA concentrations. Reactions were carried out at 37° C. in 50 mM HEPES buffer, pH=7.8, with 75 mM KF and 1 mM L-Met. Error bars represent standard deviations from triplicate independent assays.

FIG. 30: Schematic representation of the fluoride-responsive genetic circuit based on the T7 phage RNA polymerase described by Calero et al. (2020) and workflow for the in vivo biofluorination assays in P. putida. The fluoride-responsive genetic circuit was induced by the addition of 15 mM NaF to the cultures at an OD₆₀₀=0.4-0.6. After incubating the cultures at 30° C. for 20 h, an aliquot was taken for metabolite extraction, biomass quantification and LC-MS analysis of fluorometabolites. Further details are provided in the Supporting Information.

FIG. 31: 5′-FDA production in cell-free extracts of engineered P. putida expressing the different fluorinase genes after 20 h of incubation at 30° C. in the presence of 200 μM SAM and 5 mM NaF. Error bars represent standard deviations. Asterisks indicate significant differences with p-values <0.1 (*) or <0.05 (**) for a two-sample, one-sided Welch's t-test.

FIG. 32: Intracellular 5′-FDA content in engineered R putida expressing different fluorinase genes. The fluorometabolite concentration was normalized by the cell dry weight (CDW). Black dots show individual values of independent experiments and error bars represent standard deviations. Asterisks indicate significant differences with p-values <0.1 (*) or <0.05 (**) for a two-sample, one-sided Welch's t-test.

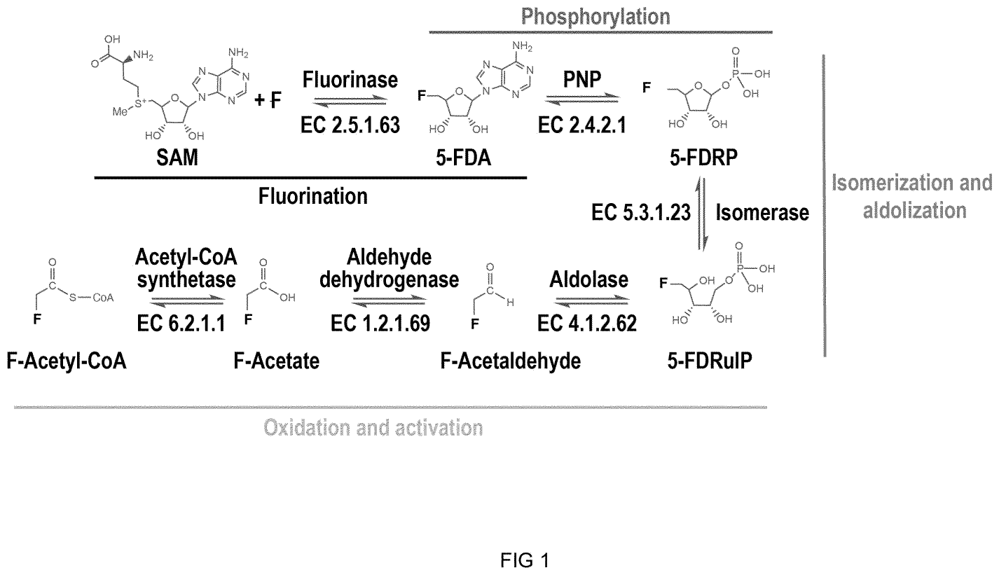

### DETAILED DESCRIPTION OF THE INVENTION

The present disclosure relates to cells capable of producing a fluorinated compound, methods for producing fluorinated compounds in a cell and expression systems therefor, in particular said fluorinated compounds are 5′-FDA, 5′-FDR, 5′-FDRP, 5′-FDRuIP, FAld, FAc, FEtOH, and/or FAcCoA and derivatives thereof.

### Definitions

Phosphorylase as term herein refers to a S-methyl-5′-thioadenosine phosphorylase (EC 2.4.2.28) and/or a purine nucleoside phosphorylase (PNP, EC 2.4.2.1). The terms phosphorylase and PNP may be used interchangeably herein. S-methyl-5′-thioadenosine phosphorylase (EC 2.4.2.28) is known to be capable of catalysing the reaction:

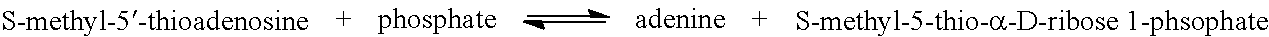

The enzyme may act on 5′-deoxyadenosine and is also capable of catalysing phosphorylation of fluorinated adenosine. PNP (EC 2.4.2.1) is known to be capable of catalysing the reactions:

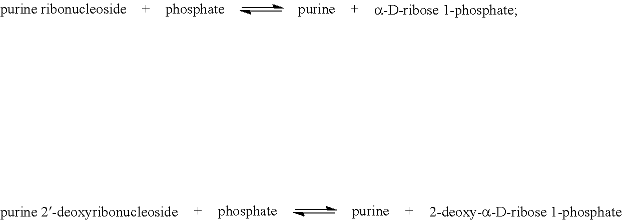

Thus, PNP may catalyse phosphorolysis of a fluorinated compound. Phosphorylase and/or PNP may be able to produce 5′-fluoro-5′-deoxy-D-ribose 1-phosphate (5′-FDRP), which may also be known as 5′-deoxy-5′-fluoro-D-ribose-1-phosphate and/or 5-FDRP and the terms may be used interchangeably herein. The terms PNP and phosphorylase may be used interchangeably herein.

Nucleosidase as term herein refers to a nucleosidase (EC 3.2.2.9), which is capable of producing a deoxyribose compound, such as a fluorinated deoxyribose compound, from a fluorinated compound. Nucleosidase is capable of catalysing the reactions:

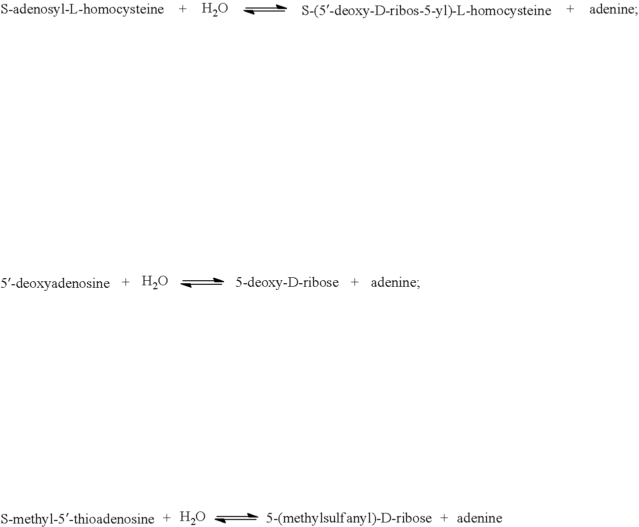 [SMILES: O ; O ; O]

Herein disclosed are nucleosidases capable of producing 5′-fluorodeoxyribose (5′-FDR) from a fluorinated compound. 5′-FDR may also be referred to as 5′-fluoro-5′-deoxyribose and/or 5-FDR.

Kinase as term herein refers to a kinase (EC 2.7.1.100), which is capable of producing a phosphorylated deoxyribose compound from a fluorinated compound. The kinase is known to be capable of catalysing the reaction:

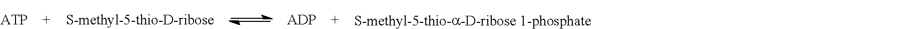

Isomerase as term herein refers to an isomerase (EC 5.3.1.23), which is capable of catalysing isomerisation of a fluorinated compound to obtain an isomer of said fluorinated compound, such as catalysing the isomerisation of 5′-fluoro-5′-deoxy-D-ribose 1-phosphate (5′-FDRP) to (3R,4S)-5′-fluoro-5′-deoxy-D-ribulose-1-phosphate (5′-FDRuIP). Isomerase is known to be capable of catalyzing the reaction: S-methyl-5-thio-α-D-ribose 1-phosphate <=>S-methyl-5-thio-D-ribulose 1-phosphate 5′-FDRP may also be referred to as 5-FDRP. 5′-FDRuIP may also be referred to as 5-FDRuIP.

Aldolase as term herein refers to an aldolase (EC 4.1.2.62), which is capable of catalysing the conversion of 5′-FDRuIP to F-acetaldehyde (FAld). Aldolase is known to be capable of catalysing the reactions:

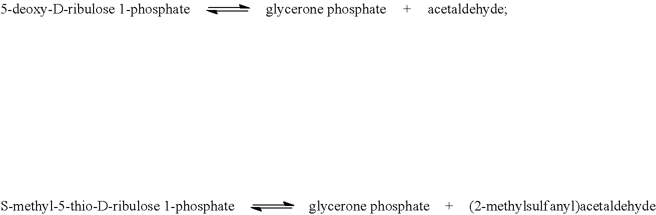

F-acetaldehyde may also herein be referred to as fluoroacetaldehyde, fluoro-acetaldehyde, FAld, and/or F-Ald, and the terms may be used interchangeably herein.

Fluoroacetaldehyde dehydrogenase as term herein refers to an acetaldehyde dehydrogenase (Aldh), such as a fluoroacetaldehyde dehydrogenase (F-Aldh, EC 1.2.1.69), which is capable of catalysing the conversion of F-acetaldehyde to fluoroacetate (FAc). Fluoroacetaldehyde dehydrogenase may also herein be referred to as F-acetaldehyde dehydrogenase, fluoro-acetaldehyde dehydrogenase, FALDH, FAldh, F-Ald dehydrogenase, Aldh or FAld dehydrogenase and the terms will be used interchangeably herein. F-Aldh is known to be capable of catalysing the reaction:

Fluoroacetaldehyde+NAD⁺+H₂O<=>fluoroacetate+NADH

F-Aldh may also have an EC number EC 1.2.1.3. Fluoroacetate may be referred to as fluoro-acetate, fluorinated acetate, Facetate, F-acetate, F-Ac and/or FAc herein, and the terms may be used interchangeably herein.

Acetyl-CoA synthetase as term herein refers to an acetyl-CoA synthetase (Acs, EC 6.2.1.1), which is capable of converting FAc to F-acetyl-CoA (FAcCoA). Acetyl-CoA synthetase may also herein be referred to as acetyl-CoA synthase, ACS or Acs, and the terms will be used interchangeably. Acs is known to be capable of catalysing the reaction: ATP+acetate+CoA<=>AMP+diphosphate+acetyl-CoA

Fluoro-acetyl-CoA may be referred to as fluoroacetyl-CoA, fluoroacetyl-coenzyme A, F-acetyl-CoA, F-Acetyl-CoA, F-acetylCoA, FAcCoA, F-AcCoA, FAc-CoA, F-AcCoA and/or F-Ac-CoA, and the terms may be used interchangeably herein.

Acetylatinq acetaldehyde dehydrogenase as term herein refers to an acetylating acetaldehyde dehydrogenase (AcAldh, EC 1.2.1.10), which is capable of converting FAld to FAcCoA. AcAldh may herein also be referred to as acylating acetaldehyde dehydrogenase, acetylating aldehyde dehydrogenase, acetaldehyde dehydrogenase (acetylating), acetaldehyde dehydrogenase, acetaldehyde-alcohol dehydrogenase, acetyl-CoA reductase, acylating acetaldehyde dehydrogenase, Ac-Aldh, AcALDH, ACALDH, Aldh and/or aldehyde dehydrogenase (acylating), and the terms may be used interchangeable herein. AcAldh is known to be capable of catalysing the reaction:

Acetaldehyde+CoA+NAD⁺<=>acetyl-CoA+NADH

Alcohol dehydrogenase as term herein refers to an alcohol dehydrogenase (ADH, EC 1.1.1.1), which is capable of converting FAld to FEtOH. ADH may also herein be referred to as Adh and/or ADH, and the terms may be used interchangeably. ADH is known to be capable of catalyzing the reactions:

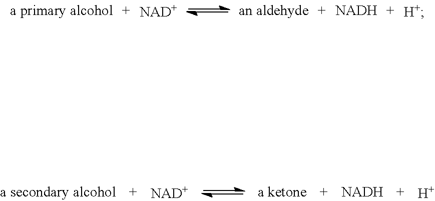

The alcohol may be F-ethanol (FEtOH). F-ethanol may be referred to as fluoro-ethanol, fluoro-EtOH, fluoroethanol, Fethanol, F-EtOH, and/or FEtOH, and the terms may be used interchangeably herein.

### Fluorinase

The fluorinase enzyme (EC 2.5.1.63, also known as adenosyl-fluoride synthase, sometimes referred to herein as FlA) catalyses the reaction between fluoride ion and the co-factor S-adenosyl-L-methionine (SAM) to generate L-methionine (L-met, L-Met, L-met, or L-Met) and 5′-fluoro-5′-deoxyadenosine (5′-FDA), the first committed product of the fluorometabolite biosynthesis pathway. Fluorinase was originally isolated from the soil bacterium Streptomyces cattleya, and is the only known enzyme capable of catalysing the formation of a carbon-fluorine bond, the strongest single bond in organic chemistry. Fluorinase catalyses the reaction:

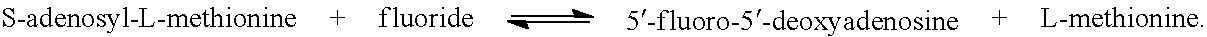

5′-fluoro-5′-deoxyadenosine (5′-FDA) may also be known as 5′-deoxy-5′-fluoroadenosine, 5′-fluoroadenosine, 5-fluoro-5-deoxyadenosine, 5-deoxy-5-fluoroadenosine, 5-fluoroadenosine, and the terms may be used interchangeably herein.

Fluorinase can however also act on other substrates besides SAM and fluoride, for example methylaza-SAM derivatives. Fluorinase may also catalyse the following reactions:

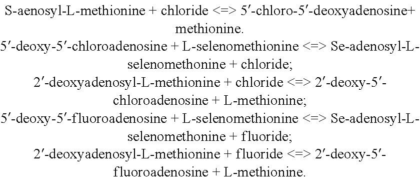

Fluorinases can thus catalyse fluorination reactions (addition of an F atom to a compound using fluoride as co-substrate) and chlorination reactions (addition of a Cl atom to a compound using chloride as co-substrate). Fluorinase catalyses the addition of F or Cl atoms at the C5′ position of SAM and SAM derivatives.

Homology, identity or similarity as terms, with respect to a polynucleotide or polypeptide, are defined herein as the percentage of nucleotides or amino acids in the candidate sequence that are identical, homologous or similar, respectively, to the residues of a corresponding native nucleotide or amino acid sequence, after aligning the sequences and introducing gaps, if necessary, to achieve the maximum percent identity/similarity, and considering any conservative substitutions according to the NCIUB rules ([https://iubmb.qmul.ac.uk/misc/naseq.html; NC-IUB, Eur J Biochem (1985)]) as part of the sequence identity. In particular, the percentage of similarity refers to the percentage of residues conserved with similar physiochemical properties. Neither 5′ or 3′ extensions nor insertions (for nucleic acids) or N′ or C′ extensions nor insertions (for polypeptides) result in a reduction of identity, similarity or homology. Methods and computer programs for the alignments are well known in the art. Generally, a given similarity between two sequences implies that the identity between these sequences is at least equal to the similarity; for example, if two sequences are 70% similar to one another, they cannot be less than 70% identical to one another—but could be sharing 80% identity. Thus, throughout the present disclosure, it will be understood that any variant, such as a functional variant, variant, or homologue said to have at least 70% identity, homology or similarity to a specified sequence (polynucleotide (nucleic acid) or polypeptide) refers to a sequence having at least 70%, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% identity, similarity or homology thereto.

Functional variant as term refers herein to functional variants of an enzyme, which retain at least some of the activity of the parent enzyme. Thus, a functional variant of a fluorinase, a phosphorylase, a PNP, a nucleosidase, a kinase, an isomerase, an aldolase, an ADH, an F-Aldh, an Aldh, an AcAldh or an Acs can catalyse the same conversion as a fluorinase, a phosphorylase, a nucleosidase, a kinase, an isomerase, an aldolase, an ADH, an F-Aldh, an Aldh, an AcAldh or an Acs, respectively, from which they are derived, although the efficiency of the conversion reaction may be different, e.g. the efficiency is decreased or increased compared to the parent enzyme or the substrate specificity is modified.

Heterologous as term when referring to a polypeptide, such as a protein or an enzyme, or to a polynucleotide, shall herein be construed to refer to a polypeptide or a polynucleotide which is not naturally present in a wild type cell. For example, the term “heterologous fluorinase” when applied to Pseudomonas putida refers to a fluorinase which is not naturally present in a wild type P. putida cell, e.g. a fluorinase derived from Methanosaeta sp. PtaU1.Bin055.

Nucleic acid as term herein may refer to a gene, a coding-sequence (CDS), an open-reading frame (ORF), nucleic acid sequence and/or a nucleic acid construct, such as RNA or DNA, comprising any of the latter and/or encoding a polypeptide, such as an enzyme, protein, riboswitch and/or amino acid sequence. The opposite is also true, such that for example the term gene herein may be referred to as nucleic acid, nucleic acid sequence and/or nucleic acid construct.

Native as term when referring to a polypeptide, such as a protein or an enzyme, or to a polynucleotide, such as a gene, open-reading frame, nucleic acid sequence and/or DNA, shall herein be construed to refer to a polypeptide or a polynucleotide which is naturally present in a wild type cell.

Derived from as term, when referring to a polypeptide or a polynucleotide derived from an organism, means that said polypeptide and/or polynucleotide is native to said organism, i.e. that it is naturally found in said organism.

Derivative as term herein refers to a first molecule, metabolite, compound or product that has undergone any conversion, either through a chemical reaction (chemical synthesis or catalysis), catalysed by one or more enzymes (enzymatic conversion) or a combination thereof, whereby a second molecule, metabolite, compound or product is produced or synthesised. Said first and/or second molecule, metabolite, compound or product may be volatile or non-volatile, halogenated, such as fluorinated, or non-halogenated, and/or unstable or stable. In the context of a metabolic pathway, a derivative of a given compound of interest is preferably obtained in a downstream part of the pathway. A precursor on the other hand is preferably a compound from which the given compound of interest is a derivative, i.e. the precursor is preferably involved in an upstream part of the pathway. The pathway may be a pathway existing in nature or a non-natural pathway, e.g. synthetic pathway. For example 5′-FDA is a precursor of 5′-FDRP and/or 5′-FDRuIP, 5′-FDRP is a precursor 5′-FDRuIP, 5′-FDRulp is a derivative of both 5′-FDRP and/or 5′-FDA.

Titer as term, such as the titer of a compound, refers herein to the produced, obtained and/or measured concentration of a compound. When the compound is produced by a cell, the term refers to the total concentration produced by the cell, i.e. the total amount of the compound divided by the volume of the culture medium. When the compound is produced in vitro such as by a purified enzyme, the term refers to the total concentration of a compound produced by the enzyme, i.e. the total amount of the compound divided by the volume of the reaction mixture. In some embodiments, the titer is divided by the cell dry weight (CDW) of the culture.

Fluorinated as term refers to a compound containing one or more fluorine (F) atoms.

Fluorinated compound as term herein refers to a compound containing one or more fluorine (F) atoms. The term may be used interchangeably with fluorinated product and/or fluorometabolite. Fluorometabolite may in particular be applied as term for any fluorinated compound of the (bio-) fluorination reactions and/or pathways disclosed herein, such as for example the fluorinated compounds 5′-FDA, 5′-FDR, 5′-FDRP, 5-FDRuIP, FAld, FAc, FEtOH, and/or FAcCoA, and derivatives thereof, for example fluorinated building blocks may also be fluorometabolites. Fluorinated compound may herein refer to any product of a fluorination reaction of any of the substrates disclosed herein and/or to any of the compounds 5′-FDA, 5′-FDR, 5′-FDRP, 5-FDRuIP, FAld, FAc, FEtOH, and/or FAcCoA, and derivatives thereof.

Fluorinated building block as term refers herein to any fluorinated molecule, compound, fluorinated chemical structure and/or fluorinated substance that can be converted by chemical and/or biological methods, such as by chemical synthesis, enzymatic activity, in vivo by a cell or by a combination of methods, into another molecule, compound, chemical structure and/or substance of interest. In other words, a building block is a precursor. Thus, a fluorinated building block is a fluorinated precursor for another molecule, compound, chemical structure and/or substance of interest to which it can be converted. The other molecule, compound, chemical structure and/or substance of interest can be fluorinated. The other molecule, compound, chemical structure and/or substance of interest can be fluorinated in addition to other modifications. 5′-chloro-5′-deoxyadenosine is sometimes herein and elsewhere also referred to as 5′-CIDA. The names are used interchangeably throughout the present disclosure.

### Cell

In a first aspect provided is a cell capable of producing FAld and optionally FAc, FEtOH and/or FAcCoA and/or one or more derivatives thereof from a fluorinated compound, said cell expressing:
i. an isomerase (EC 5.3.1.23); and/or
ii. an aldolase (EC 4.1.2.62);
   whereby said cell is capable of catalysing formation of FAld and optionally FEtOH; and
iii. optionally, said cell further expressing:
   a. an AcAldh (EC 1.2.1.10); and/or
   b. an F-Aldh (EC 1.2.1.69), and/or an Acs (EC 6.2.1.1);
      whereby said cell is capable of catalysing formation of FAcCoA, FEtOH, and/or FAc, and/or one or more derivatives of FAld, FAc, FEtOH, and/or FAcCoA.

The cell may further express a fluorinase (EC 2.5.1.63), such as a fluorinase selected from the group consisting of FlA_{PtaU1} as set forth in SEQ ID NO: 1 and FlA1_{MA37} as set forth in SEQ ID NO: 30, or a functional variant thereof having at least 70% homology, identity or similarity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology, identity or similarity to SEQ ID NO: 1 or SEQ ID NO: 30, respectively, and may further express:
i. a PNP and/or phosphorylase (EC 2.4.2.1 and/or EC 2.4.2.28); and/or
ii. a nucleosidase (EC 3.2.2.9) and/or a kinase (EC 2.7.1.100),
   whereby said cell is capable of fluorinating a substrate in the presence of fluoride, thereby obtaining the fluorinated compound.

Thus, for the cell to be able to produce a fluorinated compound such as 5′-fluoro-5′-deoxyadenosine (5′-FDA) from a substrate that is not fluorinated, i.e. fluorinate said substrate, the cell may express a fluorinase, said fluorinase being capable of catalysing fluorination of a substrate in the presence of fluoride, whereby the cell is capable of producing a fluorinated compound such as 5′-FDA. The cell and/or fluorinase may be as described herein, for example in “Fluorinase”.

In the presence of a fluorinated compound, such as upon producing a fluorinated compound, the cell may catalyse the conversion of said fluorinated compound into another fluorinated compound. In other words, in the presence of a first fluorinated compound, the cell may catalyse the conversion of said first fluorinated compound into a second fluorinated compound. Said another fluorinated compound may also be referred to as a fluorinated product. The first fluorinated compound and the enzymes expressed by the cell determines the produced another fluorinated compound, but may be any of the fluorinated molecules disclosed herein, in particular 5′-FDA, 5′-FDR, 5′-FDRP, 5′-FDRuIP, FAld, FAc, FEtOH and FAcCoA

In the presence of a fluorinated adenosine, for example 5′-FDA, and expression of at least a PNP, wherein the PNP is capable of catalysing phosphorylation of a fluorinated compound such as a fluorinated adenosine, preferably 5′-FDA, the cell may be capable of producing a phosphorylated and fluorinated compound such as 5′-fluoro-5′-deoxy-D-ribose 1-phosphate (5′-FDRP). The cell and/or PNP may be as described herein, for example in “Phosphorylase and/or PNP”.

In the presence of 5′-FDA, such as upon producing 5′-FDA, and expression of at least a nucleosidase, wherein the nucleosidase is capable of catalysing the conversion of 5′-FDA to 5′-fluorodeoxyribose (5′-FDR), the cell may be capable of producing 5′-FDR. The cell and/or nucleosidase may be as described herein, for example in “Nucleosidase” or “Phosphorylation bypass”.

In the presence of 5′-FDR, such as upon producing 5′-FDR, and expression of at least a kinase, wherein the kinase is capable of catalysing the conversion of 5′-FDR to 5′-FDRP, the cell may be capable of producing 5′-FDRP. The cell and/or kinase may be as described herein, for example in “Phosphorylation bypass” and/or “Nucleosidase”.

The consecutive action, i.e. two-step action, of a nucleosidase and a kinase to convert 5′-FDA to 5′-FDRP, may also be referred to as the phosphorylation bypass pathway. Said phosphorylation bypass pathway may substitute or complement the cleavage and/or phosphorylation of a fluorinated compound, i.e. 5′-FDA, by PNP.

In the presence of 5′-FDRP, such as upon producing 5′-FDRP, and expression of at least an isomerase, wherein the isomerase is capable of catalysing the isomerisation of 5′-FDRP, the cell may be capable of producing a 5′-FDRP isomer such as (3R,4S)-5′-fluoro-5′-deoxy-D-ribulose-1-phosphate (5′-FDRuIP). The cell and/or isomerase may be as described herein, for example in “Isomerase”.

In the presence of 5′-FDRuIP, such as upon producing 5′-FDRuIP, and expression of at least an aldolase, wherein the aldolase is capable of catalysing the conversion of 5′-FDRuIP to FAld, the cell may be capable of producing FAld. The cell and/or aldolase may be as described herein, for example in “Aldolase”.

FAld may be converted into different fluorinated compounds, for example FAcCoA, FAc and/or FEtOH. Thus, in the presence of FAld, such as upon producing FAld, and expression of an acetylating acetaldehyde dehydrogenase (AcAldh), wherein the AcAldh is capable of catalysing the conversion of FAld to FAcCoA, the cell may be capable of producing FAcCoA. The cell and/or AcAldh may be as described herein, for example in “Acetylating acetaldehyde dehydrogenase”.

With regards to FAc, in the presence of FAld, such as upon producing FAld, and expression of at least a fluoroacetaldehyde dehydrogenase (F-Aldh), wherein the F-Aldh is capable of catalysing the conversion of FAld to FAc, the cell may be capable of producing FAc. The cell and/or F-Aldh may be as described herein, for example in “Acetaldehyde dehydrogenase” and “Fluoroacetaldehyde dehydrogenase”.

The F-Aldh and the AcAldh may be are identical, i.e. the same enzyme, for example an acetaldehyde dehydrogenase (Aldh) may be capable of catalysing the conversion of FAld to FAc and FAcCoA. Thus, in the presence of FAld, such as upon producing FAld, and expression of at least an Aldh, wherein the Aldh is capable of catalysing the conversion of FAld to FAc and FAcCoA, the cell may be capable of producing FAc and FAcCoA.

With regards to FEtOH, in the presence of FAld, such as upon producing FAld, and expression of at least an alcohol dehydrogenase (ADH), wherein the ADH is capable of catalysing the conversion of FAld to FEtOH, the cell may be capable of producing FEtOH. The cell and/or ADH may be as described herein, for example in “Alcohol dehydrogenase”.

In the presence of FAc, such as upon producing FAC, and expression of at least an acetyl-CoA synthetase (Acs), wherein the Acs is capable of catalysing the conversion of FAc to FAcCoA, the cell may be capable of producing FAcCoA. The cell and/or Acs may be as described herein, for example in “Acetyl-CoA synthetase”.

In another aspect, is provided a cell capable of producing a fluorinated compound from a substrate in the presence of fluoride, said cell comprising:
a) a first nucleic acid comprising a first promoter and a fluorinase gene encoding a fluorinase (EC 2.5.1.63) capable of catalysing the formation of a C—F bond, said fluorinase gene being under the control of the first promoter;
b) optionally a second nucleic acid comprising a riboswitch, wherein transcription of the fluorinase gene from the first promoter is induced in the presence of an inducer, wherein said riboswitch is responsive to said inducer;
   wherein the cell is capable of expressing the fluorinase at least in the presence of said inducer,
   wherein the fluorinase is the fluorinase of Methanosaeta sp. PtaU1.Bin055 as set forth in SEQ ID NO: 1 (FlA_{PtaU1}), or a functional variant thereof having at least 70% sequence homology, similarity or identity thereto, such as at least 75%, such as at least 80%, such as at least 85%, such as at least 90%, such as at least 95%, such as at least 100% sequence homology, similarity or identity to SEQ ID NO: 1.

Also provided is a cell capable of producing a fluorinated compound from a substrate in the presence of fluoride, said cell comprising:
a) a first nucleic acid comprising a first promoter and a fluorinase gene encoding a fluorinase (EC 2.5.1.63) capable of catalysing the formation of a C—F bond, said fluorinase gene being under the control of the first promoter;
b) optionally a second nucleic acid comprising a riboswitch, a second promoter and a gene encoding an activator of transcription, said gene being under the control of the second promoter, wherein transcription of the activator of transcription can be induced by an inducer, wherein the activator of transcription upon expression activates transcription from the first promoter;
wherein the cell is capable of expressing the activator of transcription and the fluorinase at least in the presence of said inducer,
wherein the fluorinase is the fluorinase of Methanosaeta sp. PtaU1.Bin055 as set forth in SEQ ID NO: 1 (FlA_{PtaU1}), or a functional variant thereof having at least 70% sequence homology, similarity or identity thereto, such as at least 75%, such as at least 80%, such as at least 85%, such as at least 90%, such as at least 95%, such as at least 100% sequence homology, similarity or identity to SEQ ID NO: 1.

Throughout the disclosure, a cell capable of producing a fluorinated compound from a substrate in the presence of fluoride will refer to a cell capable of converting a substrate into a fluorinated compound in the presence of fluoride. Similarly, a cell capable of producing a fluorinated compound from a first fluorinated compound will refer to a cell capable of converting a first fluorinated compound into another fluorinated compound in the presence of said first fluorinated compound. The first fluorinated compound and/or the substrate may be provided to the cell, such as supplemented in the cultivation medium, or produced by the cell.

Preferably, the cell can tolerate toxic compounds, such as fluorinated compounds. In some embodiments, the cell is a cell of a non-pathogenic organism. For example, the cell is a cell of a GRAS (Generally Regarded As Safe) organism.

Cells useful for performing the methods disclosed herein may be mammalian cells, plant cells, insect cells, yeast cells or bacterial cells.

In embodiments where the cell is a yeast cell, preferably the yeast is of the genus Saccharomyces, Pichia, Yarrowia, Kluyveromyces, Candida, Rhodotorula, Rhodosporidium, Cryptococcus, Trichosporon or Lipomyces. For example, the yeast is selected from Saccharomyces cerevisiae, Pichia pastoris, Kluyveromyces marxianus, Cryptococcus albidus, Lipomyces lipofer, Lipomyces starkeyi, Rhodosporidium toruloides, Rhodotorula glutinis, Trichosporon pullulans and Yarrowia lipolytica.

In embodiments where the cell is a bacterial cell, preferably the cell is a Gram-negative bacterial cell. The skilled person will not have difficulties in identifying Gram-negative bacterial cells. In some embodiments, the cell is a bacterial cell of the Pseudomonas genus, the Bacillus genus, the Streptomyces genus, the Vibrio genus or the Escherichia genus. For example, the bacterial cell is selected from Pseudomonas putida, Pseudomonas fluorescens, Pseudomonas taiwanensis, Pseudomonas syringae, Pseudomonas stutzeri, Pseudomonas oleovorans, Pseudomonas mendocina, Bacillus subtilis, Bacillus cereus, Bacillus megaterium, Streptomyces albus, Streptomyces venezuelae, Streptomyces coelicolor, Vibrio natriegens and Escherichia coli. In preferred embodiments, the bacterial cell is a Pseudomonas putida cell, for example Pseudomonas putida KT2440.

In preferred embodiments, the cell is a Pseudomonas putida KT2440 cell. In some embodiments, the cell is evolved and/or engineered from a P. putida KT2440 cell.

The cell is used as production organism for fluorinated compounds as described herein. Alternatively, the cell may be used as source of the catalytic enzymes for production of fluorinated compounds described herein.

The cell may comprise a first nucleic acid comprising a first promoter and a fluorinase gene encoding a fluorinase, and optionally a second nucleic acid comprising a riboswitch. In some embodiments the first nucleic acid and the second nucleic acid are comprised within the same nucleic acid molecule.

In other embodiments, the first nucleic acid and the second nucleic acid are different, i.e. they are not comprised within the same nucleic acid molecule. In such embodiments, the cell may comprise a first nucleic acid comprising a first promoter and a fluorinase gene encoding a fluorinase, and a second nucleic acid comprising a riboswitch, a second promoter and a gene encoding an activator of transcription.

The titer(s) produced by any of the cells of the present disclosure may be as described in the section “Titer” herein below.

### Fluorinase

Fluorinases are enzymes which are capable of catalysing the formation of a C—F bond in the presence of fluoride. Fluorinases expressed in the cell of the present disclosure are capable of catalysing fluorination of a substrate to obtain a fluorinated compound, as will be detailed further below herein. The fluorinase thus is capable of catalysing fluorination of a substrate to obtain a fluorinated compound. In relation to the fluorinase, said fluorinated compound may be any compound obtained from the fluorination of any of the substrates disclosed in the section “Substrates” herein below. In some embodiments, the fluorinated compound is 5′-FDA.

The fluorinase may be a heterologous fluorinase, i.e. a fluorinase that is not native to or expressed endogenously in the cell. In preferred embodiments, the fluorinase is a fluorinase native to Methanosaeta such as Methanosaeta sp. PtaU1.Bin055. In other embodiments, the fluorinase is a fluorinase native to Streptomyces such as Streptomyces sp. MA37, Streptomyces sp. SAJ15, S. cattleya, S. xinghaiensis, or a fluorinase native to Nocardia such as N. brasiliensis or a fluorinase native to Actinoplanes such as Actinoplanes sp. N902-109.

In some embodiments, the fluorinase is the fluorinase of Methanosaeta sp. PtaU1.Bin055 as set forth in SEQ ID NO: 1 (FlA_{PtaU1}), or a functional variant thereof having at least 70% sequence homology, similarity or identity thereto, such as at least 71%, such as at least 72%, such as at least 73%, such as at least 74%, such as at least 75%, such as at least 76%, such as at least 77%, such as at least 78%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence homology, similarity or identity to SEQ ID NO: 1. The fluorinase of Methanosaeta sp. PtaU1.Bin055 as set forth in SEQ ID NO: 1 (FlA_{PtaU1}) may also sometimes herein be referred to as FlA^{PtaU1}.

In some embodiments, the fluorinase is a fluorinase native to Streptomyces sp. MA37, such as FlA1_{MA37} as set forth in SEQ ID NO: 30, or a functional variant thereof having at least 70% homology, identity or similarity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology, identity or similarity to SEQ ID NO: 30. The fluorinase of Streptomyces sp. MA37 as set forth in SEQ ID NO: 30 (FlA1_{MA37}) may also sometimes herein be referred to as FlA^{MA37}. Likewise, the fluorinase gene(s) encoding FlA1_{MA37} of Streptomyces sp. MA37 may also sometimes herein be referred to as flA1_{MA37} and flA^{MA37}, and the terms will be used interchangeably.

In some embodiments, the fluorinase is a fluorinase native to Streptomyces sp. SAJ15, such as FlA_{SAJ15} (accession number: WP_144383880.1), or a functional variant thereof having at least 70% homology, identity or similarity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology, identity or similarity to FlA_{SAJ15} (accession number: WP_144383880.1). The fluorinase FlA_{SAJ15} (accession number: WP_144383880.1) of Streptomyces sp. SAJ15, may also sometimes herein be referred to as FlA^{SAJ15} and the terms will be used interchangeably.

In some embodiments, the fluorinase is a fluorinase native to Streptomyces xinghaiensis, such as FlA_{Sxin} (accession number: WP_019711456.1), or a functional variant thereof having at least 70% homology, identity or similarity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology, identity or similarity to FlA_{Sxin} (accession number: WP_019711456.1). The fluorinase FlA_{Sxin} (accession number: WP_144383880.1) of S. xinghaiensis, may also sometimes herein be referred to as FlA_{Sxin}, and the terms will be used interchangeably.

The activity of a fluorinase can be measured by assessing the amount of fluorometabolites produced by the enzyme in an in vitro system. For example, the purified fluorinase enzyme can be exposed to SAM and fluoride, and the formation of 5′-FDA can be assessed by HPLC. Kinetic parameters (Michaelis-Menten constant, K_{M}, and turnover number, k_{cat}) can be derived from these measurements by using different substrate concentrations.

The fluorinase gene encodes a fluorinase (EC 2.5.1.63); the fluorinase may be the fluorinase of Methanosaeta sp. PtaU1.Bin055 as set forth in SEQ ID NO: 1 (FlA_{PtaU1}), or a functional variant thereof having at least 70% sequence homology, similarity or identity thereto, such as at least 75%, such as at least 80%, such as at least 85%, such as at least 90%, such as at least 95%, such as at least 100% sequence homology, similarity or identity to SEQ ID NO: 1. The fluorinase may also be the fluorinase of Streptomyces sp. MA37 as set forth in SEQ ID NO: 30 (FlA1_{MA37}), or a functional variant thereof having at least 70% sequence homology, similarity or identity thereto, such as at least 75%, such as at least 80%, such as at least 85%, such as at least 90%, such as at least 95%, such as at least 100% sequence homology, similarity or identity to SEQ ID NO: 30.

In some embodiments, the fluorinase gene encoding the fluorinase is as set forth in SEQ ID NO: 2, or a homologue thereof having at least 70% sequence homology, similarity or identity thereto, such as at least 71%, such as at least 72%, such as at least 73%, such as at least 74%, such as at least 75%, such as at least 76%, such as at least 77%, such as at least 78%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence homology, similarity or identity to SEQ ID NO: 2.

As the fluorinase gene may be a heterologous gene, it may be codon-optimised to improve transcription in the cell in which it is to be expressed, as is known in the art.

Accordingly, in some embodiments, the fluorinase gene encoding the fluorinase is as set forth in SEQ ID NO: 3, or a homologue thereof having at least 70% sequence homology, similarity or identity thereto, such as at least 71%, such as at least 72%, such as at least 73%, such as at least 74%, such as at least 75%, such as at least 76%, such as at least 77%, such as at least 78%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence homology, similarity or identity to SEQ ID NO: 3. SEQ ID NO: 3 encodes FlA_{PtaU1} of Methanosaeta sp. PtaU1.Bin055 (SEQ ID NO: 1) and has been codon optimised for expression in P. putida. The fluorinase gene(s) encoding FlA_{PtaU1} of Methanosaeta sp. PtaU1.Bin055 may also sometimes herein be referred to as flA_{PtaU1}, and flA^{PtaU1}, and the terms will be used interchangeably.

Expression of the fluorinase gene may be under the control of the first promoter comprised in the first nucleic acid. The fluorinase gene encodes a fluorinase or a functional variant thereof, such as a mutant, which retains the activity of the fluorinase but may have modified properties, such as modified substrate preferences, modified efficiency, and others. For example, fluorinases have been modified by directed evolution and rational design approaches to improve catalytic efficiency on non-native substrates (Sun et al., 2016; Thomsen et al., 2013). Such mutant fluorinases may also be used in the context of the present disclosure.

### Substrates

Fluorinase can convert S-adenosyl-L-methionine (SAM) to 5′-fluoro-5′-deoxyadenosine (5′-FDA) in the presence of inorganic fluoride (F⁻). The reaction is reversible. The enzyme can also convert other substrates to yield other fluorinated compound. For example, it can act on derivatives of SAM, such as a methylaza derivative, 5′-chloro-5′-deoxyadenosine, a 2-deoxyadenosine analogue, or L-methionine analogues, di-cyclic peptide conjugates of 5′-chlorodeoxy-2-ethynyladenosine, tri-cyclic peptide conjugates of 5′-chlorodeoxy-2-ethynyladenosine, fluoride, and ¹⁸F.

In some embodiments, the substrate is a SAM derivative of formula (I):

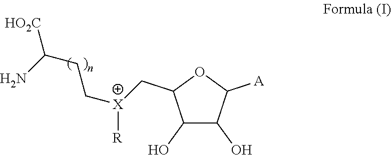

wherein:
n is 0, 1 or 2;
X is selected from the group consisting of: S, Se and NMe;
R is selected from the group consisting of: Me and propargyl;
A is a heterocycle.

The SAM derivative may thus be of formula (II):

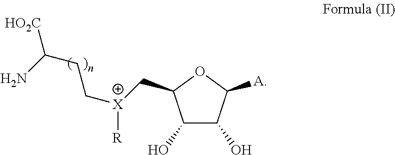

wherein X is selected from the group consisting of: S, Se and NMe; R is selected from the group consisting of: Me and propargyl; and A is a heterocycle.

In some embodiments, A is selected from the group consisting of

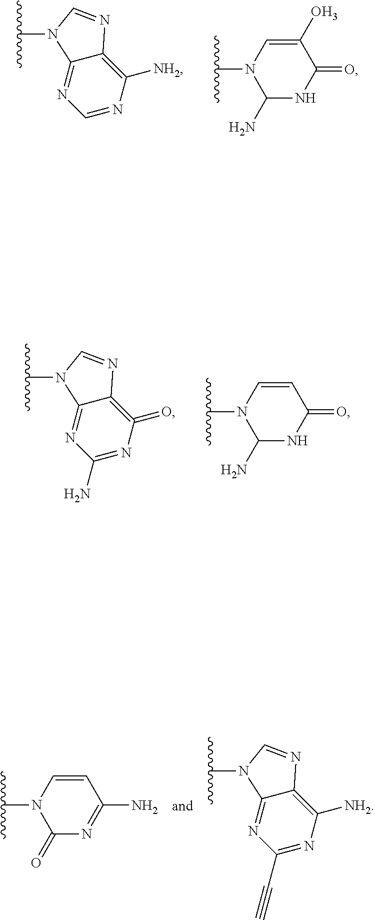

In some embodiments, the SAM derivative of formula (I) is of formula (III):

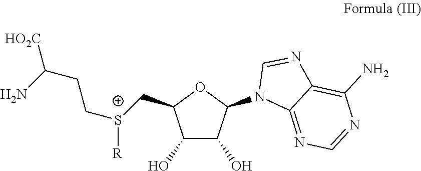

wherein R is selected from the group consisting of: Me and propargyl.

In preferred embodiments, the substrate is SAM.

In the presence of fluoride (F⁻) the fluorinase catalyses fluorination of the substrate.

This is the preferred reaction catalysed by the enzyme.

The fluorination reaction may be guided, such as initiated, by the use of a co-substrate.

The co-substrates may be provided in the form of salts. Preferred salts are soluble salts. In some embodiments, the reaction is a fluorination reaction and the co-substrate is a fluoride salt. Preferred fluoride salts are NaF or KF; less soluble salts such as CaF₂ may also be used.

In some embodiments, the fluorinase is capable of converting one or more of:

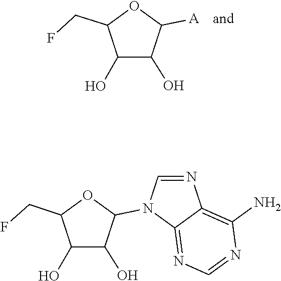

### Activator of Transcription and First Promoter

The cell may in some embodiments further comprise an activator of transcription, which can bind the first promoter. The first promoter controls expression of the fluorinase gene. In some embodiments, the activator of transcription is the T7 RNA polymerase and the first promoter is a T7 promoter, which is recognised by the T7 RNA polymerase. Accordingly, the fluorinase gene is expressed in the cell provided that the activator of transcription is expressed and active.

### Riboswitch and Second Promoter

The present inventors have found that in order to obtain tight and precise regulation of the expression of the fluorinase gene, it is advantageous to tightly regulate expression of the activator of transcription. This surprisingly results in an efficient system which can be tightly regulated to precisely control the fluorination reaction. As shown in the examples and as also demonstrated in the application WO2020/083958 entitled “In vivo fluorination, chlorination and bromination” filed on 23 Oct. 2019 by same applicant, the use of a riboswitch together with a second promoter to control expression of the activator of transcription is particularly advantageous. However, the riboswitch may control expression of the fluorinase gene from the first promoter by activating transcription from the first promoter directly.

The riboswitch is activated by an inducer, as described herein below. In some embodiments, the inducer is a co-substrate of the reaction. For example, for fluorination of a substrate, the inducer can be the co-substrate, i.e. fluoride.

Preferably, a fluoride-responsive riboswitch (FRS) is used. For example, the FRS from Pseudomonas syringae is used. In its natural context this riboswitch drives the expression of the gene coding for the F⁻ transporter EriC^{F} in the presence of F⁻.

In embodiments where it is desirable to obtain fluorinated compounds, the riboswitch may be fluoride-responsive, i.e. it is activated by the presence of fluoride.

In some embodiments, the riboswitch is a fluoride-responsive riboswitch, such as the FRS riboswitch from P. syringae (SEQ ID NO: 4). In some embodiments, the cell is a Pseudomonas putida cell, in particular a P. putida KT2440 cell. In some embodiments, the activator of transcription is the T7 RNA polymerase and the first promoter is a T7 promoter.

In some embodiments, the second nucleic acid thus comprises a riboswitch having the sequence as set forth in SEQ ID NO: 4, or a homologue thereof having at least 70% sequence homology, similarity or identity thereto, such as at least 71%, such as at least 72%, such as at least 73%, such as at least 74%, such as at least 75%, such as at least 76%, such as at least 77%, such as at least 78%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence homology, similarity or identity thereto. The term “functional variant” of an FRS refers to a sequence which retains the ability to drive expression of a gene downstream of it in response to fluoride.

The second nucleic acid in some embodiments may also comprise a second promoter. The second promoter may be the native promoter normally found with the riboswitch used. For example, the riboswitch is the FRS riboswitch from P. syringae as described above, and the second promoter is the native promoter of the eriC^{F} gene of P. syringae. In other embodiments, the second promoter is a constitutive promoter, such as a synthetic promoter, for example a P_{EM7} promoter, a P_{BG42} promoter, a P_{tetA} promoter, or any constitutive promoter as known in the art.

Without being bound by theory, the use of a two-step regulation system, i.e. a second nucleic acid comprising a riboswitch and a promoter controlling expression of a first nucleic acid comprising a first promoter and a fluorinase gene, is expected to amplify the transcriptional signal and result in increased expression of the fluorinase gene in an inducible manner. However, as can be seen from the examples below, a one-step regulation system, i.e. wherein the riboswitch directly controls expression of the fluorinase from the first promoter, also leads to expression of fluorinase in an inducible manner.

In some embodiments, the second nucleic acid comprises or consists of the FRS sequence as set forth in SEQ ID NO: 4 or a homologue thereof and of the native promoter of the eriC^{F} gene of P. syringae. The sequence of the corresponding second nucleic acid is set forth in SEQ ID NO: 6. In some embodiments, the second nucleic acid comprises or consists of SEQ ID NO: 6 or a functional variant thereof having at 70% sequence homology, similarity or identity thereto, such as at least 71%, such as at least 72%, such as at least 73%, such as at least 74%, such as at least 75%, such as at least 76%, such as at least 77%, such as at least 78%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence homology, similarity or identity thereto.

In other embodiments, the second nucleic acid comprises or consists of the FRS sequence as set forth in SEQ ID NO: 4 or a homologue thereof and of the native promoter of the eriC^{F} gene of P. syringae, and further comprises one or more of the 5′-terminal codons of the eriC^{F} gene, such as one, two, three, four, five, six, seven, eight, nine or ten 5′-terminal codons of the eriC^{F} gene. In a particular embodiment, the second nucleic acid comprises or consists of the FRS sequence as set forth in SEQ ID NO: 4 or a functional variant thereof and of the native promoter of the eriC^{F} gene of P. syringae, and further comprises the eight 5′-terminal codons of the eriC^{F} gene. The corresponding sequence is as set forth in SEQ ID NO: 5. The second nucleic acid may comprise a sequence having at least 70% sequence homology, similarity or identity thereto, such as at least 71%, such as at least 72%, such as at least 73%, such as at least 74%, such as at least 75%, such as at least 76%, such as at least 77%, such as at least 78%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence homology, similarity or identity to SEQ ID NO: 5.

In some embodiments, the second nucleic acid comprises or consists of the FRS sequence as set forth in SEQ ID NO: 4 or a homologue thereof and of the P_{EM7} promoter. The sequence of the corresponding second nucleic acid is set forth in SEQ ID NO: 8. In some embodiments, the second nucleic acid comprises or consists of SEQ ID NO: 8 or a homologue thereof having at least 70% sequence homology, similarity or identity thereto, such as at least 71%, such as at least 72%, such as at least 73%, such as at least 74%, such as at least 75%, such as at least 76%, such as at least 77%, such as at least 78%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence homology, similarity or identity to SEQ ID NO: 8.

In other embodiments, the second nucleic acid comprises or consists of the FRS sequence as set forth in SEQ ID NO: 4 or a homologue thereof and of the P_{EM7} promoter, and further comprises one or more of the 5′-terminal codons of the eriC^{F} gene, such as one, two, three, four, five, six, seven, eight, nine or ten 5′-terminal codons of the eriC^{F} gene. In a particular embodiment, the second nucleic acid comprises the FRS sequence as set forth in SEQ ID NO: 4 or a homologue thereof and of the P_{EM7} promoter, and further comprises the eight 5′-terminal codons of the eriC^{F} gene. The corresponding sequence is set forth in SEQ ID NO: 7. The second nucleic acid may thus have at least 70% sequence homology, similarity or identity to SEQ ID NO: 7, such as at least 71%, such as at least 72%, such as at least 73%, such as at least 74%, such as at least 75%, such as at least 76%, such as at least 77%, such as at least 78%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence homology, similarity or identity to SEQ ID NO: 7.

Accordingly, in some embodiments the second nucleic acid is as set forth in SEQ ID NO: 5, SEQ ID NO: 6, SEQ ID NO: 7 or SEQ ID NO: 8, preferably as set forth in SEQ ID NO: 5, SEQ ID NO: 6 or SEQ ID NO: 8, most preferably as set forth in SEQ ID NO: 5 or SEQ ID NO: 6; or the nucleic acid comprises or consists of a sequence having at least 70% sequence homology, similarity or identity to SEQ ID NO: 5, SEQ ID NO: 6, SEQ ID NO: 7 or SEQ ID NO: 8, such as at least 71%, such as at least 72%, such as at least 73%, such as at least 74%, such as at least 75%, such as at least 76%, such as at least 77%, such as at least 78%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence homology, similarity or identity to SEQ ID NO: 5, SEQ ID NO: 6, SEQ ID NO: 7 or SEQ ID NO: 8, preferably to SEQ ID NO: 5, SEQ ID NO: 6 or SEQ ID NO: 8, most preferably to SEQ ID NO: 5 or SEQ ID NO: 6.

In some embodiments, the second nucleic acid comprises or consists of SEQ ID NO: 5.

Alternatively, the expression of the activator of transcription of the first promoter can be placed under the control of any inducible system known in the art, such as Lacl^{Q}/P_{trc}, Lacl^{Q}/P_{tac}, XylS/Pm, ChnR/P_{chnB}, AlkS/P_{alkB}, CprK/P_{DB3}, AraC/P_{BAD} or RhaRS/P_{rhaBAD}.

Such inducible systems may also be used to control the expression of any of the fluorinase, PNP, nucleosidase, kinase, isomerase, aldolase, Aldh, ADH, AcAldh, F-Aldh, and/or Acs, either independently or in combination, for example control the transcription of one or more promoters that control expression of the fluorinase, PNP, nucleosidase, kinase, isomerase, aldolase, Aldh, ADH, AcAldh, F-Aldh, and/or Acs. Such one or more promoters are described elsewhere herein, for example in “Nucleic acids”.

Phosphorylase and/or PNP

In some embodiments, the cell may further express a phosphorylase, such as a purine nucleoside phosphorylase (PNP), with EC number EC 2.4.2.28 and/or EC 2.4.2.1, and is thus capable of producing a phosphorylated compound from a fluorinated compound, such as producing a phosphorylated and fluorinated compound. In some embodiments, the cell is capable of producing a phosphorylated and fluorinated compound from a substrate in the presence of fluoride. In relation to the phosphorylase and/or PNP, said phosphorylated and fluorinated compound may be any compound obtained from the fluorination and phosphorylation of any of the substrates disclosed in the section “Substrates” herein below. In some embodiments, the fluorinated compound is 5′-FDA and the phosphorylated and fluorinated compound is 5′-FDRP. The phosphorylated and fluorinated compound may also be referred to as a fluorinated and phosphorylated compound, a phosphorylated fluorinated compound or fluorinated phosphorylated compound.

In some embodiments, the cell is capable of producing a fluorinated compound and is further capable of phosphorylating said compound. In some embodiments, the cell is capable of producing 5′-FDA and is further capable of converting said 5′-FDA to 5′-FDRP. In some embodiments, the phosphorylase and/or PNP gene and the fluorinase gene are both controlled by the riboswitch. In other words, the phosphorylase and/or PNP and the fluorinase gene are present within a same nucleic acid molecule (here the first nucleic acid), for example a vector.

In some embodiments, the cell is capable of producing a fluorinated and phosphorylated compound from a substrate in the presence of fluoride, said cell comprising:
a) a first nucleic acid comprising a first promoter, a fluorinase gene encoding a fluorinase (EC 2.5.1.63) capable of catalysing the formation of a C—F bond, and a gene encoding a phosphorylase (EC 2.4.2.28 and/or EC 2.4.2.1) such as an flB1 gene or a deoD gene, said phosphorylase being capable of catalysing the phosphorylation of a fluorinated adenosine, said fluorinase gene and said phosphorylase gene being under control of the first promoter, wherein the first promoter preferably is a T7 promoter; and wherein the fluorinase is the fluorinase of Methanosaeta sp. PtaU1.Bin055 as set forth in SEQ ID NO: 1 (FlA_{PtaU1}), or a functional variant thereof having at least 70% sequence homology, similarity or identity thereto, such as at least 75%, such as at least 80%, such as at least 85%, such as at least 90%, such as at least 95%, such as at least 100% sequence homology, similarity or identity to SEQ ID NO: 1;
b) optionally a second nucleic acid comprising a fluoride-responsive riboswitch such as the FRS riboswitch from P. syringae (SEQ ID NO: 4),
   wherein the cell is capable of expressing the fluorinase and the phosphorylase at least in the presence of fluoride. In some embodiments, the cell is a Pseudomonas putida cell, in particular a P. putida KT2440 cell, which may further comprise a mutation of a gene encoding a fluoride transporter resulting in a partial or total loss of activity of the fluoride transporter.

In specific embodiments, the cell is capable of producing a fluorinated and phosphorylated compound from a substrate in the presence of fluoride, said cell comprising:
a) a first nucleic acid comprising a first promoter, a fluorinase gene encoding a fluorinase (EC 2.5.1.63) capable of catalysing the formation of a C—F bond, and a gene encoding a phosphorylase (EC 2.4.2.28 and/or EC 2.4.2.1) such as an flB1 gene or a deoD gene, said phosphorylase being capable of catalysing the phosphorylation of a fluorinated adenosine, said fluorinase gene and said phosphorylase gene being under control of the first promoter, wherein the first promoter preferably is a T7 promoter; and wherein the fluorinase is the fluorinase of Methanosaeta sp. PtaU1.Bin055 as set forth in SEQ ID NO: 1 (FlA_{PtaU1}), or a functional variant thereof having at least 70% sequence homology, similarity or identity thereto, such as at least 75%, such as at least 80%, such as at least 85%, such as at least 90%, such as at least 95%, such as at least 100% sequence homology, similarity or identity to SEQ ID NO: 1;
b) optionally a second nucleic acid comprising a fluoride-responsive riboswitch such as the FRS riboswitch from P. syringae (SEQ ID NO: 4), a second promoter such as the native promoter of the eriC^{F} gene of P. syringae or the P_{EM7} promoter, and a gene encoding an activator of transcription, said gene being under the control of the second promoter, wherein transcription of the activator of transcription can be induced by an inducer, wherein the activator of transcription preferably is the T7 RNA polymerase, said gene being under the control of the second promoter, wherein transcription of the T7 RNA polymerase can be induced by fluoride, wherein T7 RNA polymerase upon expression activates transcription from the T7 promoter;
   wherein the cell is capable of expressing the activator of transcription, the fluorinase, and the phosphorylase at least in the presence of fluoride. In some embodiments, the cell is a Pseudomonas putida cell, in particular a P. putida KT2440 cell, which may further comprise a mutation of a gene encoding a fluoride transporter resulting in a partial or total loss of activity of the fluoride transporter.

The phosphorylase and/or PNP, may be a heterologous phosphorylase and/or PNP, i.e. a phosphorylase and/or PNP that is not native to or expressed endogenously in the cell. In some embodiments, the phosphorylase and/or PNP is a phosphorylase and/or PNP native to Streptomyces such as Streptomyces sp. MA37, S. xinghaeiensis or S. cattleya, or a PNP native to Escherichia such as E. coli.

In some embodiments, the phosphorylase and/or PNP gene is the flB1 gene from Streptomyces sp. MA37 as set forth in SEQ ID NO: 12 or SEQ ID NO: 13, encoding FlB1 as set forth in SEQ ID NO: 11. In some embodiments, the phosphorylase and/or PNP gene is a homologue of flB1 having at least 70% sequence homology, similarity or identity thereto, such as at least 71%, such as at least 72%, such as at least 73%, such as at least 74%, such as at least 75%, such as at least 76%, such as at least 77%, such as at least 78%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence homology, similarity or identity to SEQ ID NO: 12 and/or SEQ ID NO:13.

In some embodiments, the phosphorylase and/or PNP is a functional variant of FlB1 as set forth in SEQ ID NO: 11, such as a functional variant having at least 70% sequence homology, similarity or identity thereto, such as at least 71%, such as at least 72%, such as at least 73%, such as at least 74%, such as at least 75%, such as at least 76%, such as at least 77%, such as at least 78%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence homology, similarity or identity to SEQ ID NO: 11.

In some embodiments, the phosphorylase and/or PNP gene is the deoD gene from Escherichia coli as set forth in SEQ ID NO: 14, encoding DeoD as set forth in SEQ ID NO: 15. In some embodiments, the phosphorylase and/or PNP gene is a homologue of deoD having at least 70% sequence homology, similarity or identity thereto, such as at least 71%, such as at least 72%, such as at least 73%, such as at least 74%, such as at least 75%, such as at least 76%, such as at least 77%, such as at least 78%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence homology, similarity or identity to SEQ ID NO: 14.

In some embodiments, the phosphorylase and/or PNP is a functional variant of DeoD as set forth in SEQ ID NO: 15, such as a functional variant having at least 70% sequence homology, similarity or identity thereto, such as at least 71%, such as at least 72%, such as at least 73%, such as at least 74%, such as at least 75%, such as at least 76%, such as at least 77%, such as at least 78%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence homology, similarity or identity to SEQ ID NO: 15.

The phosphorylase and/or PNP may be expressed in the cell by introducing a nucleic acid sequence or gene as detailed further below, which encodes a phosphorylase and/or PNP, such as FlB1 or DeoD, for example SEQ ID NO: 12, SEQ ID NO: 13 and/or SEQ ID NO: 14 or a homologue thereof having at least 70% homology, identity or similarity thereto. The phosphorylase and/or PNP gene or nucleic acid sequence, such as the flB1 gene and/or the deoD gene, may be codon-optimised as is known in the art.

In some embodiments, the cell is capable of producing a fluorinated compound, such as 5′-FDA and is further capable of converting said compound into 5′-FDRP. Thus in embodiments where the cell is capable of expressing a phosphorylase and/or PNP as described above, such as FlB1 or DeoD, the cell is capable of converting 5′-FDA to 5′-FDRP in the presence of 5′-FDA. The cell is thus capable of producing 5′-FDRP in the presence of 5′-FDA and/or a fluorinated compound. In a preferred embodiment, the cell expresses FlA_{PtaU1} and DeoD, or a functional variant thereof having at least 70% homology, identity or similarity thereto. In another preferred embodiment, the cell expresses FlA_{PtaU1} (SEQ ID NO: 1) and FlB1 (SEQ ID NO: 11), or a functional variant thereof having at least 70% homology, identity or similarity to SEQ ID NO: 1 and/or SEQ ID NO: 11. In a preferred embodiment, the cell expresses FlA1_{MA37} and DeoD, or a functional variant thereof having at least 70% homology, identity or similarity thereto. In another preferred embodiment, the cell expresses FlA1_{MA37} and FlB1, or a functional variant thereof having at least 70% homology, identity or similarity thereto. In some embodiments, the cell does not express the fluorinase and is provided with 5′-FDA, such as propagated in medium supplied with 5′-FDA.

Functional variants of the phosphorylase and/or PNP may be identified by expressing said variant enzyme in a host cell, purifying the variant enzyme and performing an assay, e.g. an enzyme assay, to measure production of 5′-FDRP, for example measure the conversion of 5′-FDA to 5′-FDRP. 5′-FDA and 5′-FDRP can be measured by LC-MS as described by Calero et al. (2020).

### Nucleosidase

In some embodiments, the cell may further express a nucleosidase (EC 3.2.2.9), and is thus capable of producing a deoxyribose compound from a fluorinated compound. The nucleosidase catalyses the reaction:

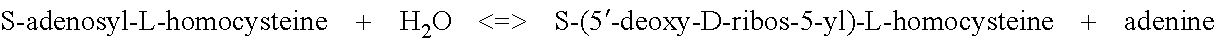

In some embodiments, the cell is capable of producing a 5′-fluorodeoxyribose (5′-FDR) from a substrate in the presence of fluoride. Thus, in this section fluorinated compound may refer to 5′-FDA.

In some embodiments, the cell is capable of producing a fluorinated compound and is further capable of converting said compound into a 5′-FDR. In some embodiments, the nucleosidase gene and the fluorinase gene are both controlled by the riboswitch. In other words, the nucleosidase and the fluorinase gene are present within a same nucleic acid molecule (here the first nucleic acid), for example a vector.

In some embodiments, the cell is capable of producing 5′-FDR in the presence of fluoride, said cell comprising:
a) a first nucleic acid comprising a first promoter, a gene encoding a fluorinase (EC 2.5.1.63) capable of catalysing the formation of a C—F bond, and a gene encoding a nucleosidase (EC 3.2.2.9) such as Pfs, said nucleosidase being capable of catalysing the conversion of 5′-FDA to 5′-FDR, said fluorinase gene and said nucleosidase gene being under control of the first promoter, wherein the first promoter preferably is a T7 promoter; and wherein the fluorinase is the fluorinase of Methanosaeta sp. PtaU1.Bin055 as set forth in SEQ ID NO: 1 (FlA_{PtaU1}), or a functional variant thereof having at least 70% sequence homology, similarity or identity thereto, such as at least 75%, such as at least 80%, such as at least 85%, such as at least 90%, such as at least 95%, such as at least 100% sequence homology, similarity or identity to SEQ ID NO: 1;
b) optionally a second nucleic acid comprising a fluoride-responsive riboswitch such as the FRS riboswitch from P. syringae (SEQ ID NO: 4),
   wherein the cell is capable of expressing the fluorinase and the nucleosidase at least in the presence of fluoride. In some embodiments, the cell is a Pseudomonas putida cell, in particular a P. putida KT2440 cell, which may further comprise a mutation of a gene encoding a fluoride transporter resulting in a partial or total loss of activity of the fluoride transporter.

In specific embodiments, the cell is capable of producing 5′-FDR in the presence of fluoride, said cell comprising:
a) a first nucleic acid comprising a first promoter, a fluorinase gene encoding a fluorinase (EC 2.5.1.63) capable of catalysing the formation of a C—F bond, and a gene encoding a nucleosidase (EC 3.2.2.9) such as Pfs, said nucleosidase being capable of catalysing the conversion of 5′-FDA to 5′-FDR, said fluorinase gene and said nucleosidase gene being under control of the first promoter, wherein the first promoter preferably is a T7 promoter; and wherein the fluorinase is the fluorinase of Methanosaeta sp. PtaU1.Bin055 as set forth in SEQ ID NO: 1 (FlA_{PtaU1}), or a functional variant thereof having at least 70% sequence homology, similarity or identity thereto, such as at least 75%, such as at least 80%, such as at least 85%, such as at least 90%, such as at least 95%, such as at least 100% sequence homology, similarity or identity to SEQ ID NO: 1;
b) optionally a second nucleic acid comprising a fluoride-responsive riboswitch such as the FRS riboswitch from P. syringae (SEQ ID NO: 4), a second promoter such as the native promoter of the eriC^{F} gene of P. syringae or the P_{EM7} promoter, and a gene encoding an activator of transcription, said gene being under the control of the second promoter, wherein transcription of the activator of transcription can be induced by an inducer, wherein the activator of transcription preferably is the T7 RNA polymerase, said gene being under the control of the second promoter, wherein transcription of the T7 RNA polymerase can be induced by fluoride, wherein T7 RNA polymerase upon expression activates transcription from the T7 promoter;
   wherein the cell is capable of expressing the activator of transcription, the fluorinase, and the nucleosidase at least in the presence of fluoride. In some embodiments, the cell is a Pseudomonas putida cell, in particular a P. putida KT2440 cell, which may further comprise a mutation of a gene encoding a fluoride transporter resulting in a partial or total loss of activity of the fluoride transporter.

In some embodiments, the nucleosidase gene is the pfs gene from Escherichia coli as set forth in SEQ ID NO: 16, encoding the Pfs nucleosidase as set forth in SEQ ID NO: 17. In some embodiments, the nucleosidase gene is a homologue of pfs having at least 70% sequence homology, similarity or identity thereto, such as at least 71%, such as at least 72%, such as at least 73%, such as at least 74%, such as at least 75%, such as at least 76%, such as at least 77%, such as at least 78%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence homology, similarity or identity to SEQ ID NO: 16.

In some embodiments, the nucleosidase is a functional variant of Pfs as set forth in SEQ ID NO: 17, such as a functional variant having at least 70% sequence homology, similarity or identity thereto, such as at least 71%, such as at least 72%, such as at least 73%, such as at least 74%, such as at least 75%, such as at least 76%, such as at least 77%, such as at least 78%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence homology, similarity or identity to SEQ ID NO: 17.

The nucleosidase may be expressed in the cell by introducing a nucleic acid sequence or gene as detailed further below, which encodes a nucleosidase, such as Pfs, for example SEQ ID NO: 16 or a homologue thereof having at least 70% homology, identity or similarity thereto. The nucleosidase gene or nucleic acid sequence, such as the pfs gene, may be codon-optimised as is known in the art.

Thus, in embodiments where the cell is capable of expressing a nucleosidase as described above, such as Pfs nucleosidase, the cell is capable of converting 5′-FDA to 5′-FDR. The cell is thus capable of producing 5′-FDR.

### Phosphorylation Bypass

As an alternative or as a supplementary pathway for the conversion of 5′-FDA to 5′-FDRP by a PNP as described herein above, a two-step pathway may be employed. The two-step pathway may also be referred to as phosphorylation bypass or phosphorylation bypass pathway, and it may comprise at least a nucleosidase and a kinase that together catalyse the overall conversion of a fluorinated compound into a phosphorylated and fluorinated compound and/or product, such as the conversion of 5′-FDA into 5′-FDRP. The phosphorylated and fluorinated compound and/or product may also be referred to as a fluorinated and phosphorylated compound and/or product.

### Nucleosidase

The first step of the phosphorylation pathway may be catalysed by a nucleosidase (EC 3.2.2.9), said nucleosidase catalyses the reaction:

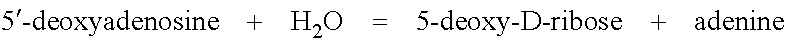

In some embodiments, the cell may further express a nucleosidase, thus being capable of producing a deoxyribose compound from a fluorinated compound. In some other embodiments, the cell is capable of producing a 5′-FDR from a substrate in the presence of fluoride. In some embodiments, the cell is capable of producing a fluorinated compound and is further capable of converting said compound into a 5′-FDR. In this section the fluorinated compound may be 5′-FDA.

Thus, in embodiments where the cell is capable of expressing a nucleosidase as described above, such as Pfs nucleosidase, the cell is capable of converting 5′-FDA to 5′-FDR. The cell is thus capable of producing 5′-FDR.

The nucleosidase may be a heterologous nucleosidase, i.e. a nucleosidase that is not native to or expressed endogenously in the cell. In one embodiment, the nucleosidase is a nucleosidase native to Escherichia such as Escherichia coli. In one embodiment, the nucleosidase is Pfs as set forth in SEQ ID NO: 17, or a functional variant thereof having at least 70% sequence homology, similarity or identity thereto such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology, identity or similarity to SEQ ID NO: 17. In another embodiment, the nucleosidase may be encoded by pfs from Escherichia coli as set forth in SEQ ID NO: 16, or a homologue thereof having at least 70% sequence homology, similarity or identity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology, identity or similarity to SEQ ID NO: 16. The nucleosidase, nucleosidase gene or nucleic acid encoding the nucleic acid is further described elsewhere herein, for example in “Nucleosidase” and “Nucleic acids”.

In one embodiment, the cell expresses FlA_{PtaU1} (SEQ ID NO: 1) and Pfs (SEQ ID NO: 17), or functional variants thereof having at least 70% homology, similarity or identity to SEQ ID NO: 1, and/or SEQ ID NO: 17. In further one embodiment, the cell expresses FlA1_{MA37} (SEQ ID NO: 30) and Pfs (SEQ ID NO: 17), or functional variants thereof having at least 70% homology, similarity or identity to SEQ ID NO: 1, and/or SEQ ID NO: 17.

Functional variants of the nucleosidase may be identified by expressing said variant enzyme in a host cell, purifying the variant enzyme and performing an assay, e.g. an enzyme assay, to measure production of 5′-FDR, for example measure the conversion of 5′-FDA to 5′-FDR. 5′-FDR can be detected and quantified by ¹⁹F-NMR or LC-MS.

### Kinase

The second step of the phosphorylation pathway may be catalysed by a kinase (EC 2.7.1.100). Thus, disclosed herein are kinases capable of catalysing the phosphorylation of a fluorinated compound to fluorinated and phosphorylated compound, such as 5′-FDRP.

Thus, in some embodiments, the fluorinated compound is 5′-FDR and the kinase is capable of converting said 5′-FDR to 5′-FDRP, thus when expressed in a cell said kinase allows the cell to produce 5′-FDRP in the presence of 5′-FDR. Indeed, in this section in relation to the kinase the fluorinated compound may be 5′-FDR.

In some embodiments, the cell may further express a kinase, thus being capable of producing a fluorinated and phosphorylated compound and/or product from a fluorinated compound. In some other embodiments, the cell is capable of producing a 5′-FDRP from a fluorinated compound. In some embodiments, the cell is capable of producing a fluorinated compound and is further capable of converting said compound into a 5′-FDRP.

The kinase may be a heterologous kinase, i.e. a kinase that is not native to or expressed endogenously in the cell. In one embodiment, the kinase is a kinase native to Bacillus such as Bacillus thuringiensis.

In one embodiment, the kinase is KinBt, also known as DrdK, as set forth in SEQ ID NO: 33, or a functional variant thereof having at least 70% homology, identity or similarity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology, identity or similarity to SEQ ID NO: 33.

The kinase may be expressed in the cell by introducing a nucleic acid sequence or gene as detailed further below, which encodes a kinase, such as KinBt, for example SEQ ID NO: 34 or a homologue thereof having at least 70% homology, identity or similarity thereto. The kinase gene or nucleic acid sequence, such as kinBt (drdK), may be codon-optimised as is known in the art.

In some embodiments, the cell is capable of producing a fluorinated compound, such as 5′-FDR, and is further capable of phosphorylating said compound, for example converting said 5′-FDR into 5′-FDRP. In one embodiment, the cell expresses Pfs (SEQ ID NO: 17) and KinBt (SEQ ID NO: 33), or functional variants thereof having at least 70% homology, similarity or identity thereto, such as at least 75%, such as at least 80%, such as at least 85%, such as at least 90%, such as at least 95%, such as at least 100% sequence homology, similarity or identity to SEQ ID NO: 17 and/or SEQ ID NO: 33. In a preferred embodiment, the cell expresses FlA_{PtaU1} (SEQ ID NO: 1), Pfs (SEQ ID NO: 17), and KinBt (SEQ ID NO: 33), or functional variants thereof having at least 70% homology, similarity or identity thereto, such as at least 75%, such as at least 80%, such as at least 85%, such as at least 90%, such as at least 95%, such as at least 100% sequence homology, similarity or identity to SEQ ID NO: 1, SEQ ID NO: 17 and/or SEQ ID NO: 33. In another preferred embodiment, the cell expresses FlA1_{MA37}, Pfs, and KinBt, or functional variants thereof having at least 70% homology, similarity or identity thereto. In some embodiments, the cell does not express the fluorinase and is provided with 5′-FDA, such as propagated in medium supplied with 5′-FDA.

Functional variants of the kinase may be identified by expressing said variant enzyme in a host cell, purifying the variant enzyme and performing an assay, e.g. an enzyme assay, to measure production of 5′-FDRP, for example measure the conversion of 5′-FDA to 5′-FDR. 5′-FDR can be detected and quantified by ¹⁹F-NMR or LC-MS.

### Isomerase

In some embodiments, the cell is capable of producing a fluorinated compound, such as 5′-FDRP as disclosed herein above, and is further capable of converting said compound into 5′-FDRuIP.

Disclosed herein are isomerases capable of catalysing the isomerisation of a fluorinated compound to an isomer of said fluorinated compound. In some embodiments, the cell may further express an isomerase, thus being capable of producing 5′-FDRuIP from a fluorinated compound. In some embodiments, the fluorinated compound is 5′-FDRP and the isomerase is capable of converting said 5′-FDRP to (3R,4S)-5′-fluoro-5′-deoxy-D-ribulose-1-phosphate (5′-FDRuIP), thus when expressed in a cell said isomerase allows the cell to produce 5′-FDRuIP in the presence of 5′-FDRP. Thus, in this section and in relation to the isomerase the fluorinated compound may be 5′-FDRP.

The isomerase may be a heterologous isomerase, i.e. an isomerase that is not native to or expressed endogenously in the cell. In some embodiments, the isomerase is an isomerase native to Bacillus such as B. thuringiensis, or an isomerase native to Streptomyces such as S. cattleya.

In one embodiment, the isomerase is IsoBt, also known as Drdl, as set forth in SEQ ID NO: 35, or a functional variant thereof having at least 70% homology, identity or similarity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology, identity or similarity to SEQ ID NO: 35.

In another embodiment, the isomerase is IsoSc as set forth in SEQ ID NO: 37, or a functional variant thereof having at least 70% homology, identity or similarity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology, identity or similarity to SEQ ID NO: 37.

The isomerase may be expressed in the cell by introducing a nucleic acid sequence or gene as detailed further below, which encodes an isomerase, such as IsoBt and/or IsoSc, for example SEQ ID NO: 36 and/or SEQ ID NO: 38 or a homologue thereof having at least 70% homology, identity or similarity thereto. The isomerase gene or nucleic acid sequence, such as isoBt (drdI) and/or isoSc, may be codon-optimised as is known in the art.

In some embodiments, the cell is capable of producing a fluorinated compound, such as 5′-FDRP and is further capable of converting said compound into 5′-FDRuIP. In a preferred embodiment, the cell expresses FlA_{PtaU1}, DeoD and IsoBt, or functional variants thereof having at least 70% homology, similarity or identity thereto. In another preferred embodiment, the cell expresses FlA1_{MA37}, DeoD, and IsoBt, or functional variants thereof having at least 70% homology, similarity or identity thereto. In a preferred embodiment, the cell expresses FlA_{PtaU1}, DeoD, and IsoSc, or functional variants thereof having at least 70% homology, similarity or identity thereto. In another preferred embodiment, the cell expresses FlA1_{MA37}, DeoD, and IsoSc, or functional variants thereof having at least 70% homology, similarity or identity thereto. In a preferred embodiment, the cell expresses FlA_{PtaU1}, FlB1, and IsoBt, or functional variants thereof having at least 70% homology, similarity or identity thereto. In another preferred embodiment, the cell expresses FlA1_{MA37}, FlB1, and IsoBt, or functional variants thereof having at least 70% homology, similarity or identity thereto. In a preferred embodiment, the cell expresses FlA_{PtaU1} (SEQ ID NO: 1), FlB1 (SEQ ID NO: 11), and IsoSc (SEQ ID NO: 37), or functional variants thereof having at least 70% homology, similarity or identity thereto, such as at least 75%, such as at least 80%, such as at least 85%, such as at least 90%, such as at least 95%, such as at least 100% sequence homology, similarity or identity to SEQ ID NO: 1, SEQ ID NO: 11 and/or SEQ ID NO: 37. In another preferred embodiment, the cell expresses FlA1_{MA37}, FlB1, and IsoSc, or functional variants thereof having at least 70% homology, similarity or identity thereto. In a preferred embodiment, the cell expresses FlA_{PtaU1}, Pfs, KinBt, and IsoBt, or functional variants thereof having at least 70% homology, similarity or identity thereto. In another preferred embodiment, the cell expresses FlA1_{MA37}, Pfs, KinBt, and IsoBt, or functional variants thereof having at least 70% homology, similarity or identity thereto. In a preferred embodiment, the cell expresses FlA_{PtaU1} (SEQ ID NO: 1), Pfs (SEQ ID NO: 17), KinBt (SEQ ID NO: 33), and IsoSc (SEQ ID NO: 37), or functional variants thereof having at least 70% homology, similarity or identity thereto, such as at least 75%, such as at least 80%, such as at least 85%, such as at least 90%, such as at least 95%, such as at least 100% sequence homology, similarity or identity to SEQ ID NO: 1, SEQ ID NO: 17, SEQ ID NO: 33 and/or SEQ ID NO: 37. In another preferred embodiment, the cell expresses FlA1_{MA37}, Pfs, KinBt, and IsoSc, or functional variants thereof having at least 70% homology, similarity or identity thereto. In some embodiments, the cell does not express the fluorinase and is provided with 5′-FDA, such as propagated in medium supplied with 5′-FDA.

In another embodiment, the cell expresses Pfs (SEQ ID NO: 17), KinBt (SEQ ID NO: 33), and IsoSc (SEQ ID NO: 37), or functional variants thereof having at least 70% homology, similarity or identity thereto, such as at least 75%, such as at least 80%, such as at least 85%, such as at least 90%, such as at least 95%, such as at least 100% sequence homology, similarity or identity to SEQ ID NO: 17, SEQ ID NO: 33 and/or SEQ ID NO: 37. In a preferred embodiment, the cell expresses FlB1 (SEQ ID NO: 11), and IsoSc (SEQ ID NO: 37), or functional variants thereof having at least 70% homology, similarity or identity thereto, such as at least 75%, such as at least 80%, such as at least 85%, such as at least 90%, such as at least 95%, such as at least 100% sequence homology, similarity or identity to SEQ ID NO: 11 and/or SEQ ID NO: 37.

Functional variants of the isomerase may be identified by expressing said variant enzyme in a host cell, purifying the variant enzyme and performing an assay, e.g. an enzyme assay, to measure the isomerisation of 5′-FDRP to 5′-FDRuIP, for example as described in “Example 1—FAld, FAc and FAcCoA production” herein. 5′-FDRuIP can detected and quantified by ¹⁹F-NMR or LC-MS as described by Calero et al. (2020).

### Aldolase

In some embodiments, the cell is capable of producing a fluorinated compound, such as 5′-FDRuIP as disclosed herein above, and is further capable of converting said fluorinated compound into FAld.

With respect to the fluorinated compound and the aldolases disclosed herein, and without being bound by theory, said fluorinated compound may be any fluorinated compound that can be converted into another fluorinated compound by the aldolase. In particular, said fluorinated compound may be 5′-FDRuIP. Disclosed herein are aldolases capable of catalysing the conversion of 5′-FDRuIP to FAld. In some embodiments, the cell may further express an aldolase, thus being capable of producing FAld from a fluorinated compound. Thus, in some embodiments, the fluorinated compound is 5′-FDRuIP and the aldolase is capable of converting 5′-FDRuIP to FAld, thus when expressed in a cell said aldolase allows the cell to produce FAld in the presence of 5′-FDRuIP.

The aldolase may be a heterologous aldolase, i.e. an aldolase that is not native to or expressed endogenously in the cell. In some embodiments, the aldolase is an aldolase native to Bacillus such as B. thuringiensis, or an aldolase native to Streptomyces such as S. coelicolor. In one embodiment, the aldolase is AldBt, also known as DrdA, as set forth in SEQ ID NO: 29, or a functional variant thereof having at least 70% homology, identity or similarity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology, identity or similarity to SEQ ID NO: 29.

In another embodiment, the aldolase is AldSc as set forth in SEQ ID NO: 19, or a functional variant thereof having at least 70% homology, identity or similarity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology, identity or similarity to SEQ ID NO: 19.

The aldolase may be expressed in the cell by introducing a nucleic acid sequence or gene as detailed further below, which encodes an aldolase, such as AldBt and/or AldSc, for example SEQ ID NO: 18 and/or SEQ ID NO: 20 or a homologue thereof having at least 70% homology, identity or similarity thereto. The aldolase gene or nucleic acid sequence, such as aldBt (drdA) and/or aldSc, may be codon-optimised as is known in the art.

In some embodiments, the cell is capable of producing a fluorinated compound, such as 5′-FDRuIP and is further capable of converting said compound into FAld. In a preferred embodiment, the cell expresses FlA_{PtaU1}, DeoD, IsoBt, AldBt, or functional variants thereof having at least 70% homology, similarity or identity thereto. In another preferred embodiment, the cell expresses FlA_{PtaU1}, DeoD, IsoBt, AldSc, or functional variants thereof having at least 70% homology, similarity or identity thereto. In a preferred embodiment, the cell expresses FlA_{PtaU1}, DeoD, IsoSc, AldBt, or functional variants thereof having at least 70% homology, similarity or identity thereto. In another preferred embodiment, the cell expresses FlA_{PtaU1}, DeoD, IsoSc, AldSc, or functional variants thereof having at least 70% homology, similarity or identity thereto. In a preferred embodiment, the cell expresses FlA_{PtaU1}, FlB1, IsoBt, AldBt, or functional variants thereof having at least 70% homology, similarity or identity thereto. In another preferred embodiment, the cell expresses FlA_{PtaU1}, FlB1, IsoBt, AldSc, or functional variants thereof having at least 70% homology, similarity or identity thereto. In a preferred embodiment, the cell expresses FlA_{PtaU1}, FlB1, IsoSc, AldBt, or functional variants thereof having at least 70% homology, similarity or identity thereto. In another preferred embodiment, the cell expresses FlA_{PtaU1} (SEQ ID NO: 1), FlB1 (SEQ ID NO: 11), IsoSc (SEQ ID NO: 37), AldSc (SEQ ID NO: 19), or functional variants thereof having at least 70% homology, similarity or identity thereto, such as at least 75%, such as at least 80%, such as at least 85%, such as at least 90%, such as at least 95%, such as at least 100% sequence homology, similarity or identity to SEQ ID NO: 1, SEQ ID NO: 11, SEQ ID NO: 37 and/or SEQ ID NO: 19. In a preferred embodiment, the cell expresses FlA_{PtaU1}, Pfs, KinBt, IsoBt, AldBt, or functional variants thereof having at least 70% homology, similarity or identity thereto. In another preferred embodiment, the cell expresses FlA_{PtaU1}, Pfs, KinBt, IsoBt, AldSc, or functional variants thereof having at least 70% homology, similarity or identity thereto. In a preferred embodiment, the cell expresses FlA_{PtaU1}, Pfs, KinBt, IsoSc, AldBt, or functional variants thereof having at least 70% homology, similarity or identity thereto. In another preferred embodiment, the cell expresses FlA_{PtaU1} (SEQ ID NO: 1), Pfs (SEQ ID NO: 17), KinBt (SEQ ID NO: 33), IsoSc (SEQ ID NO: 37), AldSc (SEQ ID NO: 19), or functional variants thereof having at least 70% homology, similarity or identity thereto, such as at least 75%, such as at least 80%, such as at least 85%, such as at least 90%, such as at least 95%, such as at least 100% sequence homology, similarity or identity to SEQ ID NO: 1, SEQ ID NO: 17, SEQ ID NO: 33, SEQ ID NO: 37 and/or SEQ ID NO: 19. In a preferred embodiment, the cell expresses FlA1_{MA37}, DeoD, IsoBt, AldBt, or functional variants thereof having at least 70% homology, similarity or identity thereto. In another preferred embodiment, the cell expresses FlA1_{MA37}, DeoD, IsoBt, AldSc, or functional variants thereof having at least 70% homology, similarity or identity thereto. In a preferred embodiment, the cell expresses FlA1_{MA37}, DeoD, IsoSc, AldBt, or functional variants thereof having at least 70% homology, similarity or identity thereto. In another preferred embodiment, the cell expresses FlA1_{MA37}, DeoD, IsoSc, AldSc, or functional variants thereof having at least 70% homology, similarity or identity thereto. In a preferred embodiment, the cell expresses FlA1_{MA37}, FlB1, IsoBt, AldBt, or functional variants thereof having at least 70% homology, similarity or identity thereto. In another preferred embodiment, the cell expresses FlA1_{MA37}, FlB1, IsoBt, AldSc, or functional variants thereof having at least 70% homology, similarity or identity thereto. In a preferred embodiment, the cell expresses FlA1_{MA37}, FlB1, IsoSc, AldBt, or functional variants thereof having at least 70% homology, similarity or identity thereto. In another preferred embodiment, the cell expresses FlA1_{MA37}, FlB1, IsoSc, AldSc, or functional variants thereof having at least 70% homology, similarity or identity thereto. In a preferred embodiment, the cell expresses FlA1_{MA37}, Pfs, KinBt, IsoBt, AldBt, or functional variants thereof having at least 70% homology, similarity or identity thereto. In another preferred embodiment, the cell expresses FlA1_{MA37}, Pfs, KinBt, IsoBt, AldSc, or functional variants thereof having at least 70% homology, similarity or identity thereto. In a preferred embodiment, the cell expresses FlA1_{MA37}, Pfs, KinBt, IsoSc, AldBt, or functional variants thereof having at least 70% homology, similarity or identity thereto. In another preferred embodiment, the cell expresses FlA1_{MA37}, Pfs, KinBt, IsoSc, AldSc, or functional variants thereof having at least 70% homology, similarity or identity thereto. In some embodiments, the cell does not express the fluorinase and is provided with 5′-FDA, such as propagated in medium supplied with 5′-FDA.

In another embodiment, the cell expresses Pfs (SEQ ID NO: 17), KinBt (SEQ ID NO: 33), IsoSc (SEQ ID NO: 37) and AldSc (SEQ ID NO: 19), or functional variants thereof having at least 70% homology, similarity or identity thereto, such as at least 75%, such as at least 80%, such as at least 85%, such as at least 90%, such as at least 95%, such as at least 100% sequence homology, similarity or identity to SEQ ID NO: 17, SEQ ID NO: 33, SEQ ID NO: 37 and/or SEQ ID NO: 19. In another further embodiment, the cell expresses FlB1 (SEQ ID NO: 11), IsoSc (SEQ ID NO: 37), AldSc (SEQ ID NO: 19), or functional variants thereof having at least 70% homology, similarity or identity thereto, such as at least 75%, such as at least 80%, such as at least 85%, such as at least 90%, such as at least 95%, such as at least 100% sequence homology, similarity or identity to SEQ ID NO: 11, SEQ ID NO: 37 and/or SEQ ID NO: 19.

In other embodiments, the cell expresses IsoSc (SEQ ID NO: 37) and AldSc (SEQ ID NO: 19), or a functional variant thereof having at least 70% homology, similarity or identity thereto, such as at least 75%, such as at least 80%, such as at least 85%, such as at least 90%, such as at least 95%, such as at least 100% sequence homology, similarity or identity to SEQ ID NO: 37 and/or SEQ ID NO: 19, respectively.

In further other embodiments, the cell expresses IsoBt (SEQ ID NO: 35) and AldSc (SEQ ID NO: 19), or a functional variant thereof having at least 70% homology, similarity or identity thereto, such as at least 75%, such as at least 80%, such as at least 85%, such as at least 90%, such as at least 95%, such as at least 100% sequence homology, similarity or identity to SEQ ID NO: 35 and/or SEQ ID NO: 19, respectively.

In some other embodiments, the cell expresses IsoBt (SEQ ID NO: 35) and AldBt (SEQ ID NO: 29), or a functional variant thereof having at least 70% homology, similarity or identity thereto, such as at least 75%, such as at least 80%, such as at least 85%, such as at least 90%, such as at least 95%, such as at least 100% sequence homology, similarity or identity to SEQ ID NO: 35 and/or SEQ ID NO: 29, respectively.

In further other embodiments, the cell expresses IsoSc (SEQ ID NO: 37) and AldBt (SEQ ID NO: 29), or a functional variant thereof having at least 70% homology, similarity or identity thereto, such as at least 75%, such as at least 80%, such as at least 85%, such as at least 90%, such as at least 95%, such as at least 100% sequence homology, similarity or identity to SEQ ID NO: 37 and/or SEQ ID NO: 29, respectively.

Functional variants of aldolase may be identified by expressing said variant enzyme in a host cell, purifying the variant enzyme and performing an assay, e.g. an enzyme assay, to measure the conversion of 5′-FDRuIP to FAld, for example as described in “Example 1—FAld, FAc and FAcCoA production” herein. FAld can be detected and quantified by ¹⁹F-NMR, either in the free form or in conjugates with other molecules.

### Alcohol Dehydrogenase

In some embodiments, the cell is capable of producing a fluorinated compound, such as FAld as disclosed herein above, and is further capable of converting said compound into FEtOH.

Disclosed herein are alcohol dehydrogenases (ADHs) capable of catalysing the conversion of FAld to FEtOH. In some embodiments, the cell may further express an ADHs, thus being capable of producing FEtOH from a fluorinated compound. In this section and without being bound by theory, said fluorinated compound may be FAld, FAc and/or FAcCoA. Thus, in some embodiments, the fluorinated compound is FAld and the ADH is capable of converting FAld to FEtOH, thus when expressed in a cell said ADH allows the cell to produce FEtOH in the presence of FAld. The ADH may be an NAD(P)-dependent alcohol dehydrogenase with EC number EC 1.1.1.1 capable of converting acetaldehyde to ethanol using NADH, such as FAld to FEtOH using NADH.

The ADH may be an endogenous, i.e. native ADH, or a heterologous ADH, i.e. an ADH that is not native to or expressed endogenously in the cell. In some embodiments, the ADH is an ADH native to Pseudomonas such as P. putida, for example P. putida KT2440. In other embodiments, the ADH is an ADH native to Escherichia, such as E. coli, for example E. coli MG1655.

Without being bound by theory, the ADH may be a P. putida ADH, such as an ADH encoded by and/or comprised within the locus tag PP_4760 (RefSeq Accession NP_746866.1), PP_1816 (RefSeq Accession NP_743971.1), PP_2827 (RefSeq Accession NP_744971.1), PP_2962 (RefSeq Accession NP_745106.1), PP_1720 (RefSeq Accession NP_743877.1), PP_5210 (RefSeq Accession NP_747311.1), PP_2049 (RefSeq Accession NP_744199.1), PP_2953 (RefSeq Accession NP_745097.1), or PP_2988 (RefSeq Accession NP_745132.1), or a functional variant thereof having at least 70% homology, identity or similarity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology, identity or similarity thereto.

In some embodiments, and without being bound by theory, the ADH is AdhP (locus tag PP_3839, RefSeq Accession NP_745969.1) from P. putida and/or YqhD (locus tag PP_2492, RefSeq Accession NP_744640.1) from P. putida, or a functional variant thereof having at least 70% homology, identity or similarity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology, identity or similarity thereto.

In some embodiments, the cell is capable of producing a fluorinated compound, such as FAld, and is further capable of converting said compound into FEtOH. In a preferred embodiment, the cell expresses FlA_{PtaU1}, DeoD, IsoBt, AldBt, and an ADH, or a functional variant thereof having at least 70% homology, identity or similarity thereto. In another preferred embodiment, the cell expresses FlA_{PtaU1}, DeoD, IsoBt, AldSc, and an ADH, or a functional variant thereof having at least 70% homology, identity or similarity thereto. In a preferred embodiment, the cell expresses FlA_{PtaU1}, DeoD, IsoSc, AldBt, and an ADH, or a functional variant thereof having at least 70% homology, identity or similarity thereto. In another preferred embodiment, the cell expresses FlA_{PtaU1}, DeoD, IsoSc, AldSc, and an ADH, or a functional variant thereof having at least 70% homology, identity or similarity thereto. In a preferred embodiment, the cell expresses FlA_{PtaU1}, FlB1, IsoBt, AldBt, and an ADH, or a functional variant thereof having at least 70% homology, identity or similarity thereto. In another preferred embodiment, the cell expresses FlA_{PtaU1}, FlB1, IsoBt, AldSc, and an ADH, or a functional variant thereof having at least 70% homology, identity or similarity thereto. In a preferred embodiment, the cell expresses FlA_{PtaU1}, FlB1, IsoSc, AldBt, and an ADH, or a functional variant thereof having at least 70% homology, identity or similarity thereto. In another preferred embodiment, the cell expresses FlA_{PtaU1}, FlB1, IsoSc, AldSc, and an ADH, or a functional variant thereof having at least 70% homology, identity or similarity thereto. In a preferred embodiment, the cell expresses FlA_{PtaU1}, Pfs, KinBt, IsoBt, AldBt, and an ADH, or a functional variant thereof having at least 70% homology, identity or similarity thereto. In another preferred embodiment, the cell expresses FlA_{PtaU1}, Pfs, KinBt, IsoBt, AldSc, and an ADH, or a functional variant thereof having at least 70% homology, identity or similarity thereto. In a preferred embodiment, the cell expresses FlA_{PtaU1}, Pfs, KinBt, IsoSc, AldBt, and an ADH, or a functional variant thereof having at least 70% homology, identity or similarity thereto. In another preferred embodiment, the cell expresses FlA_{PtaU1}, Pfs, KinBt, IsoSc, AldSc, and an ADH, or a functional variant thereof having at least 70% homology, identity or similarity thereto. In a preferred embodiment, the cell expresses FlA1_{MA37}, DeoD, IsoBt, AldBt, and an ADH, or a functional variant thereof having at least 70% homology, identity or similarity thereto. In another preferred embodiment, the cell expresses FlA1_{MA37}, DeoD, IsoBt, AldSc, and an ADH, or a functional variant thereof having at least 70% homology, identity or similarity thereto. In a preferred embodiment, the cell expresses FlA1_{MA37}, DeoD, IsoSc, AldBt, and an ADH, or a functional variant thereof having at least 70% homology, identity or similarity thereto. In another preferred embodiment, the cell expresses FlA1_{MA37}, DeoD, IsoSc, AldSc, and an ADH, or a functional variant thereof having at least 70% homology, identity or similarity thereto. In a preferred embodiment, the cell expresses FlA1_{MA37}, FlB1, IsoBt, AldBt, and an ADH, or a functional variant thereof having at least 70% homology, identity or similarity thereto. In another preferred embodiment, the cell expresses FlA1_{MA37}, FlB1, IsoBt, AldSc, and an ADH, or a functional variant thereof having at least 70% homology, identity or similarity thereto. In a preferred embodiment, the cell expresses FlA1_{MA37}, FlB1, IsoSc, AldBt, and an ADH, or a functional variant thereof having at least 70% homology, identity or similarity thereto. In another preferred embodiment, the cell expresses FlA1_{MA37}, FlB1, IsoSc, AldSc, and an ADH, or a functional variant thereof having at least 70% homology, identity or similarity thereto. In a preferred embodiment, the cell expresses FlA1_{MA37}, Pfs, KinBt, IsoBt, AldBt, and an ADH, or a functional variant thereof having at least 70% homology, identity or similarity thereto. In another preferred embodiment, the cell expresses FlA1_{MA37}, Pfs, KinBt, IsoBt, AldSc, and an ADH, or a functional variant thereof having at least 70% homology, identity or similarity thereto. In a preferred embodiment, the cell expresses FlA1_{MA37}, Pfs, KinBt, IsoSc, AldBt, and an ADH, or a functional variant thereof having at least 70% homology, identity or similarity thereto. In another preferred embodiment, the cell expresses FlA1_{MA37}, Pfs, KinBt, IsoSc, AldSc, and an ADH, or a functional variant thereof having at least 70% homology, identity or similarity thereto. Said AHD may be encoded by and/or comprised by PP_4760, PP_1816, PP_2827, PP_2962, PP_1720, PP_5210, PP_2049, PP_2953, PP_2988, AdhP and YqhD as described herein above. In some embodiments, the cell does not express the fluorinase and is provided with 5′-FDA, such as propagated in medium supplied with 5′-FDA.

Functional variants of the ADH may be identified by expressing said variant enzyme in a host cell, purifying the variant enzyme and performing an assay, e.g. an enzyme assay, to measure the conversion of a fluorinated compound, for example FAld, to FEtOH, for example as described in “Example 1—FAld, FAc and FAcCoA production” herein. FEtOH can be detected and measured by ¹⁹F-NMR and LC-MS.

### Acetaldehyde Dehydrogenase

In some embodiments, the cell is capable of producing a fluorinated compound, such as FAld as disclosed herein above, and is further capable of converting said compound into FAc and/or FAcCoA. In this section, said fluorinated compound may be FAld and/or FAc. An Aldh may also have Acs activity and Acs activity is described further in the section “Acetyl-CoA synthetase” herein below.

Disclosed herein are acetaldehyde dehydrogenases (Aldhs) capable of catalysing the conversion of FAld to FAc and/or FAcCoA. In some embodiments, the cell may further express an Aldh, thus being capable of producing FAc and/or FAcCoA from a fluorinated compound. Thus, in some embodiments, the fluorinated compound is FAld and the Aldh is capable of converting FAld to FAc and/or FAcCoA, thus when expressed in a cell said Aldh allows the cell to produce FAc and/or FAcCoA in the presence of FAld.

In some embodiments, said Aldh being capable of catalysing the conversion of FAld to FAc and/or FAcCoA, is an aldehyde dehydrogenase (NAD⁺) with EC 1.2.1.3. Aldehyde dehydrogenase (NAD⁺) (EC 1.2.1.3) catalyses the reaction:

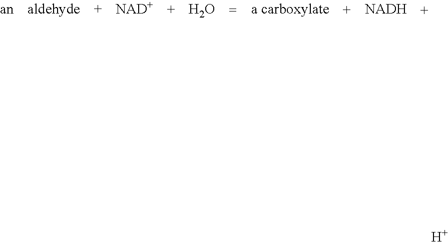

In some other embodiments, said Aldh being capable of catalysing the conversion of FAld to FAc and/or FAcCoA, is a fluoroacetaldehyde dehydrogenase (F-Aldh) with EC 1.2.1.69. In some other embodiments, said Aldh being capable of catalysing the conversion of FAld to FAc and/or FAcCoA, is an acetylating acetaldehyde dehydrogenase (AcAldh) with EC 1.2.1.10. In further some embodiments, the F-Aldh (EC 1.2.1.69) and the AcAldh (EC 1.2.1.10) are identical. In other words, in some embodiments the F-Aldh and the AcAldh may be an Aldh capable of catalysing the conversion of FAld to FAc and FAcCoA. F-Aldh and/or AcAldh may also be referred to as Aldh herein.

The AcAldh may be an endogenous, i.e. native Aldh, or a heterologous Aldh, i.e. an Aldh that is not native to or expressed endogenously in the cell. In some embodiments, the Aldh, F-Aldh and/or AcAldh is an Aldh, F-Aldh and/or AcAldh native to Pseudomonas such as P. aeruginosa, preferably P. aeruginosa 1984 or P. aeruginosa 4022, or an Aldh, F-Aldh and/or AcAldh native to Escherichia such as E. coli, or an Aldh, F-Aldh and/or AcAldh native to Moorella thermoacetica such as M. thermoacetica.

### Fluoroacetaldehyde Dehydrogenase

In some embodiments, the cell is capable of producing a fluorinated compound, such as FAld as disclosed herein above, and is further capable of converting said compound into FAc.

Disclosed herein are fluoroacetaldehyde dehydrogenases (F-Aldhs) capable of catalysing the conversion of FAld to FAc. In some embodiments, the cell may further express an F-Aldh, thus being capable of producing FAc from a fluorinated compound. In some embodiments, the fluorinated compound is FAld and the F-Aldh is capable of converting said FAld to FAc, thus when expressed in a cell said F-Aldh allows the cell to produce FAc in the presence of FAld. As explained above, an Aldh may be an F-Aldh and/or an AcAldh, and therefore an F-Aldh may be an Aldh and/or an AcAldh.

The F-Aldh may be an endogenous, i.e. a native Aldh, or a heterologous Aldh, i.e. an Aldh that is not native to or expressed endogenously in the cell. In some embodiments, the F-Aldh is an F-Aldh native to Pseudomonas such as P. putida, for example P. putida KT2440. In other embodiments, the F-Aldh is an F-Aldh or Aldh native to Escherichia, such as E. coli, for example E. coli MG1655.

In a preferred embodiment, the cell expresses FlA_{PtaU1}, DeoD, IsoBt, AldBt, and an F-Aldh, or functional variants thereof having at least 70% homology, similarity or identity thereto. In another preferred embodiment, the cell expresses FlA_{PtaU1}, DeoD, IsoBt, AldSc, and an F-Aldh, or functional variants thereof having at least 70% homology, similarity or identity thereto. In a preferred embodiment, the cell expresses FlA_{PtaU1}, DeoD, IsoSc, AldBt, and an F-Aldh, or functional variants thereof having at least 70% homology, similarity or identity thereto. In another preferred embodiment, the cell expresses FlA_{PtaU1}, DeoD, IsoSc, AldSc, and an F-Aldh, or functional variants thereof having at least 70% homology, similarity or identity thereto. In a preferred embodiment, the cell expresses FlA_{PtaU1}, FlB1, IsoBt, AldBt, and an F-Aldh, or functional variants thereof having at least 70% homology, similarity or identity thereto. In another preferred embodiment, the cell expresses FlA_{PtaU1}, FlB1, IsoBt, AldSc, and an F-Aldh, or functional variants thereof having at least 70% homology, similarity or identity thereto. In a preferred embodiment, the cell expresses FlA_{PtaU1}, FlB1, IsoSc, AldBt, and an F-Aldh, or functional variants thereof having at least 70% homology, similarity or identity thereto. In another preferred embodiment, the cell expresses FlA_{PtaU1}, FlB1, IsoSc, AldSc, and an F-Aldh, or functional variants thereof having at least 70% homology, similarity or identity thereto. In a preferred embodiment, the cell expresses FlA_{PtaU1}, Pfs, KinBt, IsoBt, AldBt, and an F-Aldh, or functional variants thereof having at least 70% homology, similarity or identity thereto. In another preferred embodiment, the cell expresses FlA_{PtaU1}, Pfs, KinBt, IsoBt, AldSc, and an F-Aldh, or functional variants thereof having at least 70% homology, similarity or identity thereto. In a preferred embodiment, the cell expresses FlA_{PtaU1}, Pfs, KinBt, IsoSc, AldBt, and an F-Aldh, or functional variants thereof having at least 70% homology, similarity or identity thereto. In another preferred embodiment, the cell expresses FlA_{PtaU1}, Pfs, KinBt, IsoSc, AldSc, and an F-Aldh, or functional variants thereof having at least 70% homology, similarity or identity thereto. In a preferred embodiment, the cell expresses FlA1_{MA37}, DeoD, IsoBt, AldBt, and an F-Aldh, or functional variants thereof having at least 70% homology, similarity or identity thereto. In another preferred embodiment, the cell expresses FlA1_{MA37}, DeoD, IsoBt, AldSc, and an F-Aldh, or functional variants thereof having at least 70% homology, similarity or identity thereto. In a preferred embodiment, the cell expresses FlA1_{MA37}, DeoD, IsoSc, AldBt, and an F-Aldh, or functional variants thereof having at least 70% homology, similarity or identity thereto. In another preferred embodiment, the cell expresses FlA1_{MA37}, DeoD, IsoSc, AldSc, and an F-Aldh, or functional variants thereof having at least 70% homology, similarity or identity thereto. In a preferred embodiment, the cell expresses FlA1_{MA37}, FlB1, IsoBt, AldBt, and an F-Aldh, or functional variants thereof having at least 70% homology, similarity or identity thereto. In another preferred embodiment, the cell expresses FlA1_{MA37}, FlB1, IsoBt, AldSc, and an F-Aldh, or functional variants thereof having at least 70% homology, similarity or identity thereto. In a preferred embodiment, the cell expresses FlA1_{MA37}, FlB1, IsoSc, AldBt, and an F-Aldh, or functional variants thereof having at least 70% homology, similarity or identity thereto. In another preferred embodiment, the cell expresses FlA1_{MA37}, FlB1, IsoSc, AldSc, and an F-Aldh, or functional variants thereof having at least 70% homology, similarity or identity thereto. In a preferred embodiment, the cell expresses FlA1_{MA37}, Pfs, KinBt, IsoBt, AldBt, and an F-Aldh, or functional variants thereof having at least 70% homology, similarity or identity thereto. In another preferred embodiment, the cell expresses FlA1_{MA37}, Pfs, KinBt, IsoBt, AldSc, and an F-Aldh, or functional variants thereof having at least 70% homology, similarity or identity thereto. In a preferred embodiment, the cell expresses FlA1_{MA37}, Pfs, KinBt, IsoSc, AldBt, and an F-Aldh, or functional variants thereof having at least 70% homology, similarity or identity thereto. In another preferred embodiment, the cell expresses FlA1_{MA37}, Pfs, KinBt, IsoSc, AldSc, and an F-Aldh, or functional variants thereof having at least 70% homology, similarity or identity thereto. In some embodiments, the cell does not express the fluorinase and is provided with 5′-FDA, such as propagated in medium supplied with 5′-FDA.

Functional variants of F-Aldh may be identified by expressing said variant enzyme in a host cell, purifying the variant enzyme and performing an assay, e.g. an enzyme assay, to measure the conversion of FAld to FAc, for example as described in “Example 1—FAld, FAc and FAcCoA production” herein. FAc is detected and measured by LC-MS.

### Acetylating Acetaldehyde Dehydrogenase

In some embodiments, the cell is capable of producing a fluorinated compound, such as FAld as disclosed herein above, and is further capable of converting said compound into FAcCoA.

Disclosed herein are AcAldhs capable of catalysing the conversion of FAld to FAcCoA. In some embodiments, the cell may further express an AcAldh, thus being capable of producing FAc from a fluorinated compound. In some embodiments, the fluorinated compound is FAld and the AcAldh is capable of converting said FAld to FAcCoA, thus when expressed in a cell said AcAldh allows the cell to produce FAcCoA in the presence of FAld. As explained above, an Aldh may be an AcAldh and/or an F-Aldh, and therefore an AcAldh may be an Aldh and/or an F-Aldh.

The AcAldh may be an endogenous, i.e. a native AcAldh, or a heterologous AcAldh, i.e. an AcAldh that is not native to or expressed endogenously in the cell. In some embodiments, the AcAldh is an AcAldh native to Pseudomonas such as P. aeruginosa, preferably P. aeruginosa 1984 or P. aeruginosa 4022, or an AcAldh native to Escherichia such as E. coli, or an AcAldh native to Moorella thermoacetica such as M. thermoacetica.

In one embodiment, the Aldh is ExaC as set forth in SEQ ID NO: 21, or a functional variant thereof having at least 70% homology, identity or similarity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology, identity or similarity to SEQ ID NO: 21. In another embodiment, the Aldh is HdhA as set forth in SEQ ID NO: 23, or a functional variant thereof having at least 70% homology, identity or similarity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology, identity or similarity to SEQ ID NO: 23. In further one embodiment, the Aldh is EutE as set forth in SEQ ID NO: 25, or a functional variant thereof having at least 70% homology, identity or similarity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology, identity or similarity to SEQ ID NO: 25. In further another embodiment, the Aldh is Moth_1776p, acetaldehyde dehydrogenase from M. thermoacetica, as set forth in SEQ ID NO: 27, or a functional variant thereof having at least 70% homology, identity or similarity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology, identity or similarity to SEQ ID NO: 27.

The Aldh, such as the AcAldh and/or FAldh, may be expressed in the cell by introducing a nucleic acid sequence or gene as detailed further below, which encodes an Aldh, AcAldh and/or FAldh, such as ExaC, HdhA, EutE or Moth_1776p, for example SEQ ID NO: 21, SEQ ID NO: 23, SEQ ID NO: 25 and/or SEQ ID NO: 27 or a homologue thereof having at least 70% homology, identity or similarity thereto. The Aldh, AcAldh and/or FAldh gene or nucleic acid sequence, such as the exaC gene, hdhA gene, eutE gene and/or Moth_1776 gene, may be codon-optimised as is known in the art.

In some embodiments, the cell is capable of producing a fluorinated compound, such as FAld and is further capable of converting said compound into FAc and/or FAcCoA. In a preferred embodiment, the cell expresses FlA_{PtaU1}, DeoD, IsoBt, AldBt, and an Aldh selected from ExaC, HdhA, EutE and Moth_1776p, or functional variants thereof having at least 70% homology, similarity or identity thereto. In another preferred embodiment, the cell expresses FlA_{PtaU1}, DeoD, IsoBt, AldSc, and an Aldh selected from ExaC, HdhA, EutE and Moth_1776p, or functional variants thereof having at least 70% homology, similarity or identity thereto. In a preferred embodiment, the cell expresses FlA_{PtaU1}, DeoD, IsoSc, AldBt, and an Aldh selected from ExaC, HdhA, EutE and Moth_1776p, or functional variants thereof having at least 70% homology, similarity or identity thereto. In another preferred embodiment, the cell expresses FlA_{PtaU1}, DeoD, IsoSc, AldSc, and an Aldh selected from ExaC, HdhA, EutE and Moth_1776p, or functional variants thereof having at least 70% homology, similarity or identity thereto. In a preferred embodiment, the cell expresses FlA_{PtaU1}, FlB1, IsoBt, AldBt, and an Aldh selected from ExaC, HdhA, EutE and Moth_1776p, or functional variants thereof having at least 70% homology, similarity or identity thereto. In another preferred embodiment, the cell expresses FlA_{PtaU1}, FlB1, IsoBt, AldSc, and an Aldh selected from ExaC, HdhA, EutE and Moth_1776p, or functional variants thereof having at least 70% homology, similarity or identity thereto. In a preferred embodiment, the cell expresses FlA_{PtaU1}, FlB1, IsoSc, AldBt, and an Aldh selected from ExaC, HdhA, EutE and Moth_1776p, or functional variants thereof having at least 70% homology, similarity or identity thereto. In another preferred embodiment, the cell expresses FlA_{PtaU1}, FlB1, IsoSc, AldSc, and an Aldh selected from ExaC, HdhA, EutE and Moth_1776p, or functional variants thereof having at least 70% homology, similarity or identity thereto. In a preferred embodiment, the cell expresses FlA_{PtaU1}, Pfs, KinBt, IsoBt, AldBt, and an Aldh selected from ExaC, HdhA, EutE and Moth_1776p, or functional variants thereof having at least 70% homology, similarity or identity thereto. In another preferred embodiment, the cell expresses FlA_{PtaU1}, Pfs, KinBt, IsoBt, AldSc, and an Aldh selected from ExaC, HdhA, EutE and Moth_1776p, or functional variants thereof having at least 70% homology, similarity or identity thereto. In a preferred embodiment, the cell expresses FlA_{PtaU1}, Pfs, KinBt, IsoSc, AldBt, and an Aldh selected from ExaC, HdhA, EutE and Moth_1776p, or functional variants thereof having at least 70% homology, similarity or identity thereto. In another preferred embodiment, the cell expresses FlA_{PtaU1}, Pfs, KinBt, IsoSc, AldSc, and an Aldh selected from ExaC, HdhA, EutE and Moth_1776p, or functional variants thereof having at least 70% homology, similarity or identity thereto. In a preferred embodiment, the cell expresses FlA1_{MA37}, DeoD, IsoBt, AldBt, and an Aldh selected from ExaC, HdhA, EutE and Moth_1776p, or functional variants thereof having at least 70% homology, similarity or identity thereto. In another preferred embodiment, the cell expresses FlA1_{MA37}, DeoD, IsoBt, AldSc, and an Aldh selected from ExaC, HdhA, EutE and Moth_1776p, or functional variants thereof having at least 70% homology, similarity or identity thereto. In a preferred embodiment, the cell expresses FlA1_{MA37}, DeoD, IsoSc, AldBt, and an Aldh selected from ExaC, HdhA, EutE and Moth_1776p, or functional variants thereof having at least 70% homology, similarity or identity thereto. In another preferred embodiment, the cell expresses FlA1_{MA37}, DeoD, IsoSc, AldSc, and an Aldh selected from ExaC, HdhA, EutE and Moth_1776p, or functional variants thereof having at least 70% homology, similarity or identity thereto. In a preferred embodiment, the cell expresses FlA1_{MA37}, FlB1, IsoBt, AldBt, and an Aldh selected from ExaC, HdhA, EutE and Moth_1776p, or functional variants thereof having at least 70% homology, similarity or identity thereto. In another preferred embodiment, the cell expresses FlA1_{MA37}, FlB1, IsoBt, AldSc, and an Aldh selected from ExaC, HdhA, EutE and Moth_1776p, or functional variants thereof having at least 70% homology, similarity or identity thereto. In a preferred embodiment, the cell expresses FlA1_{MA37}, FlB1, IsoSc, AldBt, and an Aldh selected from ExaC, HdhA, EutE and Moth_1776p, or functional variants thereof having at least 70% homology, similarity or identity thereto. In another preferred embodiment, the cell expresses FlA1_{MA37}, FlB1, IsoSc, AldSc, and an Aldh selected from ExaC, HdhA, EutE and Moth_1776p, or functional variants thereof having at least 70% homology, similarity or identity thereto. In a preferred embodiment, the cell expresses FlA1_{MA37}, Pfs, KinBt, IsoBt, AldBt, and an Aldh selected from ExaC, HdhA, EutE and Moth_1776p, or functional variants thereof having at least 70% homology, similarity or identity thereto. In another preferred embodiment, the cell expresses FlA1_{MA37}, Pfs, KinBt, IsoBt, AldSc, and an Aldh selected from ExaC, HdhA, EutE and Moth_1776p, or functional variants thereof having at least 70% homology, similarity or identity thereto. In a preferred embodiment, the cell expresses FlA1_{MA37}, Pfs, KinBt, IsoSc, AldBt, and an Aldh selected from ExaC, HdhA, EutE and Moth_1776p, or functional variants thereof having at least 70% homology, similarity or identity thereto. In another preferred embodiment, the cell expresses FlA1_{MA37}, Pfs, KinBt, IsoSc, AldSc, and an Aldh selected from ExaC, HdhA, EutE and Moth_1776p, or functional variants thereof having at least 70% homology, similarity or identity thereto. In some embodiments, the cell does not express the fluorinase and is provided with 5′-FDA, such as propagated in medium supplied with 5′-FDA.

In one embodiment, the cell expresses FlA_{PtaU1} (SEQ ID NO: 1), Pfs (SEQ ID NO: 17), KinBt (SEQ ID NO: 33), IsoSc (SEQ ID NO: 37), AldSc (SEQ ID NO: 19) and HdhA (SEQ ID NO: 23), or functional variants thereof having at least 70% homology, similarity or identity thereto, such as at least 75%, such as at least 80%, such as at least 85%, such as at least 90%, such as at least 95%, such as at least 100% sequence homology, similarity or identity to SEQ ID NO: 1, SEQ ID NO: 17, SEQ ID NO: 33, SEQ ID NO: 37, SEQ ID NO: 19 and/or SEQ ID NO: 23. In another embodiment, the cell expresses Pfs (SEQ ID NO: 17), KinBt (SEQ ID NO: 33), IsoSc (SEQ ID NO: 37), AldSc (SEQ ID NO: 19) and HdhA (SEQ ID NO: 23), or functional variants thereof having at least 70% homology, similarity or identity thereto, such as at least 75%, such as at least 80%, such as at least 85%, such as at least 90%, such as at least 95%, such as at least 100% sequence homology, similarity or identity to SEQ ID NO: 17, SEQ ID NO: 33, SEQ ID NO: 37, SEQ ID NO: 19 and/or SEQ ID NO: 23. In further one embodiment, the cell expresses FlA_{PtaU1} (SEQ ID NO: 1), FlB1 (SEQ ID NO: 11), IsoSc (SEQ ID NO: 37), AldSc (SEQ ID NO: 19), and HdhA (SEQ ID NO: 23), or functional variants thereof having at least 70% homology, similarity or identity thereto, such as at least 75%, such as at least 80%, such as at least 85%, such as at least 90%, such as at least 95%, such as at least 100% sequence homology, similarity or identity to SEQ ID NO: 1, SEQ ID NO: 11, SEQ ID NO: 37, SEQ ID NO: 19 and/or SEQ ID NO: 23. In some further embodiments, the cell expresses FlB1 (SEQ ID NO: 11), IsoSc (SEQ ID NO: 37), AldSc (SEQ ID NO: 19), and HdhA (SEQ ID NO: 23), or functional variants thereof having at least 70% homology, similarity or identity thereto, such as at least 75%, such as at least 80%, such as at least 85%, such as at least 90%, such as at least 95%, such as at least 100% sequence homology, similarity or identity to SEQ ID NO: 11, SEQ ID NO: 37, SEQ ID NO: 19 and/or SEQ ID NO: 23. In further one embodiment, the cell expresses IsoSc (SEQ ID NO: 37), AldSc (SEQ ID NO: 19), and HdhA (SEQ ID NO: 23), or functional variants thereof having at least 70% homology, similarity or identity thereto, such as at least 75%, such as at least 80%, such as at least 85%, such as at least 90%, such as at least 95%, such as at least 100% sequence homology, similarity or identity to SEQ ID NO: 37, SEQ ID NO: 19 and/or SEQ ID NO: 23. In some embodiments, the cell does not express the fluorinase and is provided with 5′-FDA, such as propagated in medium supplied with 5′-FDA. In further some embodiments, the cell further expresses an Aldh, such as an FAldh and/or an AcAldh.

In one embodiment, the cell expresses FlA_{PtaU1} (SEQ ID NO: 1), Pfs (SEQ ID NO: 17), KinBt (SEQ ID NO: 33), IsoSc (SEQ ID NO: 37), AldSc (SEQ ID NO: 19) and ExaC (SEQ ID NO: 21), or functional variants thereof having at least 70% homology, similarity or identity thereto, such as at least 75%, such as at least 80%, such as at least 85%, such as at least 90%, such as at least 95%, such as at least 100% sequence homology, similarity or identity to SEQ ID NO: 1, SEQ ID NO: 17, SEQ ID NO: 33, SEQ ID NO: 37, SEQ ID NO: 19 and/or SEQ ID NO: 21. In another embodiment, the cell expresses Pfs (SEQ ID NO: 17), KinBt (SEQ ID NO: 33), IsoSc (SEQ ID NO: 37), AldSc (SEQ ID NO: 19) and ExaC (SEQ ID NO: 21), or functional variants thereof having at least 70% homology, similarity or identity thereto, such as at least 75%, such as at least 80%, such as at least 85%, such as at least 90%, such as at least 95%, such as at least 100% sequence homology, similarity or identity to SEQ ID NO: 17, SEQ ID NO: 33, SEQ ID NO: 37, SEQ ID NO: 19 and/or SEQ ID NO: 21. In further one embodiment, the cell expresses FlA_{PtaU1} (SEQ ID NO: 1), FlB1 (SEQ ID NO: 11), IsoSc (SEQ ID NO: 37), AldSc (SEQ ID NO: 19), and ExaC (SEQ ID NO: 21), or functional variants thereof having at least 70% homology, similarity or identity thereto, such as at least 75%, such as at least 80%, such as at least 85%, such as at least 90%, such as at least 95%, such as at least 100% sequence homology, similarity or identity to SEQ ID NO: 1, SEQ ID NO: 11, SEQ ID NO: 37, SEQ ID NO: 19 and/or SEQ ID NO: 21. In another embodiment, the cell expresses FlB1 (SEQ ID NO: 11), IsoSc (SEQ ID NO: 37), AldSc (SEQ ID NO: 19), and ExaC (SEQ ID NO: 21), or functional variants thereof having at least 70% homology, similarity or identity thereto, such as at least 75%, such as at least 80%, such as at least 85%, such as at least 90%, such as at least 95%, such as at least 100% sequence homology, similarity or identity to SEQ ID NO: 11, SEQ ID NO: 37, SEQ ID NO: 19 and/or SEQ ID NO: 21. In another embodiment, the cell expresses IsoSc (SEQ ID NO: 37), AldSc (SEQ ID NO: 19), and ExaC (SEQ ID NO: 21), or functional variants thereof having at least 70% homology, similarity or identity thereto, such as at least 75%, such as at least 80%, such as at least 85%, such as at least 90%, such as at least 95%, such as at least 100% sequence homology, similarity or identity to SEQ ID NO: 37, SEQ ID NO: 19 and/or SEQ ID NO: 21. In some embodiments, the cell does not express the fluorinase and is provided with 5′-FDA, such as propagated in medium supplied with 5′-FDA. In further some embodiments, the cell further expresses an Aldh, such as an FAldh and/or an AcAldh.

Functional variants of AcAldh may be identified by expressing said variant enzyme in a host cell, purifying the variant enzyme and performing an assay, e.g. an enzyme assay or by using spectroscopy, to directly or indirectly measure the conversion of FAld to FAcCoA, for example as described in “Example 1—FAld, FAc and FAcCoA production” herein below. FAcCoA can be detected by LC-MS/MS.

### Acetyl-CoA Synthetase

In some embodiments, the cell is capable of producing a fluorinated compound, such as FAc as disclosed herein above, and is further capable of converting said compound into FAcCoA. In this section, said fluorinated compound may be FAc.

Disclosed herein are acetyl-CoA synthetases (Acs′) capable of catalysing the conversion of FAc to FAcCoA. In some embodiments, the cell may further express an Acs, thus being capable of producing FAcCoA from a fluorinated compound. Thus, in some embodiments, the fluorinated compound is FAc and the Acs is capable of converting FAc to FAcCoA, thus when expressed in a cell said Acs allows the cell to produce FAcCoA in the presence of FAc.

The Acs may be a heterologous Acs, i.e. an Acs that is not native to or expressed endogenously in the cell. In some embodiments, the Acs is an Acs native to Bacillus such as B. subtilis, or an Acs native to Streptomyces such as S. coelicolor, or an Acs native to Cupriavidus such as C. necator, or an Acs native to Pseudomonas such as P. putida or P. aeruginosa. Thus, in one embodiment the Acs is AcsBt, also known as AcsA from B. subtilis, as set forth in SEQ ID NO: 39, or a functional variant thereof having at least 70% homology, identity or similarity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology, identity or similarity to SEQ ID NO: 39. In another embodiment, the Acs is AcsCn as set forth in SEQ ID NO: 41, or a functional variant thereof having at least 70% homology, identity or similarity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology, identity or similarity to SEQ ID NO: 41. In a further embodiment, the Acs is AcsSc, also known as AcsA from S. coelicolor, as set forth in SEQ ID NO: 43, or a functional variant thereof having at least 70% homology, identity or similarity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology, identity or similarity to SEQ ID NO: 43.

The Acs may be expressed in the cell by introducing a nucleic acid sequence or gene as detailed further below, which encodes an Acs, such as AcsBt, AcsCn or AcsSc, for example SEQ ID NO: 39, SEQ ID NO: 41 and/or SEQ ID NO: 43 or a homologue thereof having at least 70% homology, identity or similarity thereto. The Acs gene or nucleic acid sequence, such as the acsBt gene, acsCn gene (acsA from B. subtilis) and/or acsSc gene (acsA from S. coelicolor), may be codon-optimised as is known in the art.

In a preferred embodiment, the cell expresses FlA_{PtaU1}, DeoD, IsoBt, AldBt, an F-Aldh, and an Acs selected from AcsBt, AcsCn and AcsSc, or functional variants thereof having at least 70% homology, similarity or identity thereto. In another preferred embodiment, the cell expresses FlA_{PtaU1}, DeoD, IsoBt, AldSc, an F-Aldh, and an Acs selected from AcsBt, AcsCn and AcsSc, or functional variants thereof having at least 70% homology, similarity or identity thereto. In a preferred embodiment, the cell expresses FlA_{PtaU1}, DeoD, IsoSc, AldBt, an F-Aldh, and an Acs selected from AcsBt, AcsCn and AcsSc, or functional variants thereof having at least 70% homology, similarity or identity thereto. In another preferred embodiment, the cell expresses FlA_{PtaU1}, DeoD, IsoSc, AldSc, an F-Aldh, and an Acs selected from AcsBt, AcsCn and AcsSc, or functional variants thereof having at least 70% homology, similarity or identity thereto. In a preferred embodiment, the cell expresses FlA_{PtaU1}, FlB1, IsoBt, AldBt, an F-Aldh, and an Acs selected from AcsBt, AcsCn and AcsSc, or functional variants thereof having at least 70% homology, similarity or identity thereto. In another preferred embodiment, the cell expresses FlA_{PtaU1}, FlB1, IsoBt, AldSc, an F-Aldh, and an Acs selected from AcsBt, AcsCn and AcsSc, or functional variants thereof having at least 70% homology, similarity or identity thereto. In a preferred embodiment, the cell expresses FlA_{PtaU1}, FlB1, IsoSc, AldBt, an F-Aldh, and an Acs selected from AcsBt, AcsCn and AcsSc, or functional variants thereof having at least 70% homology, similarity or identity thereto. In another preferred embodiment, the cell expresses FlA_{PtaU1}, FlB1, IsoSc, AldSc, an F-Aldh, and an Acs selected from AcsBt, AcsCn and AcsSc, or functional variants thereof having at least 70% homology, similarity or identity thereto. In a preferred embodiment, the cell expresses FlA_{PtaU1}, Pfs, KinBt, IsoBt, AldBt, an F-Aldh, and an Acs selected from AcsBt, AcsCn and AcsSc, or functional variants thereof having at least 70% homology, similarity or identity thereto. In another preferred embodiment, the cell expresses FlA_{PtaU1}, Pfs, KinBt, IsoBt, AldSc, an F-Aldh, and an Acs selected from AcsBt, AcsCn and AcsSc, or functional variants thereof having at least 70% homology, similarity or identity thereto. In a preferred embodiment, the cell expresses FlA_{PtaU1}, Pfs, KinBt, IsoSc, AldBt, an F-Aldh, and an Acs selected from AcsBt, AcsCn and AcsSc, or functional variants thereof having at least 70% homology, similarity or identity thereto. In another preferred embodiment, the cell expresses FlA_{PtaU1}, Pfs, KinBt, IsoSc, AldSc, an F-Aldh, and an Acs selected from AcsBt, AcsCn and AcsSc, or functional variants thereof having at least 70% homology, similarity or identity thereto. In a preferred embodiment, the cell expresses FlA1_{MA37}, DeoD, IsoBt, AldBt, an F-Aldh, and an Acs selected from AcsBt, AcsCn and AcsSc, or functional variants thereof having at least 70% homology, similarity or identity thereto. In another preferred embodiment, the cell expresses FlA1_{MA37}, DeoD, IsoBt, AldSc, an F-Aldh, and an Acs selected from AcsBt, AcsCn and AcsSc, or functional variants thereof having at least 70% homology, similarity or identity thereto. In a preferred embodiment, the cell expresses FlA1_{MA37}, DeoD, IsoSc, AldBt, an F-Aldh, and an Acs selected from AcsBt, AcsCn and AcsSc, or functional variants thereof having at least 70% homology, similarity or identity thereto. In another preferred embodiment, the cell expresses FlA1_{MA37}, DeoD, IsoSc, AldSc, an F-Aldh, and an Acs selected from AcsBt, AcsCn and AcsSc, or functional variants thereof having at least 70% homology, similarity or identity thereto. In a preferred embodiment, the cell expresses FlA1_{MA37}, FlB1, IsoBt, AldBt, an F-Aldh, and an Acs selected from AcsBt, AcsCn and AcsSc, or functional variants thereof having at least 70% homology, similarity or identity thereto. In another preferred embodiment, the cell expresses FlA1_{MA37}, FlB1, IsoBt, AldSc, an F-Aldh, and an Acs selected from AcsBt, AcsCn and AcsSc, or functional variants thereof having at least 70% homology, similarity or identity thereto. In a preferred embodiment, the cell expresses FlA1_{MA37}, FlB1, IsoSc, AldBt, an F-Aldh, and an Acs selected from AcsBt, AcsCn and AcsSc, or functional variants thereof having at least 70% homology, similarity or identity thereto. In another preferred embodiment, the cell expresses FlA1_{MA37}, FlB1, IsoSc, AldSc, an F-Aldh, and an Acs selected from AcsBt, AcsCn and AcsSc, or functional variants thereof having at least 70% homology, similarity or identity thereto. In a preferred embodiment, the cell expresses FlA1_{MA37}, Pfs, KinBt, IsoBt, AldBt, an F-Aldh, and an Acs selected from AcsBt, AcsCn and AcsSc, or functional variants thereof having at least 70% homology, similarity or identity thereto. In another preferred embodiment, the cell expresses FlA1_{MA37}, Pfs, KinBt, IsoBt, AldSc, an F-Aldh, and an Acs selected from AcsBt, AcsCn and AcsSc, or functional variants thereof having at least 70% homology, similarity or identity thereto. In a preferred embodiment, the cell expresses FlA1_{MA37}, Pfs, KinBt, IsoSc, AldBt, an F-Aldh, and an Acs selected from AcsBt, AcsCn and AcsSc, or functional variants thereof having at least 70% homology, similarity or identity thereto. In another preferred embodiment, the cell expresses FlA1_{MA37}, Pfs, KinBt, IsoSc, AldSc, an F-Aldh, and an Acs selected from AcsBt, AcsCn and AcsSc, or functional variants thereof having at least 70% homology, similarity or identity thereto. The F-Aldh may be an endogenous F-Aldh, such as an endogenous Aldh with F-Aldh activity and optionally also AcAldh activity, or a heterologous F-Aldh, such as an heterologous Aldh with F-Aldh activity and optionally also AcAldh activity, for example the F-Aldh may be selected from the group consisting of ExaC as set forth in SEQ ID NO: 21, HdhA as set forth in SEQ ID NO: 23, EutE as set forth in SEQ ID NO: 25, and Moth_1776p as set forth in SEQ ID NO: 27, or a functional variant thereof having at least 70% homology, identity or similarity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology, identity or similarity to SEQ ID NO: 21, SEQ ID NO: 23, SEQ ID NO: 25 or SEQ ID NO: 27, respectively. In some embodiments, the cell does not express the fluorinase and is provided with 5′-FDA, such as propagated in medium supplied with 5′-FDA. In further some embodiments, the cell does not express the F-Aldh. In some other embodiments, the cell does not express the F-Aldh and the fluorinase and is provided with 5′-FDA, such as propagated in medium supplied with 5′-FDA. In other embodiments, the F-Aldh is an Aldh, such as an Aldh and/or AcAldh.

In a preferred embodiment, the cell expresses FlA_{PtaU1}, DeoD, IsoBt, AldBt, ExaC, and AcsBt, or functional variants thereof having at least 70% homology, similarity or identity thereto. In another preferred embodiment, the cell expresses FlA_{PtaU1}, DeoD, IsoBt, AldSc, ExaC, and AcsBt, or functional variants thereof having at least 70% homology, similarity or identity thereto. In a preferred embodiment, the cell expresses FlA_{PtaU1}, DeoD, IsoSc, AldBt, ExaC, and AcsBt, or functional variants thereof having at least 70% homology, similarity or identity thereto. In another preferred embodiment, the cell expresses FlA_{PtaU1}, DeoD, IsoSc, AldSc, ExaC, and AcsBt, or functional variants thereof having at least 70% homology, similarity or identity thereto. In a preferred embodiment, the cell expresses FlA_{PtaU1}, FlB1, IsoBt, AldBt, ExaC, and AcsBt, or functional variants thereof having at least 70% homology, similarity or identity thereto. In another preferred embodiment, the cell expresses FlA_{PtaU1}, FlB1, IsoBt, AldSc, ExaC, and AcsBt, or functional variants thereof having at least 70% homology, similarity or identity thereto. In a preferred embodiment, the cell expresses FlA_{PtaU1}, FlB1, IsoSc, AldBt, ExaC, and AcsBt, or functional variants thereof having at least 70% homology, similarity or identity thereto. In another preferred embodiment, the cell expresses FlA_{PtaU1}, FlB1, IsoSc, AldSc, ExaC, and AcsBt, or functional variants thereof having at least 70% homology, similarity or identity thereto. In a preferred embodiment, the cell expresses FlA_{PtaU1}, Pfs, KinBt, IsoBt, AldBt, ExaC, and AcsBt, or functional variants thereof having at least 70% homology, similarity or identity thereto. In another preferred embodiment, the cell expresses FlA_{PtaU1}, Pfs, KinBt, IsoBt, AldSc, ExaC, and AcsBt, or functional variants thereof having at least 70% homology, similarity or identity thereto. In a preferred embodiment, the cell expresses FlA_{PtaU1}, Pfs, KinBt, IsoSc, AldBt, ExaC, and AcsBt, or functional variants thereof having at least 70% homology, similarity or identity thereto. In another preferred embodiment, the cell expresses FlA_{PtaU1}, Pfs, KinBt, IsoSc, AldSc, ExaC, and AcsBt, or functional variants thereof having at least 70% homology, similarity or identity thereto. In a preferred embodiment, the cell expresses FlA1_{MA37}, DeoD, IsoBt, AldBt, ExaC, and AcsBt, or functional variants thereof having at least 70% homology, similarity or identity thereto. In another preferred embodiment, the cell expresses FlA1_{MA37}, DeoD, IsoBt, AldSc, ExaC, and AcsBt, or functional variants thereof having at least 70% homology, similarity or identity thereto. In a preferred embodiment, the cell expresses FlA1_{MA37}, DeoD, IsoSc, AldBt, ExaC, and AcsBt, or functional variants thereof having at least 70% homology, similarity or identity thereto. In another preferred embodiment, the cell expresses FlA1_{MA37}, DeoD, IsoSc, AldSc, ExaC, and AcsBt, or functional variants thereof having at least 70% homology, similarity or identity thereto. In a preferred embodiment, the cell expresses FlA1_{MA37}, FlB1, IsoBt, AldBt, ExaC, and AcsBt, or functional variants thereof having at least 70% homology, similarity or identity thereto. In another preferred embodiment, the cell expresses FlA1_{MA37}, FlB1, IsoBt, AldSc, ExaC, and AcsBt, or functional variants thereof having at least 70% homology, similarity or identity thereto. In a preferred embodiment, the cell expresses FlA1_{MA37}, FlB1, IsoSc, AldBt, ExaC, and AcsBt, or functional variants thereof having at least 70% homology, similarity or identity thereto. In another preferred embodiment, the cell expresses FlA1_{MA37}, FlB1, IsoSc, AldSc, ExaC, and AcsBt, or functional variants thereof having at least 70% homology, similarity or identity thereto. In a preferred embodiment, the cell expresses FlA1_{MA37}, Pfs, KinBt, IsoBt, AldBt, ExaC, and AcsBt, or functional variants thereof having at least 70% homology, similarity or identity thereto. In another preferred embodiment, the cell expresses FlA1_{MA37}, Pfs, KinBt, IsoBt, AldSc, ExaC, and AcsBt, or functional variants thereof having at least 70% homology, similarity or identity thereto. In a preferred embodiment, the cell expresses FlA1_{MA37}, Pfs, KinBt, IsoSc, AldBt, ExaC, and AcsBt, or functional variants thereof having at least 70% homology, similarity or identity thereto. In another preferred embodiment, the cell expresses FlA1_{MA37}, Pfs, KinBt, IsoSc, AldSc, ExaC, and AcsBt, or functional variants thereof having at least 70% homology, similarity or identity thereto. In some embodiments, the cell does not express the fluorinase and is provided with 5′-FDA, such as propagated in medium supplied with 5′-FDA.

Functional variants of the Acs may be identified by expressing said variant enzyme in a host cell, purifying the variant enzyme and performing an assay, e.g. an enzyme assay, to measure the conversion of FAc to FAcCoA, for example as described in “Example 1—FAld, FAc and FAcCoA production” herein. FAcCoA can be detected by LC-MS/MS.

### Other Modifications

The intracellular availability of substrates and/or fluorinated compounds such as fluoride, SAM or any of the pathway substrates, precursors, or intermediates, such as FAld and/or FAc, may have an influence on the efficiency of production of any of the products derived from them.

Thus, in some embodiments, it may thus be advantageous to use a cell which has reduced ability to transport fluoride or pathway substrates, precursors, or intermediates out of the cell, in order to increase intracellular availability of any of them. In some other embodiments, it may be advantageous to reduce the endogenous, i.e. native, conversion of any of the pathway substrates, precursors, or intermediates to compounds that are not part of the pathway.

Hence, in some embodiments the cell may be further modified to reduce endogenous conversion of FAld to FAc. In some embodiments, said modification of the cell to reduce endogenous conversion of FAld to FAc, comprises reduction of endogenous Aldh activity.

### Transporters

The intracellular availability of the co-substrate (fluoride) or inducer (fluoride) may have an influence on the efficiency of reaction. In some embodiments, it may thus be advantageous to use a cell which has reduced ability to transport the co-substrate out of the cell, in order to increase intracellular availability of the co-substrate.

Accordingly, in some embodiments, the cell further comprises a mutation in at least one fluoride transporter gene encoding a fluoride transporter, said mutation resulting in a partial or total loss of function of said fluoride transporter.

In embodiments where the cell is used to perform in vivo fluorination, the cell thus may comprise a mutation in at least one fluoride transporter gene encoding a fluoride transporter, said mutation resulting in a partial or total loss of function of said fluoride transporter.

The mutation may be a partial or total deletion, as is known in the art.

For example, in a specific embodiment, the cell is a Pseudomonas putida cell used for in vivo fluorination of a substrate as described herein, in particular a P. putida KT2440 cell, and the cell has a mutation in the crcB gene as set forth in SEQ ID NO: 9. In some embodiments, the cell expresses a dead variant, i.e. non-functional, variant of CrcB as set forth in SEQ ID NO: 10. In one embodiment, the crcB gene has been deleted.

In specific embodiments, the cell is capable of producing a fluorinated compound from a substrate in the presence of fluoride, said cell comprising:
a) a first nucleic acid comprising a first promoter and a fluorinase gene encoding a fluorinase (EC 2.5.1.63) capable of catalysing the formation of a C—F bond, said fluorinase gene being under the control of the first promoter, wherein the fluorinase gene encodes the fluorinase of Methanosaeta sp. PtaU1.Bin055 as set forth in SEQ ID NO: 1 (FlA_{PtaU1}), or a functional variant thereof having at least 70% sequence homology, similarity or identity thereto, such as at least 75%, such as at least 80%, such as at least 85%, such as at least 90%, such as at least 95%, such as at least 100% sequence homology, similarity or identity to SEQ ID NO: 1, and wherein the first promoter is a T7 promoter;
b) optionally a second nucleic acid comprising a fluoride-responsive riboswitch such as the FRS riboswitch from P. syringae (SEQ ID NO: 4), a second promoter such as the native promoter of the eriC^{F} gene of P. syringae or the P_{EM7} promoter, and a gene encoding an activator of transcription, wherein the activator of transcription is the T7 RNA polymerase, said gene being under the control of the second promoter, wherein transcription of the T7 RNA polymerase can be induced by fluoride, wherein T7 RNA polymerase upon expression activates transcription from the T7 promoter;
   wherein the cell is capable of expressing the activator of transcription and the fluorinase at least in the presence of fluoride. In some embodiments, the cell is a Pseudomonas putida cell, in particular a P. putida KT2440 cell, which may further comprise a mutation of a gene encoding a fluoride transporter resulting in a partial or total loss of activity of the fluoride transporter.

In another embodiment, the cell is capable of producing a fluorinated compound from a substrate in the presence of fluoride, said cell comprising:
a) a first nucleic acid comprising a first promoter and a fluorinase gene encoding a fluorinase (EC 2.5.1.63) capable of catalysing the formation of a C—F bond, said fluorinase gene being under the control of the first promoter, wherein the fluorinase gene encodes the fluorinase of Methanosaeta sp. PtaU1.Bin055 as set forth in SEQ ID NO: 1 (FlA_{PtaU1}), or a functional variant thereof having at least 70% sequence homology, similarity or identity thereto, such as at least 75%, such as at least 80%, such as at least 85%, such as at least 90%, such as at least 95%, such as at least 100% sequence homology, similarity or identity to SEQ ID NO: 1, and wherein the first promoter preferably is the native promoter of the eriC^{F} gene of P. syringae or the P_{EM7} promoter;
b) optionally a second nucleic acid comprising a fluoride-responsive riboswitch such as the FRS riboswitch from P. syringae (SEQ ID NO: 4),
wherein transcription of the fluorinase gene can be induced by fluoride;
   wherein the cell is capable of expressing the fluorinase at least in the presence of fluoride. In some embodiments, the cell is a Pseudomonas putida cell, in particular a P. putida KT2440 cell, which may further comprise a mutation of a gene encoding a fluoride transporter resulting in a partial or total loss of activity of the fluoride transporter.

In specific embodiments, the cell is capable of producing a fluorinated compound from a substrate in the presence of fluoride, said cell comprising:
a) a first nucleic acid comprising a first promoter and a fluorinase gene encoding a fluorinase (EC 2.5.1.63) capable of catalysing the formation of a C—F bond, said fluorinase gene being under the control of the first promoter, wherein the fluorinase gene encodes the fluorinase of Methanosaeta sp. PtaU1.Bin055 as set forth in SEQ ID NO: 1 (FlA_{PtaU1}), or a functional variant thereof having at least 70% sequence homology, similarity or identity thereto, and wherein the first promoter is preferably the promoter of the eriC^{F} gene or the P_{EM7} promoter;
b) optionally a second nucleic acid comprising a riboswitch, wherein the cell is capable of expressing the fluorinase at least in the presence of fluoride. In some embodiments, the cell is a Pseudomonas putida cell, in particular a P. putida KT2440 cell, which may further comprise a mutation of a gene encoding a fluoride transporter resulting in a partial or total loss of activity of the fluoride transporter. In some embodiments the first nucleic acid and the second nucleic acid are the same nucleic acid, and transcription from the first promoter is induced by the riboswitch in the presence of inducer.

### Nucleic Acids

In some embodiments, the nucleic acid encoding the fluorinase is a nucleic acid native to Methanosaeta such as Methanosaeta sp. PtaU1.Bin055, or a nucleic acid native to Streptomyces such as Streptomyces sp. MA37, Streptomyces sp. SAJ15, S. cattleya, S. xinghaiensis, or a nucleic acid native to Nocardia such as N. brasiliensis or a nucleic acid native to Actinoplanes such as Actinoplanes sp. N902-109. In other embodiments, the nucleic acid encoding the fluorinase comprises or consists of SEQ ID NO: 2, SEQ ID NO: 3, SEQ ID NO: 31, or SEQ ID NO: 32, or a homologue thereof having at 70% homology, identity or similarity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology, identity or similarity to SEQ ID NO: 2, SEQ ID NO: 3, SEQ ID NO: 31 or SEQ ID NO: 32, respectively.

In further other embodiments, the nucleic acid encoding the PNP is a nucleic acid native to Streptomyces such as Streptomyces sp. MA37, S. xinghaeiensis or S. cattleya, or a nucleic acid native to Escherichia such as E. coli. In some embodiments, the nucleic acid encoding the PNP comprises or consists of flB1 as set forth in SEQ ID NO: 12 and/or SEQ ID NO: 13, and/or deoD as set forth in SEQ ID NO: 14, or a homologue thereof having at least 70% homology, identity or similarity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology, identity or similarity to SEQ ID NO: 12, SEQ ID NO: 13 and/or SEQ ID NO: 14, respectively.

In some embodiments, the nucleic acid encoding the nucleosidase is native to Escherichia such as E. coli. Thus, in further one embodiment, the nucleic acid encoding the nucleosidase, comprises or consists of pfs as set forth in SEQ ID NO: 16, or a homologue thereof having at least 70% homology, identity or similarity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology, identity or similarity to SEQ ID NO: 16.

In some embodiments, the nucleic acid encoding the kinase is native to Bacillus such as a B. thuringiensis. In another embodiment, the nucleic acid encoding the kinase comprises or consists of kinBt, also known as drdK, as set forth in SEQ ID NO: 34, or a homologue thereof having at least 70% homology, identity or similarity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology, identity or similarity to SEQ ID NO: 34.

In some embodiments, the nucleic acid encoding the isomerase is a nucleic acid native to Bacillus such as B. thuringiensis, or a nucleic acid native to Streptomyces such as S. cattleya. In one embodiments, the nucleic acid encoding the isomerase comprises or consists of isoBt, also known as drdI, as set forth in SEQ ID NO: 36 and/or isoSc as set forth in SEQ ID NO: 38, or a homologue thereof having at least 70% homology, identity or similarity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology, identity or similarity to SEQ ID NO: 36 and/or SEQ ID NO: 38, respectively.

In further some embodiments, the nucleic acid encoding the aldolase is a nucleic acid native to Bacillus such as B. thuringiensis, or a nucleic acid native to Streptomyces such as S. coelicolor. In some embodiments, the nucleic acid encoding the aldolase comprises or consists of aldBt, also known as drdA, as set forth in SEQ ID NO: 18 and/or aldSc as set forth in SEQ ID NO: 20, or a homologue thereof having at least 70% homology, identity or similarity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology, identity or similarity to SEQ ID NO: 18 and/or SEQ ID NO: 20, respectively.

In other embodiments, the nucleic acid encoding the F-Aldh and/or the AcAldh is a nucleic acid native to Pseudomonas such as P. aeruginosa, preferably P. aeruginosa 1984 and/or P. aeruginosa 4022, or a nucleic acid native to Escherichia such as E. coli or a nucleic acid native to M. thermoacetica such as M. thermoacetica. In some embodiments, the nucleic acid encoding the F-Aldh and/or the AcAldh comprises and/or consists of exaC as set forth in SEQ ID NO: 22, hdhA as set forth in SEQ ID NO: 24, eutE as set forth in SEQ ID NO: 26 and/or Moth_1776 as set forth in SEQ ID NO: 28, or a homologue thereof having at least 70% homology, identity or similarity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology, identity or similarity to SEQ ID NO: 22, SEQ ID NO: 24, SEQ ID NO: 26 or SEQ ID NO: 28, respectively.

In some embodiments, nucleic acid encoding the Acs is a nucleic acid native to Bacillus such as B. subtilis, or a nucleic acid native to Streptomyces such as S. coelicolor, or a nucleic acid native to Cupriavidus such as C. necator, or a nucleic acid native to Pseudomonas such as P. putida or P. aeruginosa. In further some embodiments, the nucleic acid encoding the Acs comprises or consists of acsBt, also known as acsA from B. subtilis, as set forth in SEQ ID NO: 40, acsCn as set forth in SEQ ID NO: 42, and/or acsSc, also known as acsA from S. coelicolor, as set forth in SEQ ID NO: 44, or a homologue thereof having at least 70% homology, identity or similarity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology, identity or similarity to SEQ ID NO: 40, SEQ ID NO: 42, and/or SEQ ID NO: 44, respectively.

In another embodiment, the nucleic acid encoding the alcohol dehydrogenase is a nucleic acid native to Pseudomonas such as P. putida, for example P. putida KT2440. Without being bound by theory, the nucleic acid encoding the alcohol dehydrogenase comprises or consists of the nucleic acid with the locus tag PP_4760 (RefSeq Accession NP_746866.1), PP_1816 (RefSeq Accession NP_743971.1), PP_2827 (RefSeq Accession NP_744971.1), PP_2962 (RefSeq Accession NP_745106.1), PP_1720 (RefSeq Accession NP_743877.1), PP_5210 (RefSeq Accession NP_747311.1), PP_2049 (RefSeq Accession NP_744199.1), PP_2953 (RefSeq Accession NP_745097.1), or PP_2988 (RefSeq Accession NP_745132.1), or a homologue thereof having at least 70% homology, identity or similarity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology, identity or similarity thereto. In some other embodiments, and without being bound by theory, the nucleic acid encoding the alcohol dehydrogenase comprises or consists of adhP (locus tag PP_3839, RefSeq Accession NP_745969.1) from P. putida or yqhD (locus tag PP_2492, RefSeq Accession NP_744640.1) from P. putida, or a homologue thereof having at least 70% homology, identity or similarity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology, identity or similarity thereto.

In some embodiments, the nucleic acids encoding the heterologous fluorinase, the nucleosidase, the phosphorylase, the PNP, the kinase, the isomerase, the aldolase, the ADH, the AcAldh, the F-Aldh, and/or the Acs are codon-optimised.

In further some embodiments, the cell further comprises one or more promoters that control expression of:
i. the fluorinase gene;
ii. the nucleosidase gene;
iii. the purine nucleoside phosphorylase (PNP) and/or phosphorylase gene;
iv. the kinase gene;
v. the isomerase gene;
vi. the aldolase gene;
vii. the alcohol dehydrogenase gene;
viii. the acetylating acetaldehyde dehydrogenase gene;
ix. the fluoroacetaldehyde dehydrogenase gene; and/or
x. the acetyl-CoA synthetase gene.

In some embodiments, the one or more promoters is a T7 promoter. In other embodiments, said one or more promoters may be inducible and/or constitutive. In further some embodiments, said one or more promoters is or comprises a T7 promoter.

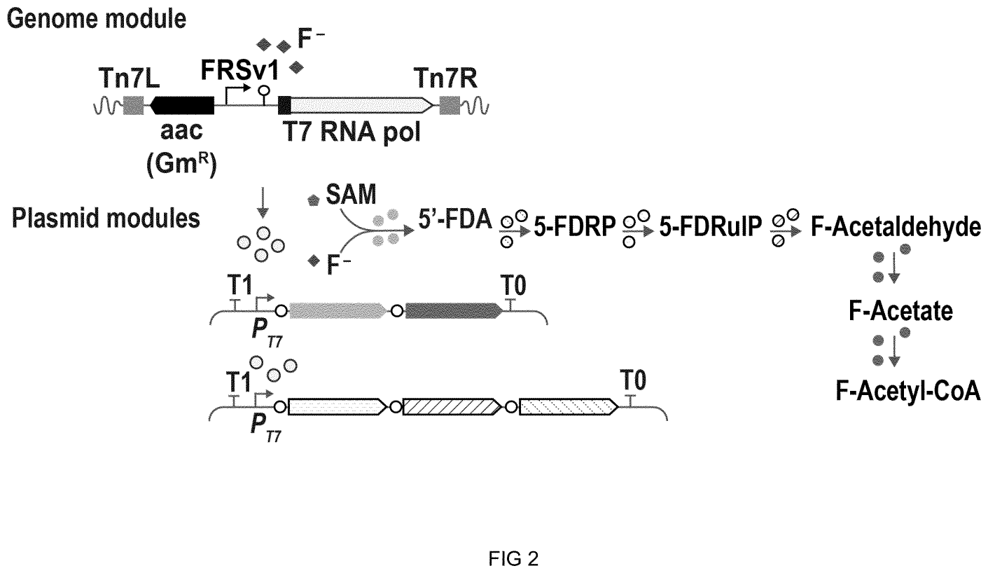

The cell may further comprises one or more riboswitches. Said one or more riboswitch may control transcription from said one or more promoters. In other words, any of the genes described herein, such as the gene(s) encoding the fluorinase, the nucleosidase, the PNP, the phosphorylase, the kinase, the isomerase, the aldolase, the ADH, the AcAldh, the F-Aldh and/or the Acs, may be under the control of said one or more riboswitches or similar expression systems known to the skilled person. Such synthetic circuit, wherein genes of the fluorination pathway are expressed using a fluoride-responsive riboswitch, is depicted in FIG. 2. Thus, in some embodiments, transcription from said one or more promoters is induced in the presence of an inducer, wherein said riboswitch is responsive to said inducer. In preferred embodiments, the inducer is a fluoride salt such as NaF or KF.

In other embodiments, said one or more riboswitches is the fluoride-responsive riboswitch (FRSv1) as set forth in SEQ ID NO: 5, or a functional variant thereof having at least 90% homology, identity or similarity to SEQ ID NO: 5, such at least 91%, such as at least 92%, such as at least 93%, such as at least 94%, such as at least 95%, such as at least 96%, such as at least 97%, such as at least 98%, such as at least 99%, such as 100% homology, identity or similarity to SEQ ID NO: 5.

In one embodiment, the cell comprises at least one nucleic acid comprising or consisting of:
SEQ ID NO: 2 and SEQ ID NO: 12 and/or SEQ ID NO: 13;
SEQ ID NO: 3 and SEQ ID NO: 12 and/or SEQ ID NO: 13;
SEQ ID NO: 16 and SEQ ID NO: 34;
SEQ ID NO: 2 and/or SEQ ID NO: 3, SEQ ID NO: 16, and SEQ ID NO: 34;
SEQ ID NO: 2, SEQ ID NO: 12 and/or SEQ ID NO: 13, and SEQ ID NO: 38;
SEQ ID NO: 3, SEQ ID NO: 12 and/or SEQ ID NO: 13, and SEQ ID NO: 38;
SEQ ID NO: 2 and/or SEQ ID NO: 3, SEQ ID NO: 16, SEQ ID NO: 34, and SEQ ID NO: 38;
SEQ ID NO: 16, SEQ ID NO: 34, and SEQ ID NO: 38;
SEQ ID NO: 2, SEQ ID NO: 12 and/or SEQ ID NO: 13, SEQ ID NO: 38 and SEQ ID NO: 20;
SEQ ID NO: 3, SEQ ID NO: 12 and/or SEQ ID NO: 13, SEQ ID NO: 38 and SEQ ID NO: 20;
SEQ ID NO: 2 and/or SEQ ID NO: 3, SEQ ID NO: 16, SEQ ID NO: 34, SEQ ID NO: 38, and SEQ ID NO: 20;
SEQ ID NO: 16, SEQ ID NO: 34, SEQ ID NO: 38 and SEQ ID NO: 20;
SEQ ID NO: 2 and/or SEQ ID NO: 3, SEQ ID NO: 16, SEQ ID NO: 34, SEQ ID NO: 38, SEQ ID NO: 20 and SEQ ID NO: 24;
SEQ ID NO: 16, SEQ ID NO: 34, SEQ ID NO: 38, SEQ ID NO: 20 and SEQ ID NO: 24;
SEQ ID NO: 2, SEQ ID NO: 12 and/or SEQ ID NO: 13, SEQ ID NO: 38, SEQ ID NO: 20 and SEQ ID NO: 24;
SEQ ID NO: 3, SEQ ID NO: 12 and/or SEQ ID NO: 13, SEQ ID NO: 38, SEQ ID NO: 20, and SEQ ID NO: 24;
SEQ ID NO: 2 and/or SEQ ID NO: 3, SEQ ID NO: 16, SEQ ID NO: 34, SEQ ID NO: 38, SEQ ID NO: 20 and SEQ ID NO: 22;
SEQ ID NO: 16, SEQ ID NO: 34, SEQ ID NO: 38, SEQ ID NO: 20, and SEQ ID NO: 22;
SEQ ID NO: 2, SEQ ID NO: 12 and/or SEQ ID NO: 13, SEQ ID NO: 38, SEQ ID NO: 20, and SEQ ID NO: 22;
SEQ ID NO: 3, SEQ ID NO: 12 and/or SEQ ID NO: 13, SEQ ID NO: 38, SEQ ID NO: 20, and SEQ ID NO: 22;
SEQ ID NO: 12 and/or SEQ ID NO: 13 and SEQ ID NO: 38;
SEQ ID NO: 12 and/or SEQ ID NO: 13, SEQ ID NO: 38, SEQ ID NO: 20;
SEQ ID NO: 38 and SEQ ID NO: 20;
SEQ ID NO: 34 and SEQ ID NO: 20;
SEQ ID NO: 34 and SEQ ID NO: 18;
SEQ ID NO: 38 and SEQ ID NO: 18;
SEQ ID NO: 12 and/or SEQ ID NO: 13, SEQ ID NO: 38, SEQ ID NO: 20, and SEQ ID NO: 24;
SEQ ID NO: 38, SEQ ID NO: 20, and SEQ ID NO: 24;
SEQ ID NO: 12 and/or SEQ ID NO: 13, SEQ ID NO: 38, SEQ ID NO: 20, and SEQ ID NO: 22; and/or
SEQ ID NO: 38, SEQ ID NO: 20, and SEQ ID NO: 22;
   or homologues thereof having at least 70% homology, identity or similarity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology, identity or similarity to SEQ ID NO: 2, SEQ ID NO: 3, SEQ ID NO: 12, SEQ ID NO: 13, SEQ ID NO: 16, SEQ ID NO: 18, SEQ ID NO: 20, SEQ ID NO: 22, SEQ ID NO: 24, SEQ ID NO: 34, and/or SEQ ID NO: 38.

In some embodiments, the cell does not comprise a nucleic acid comprising or consisting of SEQ ID NO: 2 and/or SEQ ID NO: 3.

### Expression Systems

Further disclosed herein are nucleic acids and/or expression systems useful for expression in a cell being capable of fluorinating a substrate and/or producing FAld and optionally FAcCoA, FEtOH, and/or FAc and/or one or more derivatives thereof from a fluorinated compound as disclosed herein. The present nucleic acids may be provided as one or more nucleic acid molecules, nucleic acids or polynucleotides, for example they may be comprised in one or more vectors and/or expression systems. Such nucleic acids may be introduced in the cell by methods known in the art. The terms nucleic acid(s) and polynucleotide(s) may be used interchangeably herein. Said nucleic acids, polynucleotides and/or expression systems may be useful for expression in, engineering and/or modifying a cell.

It will be understood that throughout the present disclosure, the term “nucleic acid encoding a polypeptide” and/or “polynucleotide encoding a polypeptide” shall refer to a nucleic acid such as a nucleic acid molecule capable of encoding a polypeptide, a protein or a fragment thereof. Such nucleic acid may be open reading frames or genes, or fragments thereof.

Also provided herein is a kit of parts comprising:
a) a cell disclosed herein and optionally instructions for use; and/or
b) an expression system disclosed herein, wherein said expression system is for modifying a cell, and
   optionally the cell to be modified and/or instructions for use.

### Expression System Useful for Biofluorination

The present disclosure further relates to an expression system for expression in a cell, said system comprising:
a) a first nucleic acid comprising a first promoter and a fluorinase gene encoding a fluorinase (EC 2.5.1.63) capable of catalysing the formation of a C—F bond, said fluorinase gene being under the control of the first promoter;
b) optionally a second nucleic acid comprising a riboswitch, wherein transcription of the fluorinase gene from the first promoter is induced in the presence of an inducer, wherein said riboswitch is responsive to said inducer,
   wherein the fluorinase is the fluorinase of Methanosaeta sp. PtaU1.Bin055 as set forth in SEQ ID NO: 1 (FlA_{PtaU1}), or a functional variant thereof having at least 70% sequence homology, similarity or identity thereto, such as at least 71%, such as at least 72%, such as at least 73%, such as at least 74%, such as at least 75%, such as at least 76%, such as at least 77%, such as at least 78%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence homology, similarity or identity to SEQ ID NO: 1.

In some embodiments, the disclosure further relates to an expression system for expression in a cell, said system comprising:
a) a first nucleic acid comprising a first promoter and a fluorinase gene encoding a fluorinase (EC 2.5.1.63) capable of catalysing the formation of a C—F bond, said fluorinase gene being under the control of the first promoter;
b) optionally a second nucleic acid comprising a riboswitch, a second promoter and a gene encoding an activator of transcription, said gene being under the control of the second promoter, wherein transcription of the activator of transcription can be induced by an inducer, wherein the activator of transcription upon expression activates transcription from the first promoter, wherein the fluorinase is the fluorinase of Methanosaeta sp. PtaU1.Bin055 as set forth in SEQ ID NO: 1 (FlA_{PtaU1}), or a functional variant thereof having at least 70% sequence homology, similarity or identity thereto, such as at least 71%, such as at least 72%, such as at least 73%, such as at least 74%, such as at least 75%, such as at least 76%, such as at least 77%, such as at least 78%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence homology, similarity or identity to SEQ ID NO: 1.

The elements of the expression system, i.e. the first nucleic acid, the first promoter, the fluorinase gene encoding a fluorinase, the second nucleic acid, the riboswitch, the second promoter and the gene encoding an activator of transcription, may be as described herein.

The expression system is suitable for expression of the first nucleic acid and of the second nucleic acid in a cell, where the cell may be as described herein. In embodiments where the cell is a bacterial cell, preferably the cell is a Gram-negative bacterial cell. In other some embodiments, the cell is a bacterial cell of the genera Pseudomonas, Bacillus, Streptomyces, Vibrio or Escherichia. For example, the bacterial cell is selected from Pseudomonas putida, Pseudomonas fluorescens, Pseudomonas taiwanensis, Pseudomonas syringae, Pseudomonas stutzeri, Pseudomonas oleovorans, Pseudomonas mendocina, Bacillus subtilis, Bacillus cereus, Bacillus megaterium, Streptomyces albus, Streptomyces venezuelae, Streptomyces coelicolor, Vibrio natriegens and Escherichia coli. In particular embodiments, the cell is a Pseudomonas cell, such as a Pseudomonas putida cell, such as a Pseudomonas putida KT2440 cell.

The first and the second nucleic acids may be independently comprised in a vector or integrated in the genome of the cell. In some embodiments, the first and the second nucleic acids are provided in a vector, such as in one vector. In some embodiments, the first nucleic acid is provided in a vector, and the second nucleic acid is provided in another vector. In some embodiments, the first nucleic acid is provided in a vector and the second nucleic acid is integrated in the genome of the cell. In some embodiments, the second nucleic acid is provided in a vector and the first nucleic acid is integrated in the genome of the cell. In some embodiments, the first and the second nucleic acids are integrated in the genome of the cell.

In some embodiments, the second nucleic acid comprises a riboswitch, and the second nucleic acid and the first nucleic acid are the same. Thus the present disclosure further relates to an expression system for expression in a cell, said system comprising:
a) a first nucleic acid comprising a first promoter and a fluorinase gene encoding a fluorinase (EC 2.5.1.63) capable of catalysing the formation of a C—F bond, said fluorinase gene being under the control of the first promoter and wherein the transcription of the fluorinase gene can be induced by an inducer;
b) optionally a second nucleic acid comprising a riboswitch, wherein the fluorinase is the fluorinase of Methanosaeta sp. PtaU1.Bin055 as set forth in SEQ ID NO: 1 (FlA_{PtaU1}), or a functional variant thereof having at least 70% sequence homology, similarity or identity thereto, such as at least 71%, such as at least 72%, such as at least 73%, such as at least 74%, such as at least 75%, such as at least 76%, such as at least 77%, such as at least 78%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence homology, similarity or identity to SEQ ID NO: 1.

The elements of the expression system, i.e. the first nucleic acid, the first promoter, the fluorinase gene encoding a fluorinase, the second nucleic acid and the riboswitch may be as described herein.

The expression system is suitable for expression in a cell where the cell may be as described herein. In embodiments where the cell is a bacterial cell, preferably the cell is a Gram-negative bacterial cell. In other some embodiments, the cell is a bacterial cell of the genera Pseudomonas, Bacillus, Streptomyces, Vibrio or Escherichia. For example, the bacterial cell is selected from Pseudomonas putida, Pseudomonas fluorescens, Pseudomonas taiwanensis, Pseudomonas syringae, Pseudomonas stutzeri, Pseudomonas oleovorans, Pseudomonas mendocina, Bacillus subtilis, Bacillus cereus, Bacillus megaterium, Streptomyces albus, Streptomyces venezuelae, Streptomyces coelicolor, Vibrio natriegens and Escherichia coli. In particular embodiments, the cell is a Pseudomonas cell, such as a Pseudomonas putida cell, such as a Pseudomonas putida KT2440 cell.

The first nucleic acid may be comprised in a vector or integrated in the genome of the cell. In some embodiments, the first nucleic acid is provided in a vector. In some embodiments, the first nucleic acid is integrated in the genome of the cell.

The expression system is suitable for expression of the first nucleic acid and of the second nucleic acid in a cell, where the cell may be as described herein. In particular embodiments, the cell is a Pseudomonas cell, such as a Pseudomonas putida cell, such as a Pseudomonas putida KT2440 cell.

The first and the second nucleic acids may be independently comprised in a vector or integrated in the genome of the cell. In some embodiments, the first and the second nucleic acids are provided in a vector, such as in one vector. In some embodiments, the first nucleic acid is provided in a vector, and the second nucleic acid is provided in another vector. In some embodiments, the first nucleic acid is provided in a vector and the second nucleic acid is integrated in the genome of the cell. In some embodiments, the second nucleic acid is provided in a vector and the first nucleic acid is integrated in the genome of the cell. In some embodiments, the first and the second nucleic acids are integrated in the genome of the cell.

The present disclosure further relates to an expression system for expression in a cell, said system comprising:
a) a first nucleic acid comprising a first promoter, a fluorinase gene encoding a fluorinase (EC 2.5.1.63) capable of catalysing the formation of a C—F bond, and a gene encoding a phosphorylase (EC 2.4.2.28 and/or EC 2.4.2.1) such as FlB1 or DeoD, said phosphorylase being capable of catalysing the phosphorylation of a fluorinated adenosine, said fluorinase gene and phosphorylase gene being under control of the first promoter;
b) optionally a second nucleic acid comprising a riboswitch, a second promoter and a gene encoding an activator of transcription, said gene being under the control of the second promoter, wherein transcription of the activator of transcription can be induced by an inducer, wherein the activator of transcription upon expression activates transcription from the first promoter, wherein the fluorinase is the fluorinase of Methanosaeta sp. PtaU1.Bin055 as set forth in SEQ ID NO: 1 (FlA_{PtaU1}), or a functional variant thereof having at least 70% sequence homology, similarity or identity thereto, such as at least 71%, such as at least 72%, such as at least 73%, such as at least 74%, such as at least 75%, such as at least 76%, such as at least 77%, such as at least 78%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence homology, similarity or identity to SEQ ID NO: 1.

In other embodiments, the expression system comprises:
a) a first nucleic acid comprising a first promoter, a fluorinase gene encoding a fluorinase (EC 2.5.1.63) capable of catalysing the formation of a C—F bond, and a gene encoding a phosphorylase (EC 2.4.2.28 and/or EC 2.4.2.1) such as FlB1 or DeoD, said phosphorylase being capable of catalysing the phosphorylation of a fluorinated adenosine, said fluorinase gene and phosphorylase gene being under control of the first promoter;
b) optionally a second nucleic acid comprising a riboswitch, wherein the fluorinase is the fluorinase of Methanosaeta sp. PtaU1.Bin055 as set forth in SEQ ID NO: 1 (FlA_{PtaU1}), or a functional variant thereof having at least 70% sequence homology, similarity or identity thereto, such as at least 71%, such as at least 72%, such as at least 73%, such as at least 74%, such as at least 75%, such as at least 76%, such as at least 77%, such as at least 78%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence homology, similarity or identity to SEQ ID NO: 1.

Such expression systems comprising both a fluorinase gene and a gene encoding a phosphorylase when introduced in the cell allow fluorination and phosphorylation of compounds in the presence of inducer. The elements of the expression system, i.e. the first nucleic acid, the first promoter, the fluorinase gene encoding a fluorinase, and the phosphorylase gene encoding a phosphorylase, the second nucleic acid, the riboswitch, the second promoter and the gene encoding an activator of transcription, may be as described herein.

The present disclosure further relates to an expression system for expression in a cell, said system comprising:
a) a first nucleic acid comprising a first promoter, a fluorinase gene encoding a fluorinase (EC 2.5.1.63) capable of catalysing the formation of a C—F bond, and a gene encoding a nucleosidase (EC 3.2.2.9) such as Pfs, said nucleosidase being capable of catalysing the conversion of 5′-FDA to 5′-FDR, said fluorinase gene and nucleosidase gene being under control of the first promoter;
b) optionally a second nucleic acid comprising a riboswitch, a second promoter and a gene encoding an activator of transcription, said gene being under the control of the second promoter,
   wherein transcription of the activator of transcription can be induced by an inducer, wherein the activator of transcription upon expression activates transcription from the first promoter,
   wherein the fluorinase is the fluorinase of Methanosaeta sp. PtaU1.Bin055 as set forth in SEQ ID NO: 1 (FlA_{PtaU1}), or a functional variant thereof having at least 70% sequence homology, similarity or identity thereto, such as at least 71%, such as at least 72%, such as at least 73%, such as at least 74%, such as at least 75%, such as at least 76%, such as at least 77%, such as at least 78%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence homology, similarity or identity to SEQ ID NO: 1.

In other embodiments, the expression system comprises:
a) a first nucleic acid comprising a first promoter, a fluorinase gene encoding a fluorinase (EC 2.5.1.63) capable of catalysing the formation of a C—F bond, and a gene encoding a nucleosidase (EC 3.2.2.9) such as Pfs, said nucleosidase being capable of catalysing the conversion of 5′-FDA to 5′-FDR, said fluorinase gene and nucleosidase gene being under control of the first promoter;
b) optionally a second nucleic acid comprising a riboswitch, wherein the fluorinase is the fluorinase of Methanosaeta sp. PtaU1.Bin055 as set forth in SEQ ID NO: 1 (FlA_{PtaU1}), or a functional variant thereof having at least 70% sequence homology, similarity or identity thereto, such as at least 71%, such as at least 72%, such as at least 73%, such as at least 74%, such as at least 75%, such as at least 76%, such as at least 77%, such as at least 78%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence homology, similarity or identity to SEQ ID NO: 1.

Such expression systems comprising both a fluorinase gene and a gene encoding a nucleosidase when introduced in the cell allow production of a 5′-FDR in the presence of inducer. The elements of the expression system, i.e. the first nucleic acid, the first promoter, the fluorinase gene encoding a fluorinase, and the nucleosidase gene encoding a nucleosidase, the second nucleic acid, the riboswitch, the second promoter and the gene encoding an activator of transcription, may be as described herein.

The expression system is suitable for expression of the first nucleic acid and of the second nucleic acid in a cell, where the cell may be as described herein. In embodiments where the cell is a bacterial cell, preferably the cell is a Gram-negative bacterial cell. In other some embodiments, the cell is a bacterial cell of the genera Pseudomonas, Bacillus, Streptomyces, Vibrio or Escherichia. For example, the bacterial cell is selected from Pseudomonas putida, Pseudomonas fluorescens, Pseudomonas taiwanensis, Pseudomonas syringae, Pseudomonas stutzeri, Pseudomonas oleovorans, Pseudomonas mendocina, Bacillus subtilis, Bacillus cereus, Bacillus megaterium, Streptomyces albus, Streptomyces venezuelae, Streptomyces coelicolor, Vibrio natriegens and Escherichia coli. In particular embodiments, the cell is a Pseudomonas cell, such as a Pseudomonas putida cell, such as a Pseudomonas putida KT2440 cell.

The first and the second nucleic acids may be independently comprised in a vector or integrated in the genome of the cell. In some embodiments, the first and the second nucleic acids are provided in a vector, such as in one vector. In some embodiments, the first nucleic acid is provided in a vector, and the second nucleic acid is provided in another vector. In some embodiments, the first nucleic acid is provided in a vector and the second nucleic acid is integrated in the genome of the cell. In some embodiments, the second nucleic acid is provided in a vector and the first nucleic acid is integrated in the genome of the cell. In some embodiments, the first and the second nucleic acids are integrated in the genome of the cell.

### Expression System Useful for Production of Fluorinated Compounds

Further provided herein is an expression system for expression in a cell, said expression system comprising:
i. a nucleic acid encoding a fluorinase;
ii. a nucleic acid encoding a purine nucleoside phosphorylase (PNP) and/or phosphorylase;
iii. a nucleic acid encoding a nucleosidase;
iv. a nucleic acid encoding a kinase;
v. a nucleic acid encoding an isomerase;
vi. a nucleic acid encoding an aldolase;
vii. a nucleic acid encoding an alcohol dehydrogenase (ADH);
viii. an nucleic acid encoding an acetylating acetaldehyde dehydrogenase (AcAldh);
ix. a nucleic acid encoding a fluoroacetaldehyde dehydrogenase (F-Aldh); and/or
x. a nucleic acid encoding an acetyl-CoA synthetase (Acs),
   whereby said cell is capable of producing a fluorinated compound from a substrate and/or a first fluorinated compound, and/or catalysing formation of FAld, FAcCoA, FEtOH, and/or FAc and/or one or more derivatives thereof from said substrate and/or fluorinated compound and/or a derivative thereof.

The nucleic acid encoding the fluorinase, the PNP and/or the nucleosidase, may be as described elsewhere herein, such as in the section “Expression system useful for biofluorination” above.

The elements of the expression system, i.e. the nucleic acids encoding a fluorinase, a PNP, a nucleosidase, a kinase, an isomerase, an aldolase, an ADH, an AcAldh, an F-Aldh, and/or an Acs, may be as described herein.

The expression system is suitable for expression of one or more of the nucleic acids encoding a fluorinase, a PNP, a nucleosidase, a kinase, an isomerase, an aldolase, an ADH, an AcAldh, an F-Aldh, and/or an Acs in a cell, where the cell may be as described herein and in particular in the section “Cell” herein above. In preferred embodiments, the cell is a Pseudomonas cell, such as a Pseudomonas putida cell, such as a Pseudomonas putida KT2440 cell.

The nucleic acids may be independently comprised in a vector or integrated in the genome of the cell. In some embodiments the nucleic acids are provided in a vector, such as in one vector. In some embodiments, some of the nucleic acids are provided in a first vector, and those nucleic acids not comprised within said first vector is provided in one or multiple other vectors. In some embodiments, the nucleic acids are integrated in the genome of the cell.

In some embodiments, the expression system and/or nucleic acids further comprises one or more promoters. In other embodiments, said one or more promoters may be inducible and/or constitutive. In further some embodiments, said one or more promoters is or comprises a T7 promoter. T7 promoter may also herein be referred to at P_{T7} promoter.

In other embodiments, the expression system is as described herein, and transcription of one or more of the nucleic acids are:
i. controlled by the same promoter; and
ii. optionally said same promoter is induced by a riboswitch,
   whereby said cell is capable of producing a fluorinated compound from a substrate and/or a first fluorinated compound, respectively, and/or catalysing formation of FAld, FAc, FEtOH, and/or FAcCoA and/or one or more derivatives thereof from said fluorinated compound.

In some other embodiments, the expression system may further comprise one or more riboswitches. In some embodiments, transcription from the one or more promoters is induced in the presence of an inducer, wherein said one or more riboswitches are responsive to said inducer; whereby the cell is capable of expressing the one or more genes at least in the presence of said inducer.

In some embodiments, the one or more riboswitches is the fluoride-responsive riboswitch (FRSv1) as set forth in SEQ ID NO: 5, or a functional variant thereof having at least 90% homology, identity or similarity to SEQ ID NO: 5, such at least 91%, such as at least 92%, such as at least 93%, such as at least 94%, such as at least 95%, such as at least 96%, such as at least 97%, such as at least 98%, such as at least 99%, such as 100% homology, identity or similarity to SEQ ID NO: 5.

In further some embodiments, one or more of the nucleic acids encoding a fluorinase, a PNP, a nucleosidase, a kinase, an isomerase, an aldolase, an ADH, an AcAldh, an F-Aldh, and an Acs are comprised within the genome of the cell and/or within an expression system comprised within the cell, where the cell may be as described herein and in particular in the section “Cell” herein above. In preferred embodiments, the cell is a Pseudomonas cell, such as a Pseudomonas putida cell, such as a Pseudomonas putida KT2440 cell.

Methods for introducing vectors in a cell and methods for integrating nucleic acids in the genome of a cell are known in the art.

Methods for Production of Fluorinated Compounds and/or Products

The present disclosure relates to methods for in vivo fluorination of a substrate and methods for producing a fluorinated compound, such as FAld, FAcCoA, FEtOH, and/or FAc and/or one or more derivatives thereof from a first fluorinated compound. The cells, expression systems and nucleic acids disclosed herein are useful for microbial-based production of a fluorinated compound from a substrate and/or a first fluorinated compound, and microbial-based production of FAld and optionally FAcCoA, FEtOH, and/or FAc and/or one or more derivatives thereof from a fluorinated compound, such as a first fluorinated compound and/or a substrate.

Throughout the present disclosure, it will be understood that the cells disclosed herein can produce the fluorinated compound(s) and/or product(s) of interest mentioned herein when incubated in a medium, also known as a cultivation medium, under conditions that enable the cells to grow and produce the desired fluorinated compound(s) and/or product(s), either from a substrate and/or a first fluorinated product. From the description of the cells, also known as production organisms, production host cells, production hosts or host cells, provided herein, and knowing the type of host cell used, the skilled person will not have difficulties in identifying suitable cultivation media and/or conditions to achieve production of said fluorinated compounds and/or products. Thus, the type of medium used for propagating the cell will depend on the type of cell used for the method. The medium used is as known in the art.

In particular, the cultivation may be performed at temperatures and at pH suitable for supporting growth of the cells. The cultivation medium should include the required nutrients, and may be supplemented with precursors, inducer(s) and/or co-substrate(s) as applicable, i.e. at any stage prior to or during the cultivation. The time of cultivation will vary depending on which cell is used and what product may be produced by said cell, but can easily be adapted by the skilled person.

The titer(s) obtained with any of the methods of the present disclosure may be as described in the section “Titer” herein below.

The fluorinated compound(s) may be selected from 5′-FDA, 5′-FDR, 5′-FDRP, 5′-FDRuIP, FAld, FAc, FEtOH and FAcCoA.

### Method for In Vivo Fluorination of a Substrate

In one aspect, the present disclosure provides a method for in vivo fluorination of a substrate, comprising the steps of:
i) propagating a cell in a medium, said cell comprising:
   a) a first nucleic acid comprising a first promoter and a fluorinase gene encoding a fluorinase (EC 2.5.1.63) capable of catalysing the formation of a C—F bond, said fluorinase gene being under the control of the first promoter; and wherein the fluorinase is the fluorinase of Methanosaeta sp. PtaU1.Bin055 as set forth in SEQ ID NO: 1 (FlA_{PtaU1}), or a functional variant thereof having at least 70% sequence homology, similarity or identity thereto such as at least 75%, such as at least 80%, such as at least 85%, such as at least 90%, such as at least 95%, such as at least 100% sequence homology, similarity or identity to SEQ ID NO: 1;
   b) optionally a second nucleic acid comprising a second promoter and a gene encoding an activator of transcription, said gene being under the control of the second promoter, wherein transcription of the activator of transcription can be induced by an inducer, wherein the activator of transcription upon expression activates transcription from the first promoter;
ii) adding the inducer to the medium and incubating the cell in the presence of inducer and a co-substrate such as a fluoride salt,
   whereby transcription of the gene encoding an activator of transcription is induced,
   thereby inducing transcription of the fluorinase gene,
      thereby inducing fluorination of the substrate to yield a fluorinated compound.

In one embodiment, the method is a method for in vivo fluorination of a substrate, comprising the steps of:
i) propagating a cell in a medium, said cell comprising:
   a) a first nucleic acid comprising a first promoter and a fluorinase gene encoding a fluorinase (EC 2.5.1.63) capable of catalysing the formation of a C—F bond, said fluorinase gene being under the control of the first promoter and wherein the transcription of the fluorinase gene can be induced by an inducer; and wherein the fluorinase is the fluorinase of Methanosaeta sp. PtaU1.Bin055 as set forth in SEQ ID NO: 1 (FlA_{PtaU1}), or a functional variant thereof having at least 70% sequence homology, similarity or identity thereto, such as at least 75%, such as at least 80%, such as at least 85%, such as at least 90%, such as at least 95%, such as at least 100% sequence homology, similarity or identity to SEQ ID NO: 1;
   b) optionally a second nucleic acid comprising a riboswitch,
ii) adding the inducer to the medium and incubating the cell in the presence of inducer and a co-substrate such as a fluoride salt,
   whereby transcription of the fluorinase gene is induced,
   thereby inducing fluorination of the substrate to yield a fluorinated compound.

In this embodiment, the first nucleic acid and the second nucleic acid may be the same molecule, i.e. the riboswitch controls transcription from the first promoter directly.

In other embodiments, the method is a method for producing a phosphorylated and fluorinated compound, and the expression system comprises a gene encoding a phosphorylase (EC 2.4.2.28 and/or EC 2.4.2.1).

In one such embodiment, the method is a method for in vivo phosphorylation of a In one embodiment, the method is a method for in vivo fluorination and phosphorylation, comprising the steps of:
i) propagating a cell in a medium, said cell comprising:
   a) a first nucleic acid comprising a first promoter, a fluorinase gene encoding a fluorinase (EC 2.5.1.63) capable of catalysing the formation of a C—F bond, and a gene encoding a phosphorylase (EC 2.4.2.28 and/or EC 2.4.2.1) such as FlB1 or DeoD, said phosphorylase being capable of catalysing the phosphorylation of a fluorinated adenosine, said fluorinase gene and phosphorylase gene being under control of the first promoter; and wherein the fluorinase is the fluorinase of Methanosaeta sp. PtaU1.Bin055 as set forth in SEQ ID NO: 1 (FlA_{PtaU1}), or a functional variant thereof having at least 70% sequence homology, similarity or identity thereto, such as at least 75%, such as at least 80%, such as at least 85%, such as at least 90%, such as at least 95%, such as at least 100% sequence homology, similarity or identity to SEQ ID NO: 1;
   b) optionally a second nucleic acid comprising a riboswitch;
ii) adding the inducer to the medium and incubating the cell in the presence of inducer and a co-substrate such as a fluoride salt,
   whereby transcription of the genes encoding the fluorinase and the phosphorylase is induced,
      thereby inducing fluorination and phosphorylation of the substrate to yield a fluorinated and phosphorylated product.

In one embodiment, the method is a method for in vivo fluorination and phosphorylation, comprising the steps of:
i) propagating a cell in a medium, said cell comprising:
   a) a first nucleic acid comprising a first promoter, a fluorinase gene encoding a fluorinase (EC 2.5.1.63) capable of catalysing the formation of a C—F bond, and a gene encoding a phosphorylase (EC 2.4.2.28 and/or EC 2.4.2.1) such as FlB1 or DeoD, said phosphorylase being capable of catalysing the phosphorylation of a fluorinated adenosine, said fluorinase gene and phosphorylase gene being under control of the first promoter; and wherein the fluorinase is the fluorinase of Methanosaeta sp. PtaU1.Bin055 as set forth in SEQ ID NO: 1 (FlA_{PtaU1}), or a functional variant thereof having at least 70% sequence homology, similarity or identity thereto, such as at least 75%, such as at least 80%, such as at least 85%, such as at least 90%, such as at least 95%, such as at least 100% sequence homology, similarity or identity to SEQ ID NO: 1;
   b) a second nucleic acid comprising a riboswitch, a second promoter and a gene encoding an activator of transcription, said gene being under the control of the second promoter, wherein transcription of the activator of transcription can be induced by an inducer, wherein the activator of transcription upon expression activates transcription from the first promoter;
ii) adding the inducer to the medium and incubating the cell in the presence of inducer and a co-substrate such as a fluoride salt,
   whereby transcription of the gene encoding an activator of transcription is induced,
   thereby inducing transcription of the fluorinase gene and the phosphorylase gene,
      thereby inducing fluorination and phosphorylation of the substrate to yield a fluorinated and phosphorylated product.

In some embodiments, the inducer and the co-substrate are identical.

In some embodiments, the fluorinated substrate such as a fluorinated adenosine is provided in the medium. In other embodiments, the cell is capable of synthesising the fluorinated substrate which is to be phosphorylated.

In other embodiments, the method is a method for producing a 5′-FDR, and the expression system comprises a gene encoding a nucleosidase (EC 3.2.2.9).

In one embodiment, the method is a method for in vivo production of 5′-FDR, comprising the steps of:
i) propagating a cell in a medium, said cell comprising:
   a) a first nucleic acid comprising a first promoter, a fluorinase gene encoding a fluorinase (EC 2.5.1.63) capable of catalysing the formation of a C—F bond, and a gene encoding a nucleosidase (EC 3.2.2.9) such as Pfs, said nucleosidase being capable of catalysing the conversion of 5′-FDA to 5′-FDR, said fluorinase gene and nucleosidase gene being under control of the first promoter; and wherein the fluorinase is the fluorinase of Methanosaeta sp. PtaU1.Bin055 as set forth in SEQ ID NO: 1 (FlA_{PtaU1}), or a functional variant thereof having at least 70% sequence homology, similarity or identity thereto, such as at least 75%, such as at least 80%, such as at least 85%, such as at least 90%, such as at least 95%, such as at least 100% sequence homology, similarity or identity to SEQ ID NO: 1;
   b) optionally a second nucleic acid comprising a riboswitch;
ii) adding the inducer to the medium and incubating the cell in the presence of inducer and a co-substrate such as a fluoride salt,
   whereby transcription of the genes encoding the fluorinase and the nucleosidase is induced,
      thereby inducing the production of 5′-FDA and its conversion to 5′-FDR.

In one embodiment, the method is a method for in vivo production of 5′-FDR, comprising the steps of:
i) propagating a cell in a medium, said cell comprising:
   a) a first nucleic acid comprising a first promoter, a fluorinase gene encoding a fluorinase (EC 2.5.1.63) capable of catalysing the formation of a C—F bond, and a gene encoding a nucleosidase (EC 3.2.2.9) such as Pfs, said nucleosidase being capable of catalysing the conversion of 5′-FDA to 5′-FDR, said fluorinase gene and nucleosidase gene being under control of the first promoter; and wherein the fluorinase is the fluorinase of Methanosaeta sp. PtaU1.Bin055 as set forth in SEQ ID NO: 1 (FlA_{PtaU1}), or a functional variant thereof having at least 70% sequence homology, similarity or identity thereto, such as at least 75%, such as at least 80%, such as at least 85%, such as at least 90%, such as at least 95%, such as at least 100% sequence homology, similarity or identity to SEQ ID NO: 1;
   b) optionally a second nucleic acid comprising a riboswitch, a second promoter and a gene encoding an activator of transcription, said gene being under the control of the second promoter, wherein transcription of the activator of transcription can be induced by an inducer, wherein the activator of transcription upon expression activates transcription from the first promoter;
ii) adding the inducer to the medium and incubating the cell in the presence of inducer and a co-substrate such as a fluoride salt,
   whereby transcription of the gene encoding an activator of transcription is induced,
   thereby inducing transcription of the fluorinase gene and the nucleosidase gene,
      thereby inducing the production of 5′-FDA and its conversion to 5′-FDR.

In preferred embodiments, the method is for producing 5′-FDA and converting it to 5′-FDR.

In some embodiments, the inducer and the co-substrate are identical.

In one embodiment, the method is a method for in vivo fluorination of a substrate, comprising the steps of:
i) propagating a cell in a medium, said cell comprising:
   a) a first nucleic acid comprising a first promoter and a fluorinase gene encoding a fluorinase (EC 2.5.1.63) capable of catalysing the formation of a C—F bond, said fluorinase gene being under the control of the first promoter; and wherein the fluorinase is the fluorinase of Methanosaeta sp. PtaU1.Bin055 as set forth in SEQ ID NO: 1 (FlA_{PtaU1}), or a functional variant thereof having at least 70% sequence homology, similarity or identity thereto, such as at least 75%, such as at least 80%, such as at least 85%, such as at least 90%, such as at least 95%, such as at least 100% sequence homology, similarity or identity to SEQ ID NO: 1;
   b) optionally a second nucleic acid comprising a second promoter and a gene encoding an activator of transcription, said gene being under the control of the second promoter, wherein transcription of the activator of transcription can be induced by an inducer, wherein the activator of transcription upon expression activates transcription from the first promoter;
ii) adding the inducer to the medium and incubating the cell in the presence of inducer and a fluoride salt,
   whereby transcription of the gene encoding an activator of transcription is induced,
   thereby inducing transcription of the fluorinase gene,
      thereby inducing fluorination of the substrate to yield a fluorinated compound.

In one embodiment, the method is a method for in vivo fluorination of a substrate, comprising the steps of:
i) propagating a cell in a medium, said cell comprising:
   a) a first nucleic acid comprising a first promoter and a fluorinase gene encoding a fluorinase (EC 2.5.1.63) capable of catalysing the formation of a C—F bond, said fluorinase gene being under the control of the first promoter and wherein the transcription of the fluorinase gene can be induced by an inducer; and wherein the fluorinase is the fluorinase of Methanosaeta sp. PtaU1.Bin055 as set forth in SEQ ID NO: 1 (FlA_{PtaU1}), or a functional variant thereof having at least 70% sequence homology, similarity or identity thereto, such as at least 75%, such as at least 80%, such as at least 85%, such as at least 90%, such as at least 95%, such as at least 100% sequence homology, similarity or identity to SEQ ID NO: 1;
   b) optionally a second nucleic acid comprising a riboswitch,
ii) adding the inducer to the medium and incubating the cell in the presence of inducer and a fluoride salt,
   whereby transcription of the fluorinase gene is induced,
   thereby inducing fluorination of the substrate to yield a fluorinated compound.

In one embodiment, the method is a method for in vivo fluorination and phosphorylation of a substrate, comprising the steps of:
i) propagating a cell in a medium, said cell comprising:
   a) a first nucleic acid comprising a first promoter, a fluorinase gene encoding a fluorinase (EC 2.5.1.63) capable of catalysing the formation of a C—F bond, and a gene encoding a phosphorylase (EC 2.4.2.28 and/or EC 2.4.2.1) such as FlB1 or DeoD, said phosphorylase being capable of catalysing the phosphorylation of a fluorinated substrate, said fluorinase gene and phosphorylase gene being under control of the first promoter; and wherein the fluorinase is the fluorinase of Methanosaeta sp. PtaU1.Bin055 as set forth in SEQ ID NO: 1 (FlA_{PtaU1}), or a functional variant thereof having at least 70% sequence homology, similarity or identity thereto, such as at least 75%, such as at least 80%, such as at least 85%, such as at least 90%, such as at least 95%, such as at least 100% sequence homology, similarity or identity to SEQ ID NO: 1;
   b) optionally a second nucleic acid comprising a riboswitch;
ii) adding the inducer to the medium and incubating the cell in the presence of inducer and a fluoride salt,
   thereby inducing transcription of the fluorinase gene and the phosphorylase gene,
      thereby inducing fluorination and phosphorylation of the substrate to yield a fluorinated and phosphorylated product.

In one embodiment, the method is a method for in vivo fluorination and phosphorylation of a substrate, comprising the steps of:
i) propagating a cell in a medium, said cell comprising:
   a) a first nucleic acid comprising a first promoter, a fluorinase gene encoding a fluorinase (EC 2.5.1.63) capable of catalysing the formation of a C—F bond, and a gene encoding a phosphorylase (EC 2.4.2.28 and/or EC 2.4.2.1) such as FlB1 or DeoD, said phosphorylase being capable of catalysing the phosphorylation of a fluorinated substrate, said fluorinase gene and phosphorylase gene being under control of the first promoter; and wherein the fluorinase is the fluorinase of Methanosaeta sp. PtaU1.Bin055 as set forth in SEQ ID NO: 1 (FlA_{PtaU1}), or a functional variant thereof having at least 70% sequence homology, similarity or identity thereto, such as at least 75%, such as at least 80%, such as at least 85%, such as at least 90%, such as at least 95%, such as at least 100% sequence homology, similarity or identity to SEQ ID NO: 1;
   b) optionally a second nucleic acid comprising a riboswitch, a second promoter and a gene encoding an activator of transcription, said gene being under the control of the second promoter, wherein transcription of the activator of transcription can be induced by an inducer, wherein the activator of transcription upon expression activates transcription from the first promoter;
ii) adding the inducer to the medium and incubating the cell in the presence of inducer and a fluoride salt,
   whereby transcription of the gene encoding an activator of transcription is induced,
   thereby inducing transcription of the fluorinase gene and the phosphorylase gene,
      thereby inducing fluorination and phosphorylation of the substrate to yield a fluorinated and phosphorylated product.

In one embodiment, the cell comprises:
a) a first nucleic acid comprising a first promoter and a fluorinase gene encoding a fluorinase (EC 2.5.1.63) capable of catalysing the formation of a C—F bond, said fluorinase gene being under the control of the first promoter, wherein the fluorinase gene encodes the fluorinase of Methanosaeta sp. PtaU1.Bin055 as set forth in SEQ ID NO: 1 (FlA_{PtaU1}), or a functional variant thereof having at least 70% sequence homology, similarity or identity thereto, such as at least 75%, such as at least 80%, such as at least 85%, such as at least 90%, such as at least 95%, such as at least 100% sequence homology, similarity or identity to SEQ ID NO: 1, and wherein the first promoter is a T7 promoter;
   b) optionally a second nucleic acid comprising a fluoride-responsive riboswitch such as the FRS riboswitch from P. syringae (SEQ ID NO: 4), a second promoter such as the native promoter of the eriC^{F} gene of P. syringae or the P_{EM7} promoter, and a gene encoding an activator of transcription, wherein the activator of transcription is the T7 RNA polymerase, said gene being under the control of the second promoter, wherein transcription of the T7 RNA polymerase can be induced by fluoride, wherein T7 RNA polymerase upon expression activates transcription from the T7 promoter;
      wherein the cell is capable of expressing the activator of transcription and the fluorinase at least in the presence of fluoride. In some embodiments, the cell is a Pseudomonas putida cell, in particular a P. putida KT2440 cell, which may further comprise a mutation of a gene encoding a fluoride transporter resulting in a partial or total loss of activity of the fluoride transporter.

In one embodiment, the cell comprises:
a) a first nucleic acid comprising a first promoter and a fluorinase gene encoding a fluorinase (EC 2.5.1.63) capable of catalysing the formation of a C—F bond, said fluorinase gene being under the control of the first promoter and wherein the transcription of the fluorinase gene can be induced by an inducer; and wherein the fluorinase is the fluorinase of Methanosaeta sp. PtaU1.Bin055 as set forth in SEQ ID NO: 1 (FlA_{PtaU1}), or a functional variant thereof having at least 70% sequence homology, similarity or identity thereto, such as at least 75%, such as at least 80%, such as at least 85%, such as at least 90%, such as at least 95%, such as at least 100% sequence homology, similarity or identity to SEQ ID NO: 1;
b) optionally a second nucleic acid comprising a riboswitch,
   wherein the cell is capable of expressing the fluorinase at least in the presence of fluoride. In some embodiments, the cell is a Pseudomonas putida cell, in particular a P. putida KT2440 cell, which may further comprise a mutation of a gene encoding a fluoride transporter resulting in a partial or total loss of activity of the fluoride transporter.

In one embodiment, the cell comprises:
a) a first nucleic acid comprising a first promoter, a fluorinase gene encoding a fluorinase (EC 2.5.1.63) capable of catalysing the formation of a C—F bond, and a gene encoding a phosphorylase (EC 2.4.2.28 and/or EC 2.4.2.1) such as FlB1 or DeoD, said phosphorylase being capable of catalysing the phosphorylation of a fluorinated adenosine, said fluorinase gene and phosphorylase gene being under control of the first promoter; and wherein the fluorinase is the fluorinase of Methanosaeta sp. PtaU1.Bin055 as set forth in SEQ ID NO: 1 (FlA_{PtaU1}), or a functional variant thereof having at least 70% sequence homology, similarity or identity thereto, such as at least 75%, such as at least 80%, such as at least 85%, such as at least 90%, such as at least 95%, such as at least 100% sequence homology, similarity or identity to SEQ ID NO: 1;
b) optionally a second nucleic acid comprising a riboswitch;
   wherein the cell is capable of expressing the fluorinase and the phosphorylase at least in the presence of fluoride. In some embodiments, the cell is a Pseudomonas putida cell, in particular a P. putida KT2440 cell, which may further comprise a mutation of a gene encoding a fluoride transporter resulting in a partial or total loss of activity of the fluoride transporter.

In one embodiment, the cell comprises:
a) a first nucleic acid comprising a first promoter, a fluorinase gene encoding a fluorinase (EC 2.5.1.63) capable of catalysing the formation of a C—F bond, and a gene encoding a phosphorylase (EC 2.4.2.28 and/or EC 2.4.2.1) such as FlB1 or DeoD, said phosphorylase being capable of catalysing the phosphorylation of a fluorinated adenosine, said fluorinase gene and phosphorylase gene being under control of the first promoter; and wherein the fluorinase is the fluorinase of Methanosaeta sp. PtaU1.Bin055 as set forth in SEQ ID NO: 1 (FlA_{PtaU1}), or a functional variant thereof having at least 70% sequence homology, similarity or identity thereto, such as at least 75%, such as at least 80%, such as at least 85%, such as at least 90%, such as at least 95%, such as at least 100% sequence homology, similarity or identity to SEQ ID NO: 1;
b) optionally a second nucleic acid comprising a riboswitch, a second promoter and a gene encoding an activator of transcription, said gene being under the control of the second promoter, wherein transcription of the activator of transcription can be induced by an inducer, wherein the activator of transcription upon expression activates transcription from the first promoter;
   wherein the cell is capable of expressing the activator of transcription, the fluorinase and the phosphorylase at least in the presence of fluoride. In some embodiments, the cell is a Pseudomonas putida cell, in particular a P. putida KT2440 cell, which may further comprise a mutation of a gene encoding a fluoride transporter resulting in a partial or total loss of activity of the fluoride transporter.

In one embodiment, the cell comprises:
a) a first nucleic acid comprising a first promoter, a fluorinase gene encoding a fluorinase (EC 2.5.1.63) capable of catalysing the formation of a C—F bond, and a gene encoding a nucleosidase (EC 3.2.2.9) such as Pfs, said nucleosidase being capable of catalysing the conversion of 5′-FDA to 5′-FDR, said nucleosidase, said fluorinase gene and nucleosidase gene being under control of the first promoter; and wherein the fluorinase is the fluorinase of Methanosaeta sp. PtaU1.Bin055 as set forth in SEQ ID NO: 1 (FlA_{PtaU1}), or a functional variant thereof having at least 70% sequence homology, similarity or identity thereto, such as at least 75%, such as at least 80%, such as at least 85%, such as at least 90%, such as at least 95%, such as at least 100% sequence homology, similarity or identity to SEQ ID NO: 1;
b) optionally a second nucleic acid comprising a riboswitch;
   wherein the cell is capable of expressing the fluorinase and the nucleosidase at least in the presence of inducer. In some embodiments, the cell is a Pseudomonas putida cell, in particular a P. putida KT2440 cell, which may further comprise a mutation of a gene encoding a fluoride transporter resulting in a partial or total loss of activity of the fluoride transporter.

In one embodiment, the cell comprises:
a) a first nucleic acid comprising a first promoter, a fluorinase gene encoding a fluorinase (EC 2.5.1.63) capable of catalysing the formation of a C—F bond, and a gene encoding a nucleosidase (EC 3.2.2.9) such as Pfs, said nucleosidase being capable of catalysing the conversion of 5′-FDA to 5′-FDR, said nucleosidase, said fluorinase gene and nucleosidase gene being under control of the first promoter; and wherein the fluorinase is the fluorinase of Methanosaeta sp. PtaU1.Bin055 as set forth in SEQ ID NO: 1 (FlA_{PtaU1}), or a functional variant thereof having at least 70% sequence homology, similarity or identity thereto, such as at least 75%, such as at least 80%, such as at least 85%, such as at least 90%, such as at least 95%, such as at least 100% sequence homology, similarity or identity to SEQ ID NO: 1;
b) optionally a second nucleic acid comprising a riboswitch, a second promoter and a gene encoding an activator of transcription, said gene being under the control of the second promoter, wherein transcription of the activator of transcription can be induced by an inducer, wherein the activator of transcription upon expression activates transcription from the first promoter;
   wherein the cell is capable of expressing the activator of transcription, the fluorinase and the nucleosidase at least in the presence of inducer. In some embodiments, the cell is a Pseudomonas putida cell, in particular a P. putida KT2440 cell, which may further comprise a mutation of a gene encoding a fluoride transporter resulting in a partial or total loss of activity of the fluoride transporter.

In some embodiments, the inducer is a fluoride salt.

The cell is preferably as described herein; in particular, the cell may be a Pseudomonas cell, such as a Pseudomonas putida cell, such as a Pseudomonas putida KT2440 cell. The elements of the expression system, i.e. the first nucleic acid, the first promoter, the fluorinase gene encoding a fluorinase, the second nucleic acid, the riboswitch, the second promoter and the gene encoding an activator of transcription, may be as described herein.

The cell is preferably as described herein; in particular, the cell may be a Pseudomonas cell, such as a Pseudomonas putida cell, such as a Pseudomonas putida KT2440 cell. The elements of the expression system, i.e. the first nucleic acid, the first promoter, the fluorinase gene encoding a fluorinase, and the riboswitch, may be as described herein.

The cell is preferably as described herein; in particular, the cell may be a Pseudomonas cell, such as a Pseudomonas putida cell, such as a Pseudomonas putida KT2440 cell. The elements of the expression system, i.e. the first nucleic acid, the first promoter, the gene encoding a phosphorylase, the second nucleic acid, the riboswitch, the second promoter and the gene encoding an activator of transcription, may be as described herein.

The cell is preferably as described herein; in particular, the cell may be a Pseudomonas cell, such as a Pseudomonas putida cell, such as a Pseudomonas putida KT2440 cell. The elements of the expression system, i.e. the first nucleic acid, the first promoter, the fluorinase gene encoding a fluorinase, the gene encoding a phosphorylase, the second nucleic acid, the riboswitch, the second promoter and the gene encoding an activator of transcription, may be as described herein.

The substrate and co-substrate may be as described herein.

The method may be performed by using an expression system suitable for expression of the first nucleic acid and of the second nucleic acid in a cell, as described herein. The method may be performed by using an expression system suitable for expression of the first nucleic acid in a cell, as described herein.

The fluorinated compound(s) may be selected from 5′-FDA, 5′-FDR, 5′-FDRP, 5′-FDRuIP, FAld, FAc, FEtOH and FAcCoA.

### Inducer

The in vivo fluorination of a substrate as described herein may be achieved by adding an inducer, which activates transcription of the gene encoding the activator of transcription. This in turn induces transcription of the fluorinase gene, as explained in detail herein above. As a result, the fluorinase is expressed, and can fluorinate the substrate in the presence of the appropriate co-substrate. In some embodiments, the co-substrate and the inducer are identical.

The inducer may be provided in the form of salts. Preferred salts are soluble salts. In some embodiments, the reaction is a fluorination reaction and the inducer is a fluoride salt. Preferred fluoride salts are NaF or KF; less soluble salts such as CaF₂ may also be used.

The inducer may be added at a concentration of at least 0.05 mM, such as at least 0.1 mM, such as at least 0.2 mM, such as at least 0.25 mM, such as at least 0.3 mM, such as at least 0.4 mM, such as at least 0.5 mM, such as at least 0.6 mM, such as at least 0.7 mM, such as at least 0.75 mM, such as at least 0.8 mM, such as at least 0.9 mM, such as at least 1 mM, such as at least 2 mM, such as at least 3 mM, such as at least 4 mM, such as at least 5 mM, such as at least 6 mM, such as at least 7 mM, such as at least 8 mM, such as at least 9 mM, such as at least 10 mM, such as at least 11 mM, such as at least 12 mM, such as at least 13 mM, such as at least 14 mM, such as at least 15 mM, such as at least 20 mM, such as 25 mM or more.

The time of adding the inducer may be optimised as is known in the art, and may depend on the choice of cell and/or on the choice of the fluorinase gene. In some embodiments, the inducer is added at culture onset or in mid-exponential phase. It may occur that the presence of inducer will inhibit or slow cell growth, why in some embodiments it may be advantageous to propagate the cells until mid-exponential phase prior to adding the inducer.

### Co-Substrate

The choice of co-substrate will typically be dictated by the type of in vivo reaction which is to be performed. In embodiments where in vivo fluorination of a substrate is desired, the co-substrate is fluoride.

The co-substrates may be provided in the form of salts. Preferred salts are soluble salts. In some embodiments, the reaction is a fluorination reaction and the co-substrate is a fluoride salt. Preferred fluoride salts are NaF or KF; less soluble salts such as CaF₂ may also be used.

The co-substrate, such as NaF or KF, may be added at a concentration of at least 0.05 mM, such as at least 0.1 mM, such as at least 0.2 mM, such as at least 0.25 mM, such as at least 0.3 mM, such as at least 0.4 mM, such as at least 0.5 mM, such as at least 0.6 mM, such as at least 0.7 mM, such as at least 0.75 mM, such as at least 0.8 mM, such as at least 0.9 mM, such as at least 1 mM, such as at least 2 mM, such as at least 3 mM, such as at least 4 mM, such as at least 5 mM, such as at least 6 mM, such as at least 7 mM, such as at least 8 mM, such as at least 9 mM, such as at least 10 mM, such as at least 11 mM, such as at least 12 mM, such as at least 13 mM, such as at least 14 mM, such as at least 15 mM, such as at least 20 mM, such as 25 mM or more.

The time of adding the co-substrate may be optimised as is known in the art, and may depend on the choice of cell and/or on the choice of the fluorinase gene. In some embodiments, the co-substrate is added at culture onset or in mid-exponential phase. It may occur that the presence of co-substrate will inhibit or slow cell growth, why in some embodiments it may be advantageous to propagate the cells until mid-exponential phase prior to adding the co-substrate. In other embodiments, the co-substrate is present in the medium used for propagation from the beginning of the culture and/or cultivation.

In some embodiments, the inducer is the co-substrate.

### Duration of Incubation

Once fluoride, the inducer and/or co-substrate has been added to the medium, the cell is incubated for a duration such that fluorination of the substrate is achieved to obtain the desired fluorinated compound.

The duration of the incubation may depend on the choice of cell and of the type of reaction. In some embodiments, the cell is incubated in the presence of inducer for at least 4 hours, such as at least 8 hours, such as at least 12 hours, such as at least 16 hours, such as at least 20 hours, such as at least 24 hours, such as at least 36 hours, such as at least 48 hours, such as at least 72 hours, or more. Such incubation times, which are relatively short, are particularly relevant for embodiments where the cell is a bacterial cell, such as a P. putida cell, or a eukaryotic cell such as a yeast cell. In embodiments where the cell is a eukaryotic cell such as an insect cell, a mammalian cell or a plant cell, longer incubation times may be required. The skilled person knows how to determine which incubation times are suitable.

### Methods for Bioproduction of Fluorinated Compounds

In one aspect, the present disclosure provides a method for production of FAld and optionally FAcCoA, FEtOH, and/or FAc and/or one or more derivatives thereof from a fluorinated compound selected from 5′-fluoro-5′-deoxy-D-ribose 1-phosphate (5′-FDRP) and/or (3R,4S)-5′-fluoro-5′-deoxy-D-ribulose-1-phosphate (5′-FDRuIP), said method comprising the steps of:
i. providing a cell, said cell expressing:
   a. an isomerase (EC 5.3.1.23); and
   b. an aldolase (EC 4.1.2.62);
whereby said cell is capable of catalysing formation of FAld and optionally FEtOH; and
   c. optionally, said cell further expressing:
      I. an acetylating acetaldehyde dehydrogenase (AcAldh, EC 1.2.1.10); and/or
      II. a fluoroacetaldehyde dehydrogenase (F-Aldh, EC 1.2.1.69), and/or
         an acetyl-CoA synthetase (Acs, EC 6.2.1.1); and
ii. propagating said cell in a medium, optionally wherein the medium comprises the fluorinated compound or a compound such as a substrate which can be converted to the fluorinated compound by said cell;
   whereby FAld and optionally FAcCoA, FEtOH, and/or FAc and/or one or more derivatives thereof is produced.

The cell is preferably as described herein, for example in the section “Cell”; in particular, the cell may be a Pseudomonas cell, such as a Pseudomonas putida cell, such as a Pseudomonas putida KT2440 cell. In some embodiments, the cell comprises an expression system. The elements of said expression system may be as described herein, for example in the section “Expression systems” and in particular in the section “Expression system useful for production of fluorinated”.

In some embodiments, the method further comprises a step of fluorinating a substrate, i.e. producing a fluorinated compound from a substrate. In further some embodiments, the cell may further express a fluourinase for producing a fluorinated compound from a substrate, such as 5′-FDA from SAM and fluoride, for example as described herein above in the sections “Method for in vivo fluorination of a substrate” and/or “Fluorinase”. In addition to said fluorinase, the cell may further express a phosphorylase and/or PNP or a nucleosidase and a kinase for producing a fluorinated compound from said substrate and/or for converting a fluorinated compound into a phosphorylated and fluorinated compound.

Said substrate may be as defined in the section “Substrates” herein above. Thus, fluoride may be provided to the cell, such as supplemented to the medium. In some embodiments, fluoride is provided as a fluoride salt, such as NaF and/or KF. In some embodiments, NaF and/or KF is added at a concentration of at least 0.05 mM, such as at least 0.1 mM, such as at least 0.2 mM, such as at least 0.25 mM, such as at least 0.3 mM, such as at least 0.4 mM, such as at least 0.5 mM, such as at least 0.6 mM, such as at least 0.7 mM, such as at least 0.75 mM, such as at least 0.8 mM, such as at least 0.9 mM, such as at least 1 mM, such as at least 2 mM, such as at least 3 mM, such as at least 4 mM, such as at least 5 mM, such as at least 6 mM, such as at least 7 mM, such as at least 8 mM, such as at least 9 mM, such as at least 10 mM, such as at least 11 mM, such as at least 12 mM, such as at least 13 mM, such as at least 14 mM such as at least 15 mM, such as at least 20 mM, such as 25 mM or more.

In some embodiments, said fluoride may be added at the culture onset or in mid-exponential phase. In further some embodiments, the cell is incubated in the presence of fluoride for at least 4 hours, such as at least 8 hours, such as at least 12 hours, such as at least 16 hours, such as at least 20 hours, such as at least 24 hours, such as at least 36 hours, such as at least 48 hours, or more.

In some embodiments, the method is performed in vitro, for example cell extracts are prepared from the cells. The skilled person will not have difficulties in identifying suitable methods for preparing cell extracts. In other embodiments, the method is performed in vivo.

In some embodiments, the medium may comprise a substrate, said substrate may be as defined in the section “Substrates” herein above.

### Titer

In some embodiments, where the cell is capable of expressing a fluorinase as described herein, 5′-FDA is produced with a titer of at least 1 μM.

In some embodiments, where the cell is capable of expressing the fluorinase of Methanosaeta sp. PtaU1.Bin055 as set forth in SEQ ID NO: 1 (FlA_{PtaU1}) or a functional variant thereof having at least 70% sequence homology, similarity or identity thereto, such as at least 75%, such as at least 80%, such as at least 85%, such as at least 90%, such as at least 95%, such as at least 100% sequence homology, similarity or identity to SEQ ID NO: 1, 5′-FDA is produced with a titer of at least 1 μM.

In some embodiments, where the cell is capable of expressing the fluorinase of Methanosaeta sp. PtaU1.Bin055 as set forth in SEQ ID NO: 1 (FlA_{PtaU1}), 5′-FDA is produced with a titer of at least 1 μM.

In some embodiments, where the cell is capable of expressing a fluorinase as described above or below herein, 5′-FDA is produced with a titer of 0.1-20 μM, for example 0.25-15 μM, such as 0.5-12.5 μM, for example 0.75-10 μM, such as 1-8 μM, for example 1.5-7.5 μM, such as 1.5-7 μM, for example 1.5-6.5 μM, such as 1.5-6 μM, for example 2-5 μM.

In some embodiments, where the cell is capable of expressing the fluorinase of Methanosaeta sp. PtaU1.Bin055 as set forth in SEQ ID NO: 1 (FlA_{PtaU1}) or a functional variant thereof having at least 70% sequence homology, similarity or identity thereto, such as at least 75%, such as at least 80%, such as at least 85%, such as at least 90%, such as at least 95%, such as at least 100% sequence homology, similarity or identity to SEQ ID NO: 1, 5′-FDA is produced with a titer of 0.1-100 μM, for example 0.25-50 μM, such as 0.5-25 μM, for example 0.75-12.5 μM, such as 1-10 μM, for example 1.5-7.5 μM, such as 1.5-7 μM, for example 1.5-6.5 μM, such as 1.5-6 μM, for example 2-5 μM.

In some embodiments, the titer of 5′-FDA is at least 0.1 μM, such as at least 0.2 μM, such as at least 0.5 μM, such as at least 0.75 μM, such as 1 μM, such as at least 1.5 μM, such as at least 2 μM, such as at least 2.5 μM, such as at least 3 μM, such as at least 3.5 μM, such as at least 4 μM, such as at least 4.5 μM, such as at least 5 μM, such as at least 5.5 μM, such as at least 6 μM, such as at least 6.5 μM, such as at least 7 μM, such as at least 7.5 μM, such as at least 8 μM, such as at least 8.5 μM, such as at least 9 μM, such as at least 9.5 μM, such as at least 10 μM, such as at least M, such as at least 20 μM, such as at least 25 μM, such as at least 30 μM, such as at least 35 μM, such as at least 40 μM, such as at least 45 μM, such as at least 50 μM, such as at least 55 μM, such as at least 60 μM, such as at least 70 μM, such as at least 80 μM, such as at least 90 μM, such as at least 100 μM, or more.

In some embodiments, the titer of 5′-FDA is 0.1-20 μM, for example 0.25-15 μM, such as 0.5-12.5 μM, for example 0.75-10 μM, such as 1-8 μM, for example 1.5-7.5 μM, such as 1.5-7 μM, for example 1.5-6.5 μM, such as 1.5-6 μM, for example 2-5 μM.

In some embodiments, 5′-FDR, 5′-FDRP, 5′-FDRuIP, FAld, FAcCoA, FEtOH, and/or FAc, and/or one or more derivatives thereof is produced with a titer of at least 0.01 mM, such as at least 0.02 mM, such as at least 0.05 mM, such as at least 0.1 mM, such as at least 0.2 mM, such as at least 0.3 mM, such as at least 0.4 mM, such as at least 0.5 mM, such as at least 0.7 mM, such as at least 0.8 mM, such as at least 0.9 mM, such as at least 1 mM, such as at least 1.5 mM, such as at least 2 mM, such as at least 2.5 mM, such as at least 3 mM, such as at least 3.5 mM, such as at least 4 mM, such as at least 4.5 mM, such as at least 5 mM, such as at least 5.5 mM, such as at least 6 mM, such as at least 6.5 mM, such as at least 7 mM, such as at least 7.5 mM, such as at least 8 mM, such as at least 8.5 mM, such as at least 9 mM, such as at least 9.5 mM, such as at least 10 mM, such as at least 10.5 mM, such as at least 11 mM, such as at least 13 mM, such as at least 15 mM, such as at least 20 mM, such as at least 25 mM or more.

### Compound Recovery

The method disclosed herein may further comprise a step of recovering the compound, i.e. the fluorinated compound that has either been produced from a substrate or a first fluorinated compound in a method as described herein above. The fluorinated compound may be selected from 5′-FDA, 5′-FDR, 5′-FDRP, 5′-FDRuIP, FAld, FAc, FEtOH and FAcCoA.

The product will typically be present intracellularly and can be recovered as is known in the art, for example by solvent extraction from the cells (e.g. with an ethanol solution or a methanol solution). Preparative liquid chromatography (LC) can also be used to recover the product.

In some embodiments, the method further comprises a step of converting the fluorinated compound to a downstream product, said downstream product being fluorinated and/or non-fluorinated.

### Compounds and Compositions

Also provided herein is a fluorinated compound obtainable by the methods described herein, and/or compositions comprising a fluorinated compound obtainable by the methods described herein. The fluorinated compound may be selected from 5′-FDA, 5′-FDR, 5′-FDRP, 5′-FDRuIP, FAld, FAc, FEtOH and FAcCoA.

### Methods for Manufacturing a Fluorinated Compound of Interest

Also provided herein is a method for manufacturing a fluorinated compound of interest, said method comprising the steps of:
i) providing a fluorinated compound by the methods described herein; and
ii) optionally converting said fluorinated compound to the fluorinated compound of interest.

The fluorinated compound of interest may be a downstream product obtained by the methods described herein above.

In some embodiments, the fluorinated compound obtained by the methods described herein can be used directly, i.e. without further modification.

In other embodiments, however, the fluorinated compound is further converted into a fluorinated compound of interest. Indeed, the fluorinated compound obtained by the present method may be used as a drug precursor, a building block and/or a precursor of polymers. The methods of manufacturing a fluorinated compound of interest may thus comprise a step of converting the compound obtained by the present methods into a compound of interest, such as a drug or a therapeutic compound. The compound of interest may be a fluorinated nucleoside, a deoxy-fluoronucleoside, a fluoro-ribose phosphate, a chlorinated nucleoside, a deoxy-chloronucleoside, a brominated nucleoside or a deoxy-bromonucleoside. The compound of interest could be a chlorinated or brominated or iodinated nucleoside. The compound of interest could be transformed into a different halogenated compound of interest by methods known in the art, such as by a transhalogenation reaction, for example, the fluorinase can convert a fluorinated nucleoside into a chlorinated nucleoside by incubation of the fluorinated nucleoside in the presence of chloride.

Also provided herein is the use of a fluorinated compound obtainable by the present methods or of a composition comprising such compound as a medicament or a building block or a precursor of polymers.

The fluorinated compound and/or fluorinated compound of interest may be selected from 5′-FDA, 5′-FDR, 5′-FDRP, 5′-FDRuIP, FAld, FAc, FEtOH and FAcCoA. In some embodiments, the fluorinated compound is 5′-FDA.

### Uses

Further disclosed herein is, the use of a polypeptide as set forth in SEQ ID NO: 1 or a functional variant thereof having at least 70% sequence homology, similarity or identity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology, identity or similarity to SEQ ID NO: 1, for catalysing fluorination of a substrate.

Further disclosed herein is the use of a polypeptide as set forth in SEQ ID NO: 1 or a functional variant thereof having at least 70% sequence homology, similarity or identity thereto, such as at least 75%, such as at least 80%, such as at least 85%, such as at least 90%, such as at least 95%, such as at least 100% sequence homology, similarity or identity to SEQ ID NO: 1, in a method for producing FAld and optionally FAc, FEtOH and/or FAcCoA, and/or one or more derivatives thereof.

In some embodiments, the polypeptide comprises or consists of the sequence as set forth in SEQ ID NO: 1, with the exception that at the most 30 residues are mutated, such as at the most 29 residues, for example at the most 28 residues, such as at the most 27 residues, for example at the most 26 residues, for example at the most 25 residues, such as at the most 20 residues, for example at the most 15 residues, such as at the most 10 residues, for example 5 residues, or less. In some embodiments, fluorination is performed in vitro or in vivo. In some embodiments, the substrate is as described “Substrates” herein above, or anywhere else herein.

Also provided herein is the use of nucleic acids, expression systems and/or cells disclosed anywhere herein such as in “Expression system” or “Cell” herein above, for the fluorination of a substrate, and/or production of FAld and optionally FAc, FEtOH and/or FAcCoA, and/or one or more derivatives thereof.

### EXAMPLES

### Example 1—FAld, FAc and FAcCoA Production

### Example 1.1—Materials and Methods

### Bacterial Strains, Plasmids, and Culture Conditions

P. putida KT2440 was grown at 30° C. and routinely cultured in LB medium or on LB agar plates according to standard protocols (Sambrook et al. 2000) unless otherwise stated. DeBont minimal medium containing 5 g L⁻¹ of glucose as carbon source (Hartmans et al. 1989) was used for all the expression and production assays. E. coli DH5α was used for cloning and plasmid maintenance and was grown in LB at 37° C.

Antibiotics and other supplements were used at the following concentrations: chloramphenicol (Cm) 30 μg·mL⁻¹, kanamycin (Km) 50 μg·mL⁻¹, gentamicin (Gm) 10 μg·mL⁻¹, and streptomycin (St) 100 μg·mL⁻¹. 300 mM sucrose was used for preparing electrocompetent P. putida cells. Sodium fluoride (NaF) was purchased from Sigma-Aldrich (201154).

### Plasmids and Strains Construction

Table 1, 2 and/or 3 list genes, plasmids and strains used in the example. Plasmid pFB·PT7flA1pfsBtkin was cloned by USER cloning, using as templates plasmid pFB·1F1 for the codon optimized fluorinase flA1 (Streptomyces sp. MA37 fluorinase, i.e. fIA1_{MA37}, SEQ ID NO: 32), genomic DNA of E. coli MG1655 for the nucleosidase pfs, and pET28-Btisomerase-Nterm for the kinase. Fragments were amplified using primers containing uracil, as described before. The amplified fragments were digested with DpnI and 100 ng of the corresponding DNA fragments were mixed with the USER enzyme in a final volume of 10 μl, and incubated at 37° C. for 15 minutes, 24° C. for 15 minutes, and 10° C. for 15 minutes. 5 μl of this mix was subsequently transformed by heat-shock into DH5a chemically competent cells and plated into LB agar plates with the corresponding antibiotics and incubated at 37° C. overnight. The correct constructions were checked by colony PCR and sequencing.

Ald^{S. coe} (aldSc) was codon optimized for P. putida and synthetized by Genecust, while Iso^{B. thu} (isoBt), Ald^{B. thu} (aldBt), and Kin^{B. thu} (kinBt) were gifted by Andrew D. Hanson's lab. The genes and backbone were amplified from their respective plasmids using 1U Phusion U Hot Start DNA polymerase (Thermo Scientific, F555S), HF Buffer, 0.5 μM forward and reverse Primer, 20 mM dNTPs in a 50 μl reaction. Specific amplification and length was confirmed via gel electrophoresis. PCR products were gel purified using a commercial kit (Machery Nagel, 740609.50), and cloned into vector pS231T7, following the USER cloning approach (Salomonsen et al. 2013), by mixing 100 ng of both linearized insert and vector with 1 U USER enzyme (NEB, M5508) and T4 ligase buffer. Subsequently, the samples are incubated for 25 min at 37° C., then 25 min at 25° C. and kept at 10° C. Assembled DNA fragments were transformed into E. coli DH5a and clones checked for correct insertion via colony PCR. Correct clones were sequence-verified and transformed into E. coli BL21-DE3 cells for protein expression.

For Iso^{S. cat} (isoSc), that was offered by David O'Hagan's lab, the gene was obtained by digesting the original plasmid with Fast Digest restriction enzymes (Thermo Scientific) XbaI/XhoI, and the backbone with XbaI/SalI. Both fragments were ligated using 1 U T4 DNA Ligase (Thermo Scientific) and further transformed into E. coli DH5a.

Acetaldehyde dehydrogenase (Aldh) and Acetyl-CoA synthetase (Acs) genes were cloned using genomic DNA from indicated organisms, which was isolated using standard phenol-chloroform extraction protocols. Aldh genes (Table 2) and Acs genes (Table 3) were amplified using 1 U Phusion High fidelity DNA polymerase (Thermo Scientific, F530), HF or GC Buffer, each 0.5 μM forward and reverse Primer, 20 mM dNTPs each and 3% DMSO in a 50 μl reaction. Thermal cycling program was 94° C. 2 min, 35 cycles of [94° C. 30 s-51° C. 30 s, 72° C. 2 min], 72° C. 7 min.

Specific amplification and length (1-3 kb) was confirmed via gel electrophoresis, PCR products were gel purified using commercial kits (Machery Nagel, 740609.50), digested with Fast Digest Restriction enzymes (Thermo Scientific) and ligated to the linearized pET-Duet1 vector in frame with the HIS tag (a gift from Morten Nørholm, DTU). Ligated DNA fragments were transformed into E. coli DH5a and clones checked for correct insertion via restriction digestion. Correct HIS-tagged clones were sequence-verified and transformed into E. coli BL21-DE3 cells for protein expression.

TABLE 1
Bacterial strains and plasmids.

| | | |
|---|---|---|
| | | Reference |
| Strain | Relevant characteristics^{a} | or source |

Escherichia coli

| | | |
|---|---|---|
| DH5α | Cloning host; F⁻ λ⁻ endA1 glnX44(AS) thiE1 recA1 | Lab stock |
| | relA1 spoT1 gyrA96(Nal^{R}) rfbC1 deoR nupG | |
| | Φ80(lacZΔM15) Δ(argF-lac)U169 hsdR17(r_{K}⁻ m_{K}⁺) | |
| BL21(DE3) | Protein expression host | Lab stock |
| MG1655 | Source for the nucleosidase pfs | Lab stock |

Pseudomonas putida

| | | |
|---|---|---|
| P. putida | Wild-type strain, derived from P. putida mt-2 | Bagdasarian |
| KT2440 | (Worsey and Williams, 1975) cured of the TOL | et al. (1981) |
| | plasmid pWW0 | |
| P. putida | P. putida KT2440 derivative with T7 RNA | Calero et |
| KT2440::FRS- | polymerase integrated after glmS gene | al. 2020 |
| T7RNAP | | |

Plasmids

| | | |
|---|---|---|
| pS231T7 | Derivative of vector pSEVA231 containing the T7 | Volke et |
| | promoter; KmR | al. 2020 |
| pFB•1F1 | pSEVA231-T7pr derived, PT7-fIA1 construction. | Calero et |
| | KmR (SEQ ID NO: 32) | al. 2020 |
| pFB•PT7fIA1pfsBtkin | Plasmid derived from pFB•1F1 containing the | This work |
| | nucleosidase pfs (SEQ ID NO: 16) and the kinase | |
| | kinBt (SEQ ID NO: 34). | |
| pFB•PT7AldIsoPfsKin | Derivative of the vector pS231T7 carrying the | This work |
| | aldolase aldSc (SEQ ID NO: 20) from S. coelicolor, | |
| | the isomerase isoSc (SEQ ID NO: 38) from S. | |
| | cattleya, the nucleosidase pfs (SEQ ID NO: 16) from | |
| | E. coli and the kinase kinBt (SEQ ID NO: 34) from | |
| | B. thuringiensis. | |
| pFB•PT7AldIsoFIB1 | Derivative of the vector pS231T7 carrying the | This work |
| | aldolase aldSc (SEQ ID NO: 20) from S. coelicolor, | |
| | the isomerase isoSc (SEQ ID NO: 38) from S. | |
| | cattleya, and the phosphorylase flB1 (SEQ ID NO: | |
| | 13) from Streptomyces sp. MA37 | |
| pEHISTEV- | Expression vector derived from a pET vector, | CSIC - Molecular |
| isoSc | carrying the isomerase isoSc (SEQ ID NO: 38) from | Environmental |
| | S. cattleya | Microbiology |
| | | Laboratory |
| pET28-isoBt | Derivative of the vector pET28 carrying the | Beaudoin 2018 |
| | isomerase isoBt (SEQ ID NO: 36) from B. | |
| | thuringiensis | |
| pET28-aldBt | Derivative of the vector pET28 carrying the aldolase | Beaudoin 2018 |
| | aldBt (SEQ ID NO: 18) from B. thuringiensis | |
| pFB- | Derivative of the vector pS231T7 carrying the | This work |
| PT7isoSc | isomerase isoSc (SEQ ID NO: 38) from S. cattleya | |
| pFB- | Derivative of the vector pS231T7 carrying the | This work |
| PT7isoBt | isomerase isoBt (SEQ ID NO: 36) from B. | |
| | thuringiensis | |
| pFB- | Derivative of the vector pS231T7 carrying the | This work |
| PT7aldSc | aldolase aldSc (SEQ ID NO: 20) from S. coelicolor | |
| pFB- | Derivative of the vector pS231T7 carrying the | This work |
| PT7aldBt | aldolase aldBt (SEQ ID NO: 18) from B. | |
| | thuringiensis | |
| pET-Duet1- | Expression vector, carrying the mhpF gene from E. | This work |
| EC_mhpF | coli (Acetaldehyde Dehyrogenase, acetylating) | |
| pFB-Duet1- | Expression vector, carrying the adhE gene from E. | This work |
| EC_adhE | coli (Alcohol−, Aldehyde Dehyrogenase) | |
| pFB-Duet1- | Expression vector, carrying the hdhA (SEQ ID NO: | This work |
| PA4022 | 24) gene from P. aerigunosa (Aldehyde | |
| | Dehydrogenase) | |
| pET-Duet1- | Expression vector, carrying the exaC (SEQ ID NO: | This work |
| PA1984 | 22) gene from P. aerigunosa (NAD+ dependent | |
| | Aldehyde Dehydrogenase) | |
| pFB-Duet1- | Expression vector, carrying the Moth_1776 (SEQ | This work |
| MT_1776 | ID NO: 28) gene from M. thermoacetica | |
| | (Acetaldehyde Dehydrogenase) | |
| pFB-Duet1- | Expression vector, carrying the adhE gene from C. | This work |
| CT_adhE | thermocellum (bifunctional acetaldehyde-CoA/ | |
| | alcohol dehydrogenase) | |
| pFB-Duet1- | Expression vector, carrying the eutE (SEQ ID NO: | This work |
| EC_EutE | 26) gene from E. coli (putative aldehyde | |
| | dehydrogenase, Ethanolamine utilization protein) | |
| pFB-Duet1- | Expression vector, carrying the adhE gene from S. | This work |
| SC_adhE | cattleya | |
| pET-Duet1 - | Expression vector, AcsA gene from B. subtilis | This work |
| BS-AcsA | | |
| pET-Duet1 - | Expression vector, Acs gene from S. coelicolor | This work |
| SC-AcsA | | |

^{a}Antibiotic markers: Km, kanamycin; Nal, nalidixic acid; and Rif, rifampicin.
^{b} Strain obtained from the E. coli Genetic Stock Center (Yale University, New Haven, CT, USA).

TABLE 2
Acetaldehyde dehydrogenase (Aldh) genes

| | | | | Accession |
|---|---|---|---|---|
| Organism | Locus | Gene | Gene ID | number |
| Escherichia coli | b0351 | mhpF | 945008 | AAC73454.1 |
| Escherichia coli | b1241 | adhE | 945837 | AAC74323.1 |
| Escherichia coli | b2455 | eutE | 946943 | AAC75508.1 |
| | | (SEQ ID NO: 26) | | |
| Pseudomonas | PA4022 | hdhA | 879017 | AAG07409.1 |
| aeruginosa | | (SEQ ID NO: 24) | | |
| Pseudomonas | PA1984 | exaC | 880413 | AAG05372.1 |
| aeruginosa | | (SEQ ID NO: 22) | | |
| Moorella | Moth_1776 | SEQ ID NO: 28 | 3832442 | ABC20077.1 |
| thermoacetica | | | | |
| Clostridium | CTHE_RS02195 | adhE | 35805477 | ABN51661 |
| thermocellum | | | | |
| Streptomyces | SCATT_09460 | adhE | 12647716 | AEW93317.1 |
| cattleya | | | | |

TABLE 3
Acetyl-CoA synthetase (Acs) genes

| | | | | Accession |
|---|---|---|---|---|
| Organism | Locus | Gene | Gene ID | number |
| Bacillus | BSU_29680 | acsA | 937324 | CAB14946.1 |
| subtilis | | (acsBt, SEQ | | |
| | | ID NO: 40) | | |
| Cupriavidus | CNE_RS23295 | acsCn, SEQ | | WP_013952735 |
| necator | | ID NO: 42 | | |
| Streptomyces | SCO3563 | acsA | 1098999 | CAB38500 |
| coelicolor | | (acsSc, SEQ | | |
| | | ID NO: 44) | | |

### Cell-Free Extracts Assays Preparation

Cell-free extracts of P. putida KT2440 were prepared using overnight cultures of the strains and diluting them to an OD₆₀₀ of 0.1 in fresh minimal medium with the appropriate antibiotics, in 250-ml shake flasks. Cells were grown at 30° C. with shaking of 180 rpm until they reached an OD₆₀₀ between 0.4-0.6, after which they were induced with 15 mM of NaF. After 20 hours of incubation, all the cells were harvested by centrifugation at 5000 rpm for 10 minutes at 4° C. Cellular pellets were washed with Tris-HCl 50 mM pH7.8 and resuspended in a final volume of 3 ml. Cells were disrupted using glass beads in a cell homogenizer (Precellys 24, Bertin instruments), using a program of 6000 rpm for 20 seconds. The mix was centrifuged at 17000 g for 2 minutes at 4° C., and the supernatant was transferred to a new tube. Protein concentrations were determined using a Bradford assay, yielding a concentration of at least 1 mg/ml. 1 ml of the protein fraction was then mixed with 0.2 mM of SAM and 5 mM of NaF, in Tris-HCl 50 mM pH7.8. This reaction was incubated for 20 hours at 30° C., and then inactivated by boiling the samples at 95° C. for 5 minutes and centrifuging at 17000 g for 10 minutes. The supernatant was taken and analyzed by LC-MS.

To yield enough amount of fluorometabolites to be detected by ¹⁹F-NMR, the same procedure was scaled up. 500 ml of fresh minimal medium was inoculated with overnight cultures in 2-L shake flasks, grown to and OD₆₀₀ 0.4-0.6 and then induced. After 24 hours of induction, cells were harvested by centrifugation at 4700 g for 20 minutes at 4° C., and resuspended in 35 ml of Tris-HCl 50 mM pH7.8. Cells were subsequently opened using a French press (Avestin Emulsiflex C5) and the lysates are centrifuged at 10.000 g for 20 minutes. 10 ml of the supernatants are used directly for the enzymatic reactions mixing them with 0.2 mM of SAM and 5 mM of NaF, in Tris-HCl 50 mM pH7.8, to a final volume of 20 ml. This reaction was incubated for 20 hours at 30° C., and then inactivated by boiling the samples at 95° C. for 5 minutes and centrifuging at 17000 g for 10 minutes. 100 μl of supernatant was taken and analyzed by LC-MS, and the rest was processed for ¹⁹F-NMR analysis.

### Protein Expression

Transformed BL21-DE3 cells with plasmids containing isomerases and aldolases in SEVA-derived vectors, were streaked from frozen glycerol stocks on LB agar supplemented with 50 μg/ml Kanamycin, whereas HIS-tagged versions of pET-Duet1-aldh homologues in BL21-DE3 cells were streaked on LB agar with 50 μg/ml Ampicillin. After an overnight incubation at 37° C., a single colony was picked in 15 ml liquid LB with the appropriate antibiotic and cultured at 37° C. in a shaking incubator (250 rpm, 16 h). 10 ml of the pre-culture was inoculated into 1 L LB with the appropriate antibiotic and grown at 37° C., 180 rpm until an OD₆₀₀ of 0.4-0.6 was reached. The main culture was cooled down to 16° C. and protein expression was induced with 1 mM IPTG, 16° C., 180 rpm for ˜21 h. Cells were centrifuged in an ultracentrifuge at 4° C. and pellets stored at −20° C. for further processing.

### Cell Lysis and Protein Purification

Frozen cell pellets were re-suspended on ice in 40 ml ice-cold 100 mM Phosphate Buffer (pH7.5), 150 mM NaCl, 10% Glycerol and lysed either 3 times in a French press (EmulsiFlex-05, Avestin, C519361) or with the addition of BugBuster Master Mix (Merck-Milipore, 71456). Genomic DNA was digested by addition of Benzoase Nuclease (Merck Milipore, 70746-3) in 30 minutes during centrifugation at 4° C. Supernatants were sterile filtered and purified using a HisPur™ Ni-NTA resin (Thermo Scientific, 88222), according to manufacturer's instructions.

### Acetaldehyde Dehydrogenase In Vitro Enzyme Assays

Aldh activity was tested for acetaldehyde and FAld in 200 μl in-vitro assays at 30° C. over a time course of 120 minutes. Reaction conditions were 50 mM Tris-HCl Buffer pH7.5, 500 μM NAD+, 200 μM substrate, 20 μg purified enzyme and 200 μM Coenzyme A. The reduction of NAD⁺ was followed in a microtiter plate reader at absorbance 340 nm in a Microtiter Plate reader (BioTek, Elx808).

### Acetyl-CoA Synthetase In Vitro Enzyme Assays

Acs activity was tested for Na-acetate and Na-fluoroacetate (Carbosynth, FS77305) in 300 μl in vitro assays at 30° C. over a time course of 120 minutes. Reaction conditions were 50 mM Tris-HCl Buffer pH 8, 10 mM MgCl₂, 2 mM DTT, 1 mM Coenzyme A, 10 μg purified enzyme and 10 mM ATP, which initiated the reaction. Substrate concentrations varied from 5-50 mM. 40 μl of sample was taken at indicated time points and formation of acetyl-CoA or fluoroacetyl-CoA was determined using the hydroxylamine method, adapted for microtiter plates and absorbance was measured at 490 nm in a Microtiter Plate reader (BioTek, Elx808) (Lipmann et al., 1945; Li et al., 2011). Initial reaction rates were calculated from the time course experiments for every substrate concentration. Kinetic parameters of the enzymes were determined via non-linear regression.

### Coupled Isomerase and Aldolase In Vitro Enzymatic Assay

Coupled isomerase and aldolase activity was monitored by following the consumption of DHAP, through NADH reduction at 340 nm (ε=6220 M⁻¹ cm⁻¹) via the activity of a Glycerol-3-Phosphate Dehydrogenase (GPDH, Sigma Aldrich). 5′-FDRP was produced by incubating chemically synthetized 5′-FDA (Calero et al. 2020) at 7.43 mM with 0.5 U Nucleoside Phosphorylase (PNP1, Sigma Aldrich) in 10 mM Tris buffer, for 16 h at 30° C. The assay was carried in 200 μl total volume at 30° C. over a time course of 3 hours. Reaction conditions were 50 mM Tris-HCl Buffer pH7.5, 200 μM NADH, 200 μM 5′-FDRP, 1 U GPDH and 10 μg purified of each enzyme. The reduction of NADH was followed at absorbance 340 nm in a Microtiter Plate reader (BioTek, Elx808).

### LC-MS Analysis

The metabolites were analyzed using a Prominence XR (Shimadzu, Columbia, MD) HPLC system coupled to a 5500 QTRAP mass spectrometer (Sciex, Framingham, MA). The autosampler was cooled to 15° C. A sample of 10 μL was separated on an XSelect HSS T3 150×2.1 mm²×2.5 μm (Waters®, Milford, MA) column. The column temperature was hold at 40° C. The metabolites were eluted with a constant flow rate of 0.4 ml min⁻¹. Elution began with 100% buffer A (10 mM tributylamine, 10 mM acetic acid (pH 6.86), 5% methanol, and 2% 2-propanol). Starting at 4 minutes buffer B (2-propanol) was linear increased to 15% at 8 minutes and hold at 15% till 12 minutes. Buffer B was then linear decreased to 0% till 13.5 minutes and kept at 0% till 17 minutes. The mass spectrometer was operated in negative mode with multiple reaction monitoring and unit resolution for the mass filter Q1 and Q3 was used for detection and quantification. The parameters for electrospray ionization are as following: electrospray voltage ˜4500 V, temperature 500° C., curtain gas 40 psi, CAD gas 12 psi, and gas 1 and 2 each 50 psi, collision gas high. Detection parameters were optimized for each compound. For each metabolite, a transition for quantification and a transition for qualification was used. Commercial 5′-FDA and enzymatically prepared 5′-FDRP were used as references and for external calibration curves for quantification. The purity of the enzymatically prepared 5′-FDRP was confirmed by ¹⁹F-NMR.

### ¹⁹F-NMR Analysis

Each sample was lyophilised from a frozen solution in water using a Christ Alpha 1-2 LD Plus freeze drier. To the freeze-dried sample was added a MeOD/D₂O solution, and the resulting suspension was then subjected to sonication to ensure dissolution of the fluorometabolites. ¹⁹F-NMR experiments were then recorded at 298 K on a Bruker Advance III HD 500 instrument with either a SmartProbe BBFO+ (for proton coupled experiments) or CryoProbe Prodigy TCI (for proton decoupled experiments), using CFCl₃ as an external reference. Chemical shifts are reported in parts per million (ppm).

### Data and Statistical Analysis

All experiments were independently repeated at least twice (as indicated in the corresponding figure or table legend), and the mean value of the corresponding parameter ±standard deviation is presented. In some cases, the level of significance of the differences when comparing results was evaluated by means of the Student's t test with α=0.01 or α=0.05 as indicated in the figure legends.

### Example 1.2—Results

A Nucleosidase of E. coli and a Kinase of Bacillus thuringiensis can Efficiently Recognize 5′-FDA and 5′-FDR to Produce 5′-FDRP

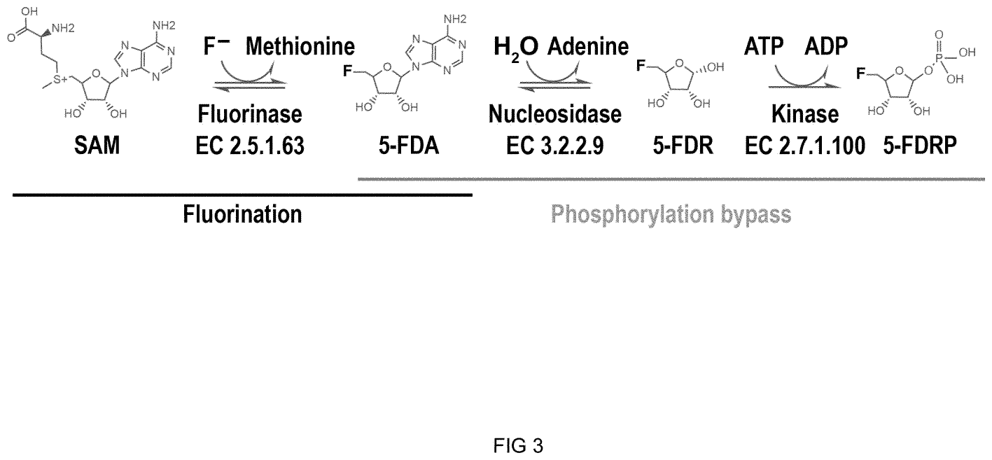

As described previously, both 5′-FDA and 5′-FDRP can be synthesized de novo and in vivo in P. putida KT2440 using a fluorinase and a PNP from Streptomyces (sp. MA37 or xinghaiensis) (Calero et al. 2020). However, the expression of the PNP that performs this step has proved to be challenging under certain conditions. Therefore, the capability of three other enzymes for using the fluorinated versions of their natural substrates was tested. Two of these enzymes belong to the SAM salvage pathway, a nucleosidase, which in its natural context recognize 5′-deoxyadenosine to produce D-5′-deoxyribose, releasing adenine, and a kinase, which converts D-5′-deoxyribose into D-5′-Deoxyribose 1-phosphate. We chose a nucleosidase from E. coli (pfs) (Iwai et al., 2011) and a kinase from B. thuringiensis (Beaudoin 2018) to test their activity using 5′-FDA and 5′-FDR, respectively, (FIG. 3). The third enzyme, it is a PNP from E. coli, DeoD, which is one of the two PNPs in this bacterium, with a wide substrate range of purines. Therefore, we tested whether this kinase can also perform the reaction with the fluorinated metabolites.

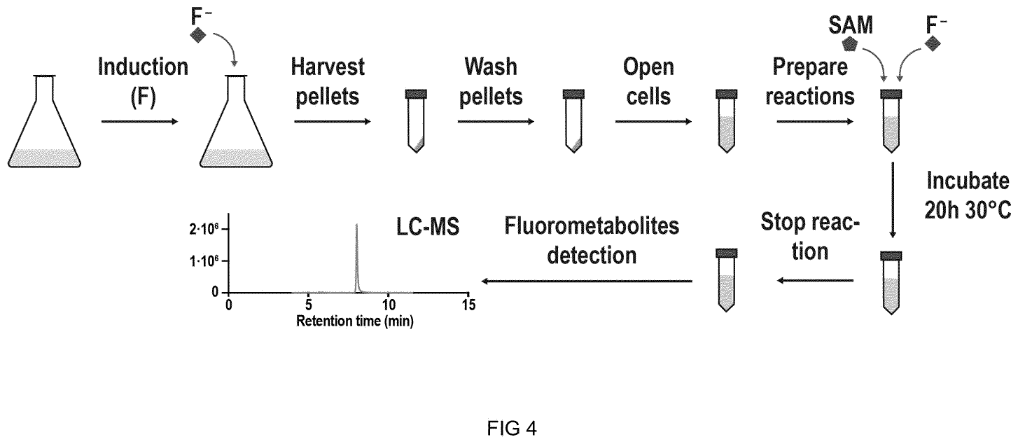

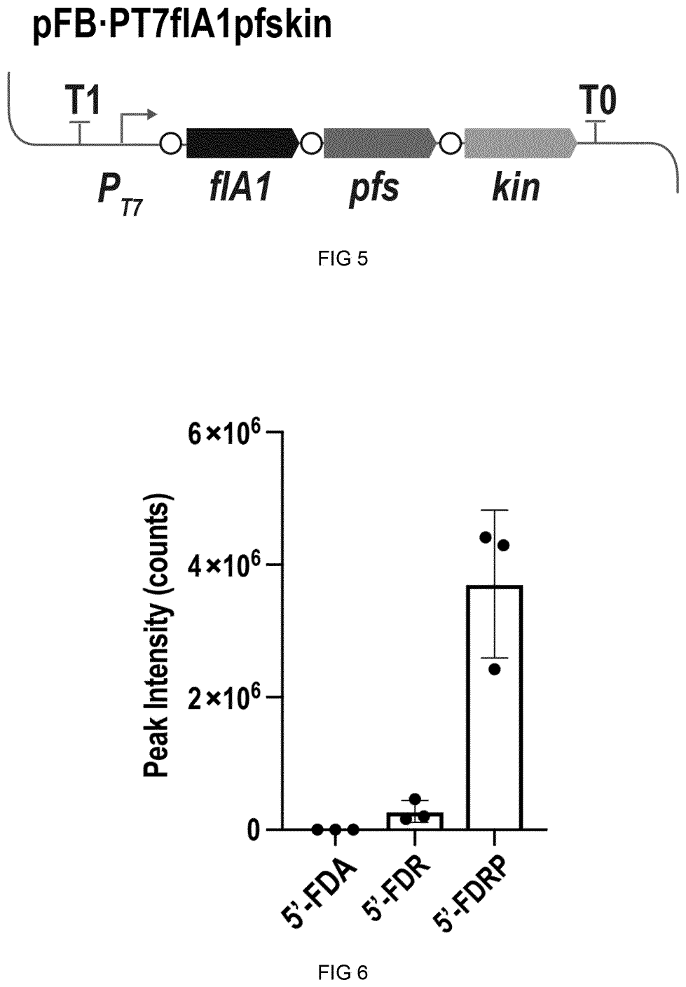

To test this hypothesis, we used a semi in vivo approach using cell-free extracts of cultures of P. putida KT2440::FRS-T7RNAP that were expressing the genes coding for the different enzymes of interest for 24 h, and incubated with the substrates SAM and NaF for 20 h, measuring the appearance of the product in LC-MS (FIG. 4). To this end, the construct used contained the Streptomyces sp. MA37 fluorinase flA1, the nucleosidase pfs (SEQ ID NO: 16) and the kinase of B. thuringiensis (kinBt, SEQ ID NO: 34) into an expression vector under the expression of the promoter PT7 (FIG. 5). FIG. 6 shows that after 20 h of incubation of the cell-free extracts with the substrates of the reaction, no 5′-FDA is detected, with most of it converted into the final product 5′-FDRP, although some residual 5′-FDR is also found. This result indicates that both nucleosidase and kinase can recognize the fluorinated versions of their original substrates and perform their conversion into other fluorometabolites.

### Novel Enzymatic Screening Method Developed for the Combined Activity of Isomerases and Aldolases

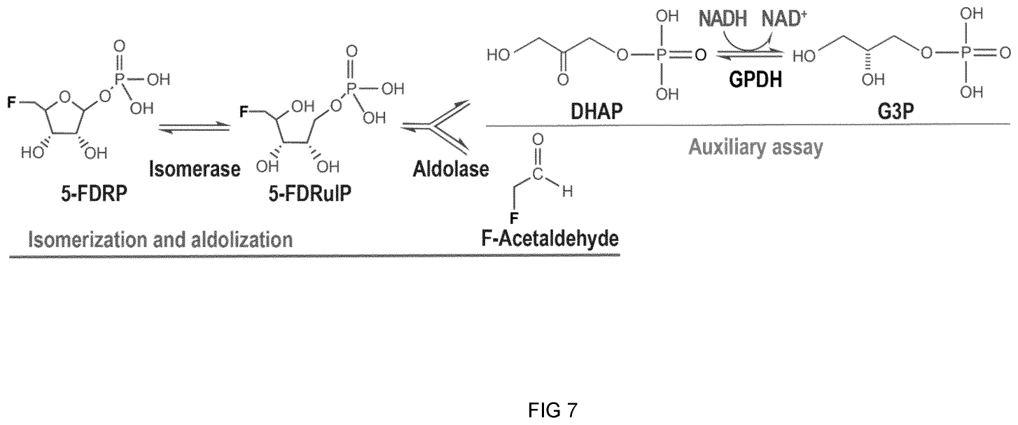

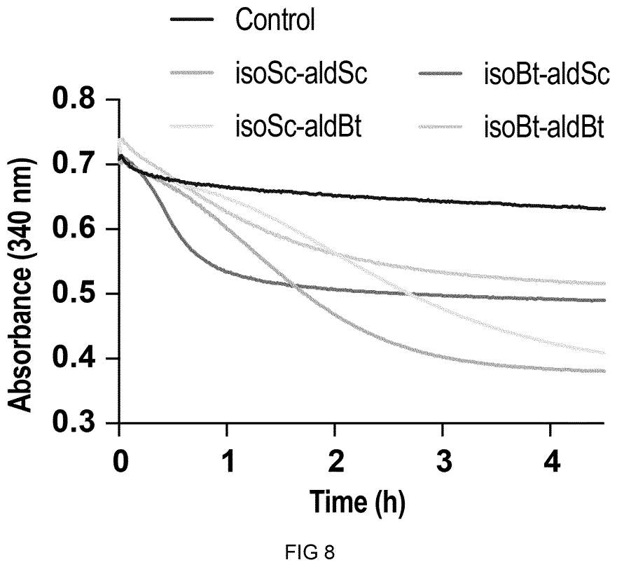

In the canonical fluorination pathway in S. cattleya, the fluorinated sugar 5′-FDRP is converted into fluoroacetaldehyde (FAld) by the consecutive reactions performed by an isomerase and an aldolase, releasing a molecule of dihydroxyacetone phosphate (DHAP) (FIG. 7). However, the monitorization of these reactions proved to be difficult, since the detection of products could not be done using sensitive techniques such as LC-MS. The detection of FAld was possible through ¹⁹F-NMR, which needs very high concentrations of metabolite for detection. These issues led us to developing an enzymatic method to indirectly monitor the synthesis of FAld in order to test different variants and combinations of enzymes to find the most efficient ones. The enzymatic reaction is based in the spectrophotometric detection of NADH, which has an absorbance peak at 340 nm, by coupling a glycerol 3-phosphate dehydrogenase (GPDH) to the reaction. GPDH converts DHAP into glycerol 3-phosphate (G3P), consuming a molecule of NADH in the process (FIG. 7). Using a commercial GPDH added in excess to the reaction mixture, the decrease in the absorbance is proportional to the production of DHAP and FAld, thus enabling rapid prototyping of enzymes relevant for assembling the fluorination pathway.

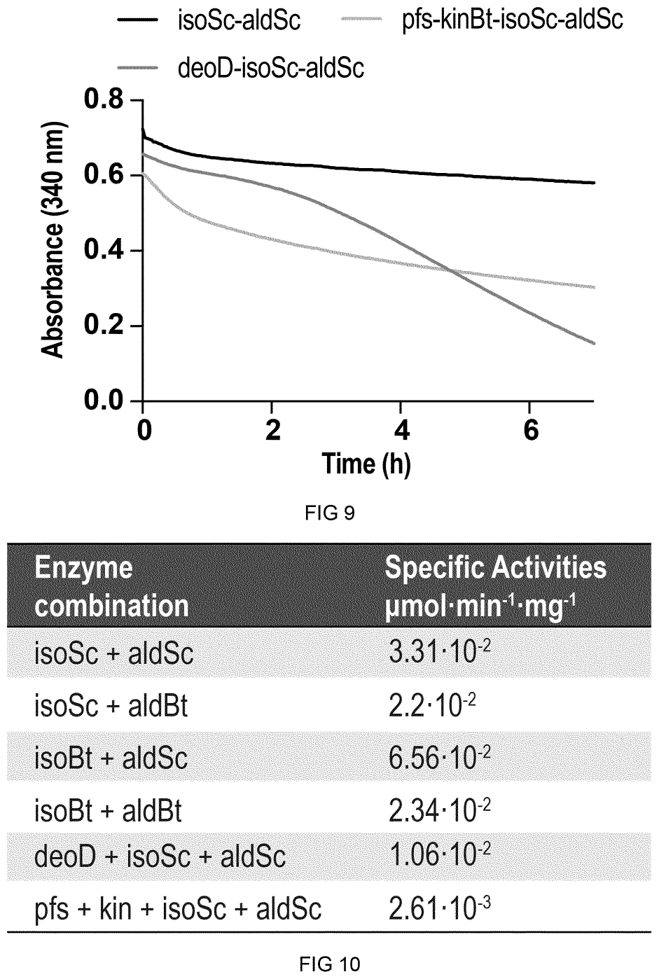

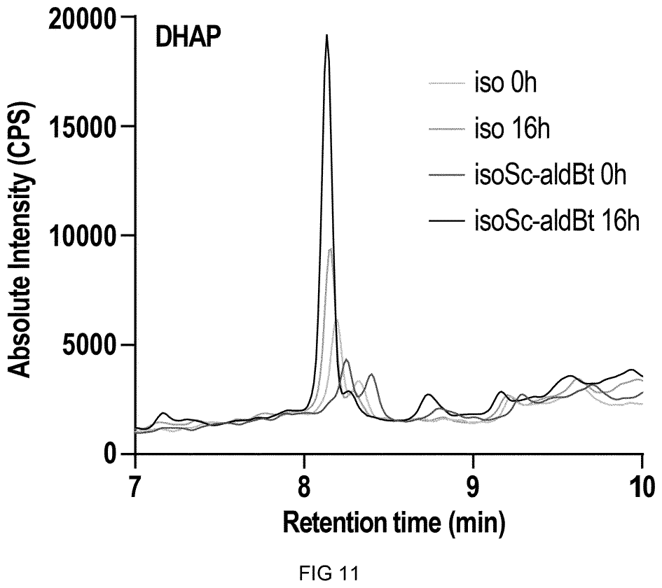

We used this enzymatic assay with cell-free extracts of cultures of P. putida KT2440::FRS-T7RNAP expressing an isomerase and an aldolase from S. cattleya (isoSc, isomerase^{S. cattleya}) and S. coelicolor (aldSc, aldolase^{S. coelicolor}), respectively, that have been previously shown that are able to use fluorinated intermediates (Deng et al., 2008), as well as both an isomerase (isoBt, isomerase^{B. thuringiensis}) and an aldolase (aldBt) from B. thuringiensis that have been previously described to perform the same reaction with the non-fluorinated metabolites (Beaudoin 2018). Genes encoding the different enzymes were cloned in an expression vector for their overexpression and further purification of the respective enzymes. Afterwards, the purified isomerases and aldolases were mixed in different combinations and tested together with the GPDH to see the depletion of NADH. For this purpose, the NADH consumption was tracked using four different combinations of isomerases and aldolases (FIG. 8), showing that even though the combination of the isomerase^{B. thuringiensis} and the aldolase^{S. coelicolor} was faster, with a specific activity of 6.56·10⁻² μmol·min⁻¹·mg⁻¹ (FIG. 10), higher conversion was achieved using the combination of isomerase^{S. cattleya} and the aldolase^{S. coelicolor}. Furthermore, the conversion of 5′-FDA into FAld was tested using cell-extracts of the best isomerase and aldolase combination i.e. isomerase^{S. cattleya} and aldolase^{S. coelicolor} together with cell-extracts of cultures of P. putida KT2440::FRS-T7RNAP expressing the nucleosidase pfs and kinase, or the PNP DeoD. Using both combinations we succeeded to see conversion into FAld, although the DeoD conversion was higher compared to a faster conversion by the phosphorylation bypass (FIG. 9). To validate the enzymatic assay, we used LC-MS detection of DHAP to see that higher concentrations of this metabolite were obtained when an isomerase and aldolase were used in an enzymatic reaction for 16 hours compared to the controls only with isomerase (FIG. 11).

The Combined Expression of an Isomerase of B. thuringiensis and an Aldolase of Streptomyces coelicolor Triggers Biosynthesis of F-Acetaldehyde

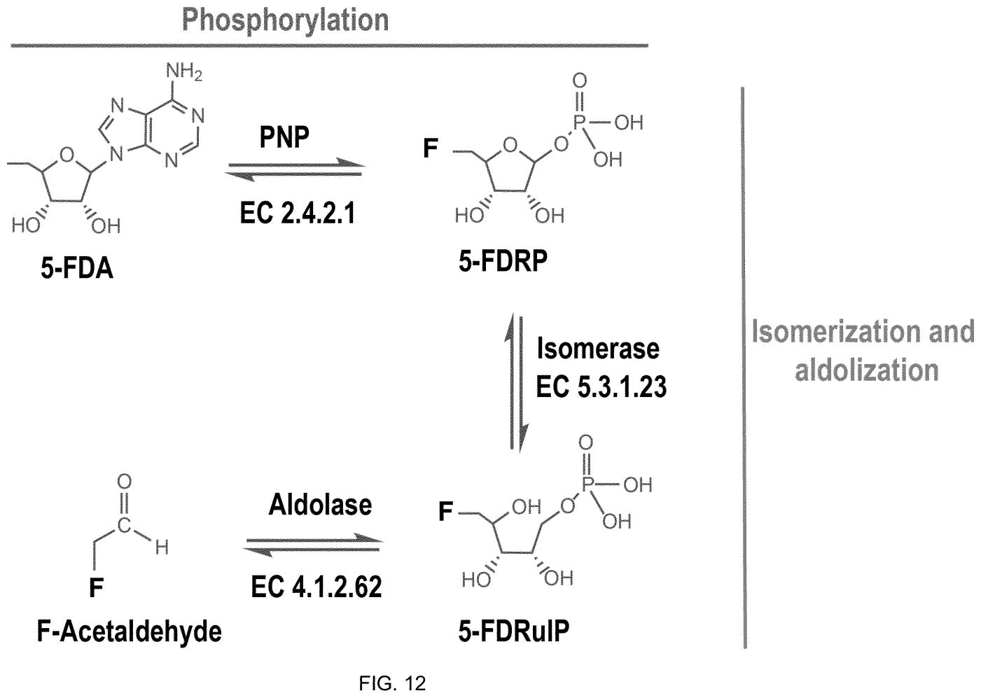

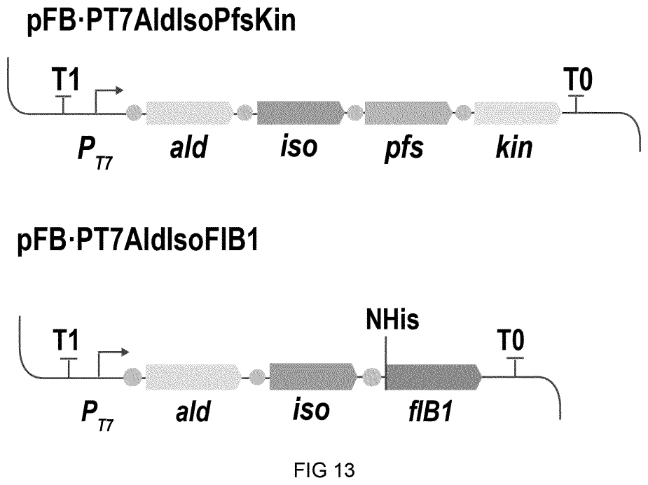

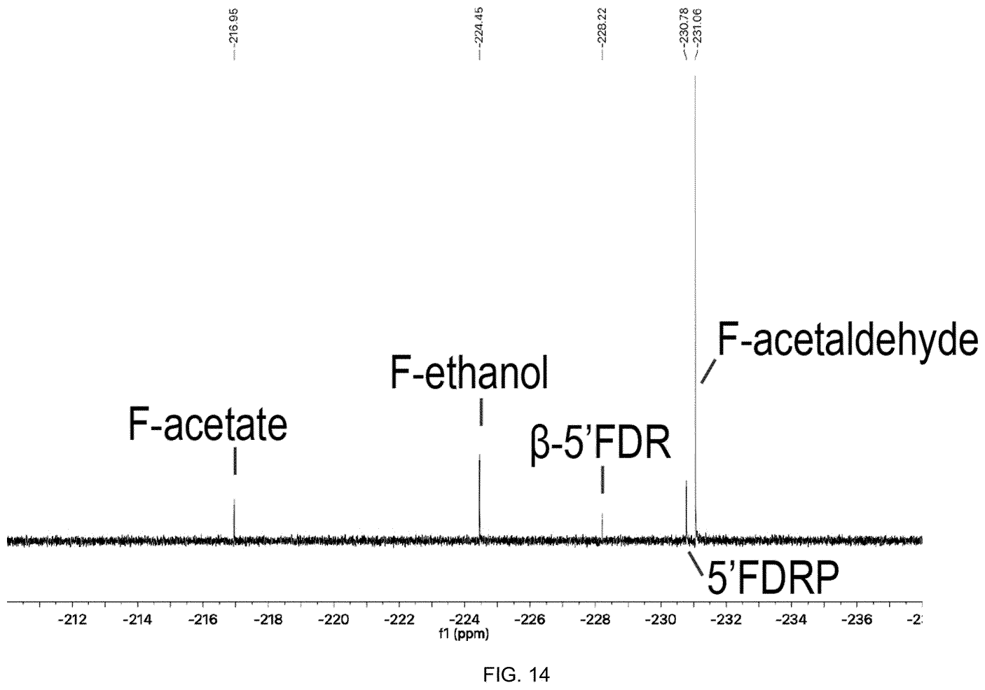

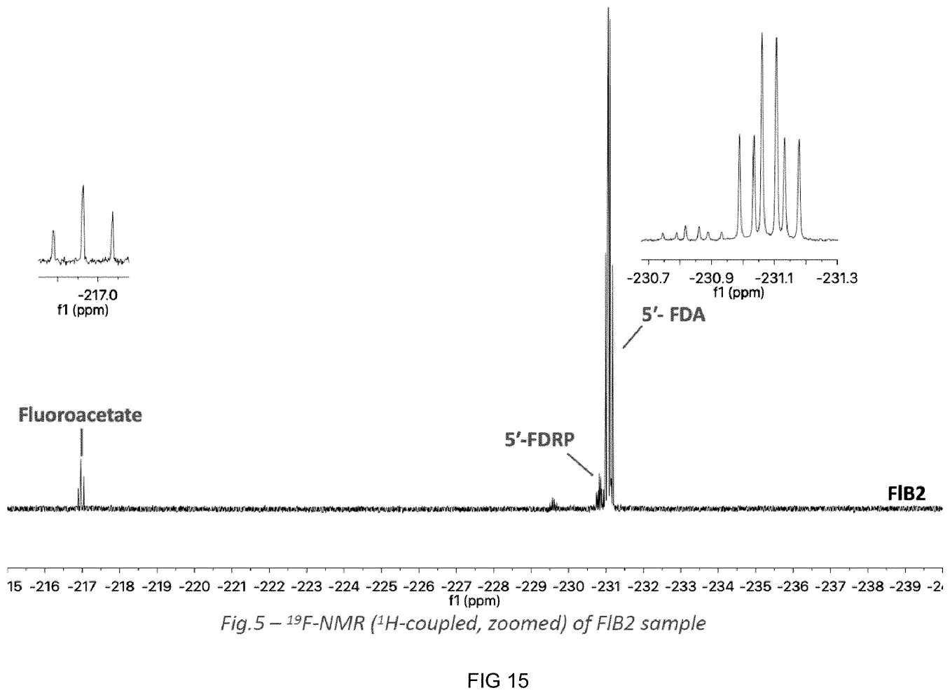

After confirming the activity of the isomerases and aldolases tested by the herein disclosed enzymatic assay, we combined the enzymes with the previous step of the fluorination pathway. This way, we tested simultaneously the expression and activity of enzymes of the phosphorylation step, both using PNPs and the phosphorylation bypass, and the isomerization plus aldolization (FIG. 12). These combinations of genes were constructed in a single plasmid, regulated as well by the PT7 promoter and transformed into P. putida KT2440::FRS-T7RNAP, to trigger the expression using F⁻ as inducer (FIG. 13). Cell-free extracts of cells expressing these genes were prepared and incubated with 5′-FDA, the substrate of the first step of the pathway. The presence of fluorometabolites by the end of the reaction was tested using ¹⁹F-NMR. FAc could be detected by ¹⁹F-NMR when a combination of isomerase and aldolase together with a PNP from Streptomyces sp. strain MA37 was used, suggesting that FAld is produced under these circumstances and that it is transformed to FAc by an endogenous acetaldehyde dehydrogenase (Aldh) of P. putida KT2440 (FIG. 15). When the combination of nucleosidase and kinase (phosphorylation bypass), isomerase and aldolase was used, both FAc and FAld could be detected, as well as FEtOH (FIG. 14).

### Acetaldehyde Dehydrogenase Catalyzes the Oxidation and CoA-Activation Reaction Using F-Acetaldehyde as Substrate

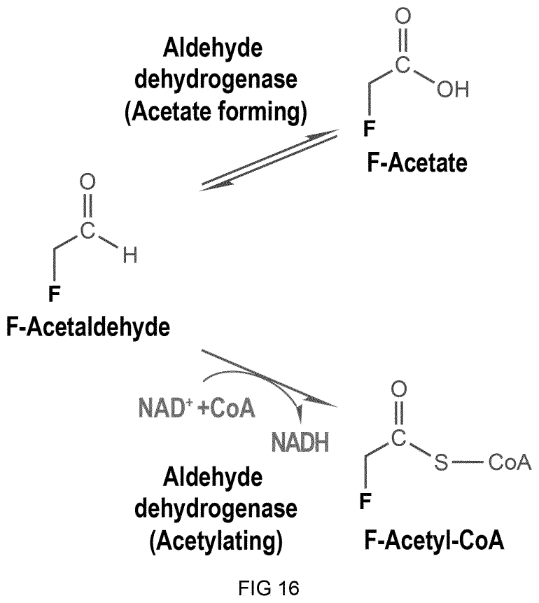

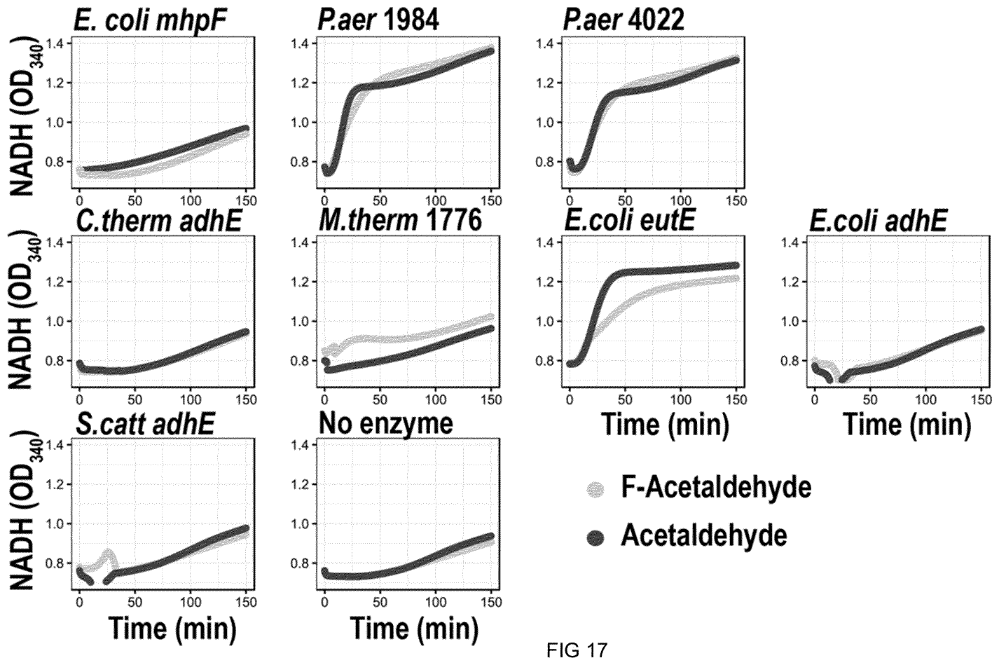

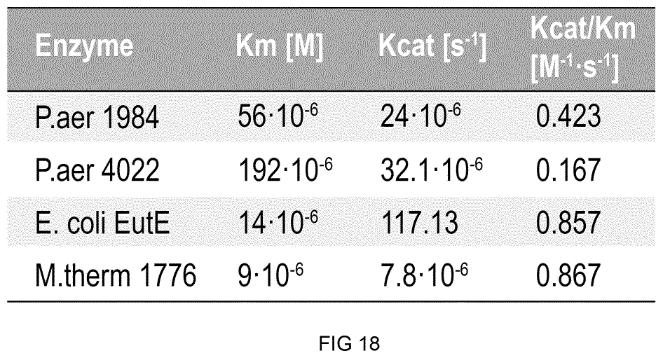

The last step of the proposed pathway for production of fluorinated building blocks is the activation of FAc to produce FAcCoA. We looked for acetaldehyde dehydrogenases (Aldhs) that may use acetaldehyde as substrate and introduce a CoA moiety by releasing a NADH molecule. Using a spectrophotometer to measure the NADH release allows screening for activity of the enzymes in order to select the best candidates for production of FAcCoA in a single step (FIG. 16). We prepared a small library of Aldh homologs from different bacteria, such as Pseudomonas aeruginosa, Moorella thermoacetica, and E. coli. The constructs containing the Aldh genes were then expressed in E. coli and the enzymes were purified. Afterwards, the purified enzymes were incubated with FAld or acetaldehyde to compare their activities using both substrates individually. This assay showed that few of the enzymes could perform the reaction using FAld (FIG. 17). Particularly, three of them, two Aldhs of P. aeruginosa, and one of E. coli, showed the best performance (FIG. 18).

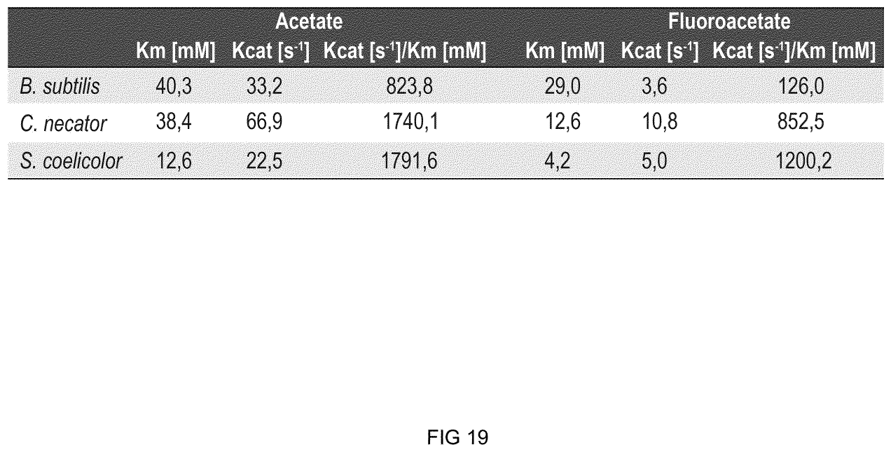

We determined the kinetic parameters of the acetyl-CoA-synthetases (Acs′) from Bacillus subtilis (BS), Cupriavidus necator (CNI) and Streptomyces coelicolor (SC) for both acetate and FAc (Table 3). The Km and Kcat values for acetate agree with the range of published results. It is interesting to note that all three enzymes were able to activate FAc with the affinity constants (Km) even lower compared than acetate, suggesting a higher affinity for FAc than acetate (FIG. 19).

### Example 2—Fluorinase

### Example 2.1—Materials and Methods

### In Silico Screening of Putative Fluorinases, Sequence Analysis, and Structure Modelling

The EnzymeMiner 1.0 online tool (https://loschmidt.chemi.muni.cz/enzymeminer/) (Hon et al., 2020) was used to search for putative fluorinases, using the FlAMA37 sequence as query (UniProt accession no. WOW999) and introducing D16, Y77, S158, D210, and N215 as essential residues. Results can be accessed through the EnzymeMiner webpage under Job ID 83urmz. Retrieved sequences were cured to remove duplicates and add missing N-terminal residues. Multiple sequence alignment and phylogeny analysis were conducted with MEGA X software using the Neighbor-Joining method. Phylogenetic analysis using 16S rRNA sequences were performed in the same way (Table 3). Sequence analysis of the fluorination gene clusters was performed by conducting tBLASTn searches against whole-genome sequences of the different organisms deposited in the National Center for Biotechnology Information (NCBI) databases, using the Fl protein sequences from S. cattleya (Table 4) and the Fdr protein sequences from Streptomyces sp. MA37 (GenBank accession no. LN612605.1) as query.

TABLE 3
NCBI accession numbers for the 16S rRNA sequences used for phylogenetic
analysis of organisms encoding fluorinase genes, as shown in FIG.
24. Numbers between brackets indicate nucleotide positions.

| Organism | Accession no. |
|---|---|
| Streptomyces sp. MA37 | HG428740 |
| Streptomyces cattleya | NC_017586.1 [2,721,506-2722810; |
| | 4,855,739-4,857,043; 3,613,701- |
| | 3,615,005; 1,079,076-1,080,380; |
| | 2,144,515-2,145,819; 802,858- |
| | 804,162] |
| Streptomyces xinghaiensis | NR_116059.1 |
| Streptomyces sp. SAJ15 | NISY01000001.1 [424-1,746] |
| Actinoplanes sp. N902-109 | CP005929.1 [183,863-185,376; |
| | 1,631,125-1,632,638; 6,234,429- |
| | 6,235,942; 6,736,828-6,738,341] |
| Actinopolyspora mzabensis | NR_109353.1 |
| Amycolatopsis bartoniae | MN399889.1 |
| Amycolatopsis sp. CA-128772 | PPHG01000117.1 [201-1,497]; |
| | PPHG01000111.1 [717-2,013]; |
| | PPHG01000090.1 [39303-40599]; |
| | PPHG01000071.1 [231,228-232,524]; |
| | PPHG01000056.1 [158-1,454]; |
| | PPHG01000030.1 [16,431-17,727] |
| Goodfellowiella sp. AN110305 | KX762322.1 |
| Nocardia brasiliensis ATCC | CP003876.1 [1,448,256-1,449,778; |
| 700358 | 2,271,961-2,273,483] |
| Chloroflexi bacterium | VBIC01000091.1 [1-430] |
| Peptococcaceae bacterium CEB3 | DXJ01000019.1 [93-1,424] |
| Thermodesulforhabdus norvegica | NR_025970.1 |
| Methanosaeta sp. PtaU1•Bin055 | AJ133791.1 |
| Salinispora tropica CNB-440 | NR_074502.1 |

TABLE 4
NCBI accession numbers for the nucleotide
sequences harboring fI gene clusters.

| ORGANISM | ACCESSION NO. |
|---|---|
| Streptomyces sp. MA37 | HG428738.2 |
| Streptomyces cattleya | NC_016111; NC_016113 |
| Streptomyces xinghaiensis | NZ_CP023202 |
| Streptomyces sp. SAJ15 | NZ_NISY01000005; |
| | NISY01000015 |
| Actinoplanes sp. N902-109 | NC_021191 |
| Actinopolyspora mzabensis | FNFM01000012; |
| | FNFM01000017 |
| Amycolatopsis bartoniae | NZ_JACHWH010000014 |
| Amycolatopsis sp. CA-128772 | NZ_PPHG01000085 |
| Goodfellowiella sp. AN110305 | NZ_VUOB01000072 |
| Nocardia brasiliensis IFM 10847 | NZ_BAUA01000190 |
| Nocardia brasiliensis NCTC 11294 | UGSN01000003 |
| Nocardia brasiliensis ATCC 700358 | NC_018681 |
| Chloroflexi bacterium | VBIC01000085 |
| Peptococcaceae bacterium CEB3 | LDXJ01000028 |
| Thermodesulforhabdus norvegica | NZ_FOUU01000001 |
| Methanosaeta sp. PtaU1•Bin055 | MVRM01000143 |
| Salinispora tropica CNB-440 | NC_009380 |

Protein structure models were created using the SWISS-MODEL server (Waterhouse et al., 2018) and visualized with PyMOL Molecular Graphics System (Schrödinger, LLC). For residue conservation visualization, the ConSurf server was used to estimate conservation scores and the results were visualized with the UCSF ChimeraX software (Ashkenazy et al., 2016, Pettersen et al., 2004).

Accession numbers to the amino acid sequence of the putative fluorinases and fluorinases are listed in Table 5.

TABLE 5
Fluorinases and putative fluorinases.

| Accession no. | Organism | Abbreviations |
|---|---|---|
| CDH39444.1 | Streptomyces sp. MA37 | FIA_{MA37}, FIA^{MA37} |
| 1RQP_A | Streptomyces cattleya | FIA_{Scat}, FIA^{Scat} |
| WP_019711456.1 | Streptomyces xinghaiensis | FIA_{Sxin}, FIA^{Sxin} |
| WP_144383880.1 | Streptomyces sp. SAJ15 | FIA_{SAJ15}, FIA^{SAJ15} |
| WP_041832040.1 | Actinoplanes sp. N902-109 | FIA_{N902}, FIA^{N902} |
| SDK90789.1 | Actinopolyspora mzabensis | FIA_{Amza}, FIA^{Amza} |
| WP_145935427.1 | Amycolatopsis bartoniae | FIA_{Abar}, FIA^{Abar} |
| WP_103354124.1 | Amycolatopsis sp. CA-128772 | FIA_{CA12}, FIA^{CA12} |
| WP_149854061.1 | Goodfellowiella sp. AN110305 | FIA_{AN11}, FIA^{AN11} |
| WP_029901962.1 | Nocardia brasiliensis IFM 10847 | FIA_{Nbra2}, FIA^{Nbra2} |
| SUB47361.1 | Nocardia brasiliensis NCTC 11294 | FIA_{Nbra3}, FIA^{Nbra3} |
| AHK61118.1 | Nocardia brasiliensis ATCC 700358 | FIA_{Nbra1}, FIA^{Nbra1} |
| TMD14026.1 | Chloroflexi bacterium | FIA_{Cbac}, FIA^{Cbac} |
| KLU63204.1 | Peptococcaceae bacterium CEB3 | FIA_{Pbac}, FIA^{Pbac} |
| WP_093392705.1 | Thermodesulforhabdus norvegica | FIA_{Tnor}, FIA^{Tnor} |
| OPY51785.1 | Methanosaeta sp. PtaU1•Bin055 | FIA_{PtaU1}, FIA^{PtaU1} |
| 2Q6L_A | Salinispora tropica CNB-440 | SalL_{Stro}, SalL^{Stro} |

Fluorinase Production in E. coli and Purification

Synthetic genes encoding the different fluorinases were codon optimized for expression in E. coli and synthesized as gBlocks from Integrated DNA Technologies, which were then inserted into modified pET-28a(+) vectors as N-terminal His-tag fusions by USER cloning. In the case of flA_{MA37}, flA_{Scat}, and flA_{Sxin}, genes that had been previously codon optimized for expression in P. putida were amplified by PCR from the corresponding plasmids and also cloned into the pET-28a(+) backbone. These modified vectors encode a TEV cleavage site between the His-tag and the protein of interest instead of the standard thrombin site. Here, the native start codon from the fluorinase genes was removed to obtain the gene fusions. Plasmid construction was performed in E. coli DH5α λpir competent cells. Following sequence verification, the plasmids were transformed into E. coli BL21(DE3) for protein production. Kanamycin (Km, 50 mg/L) was added to the media for plasmid selection. Strains were maintained at −80° C. in 25% glycerol.

Different scales were used for the production of fluorinases. In all cases, pre-cultures inoculated from glycerol stocks were grown overnight in lysogeny broth (LB)+Km at 37° C. with agitation. For 96-deep well plate production, overnight cultures were diluted 100-fold in 0.5 mL 2xYT+Km in duplicate. After incubating for 2.5 hours at 37° C. and 250 rpm, one replicate well was induced with 1 mM IPTG, after which the plate was incubated overnight at 16° C., 150 rpm. After removing an aliquot for SDS-PAGE analysis of total protein content, cells were harvested by centrifugation and lysed by adding 100 μL BugBuster Master Mix (Novagen). Following ˜20 min incubation at room temperature with shaking, plates were centrifuged again and the supernatants were collected for SDS-PAGE analysis of the soluble fraction.

Medium-scale shake-flask cultivations were carried out in 250-mL baffled flasks containing 50 mL 2xYT+Km, inoculated from overnight cultures to OD600=0.05. Cells were grown at 37° C., 250 rpm, to OD₆₀₀=0.6-0.8, induced with 1 mM IPTG, and incubated for 24 h at 16° C., 150 rpm. Thereafter, cells were harvested by centrifugation and pellets frozen at −20° C. until protein extraction. For this step, cells were resuspended in 2 mL His A buffer (300 mM NaCl, 20 mM imidazole in 10 mM HEPES pH 7.5) and disrupted with glass beads in a Precellys 24 homogenizer (Bertin Instruments) with two 20 s cycles at 6,000 rpm.

In the case of large-scale shake-flask cultivations, overnight cultures were diluted 100-fold in 50 mL 2xYT+Km in 250-mL baffled flasks and grown at 37° C., 250 rpm, to OD₆₀₀˜1. Then, the whole cultures were added to 450 mL 2xYT+Km in 2-L baffled flasks and grown at 37° C., 250 rpm, to OD₆₀₀=0.6-0.8, after which 1 mM IPTG was added and the cultures incubated for 24 h at 16° C., 150 rpm. Cells were then harvested by centrifugation and pellets frozen until protein extraction. This was performed by resuspending cells in 20 mL His A buffer and disrupting in an Avestin Emulsiflex C5 French press (ATA Scientific Instruments).

Following centrifugation and filtration of cell extracts through 0.2 μm membranes, fluorinase purification was carried out using 1-2 mL HisPur Ni-NTA Resin (Thermo Scientific) in 10-mL Pierce™ disposable columns. Non-bound proteins were washed with 20 mL His A buffer before elution with 4 mL His B buffer (300 mM NaCl, 500 mM imidazole in 10 mM HEPES pH 7.5). The buffer of the eluted fraction was exchanged to 10 mM HEPES pH 7.5 using Amicon-Ultra 10,000 MWCO centrifugal units (Millipore). Protein concentration was measured in a Nanodrop, assuming 1 AU280=1 mg/mL. For long-term storage, 1 mM dithiothreitol (DTT) was added to the purified proteins, which were aliquoted, flash-frozen, and stored at −80° C. until further use.

### Fluorinase Activity Assays

96-well activity assays were carried out in 150 μL reactions containing 50 mM HEPES pH 7.8, 75 mM KF, ˜1 μM fluorinase (considering the theoretical molecular mass), and 10, 25, 50, 100, 250, or 500 μM SAM (New England Biolabs). 96-well PCR plates were sealed and incubated for 1 h at 37° C., 5 min at 95° C., and cooled to 10° C. in a thermocycler (Bio-Rad). The plates were then centrifuged for 30 min at 2,500×g and 120 μL of supernatants were transferred to round-bottom 96-well plates (Thermo Scientific), which were sealed with a silicone lid for HPLC analysis. Transhalogenation reactions were carried out in the same way, but using increasing concentrations of 5′-CIDA (5′-chloro-5′-deoxyadenosine) in the presence of 1 mM L-Met.

Steady-state kinetics were performed in the same conditions with slight variations. Triplicate reactions (600 μL final volume) were prepared in 1.5-mL tubes and incubated for 5 min at 37° C. before addition of SAM (1.5625-800 μM), after which 90 μL aliquots were removed at different time-points (2, 5, 10, 15, 20 and 30 minutes), boiled for 5 min at 95° C., and placed on ice. Samples were centrifuged at maximum speed for 10 min at 4° C. before being transferred to 96-well plates for 5′-FDA quantitation by HPLC analysis. Product formation rates for the different substrate concentrations were calculated by least squares linear regression with at least three time-points and fitted to the Michaelis-Menten equation using the Origin software (OriginLab Corporation).

5′-FDA was quantified following absorbance at 230 nm using a Zorbax C18 column (3.5 μm 4.6×100 mm, Agilent) connected to a HPLC system (Dionex Ultimate 3000, ThermoFisher) with the following gradient (1 mL/min flow rate): 5-12% solvent B in 1.5 min, 12% solvent B for 1 min, 12-30% solvent B in 2 min, 30-70% B in 1.5 min (solvent A: 0.05% acetic acid in water; solvent B: acetonitrile). A six-point calibration curve (R2>0.99) was prepared with 0.5-50 μM 5′-FDA, which was chemically synthesized as previously described (Calero et al., 2020).

In Vitro and In Vivo Biofluorination Assays in Pseudomonas putida KT2440

Synthetic flA_{PtaU1}, flA_{SAJ15}, flA_{Sxin}, and flA_{MA37} genes, codon optimized for expression in P. putida, were amplified by PCR and cloned into pSEVA231 backbones under control of the T7 promoter (named pFB·1 in Calero et al. 2020) by digestion with NdeI and SacI and ligation with T4 DNA ligase. The resulting plasmids, encoding N-terminal His-tag fusions, were transformed into a P. putida KT2440 strain harboring a chromosomal copy of the T7 RNA polymerase gene under control of a fluoride-responsive riboswitch (P. putida attTn7[FRSv1>T7RNAP]) (Calero et al., 2020).

Strains carrying the different plasmids were grown overnight in de Bont medium (Hartmans et al., 1989) with 5 g/L glucose and 50 mg/L Km at 30° C. with shaking, and then diluted to an OD₆₀₀ of 0.1 in 50 mL of the same medium in 250-mL shake flasks. Cells were grown at 30° C. with shaking of 180 rpm to an OD₆₀₀ of 0.4-0.6, at which point 15 mM NaF was added to the cultures to induce expression of the fluorinase genes. After 20 h of incubation, 2-mL samples were removed for metabolite extraction (in vivo assay), while the remaining cultures were centrifuged at 4,500×g for 10 min at 4° C. to harvest cells for the in vitro assays.

For metabolite extraction for the in vivo assays, cells were centrifuged at 5,000×g for 10 min and the supernatants were discarded. Cells were washed once with 50 mM phosphate buffer pH 7.8 and incubated with 2 mL of extraction solution (40:40:20 acetonitrile:methanol:water) at −20° C. for 5 min and vortexed. Cells were subsequently centrifuged at 19,000×g for 1 min and the supernatant was transferred to a new tube for evaporation in a SpeedVac concentrator (Thermo-Fisher Scientific) until complete dryness. Pellets were resuspended in 100 μL of Milli-Q water, centrifuged at 19,000×g for 1 min and the supernatant was analyzed for the presence of 5′-FDA by LC-MS as previously described (Calero et al., 2020).

For the in vitro biofluorination assays, frozen (−20° C.) cell pellets were thawed, washed with 50 mM Tris-HCl buffer, pH 7.8, and resuspended in a final volume of 3 mL of the same buffer. Cells were then disrupted with glass beads in a Precellys 24 homogenizer with two 20 s cycles at 6,000 rpm. The suspension was centrifuged at 17,000×g for 2 min at 4° C. and the supernatant transferred to a new tube. A reaction mixture of a total volume of 100 μL was prepared with 50 μL cell-free extract, 200 μM SAM and 5 mM NaF in 50 mM Tris-HCl buffer, pH 7.8. These reactions were incubated for 20 h at 30° C. in a thermocycler, after which they were stopped with a 5-min incubation at 95° C. and centrifugation for 10 min at 17,000×g, 4° C. Supernatants were analyzed for the presence of 5′-FDA by LC-MS as indicated above.

### Example 2.2—In Silico Experimentation

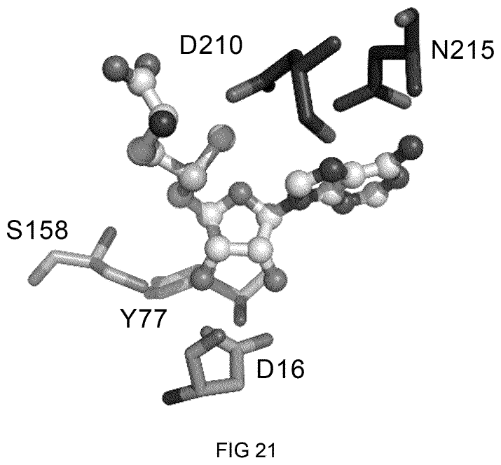

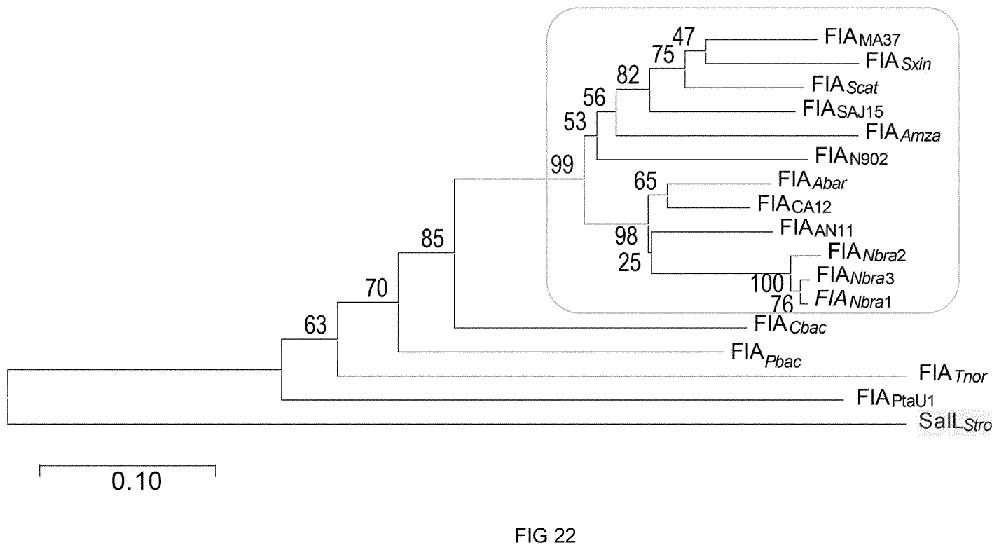

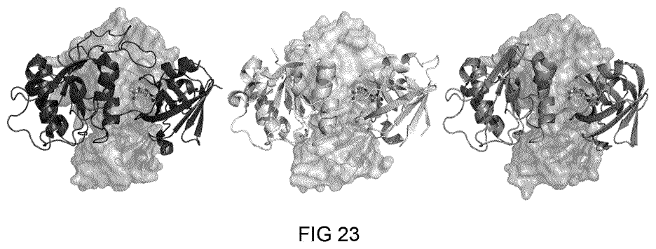

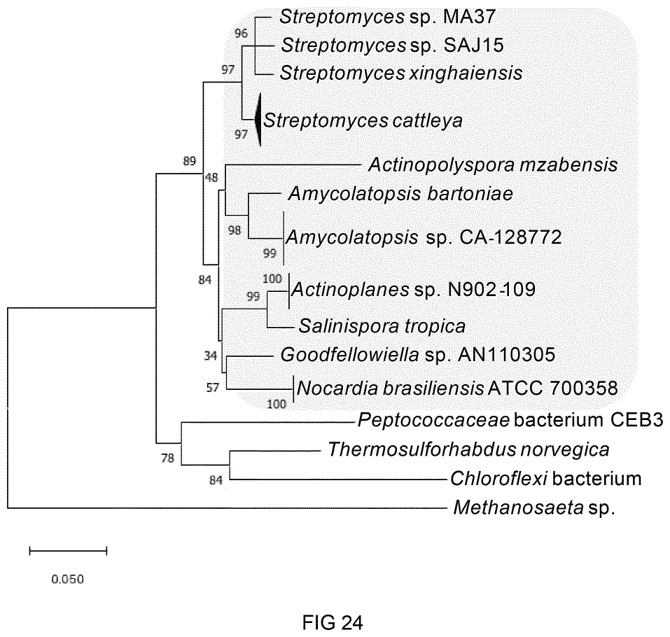

In an effort to identify “Nature's best” biocatalyst, the fluorinase from Streptomyces sp. MA37 (FlA^{MA37}) was used as the query sequence (Uni-Prot WOW999), and the residues D16, Y77, S158, D210, and N215 were specified as essential based on their implication in catalysis and substrate binding (FIG. 21). After curing redundant sequences, sixteen unique candidates were obtained (Table 6 and FIG. 22). Some of the retrieved amino acid sequences were found to be missing several N-terminal residues, which were added after manually curating the deposited genome sequences from which the fluorinase genes had been predicted. Out of the sixteen sequences retrieved, five corresponded to fluorinases previously reported in the literature (thus serving as an internal quality control of the prediction routine), while nine corresponded to new putative fluorinases. Another two sequences corresponded to the chlorinase from Salinispora tropica CNB-440 (SalL) and a putative chlorinase from the archaea Methanosaeta sp. PtaU1.Bin055 (FlA^{PtaU1}). Both of these sequences lack the 23-residue loop previously hypothesized to differentiate fluorinases from chlorinases (FIG. 23). Notably, only four of all the retrieved sequences were not sourced from Actinobacteria. These included the putative enzymes from a Chloroflexi bacterium (Chloroflexi), Peptococcaceae bacterium CEB3 (Clostridia), Thermosulforhabdus norvegica (Deltaproteobacteria) and Methanosaeta sp. PtaU1.Bin055 (Methanomicrobia). Phylogenetic analysis of the 16S rRNA sequences of the fluorinase-encoding organisms gave a similar result to that obtained when using the fluorinase amino acid sequences, except that, as expected, Salinispora tropica groups together with the other Actinomycetes, in a clade separate from the one formed by Streptomyces sp. (FIG. 24).

TABLE 6
Putative fluorinases retrieved from EnzymeMiner
search using FIA^{MA37} as query.

| Name | Organism | ID (%)^{a} |
|---|---|---|
| FIA^{MA37} | Streptomyces sp. MA37 | Query |
| FIA^{Scat} | Streptomyces cattleya | 87.6% |
| FIA^{Sxin} | Streptomyces xinghaiensis | 86.0% |
| FIA^{SAJ15} | Streptomyces sp. SAJ15 | 85.0% |
| FIA^{N902} | Actinoplanes sp. N902-109 | 80.7% |
| FIA^{Amza} | Actinopolyspora mzabensis | 78.9% |
| FIA^{Abar} | Amycolatopsis bartoniae | 79.1% |
| FIA^{CA12} | Amycolatopsis sp. CA-128772 | 78.6% |
| FIA^{AN11} | Goodfellowiella sp. AN110305 | 77.7% |
| FIA^{Nbra2} | Nocardia brasiliensis IFM 10847 | 75.7% |
| FIA^{Nbra3} | Nocardia brasiliensis NCTC 11294 | 75.3% |
| FIA^{Nbra1} | Nocardia brasiliensis ATCC 700358 | 75.3% |
| FIA^{Cbac} | Chloroflexi bacterium | 69.3% |
| FIA^{Pbac} | Peptococcaceae bacterium CEB3 | 64.8% |
| FIA^{Tnor} | Thermodesulforhabdus norvegica | 54.5% |
| FIA^{PtaU1} | Methanosaeta sp. PtaU1•Bin055 | 49.5% |
| SalL^{Stro} | Salinispora tropica CNB-440 | 35.6% |

^{a}Sequence identity. References to known FIAs are indicated.

### Example 2.3—In Vitro Fluorinase Activity and Biochemical Characterization of Fluorinases

Next, coding sequences of all FlA candidates were codon-optimized for production in Escherichia coli as N-terminal His-tag fusions—flA^{MA37}, flA^{Scat} and flA^{Sxin} had been previously codon-optimized for expression in Gram-negative hosts (Calero et al., 2020). SalL^{Stro} was not included in this experimental set since it is reportedly inactive on F⁻ (Eustaquio et al., 2008). The expression of the 16 candidate genes was initially evaluated in 96-well microtiter plate cultures. FlA^{Tnor}, FlA^{Amza} and FlA^{Pbac} could not be obtained as soluble enzymes and were not included in further analyses. Moreover, very faint bands of the expected size were observed in SDS-PAGE of E. coli extracts producing FlA^{Tnor} and FlA^{Amza}, suggesting limited expression levels (data not shown). Therefore, we proceeded to obtain the remaining 13 candidates in medium-scale shaken-flask cultures for His-tag purification and activity assays.

The purified enzymes were incubated in the presence of increasing SAM concentrations for 1 h, after which the 5′-FDA produced was measured by HPLC. 5′-FDA synthase activity could be detected for 12 out of the 13 candidates (no 5′-FDA synthase activity for Chloroflexi bacterium, data not shown). The protein concentration was normalized for these assays, although the enzymes were recovered with varying degrees of purity due to differences in solubility-typical of proteins isolated from high-G+C-content species when produced in a Gram-negative host. Notably, the enzyme from Methanosaeta sp. (FlA^{PtaU1}, predicted to be a chlorinase), was one of the top performers, together with FlA^{SAJ15}. These enzymes had specific activities similar to those of FlA^{MA37} and FlA^{Sxin}, which present the highest catalytic efficiencies on SAM fluorination reported to date.

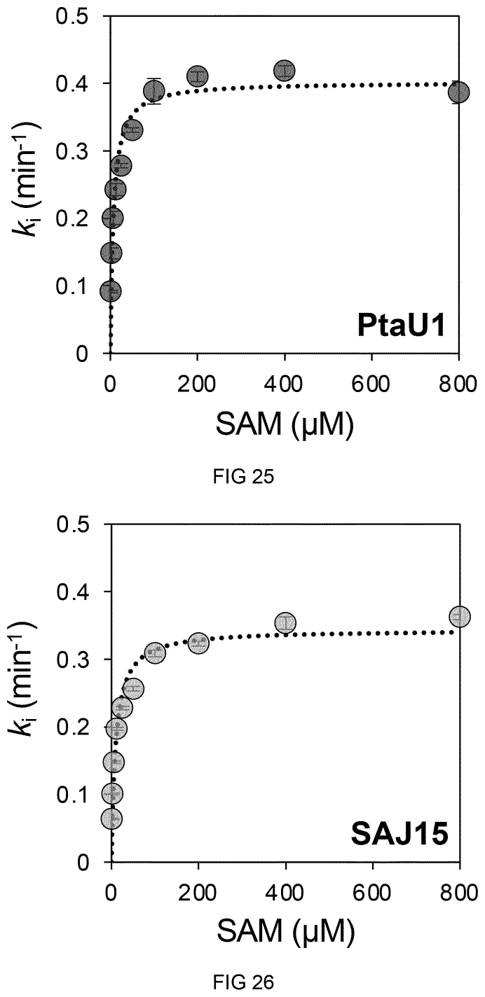

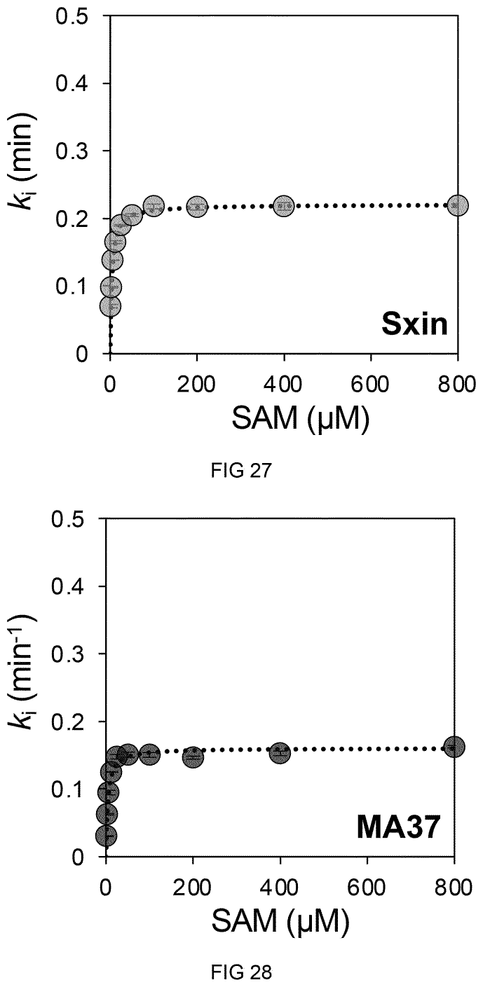

FlA^{PtaU1} and FlA^{SAJ15} were selected for large-scale shaken-flask production and a more detailed biochemical characterization. Steady-state kinetics assays with 1 μM of the purified protein, varying concentrations of SAM (1.5 to 800 μM) and 75 mM KF revealed that both of these enzymes presented higher turnover rates (k_{cat}) than FlA^{MA37} and FlA^{Sxin} (FIGS. 25, 26, 27, and 28, and Table 7). Surprisingly, K_{M}^{SAM} values were <10 μM, much lower than what had been previously reported in the literature for fluorinases (Schaffrath et al., 2003; Zhu et al., 2007; Sooklal et al., 2020). Previous studies used high enzyme concentrations (>10 μM), which impedes reaching a steady state of the reaction for substrate concentrations below 10 μM. Additionally, we have used a KF concentration that ensures F-saturation without causing any inhibitory effect (previous studies have used KF concentrations >200 mM).

TABLE 7
Michaelis-Menten kinetic constants of selected fluorinases.^{a}

| | K_{M}^{SAM} | k_{cat} | k_{cat}/K_{M}^{SAM} |
|---|---|---|---|
| Fluorinase | (μM) | (min⁻¹) | (mM⁻¹ min⁻¹) |
| FIA^{MA37} | 4.42 ± 0.58 | 0.16 ± 0.01 | 36.36 ± 4.82 |
| FIA^{Sxin} | 3.76 ± 0.15 | 0.22 ± 0.01 | 58.63 ± 2.63 |
| FIA^{SAJ15} | 9.62 ± 1.43 | 0.34 ± 0.01 | 35.81 ± 5.43 |
| FIA^{PtaU1} | 6.99 ± 1.06 | 0.41 ± 0.01 | 57.54 ± 8.85 |

^{a}Assays conducted in 50 mM HEPES, pH 7.8, with 75 mM KF and varying SAM concentrations at 37° C. Average and standard deviation are given for triplicate independent measurements.

### Example 2.4—Catalytic Characterisation of Fluorinases

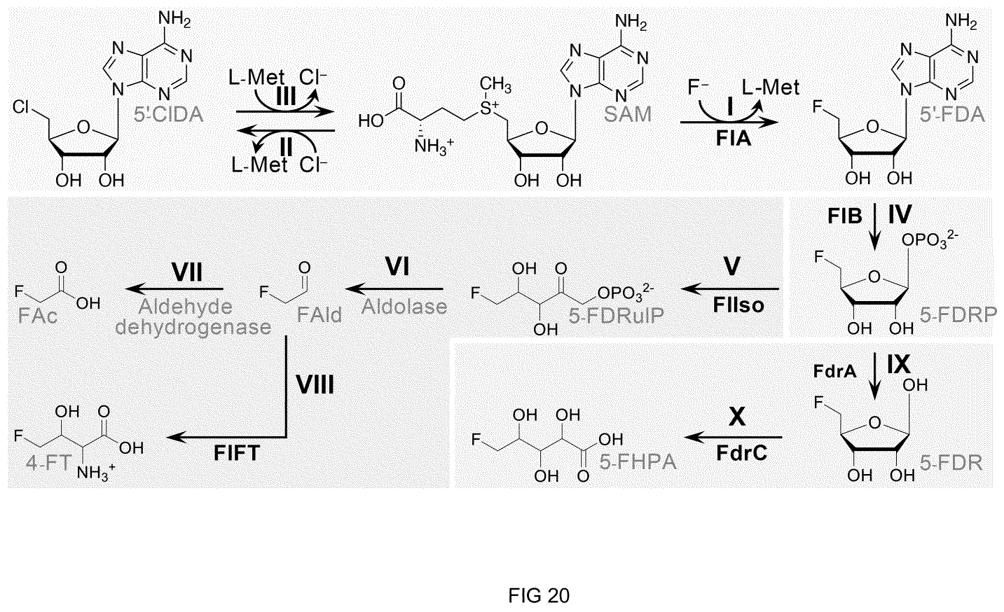

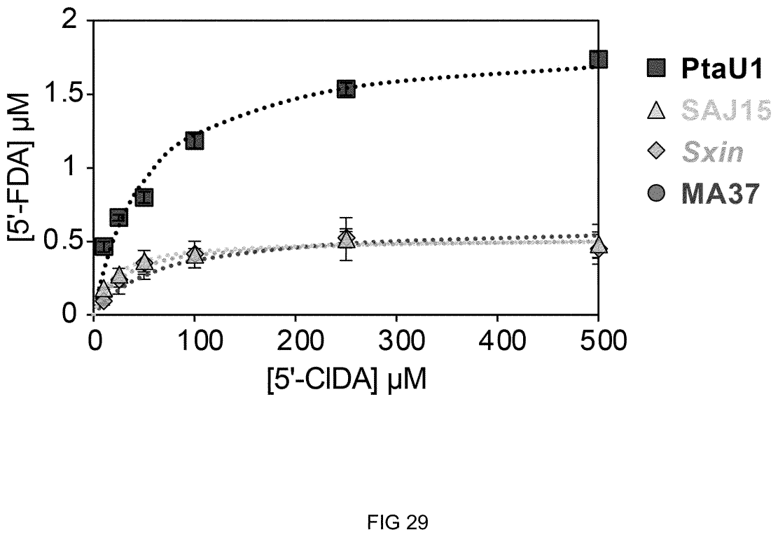

Since FlA^{PtaU1} was predicted to be a chlorinase, we evaluated whether it was also active towards S_{N}2-dependent addition of Cl⁻ onto SAM. Unexpectedly, no 5′-CIDA accumulation could be detected in enzymatic reactions in which KF was replaced by KCl—in contrast to SalL^{Stro} (Eustequio et al., 2008). Previous studies have shown that FlA^{Scat} can also catalyze the chlorination reaction (Deng et al., 2006). However, this feature requires the simultaneous removal of L-Met or 5′-CIDA, the reaction products, since the reverse dehalogenation reaction is favored. We could observe transhalogenation on 5′-CIDA (i.e. 5′-FDA production in the presence of L-Met and F⁻, steps III and I in FIG. 20, respectively; see FIG. 29). Again, FlA^{PtaU1} catalytically outperformed all other fluorinases, with a 3-fold higher Vₘₐₓ value. Although we cannot rule out that FlA^{PtaU1} could execute de novo chlorination, it is clear that the 23-residue loop reportedly found in «conventional» fluorinases is not essential for the activity towards F⁻.

### Example 2.5—In Vivo Fluorination

On this background, we tested the biosynthesis of fluorometabolites both in vitro and in vivo by engineering selected fluorinases in Pseudomonas putida. We have previously designed a fluoride-responsive genetic circuit that enabled biofluorination in this heterologous host (Calero et al., 2020). Here, this system was adapted to express either flA^{PtaU1}, flA^{MA37} flA^{Sxin} or flA^{SAJ15}, codon-optimized to facilitate expression in P. putida.

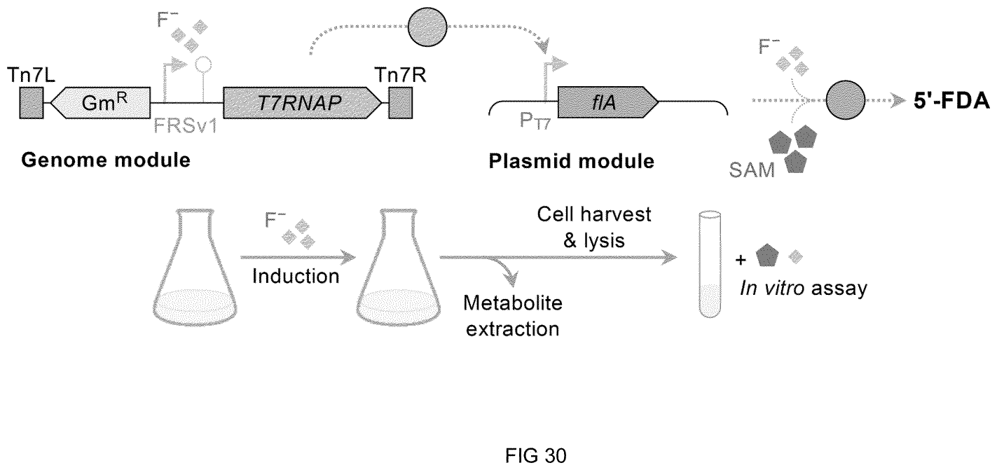

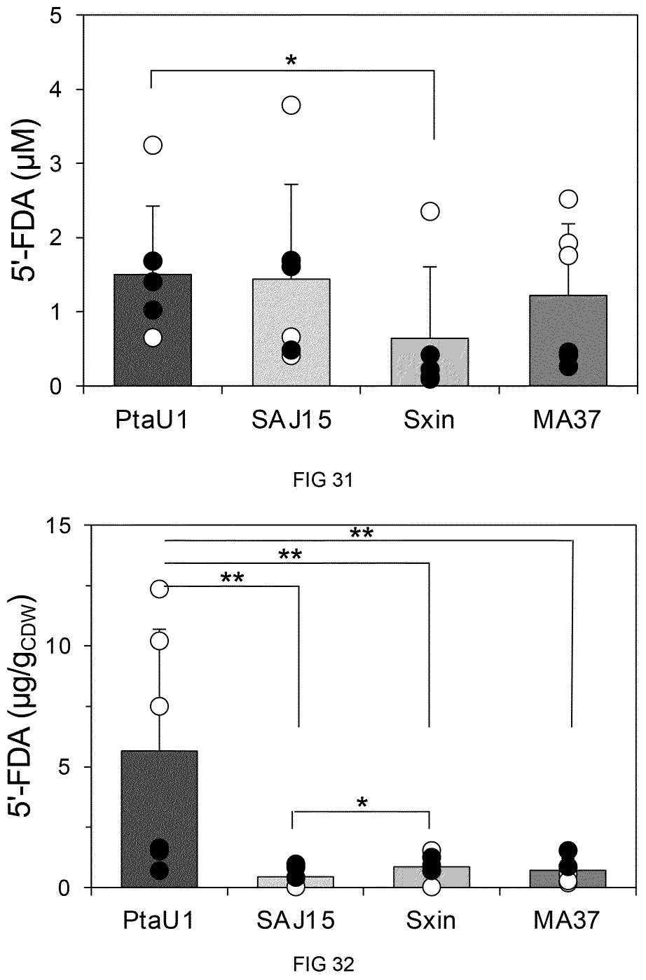

Upon induction of the system with NaF and expression of the fluorinase genes for 20 h at 30° C., production of 5′-FDA was determined by LC-MS to evaluate de novo fluorination activity in vivo (FIG. 30). Production of 5′-FDA could be detected for cells expressing either flA^{PtaU1}, flA^{SAJ15} flA^{MA37} and flA^{Sxin}. Notably, intracellular 5′-FDA, indicative of in vivo biofluorination, was 6- to 12-fold higher in cells expressing flA^{PtaU1} with respect to the other three fluorinase genes (FIG. 31). On the other hand, fluorinase activity from cell-free extracts incubated for 20 h at 30° C. in the presence of exogenously-added 200 μM SAM and 5 mM NaF was similar in all cases (FIG. 32).

In conclusion, the non-conventional FlA from the archaea Methanosaeta sp. PtaU1.Bin055 was found to present turnover rates far superior than those of all FlAs reported to date.

### Example 3—In Vivo Production of Fluorometabolites

For the in vivo synthesis of fluorometabolites, such as FAld or FAc, a strain of P. putida KT2440::FRS-T7RNAP containing one plasmid with the whole pathway, for example as illustrated in FIG. 2, or a combination of plasmids with the genes of the pathways under the regulation of PT7 promoter is used. This plasmid or combination of plasmids contains genes for a fluorinase, a PNP, or a nucleosidase and a kinase, and an isomerase, and aldolase, and an acetaldehyde dehydrogenase, such as an F-Aldh and/or an AcAldh. This strain is grown in minimal de Bont medium supplemented with glucose and the corresponding antibiotics until it reaches mid-exponential phase. After inducing the fluorometabolites production with F⁻ (addition of NaF), cells are grown overnight for production of fluorometabolites. After that, cells are harvested and centrifuged at 5000 rpm for 10 min to separate the bacterial biomass from the supernatant. Fluorometabolites in the supernatant can be detected using LC-MS. Samples may be concentrated by evaporation prior to analysis. Cells in the biomass are washed with phosphate buffer and incubated with 0.5 ml of extraction solution (60% (v/v) ethanol, 10 mM ammonium acetate, pH=7.2) at 78° C. for 1 min. Cells are subsequently centrifuged at maximum speed for 1 min and the supernatant is placed in a new Eppendorf tube. This extraction is repeated three times and then evaporated in vacuo for at least 6 h. Pellets are eluted in 100 μl of water and the presence of fluorometabolites are analyzed by LC-MS.

### Example 4—In Vivo Production of Fluorometabolites

Cells of E. coli MG1655 and P. putida KT2440 were transformed with an expression plasmid where the genes encoding the enzymes indicated in Table 8 were expressed under control of the P_{T7} promoter; the strains carried a chromosomally-integrated copy of the T7RNA polymerase gene under transcriptional control of the XylS/Pm expression system for E. coli or the FRS riboswitch for P. putida. The obtain control strains E. coli StEc_Ctrl and P. putida StPp_Ctrl, E. coli MG1655 or P. putida KT2440, respectively, were transformed with empty plasmids (Table 8). In addition to the expressed enzymes listed in Table 8, all strains, except for StEc_Ctrl and StPp_Ctrl, expressed the gene hdhA (SEQ ID NO: 24) encoding the Aldh HdhA from P. aeruginosa PP_4022 (SEQ ID NO: 23).

Recombinant cells were incubated in M9 medium (for E. coli StEc_Ctrl, StEc1 and StEc2) or de Bont minimal medium (for P. putida StPp_Ctrl, StPp1, StPp2 and StPp3) at 37° C. or 30° C., respectively, in 500-mL Erlenmeyer flasks containing 50 mL of the culture. Glucose or citrate were used as carbon source (C source) for the E. coli strains or P. putida strains, respectively, at 20 mM and all cultures were supplied with 1 mM 3-methylbenzoate at the onset of the cultivation. NaF was supplied at 15 mM for cultures of StEc2, StPp2, StPp3, StEc_Ctrl and StPp_Ctrl, and 5′-FDA was supplied at 2.5 mM for cultures of StEc1 and StPp1 (Table 8). Recombinant cells were harvested after cultivation for 24 h and both supernatants and intracellular extracts were analysed with LC-MS and ¹⁹F-NMR, as described in the above examples, for the presence of the fluorometabolites indicated in Table 8.

TABLE 8
Fluorometabolite detection (LC-MS and NMR) of cultures of E. coli StEc1 and StEc2
and P. putida StPp1, StPp2 and StPp3 upon incubation for 24 h. Culture medium of StEc1
and StPp1 was supplied with 5′-FDA. Culture medium of StEc2, StPp2, StPp3, StEc_Ctrl
and StPp_Ctrl was supplied with NaF. “Enzyme” indicates the enzymes harboured by
each strain and are coded as follows: F, fluorinase FIA_{PtaU1} from Methanosaeta
sp. PtaU1.Bin055 (SEQ ID NO: 1); P, phosphorylase FIB1 from S. cattleya
(SEQ ID NO: 11); N, nucleosidase Pfs from E. coli (SEQ ID NO: 17);
K, kinase KinBt (DrdK) from B. thuringiensis (SEQ ID NO: 33); I, isomerase IsoSc
from S. cattleya (SEQ ID NO: 37); A, aldolase AldSc from S. coelicolor (SEQ ID NO: 19).

| | | | | | | | Fluorometabolite detected (at 24 h) | | | | | |
|---|---|---|---|---|---|---|---|---|---|---|---|---|
| | Enzyme | | | | | | 5′- | 5′- | | | | |
| Strain | F | P | N | K | I | A | FDRP | FDRulP | FAld | FAc | FEtOH | FAcCoA |
| E. coli | | | | | | | | | | | | |
| MG1655 | | | | | | | | | | | | |
| StEc1 | − | − | + | + | + | + | + | + | + | + | + | + |
| StEc2 | + | − | + | + | + | + | + | + | * | + | + | * |
| StEc_Ctrl | None | | | | | | None | | | | | |
| P. putida | | | | | | | | | | | | |
| KT2440 | | | | | | | | | | | | |
| StPp1 | − | − | + | + | + | + | + | + | + | + | + | + |
| StPp2 | + | + | − | − | + | + | + | + | * | + | + | * |
| StPp3 | + | − | + | + | + | + | + | + | * | + | + | * |

| | | |
|---|---|---|
| StPp_Ctrl | None | None |

+, indicates presence of enzyme and/or fluorometabolite in supernatant and/or extract. *, positive signal within the lower limit of detection by LC-MS. All strains, except for StEc_Ctrl and StPp_Ctrl, expressed hdhA (SEQ ID NO: 24) encoding the Aldh HdhA from P. aeruginosa PP_4022 (SEQ ID NO: 23).

For all recombinant cells where a nucleosidase (N) and kinase (K) (i.e. phosphorylation bypass pathway) were expressed instead of a phosphorylase (P), a peak compatible with 5′-fluoro-5′-deoxyribose (5′-FDR) could be detected (Table 8). FAc and FEtOH were only assayed in the extracellular medium, whereas all other fluorometabolites were detected in the intracellular extract. In conclusion, all of E. coli StEc1 and StEc2 as well as P. putida StPp1, StPp2 and StPp3 produced 5′-FDRP, 5′-FDRuIP, FAld, FAc, FEtOH and FAcCoA upon supplementation of either 5′-FDA or NaF to the medium (Table 8). No fluorometabolites were detected for the control strains E. coli StEc_Ctrl and P. putida StPp_Ctrl upon supplementation of NaF. Experiments where HdhA (SEQ ID NO: 23) was replaced by ExaC (SEQ ID NO: 21) had similar results in terms of fluorometabolite distribution.

### REFERENCES

Bagdasarian et al., 1981. Specific purpose plasmid cloning vectors. II. Broad host range, high copy number, RSF1010-derived vectors, and a host-vector system for gene cloning in Pseudomonas. Gene. 16, 237-247. https://doi.org/10.1016/0378-1119(81)90080-9
Beaudoin et al., 2018. Salvage of the 5-deoxyribose byproduct of radical SAM enzymes. Nat Commun. 2018 Aug. 6; 9(1):3105. doi: 10.1038/s41467-018-05589-4.
Calero et al., 2020. A fluoride-responsive genetic circuit enables in vivo biofluorination in engineered Pseudomonas putida. Nat Commun. 2020 Oct. 7; 11(1):5045. doi: 10.1038/s41467-020-18813-x.
Deng et al., 2008. In vitro reconstituted biotransformation of 4-fluorothreonine from fluoride ion: application of the fluorinase. Chem Biol. 2008 Dec. 22; 15(12):1268-76. doi: 10.1016/j.chembiol.2008.10.012.
Eustáquio et al, 2010. Engineering fluorometabolite production: fluorinase expression in Salinispora tropica Yields Fluorosalinosporamide. J Nat Prod. 2010 Mar. 26; 73(3):378-82. doi: 10.1021/np900719u.
Iwai et al., 2011. One-pot three-step continuous enzymatic synthesis of 5-fluoro-5-deoxy-d-ribose. Journal of Molecular Catalysis B-enzymatic 73 (2011): 1-4.
Lipmann et al., 1945, A specific micromethod for the determination of acyl phosphates, Journal of Biological Chemistry, Volume 159, Issue 1, 1945, Pages 21-28. https://doi.org/10.1016/S0021-9258(19)51298-4
Salomonsen et al., 2014. USER-Derived Cloning Methods and Their Primer Design. In: Valla S., Lale R. (eds) DNA Cloning and Assembly Methods. Methods in Molecular Biology (Methods and Protocols), vol 1116. Humana Press, Totowa, NJ. https://doi.org/10.1007/978-1-62703-764-8 5
Sambrook, J. & Russell, D. W. Molecular Cloning: a Laboratory Manual, 3rd edn (Cold Spring Harbor Laboratory, Cold Spring Harbor, 2001).
Li et al., 2011. Purification and characterization of the acetyl-CoA synthetase from Mycobacterium tuberculosis, Acta Biochimica et Biophysica Sinica, Volume 43, Issue 11, November 2011, Pages 891-899, https://doi.org/10.1093/abbs/qmr076
Worsey and Williams, 1975. Metabolism of toluene and xylenes by Pseudomonas (putida (arvilla) mt-2: evidence for a new function of the TOL plasmid. Journal of Bacteriology. 1975 October; 124(1):7-13.
Ashkenazy et al., 2016. ConSurf 2016: An improved methodology to estimate and visualize evolutionary conservation in macromolecules. Nucleic Acids Res. 44: W344-W350.
Deng et al. 2006. The fluorinase from Streptomyces cattleya is also a chlorinase. Angew. Chem. Int. Ed. Engl. 45: 759-762.
Eustáquio et al., 2008. Discovery and characterization of a marine bacterial SAM-dependent chlorinase. Nat. Chem. Biol. 4: 69-74.
Hartmans et al., 1989. Metabolism of styrene oxide and 2-phenylethanol in the styrene-degrading Xanthobacter strain 124X. Appl. Environ. Microbiol. 55: 2850-2855.
Hon et al., 2020. EnzymeMiner: Automated mining of soluble enzymes with diverse structures, catalytic properties and stabilities. Nucleic Acids Res. 48: W104-W109.
Pettersen et al., 2004. UCSF Chimera-A visualization system for exploratory research and analysis. J. Comput. Chem. 25: 1605-1612.
Schaffrath et al., 2003. Isolation and characterisation of 5′-fluorodeoxyadenosine synthase, a fluorination enzyme from Streptomyces cattleya. FEBS Lett. 547: 111-114.
Sooklal et al., 2020. Identification and characterisation of a fluorinase from Actinopolyspora mzabensis. Protein Expr. Purif. 166: 105508.
Sun et al., 2016. Directed evolution of a fluorinase for improved fluorination efficiency with a non-native substrate. Angew. Chem. Int. Ed. 55: 14277-14280.
Thomsen et al., 2013. Chemoenzymatic synthesis and in situ application of S-adenosyl-L-methionine analogs. Org. Biomol. Chem. 11: 7606-7610.
Waterhouse, A., Bertoni, M., Bienert, S., Studer, G., Tauriello, G., Gumienny, R., et al. (2018) SWISS-MODEL: Homology modelling of protein structures and complexes. Nucleic Acids Res. 46: W296-W303.
Zhu, X., Robinson, D. A., McEwan, A. R., O'Hagan, D., and Naismith, J. H. (2007) Mechanism of enzymatic fluorination in Streptomyces cattleya. J. Am. Chem. Soc. 129: 14597-14604.
WO2020/083958. In vivo fluorination, chlorination and bromination. Filed on 23 Oct. 2019.

### Sequence Overview

Sequence
ID NO:

| | |
|---|---|
| SEQ ID NO: 1 | FIA_{PtaU1} from Methanosaeta sp. PtaU1•Bin055, protein |
| | sequence, EnsemblBacteria: OPY51785.1, Uniprot: |
| | A0A1V5AZT2 |
| SEQ ID NO: 2 | fIA_{Ptau1} from Methanosaeta sp. PtaU1•Bin055, nucleic |
| | acid, native, EnsemblBacteria: A4E51_01483 |
| SEQ ID NO: 3 | fIA_{PtaU1} from Methanosaeta sp. PtaU1•Bin055, nucleic |
| | acid, codon optimised |
| SEQ ID NO: 4 | FRS: Fluoride responsive riboswitch from Pseudomonas |
| | syringae |
| SEQ ID NO: 5 | FRSv1: Pseudomonas syringae FRS and first amino acids |
| | of EriC^{F} |
| SEQ ID NO: 6 | FRSv2: Pseudomonas syringae FRS |
| SEQ ID NO: 7 | FRSv3: P_{EM7} promoter, Pseudomonas syringae FRS and |
| | first codons of EriC^{F} |
| SEQ ID NO: 8 | FRSv4: P_{EM7} promoter, Pseudomonas syringae FRS |
| SEQ ID NO: 9 | crcB from Pseudomonas putida (PP_4001), native |
| | nucleic acid |
| SEQ ID NO: 10 | CrcB from Pseudomonas putida (PP_4001), protein |
| | sequence |
| SEQ ID NO: 11 | FIB1 from Streptomyces sp. MA37, protein sequence |
| SEQ ID NO: 12 | fIB1 from Streptomyces sp. MA37, native |
| SEQ ID NO: 13 | fIB1 from Streptomyces sp. MA37, codon optimized |
| SEQ ID NO: 14 | deoD from E. coli MG1655, nucleic acid |
| SEQ ID NO: 15 | DeoD from E. coli MG1655, protein sequence |
| SEQ ID NO: 16 | pfs from E. coli MG1655, nucleic acid |
| SEQ ID NO: 17 | Pfs from E. coli MG1655, protein sequence |
| SEQ ID NO: 18 | aldBt (drdA) from B. thuringiensis (aldolase), native |
| | nucleic acid, Uniprot accession number: P0DTQ0 |
| SEQ ID NO: 19 | AldSc from S. coelicolor (aldolase), protein sequence |
| SEQ ID NO: 20 | AldSc from S. coelicolor (aldolase), codon-optimised |
| | nucleic acid |
| SEQ ID NO: 21 | ExaC from P. aeruginosa 1984 (Aldh), protein sequence |
| SEQ ID NO: 22 | exaC from P. aeruginosa 1984 (Aldh), native nucleic |
| | acid |
| SEQ ID NO: 23 | HdhA from P. aeruginosa 4022 (Aldh), protein sequence |
| SEQ ID NO: 24 | hdhA from P. aeruginosa 4022 (Aldh), native nucleic |
| | acid |
| SEQ ID NO: 25 | EutE from E. coli (Aldh), protein sequence |
| SEQ ID NO: 26 | eutE from E. coli (Aldh), native nucleic acid |
| SEQ ID NO: 27 | Moth_1776p from Moorella thermoacetica 1776 (Aldh), |
| | protein sequence, Accession number: Q2RHL2 |
| SEQ ID NO: 28 | Moth_1776 from Moorella thermoacetica 1776 (Aldh), |
| | native nucleic acid, Accession number: Q2RHL2 |
| SEQ ID NO: 29 | AldBt (DrdA) from B. thuringiensis (aldolase), protein |
| | sequence, Uniprot accession number: P0DTQ0 |
| SEQ ID NO: 30 | FIA1_{MA37} from Streptomyces sp. MA37, protein sequence |
| SEQ ID NO: 31 | fIA1_{MA37} from Streptomyces sp. MA37, native nucleic |
| | acid |
| SEQ ID NO: 32 | fIA1_{MA37} from Streptomyces sp. MA37, codon optimised |
| SEQ ID NO: 33 | KinBt (DrdK) from B. thuringiensis (kinase), protein |
| | sequence, Uniprot accession number: P0DTQ2 |
| SEQ ID NO: 34 | kinBt (drdK) from B. thuringiensis (kinase), native |
| | nucleic acid, Uniprot accession number: P0DTQ2 |
| SEQ ID NO: 35 | IsoBt (Drdl) from B. thuringiensis (isomerase), |
| | protein sequence |
| SEQ ID NO: 36 | isoBt (drdl) from B. thuringiensis (isomerase), |
| | native nucleic acid |
| SEQ ID NO: 37 | IsoSc from S. cattleya (isomerase), protein sequence |
| SEQ ID NO: 38 | IsoSc from S. cattleya (isomerase), native |
| | nucleic acid |
| SEQ ID NO: 39 | AcsBt (AcsA) from B. subtilis (Acs), protein |
| | sequence, Uniprot accession number: CAB14946.1 |
| SEQ ID NO: 40 | acsBt (acsA) from B. subtilis (Acs), native |
| | nucleic acid |
| SEQ ID NO: 41 | AcsCn from Cupriavidus necator (Acs), protein |
| | sequence, Accession number: WP_013952735 |
| SEQ ID NO: 42 | acsCn from C. necator (Acs), native nucleic acid |
| SEQ ID NO: 43 | AcsSc (AcsA) from S. coelicolor (Acs), protein |
| | sequence, Accession number: CAB38500 |
| SEQ ID NO: 44 | acsSc (acsA) from S. coelicolor (Acs), native |
| | nucleic acid |

### Items 1

1. A cell capable of producing a fluorinated compound from a substrate in the presence of fluoride, said cell comprising:
   a) a first nucleic acid comprising a first promoter and a fluorinase gene encoding a fluorinase (EC 2.5.1.63) capable of catalysing the formation of a C—F bond, said fluorinase gene being under the control of the first promoter;
   b) optionally a second nucleic acid comprising a riboswitch, wherein transcription of the fluorinase gene from the first promoter is induced in the presence of an inducer, wherein said riboswitch is responsive to said inducer;
   wherein the cell is capable of expressing the activator of transcription and the fluorinase at least in the presence of said inducer,
   wherein the fluorinase is the fluorinase of Methanosaeta sp. PtaU1.Bin055 as set forth in SEQ ID NO: 1 (FlA_{PtaU1}), or a functional variant thereof having at least 70% sequence homology, similarity or identity thereto, such as at least 71%, such as at least 72%, such as at least 73%, such as at least 74%, such as at least 75%, such as at least 76%, such as at least 77%, such as at least 78%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence homology, similarity or identity to SEQ ID NO:1.
2. The cell according to item 1, wherein the second nucleic acid further comprises a second promoter and a gene encoding an activator of transcription, said gene being under the control of the second promoter, wherein transcription of the activator of transcription can be induced by the inducer, wherein the activator of transcription upon expression activates transcription from the first promoter.
3. The cell according to any one of the preceding items, wherein the first nucleic acid and the second nucleic acid are the same nucleic acid.
4. The cell according to any one of the preceding items, wherein the first nucleic acid further comprises a phosphorylase gene such as flB1 or deoD, said gene encoding a phosphorylase (EC 2.4.2.28 and/or EC 2.4.2.1) capable of catalysing the phosphorylation of a fluorinated substrate such as a fluorinated adenosine, said phosphorylase gene being under control of the first promoter.
5. The cell according to any one of the preceding items, wherein the first nucleic acid further comprises a gene encoding a nucleosidase (EC 3.2.2.9) such as Pfs, said nucleosidase being capable of catalysing the conversion of 5′-FDA to 5′-FDR, said nucleosidase gene being under control of the first promoter.
6. The cell according to any one of the preceding items, wherein the second nucleic acid further comprises a second promoter and a gene encoding an activator of transcription, said gene being under the control of the second promoter, wherein transcription of the activator of transcription can be induced by the inducer, wherein the activator of transcription upon expression activates transcription from the first promoter.
7. The cell according to any one of the preceding items, wherein the cell further comprises a phosphorylase gene such as flB1 or deoD, said gene encoding a phosphorylase (EC 2.4.2.28 and/or EC 2.4.2.1) capable of catalysing the phosphorylation of a fluorinated adenosine, said phosphorylase gene being comprised in the first nucleic acid, preferably said phosphorylase gene being under control of the first promoter, whereby said cell is capable of further catalysing phosphorylation of a fluorinated adenosine.
8. The cell according to any one of the preceding items, wherein the first nucleic acid further comprises a gene encoding a nucleosidase (EC 3.2.2.9) such as Pfs, said nucleosidase being capable of catalysing the conversion of 5′-FDA to 5′-FDR, said nucleosidase being under control of the first promoter, whereby said cell is further capable of converting 5′-FDA to 5′-FDR.
9. The cell according to any one of the preceding items, wherein the cell can tolerate toxic compounds, such as fluorinated compounds.
10. The cell according to any one of the preceding items, wherein the cell is a non-pathogenic organism.
11. The cell according to any one of the preceding items, wherein the cell is a mammalian cell, a plant cell, an insect cell, a yeast cell or a bacterial cell.
12. The cell according to any one of the preceding items, wherein the cell is a bacterial cell of the Pseudomonas genus, the Bacillus genus, the Streptomyces genus, the Vibrio genus or the Escherichia genus or a yeast cell of the genus Saccharomyces, Pichia, Yarrowia, Kluyveromyces, Candida, Rhodotorula, Rhodosporidium, Cryptococcus, Trichosporon and Lipomyces.
13. The cell according to any one of the preceding items, wherein the cell is a bacterial cell selected from Pseudomonas putida, Pseudomonas fluorescens, Pseudomonas taiwanensis, Pseudomonas syringae, Pseudomonas stutzeri, Pseudomonas oleovorans, Pseudomonas mendocina, Bacillus subtilis, Bacillus cereus, Bacillus megaterium, Streptomyces albus, Streptomyces venezuelae, Streptomyces coelicolor, Vibrio natriegens and Escherichia coli, or a yeast cell selected from Saccharomyces cerevisiae, Pichia pastoris, Kluyveromyces marxianus, Cryptococcus albidus, Lipomyces lipofer, Lipomyces starkeyi, Rhodosporidium toruloides, Rhodotorula glutinis, Trichosporon pullulans and Yarrowia lipolytica.
14. The cell according to any one of the preceding items, wherein the cell is Pseudomonas putida KT2440.
15. The cell according to any one of the preceding items, wherein the fluorinase is capable of catalysing fluorination of a substrate to obtain a fluorinated compound, such as 5′-deoxy-5′-fluoroadenosine, or 5′-deoxy-5′-fluoro-D-ribose 1-phosphate.
16. The cell according to any one of the preceding items, wherein the substrate is selected from the group consisting of S-adenosyl-L-methionine (SAM) or a derivative thereof, such as a methylaza derivative, 5′-chloro-5′-deoxyadenosine, a 2-deoxyadenosine analogue, an L-methionine analogue, a di-cyclic peptide conjugate of 5′-chlorodeoxy-2-ethynyladenosine, a tri-cyclic peptide conjugate of 5′-chlorodeoxy-2-ethynyladenosine, fluoride, ¹⁸F, 5′-deoxy-5′-fluoroadenosine, 5′-chloro-5′-deoxyadenosine, and 5′-bromo-5′-deoxyadenosine.
17. The cell according to any one of the preceding items, wherein the substrate is a SAM derivative of formula (I):

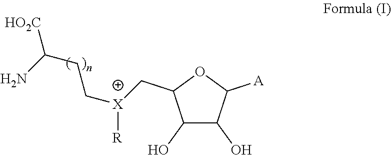

wherein:
n is 0, 1 or 2;
X is selected from the group consisting of: S, Se and NMe;
R is selected from the group consisting of: Me and propargyl;
A is a heterocycle.
18. The cell according to item 17, wherein the SAM derivative of formula (I) is of formula (II):

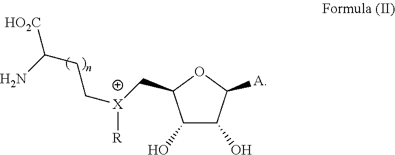

19. The cell according to any of the preceding items wherein the substrate is selected from the group consisting of

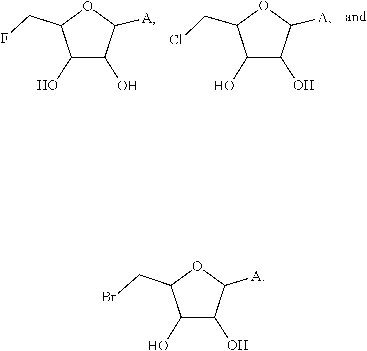

20. The cell according to any one of items 17 to 19, wherein A is selected from the group consisting of

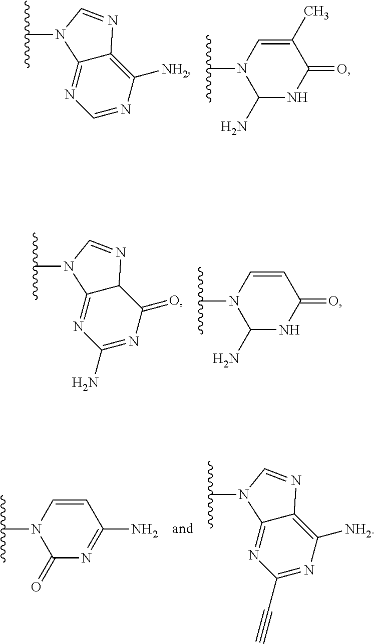

21. The cell according to any one of items 17 to 20, wherein the SAM derivative of formula (I) is of formula (III):

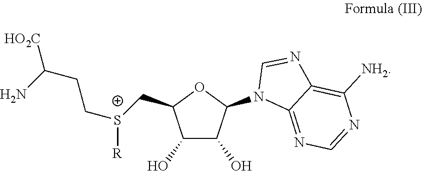

22. The cell according to any one of items 17 to 21, wherein the substrate is selected from the group consisting of

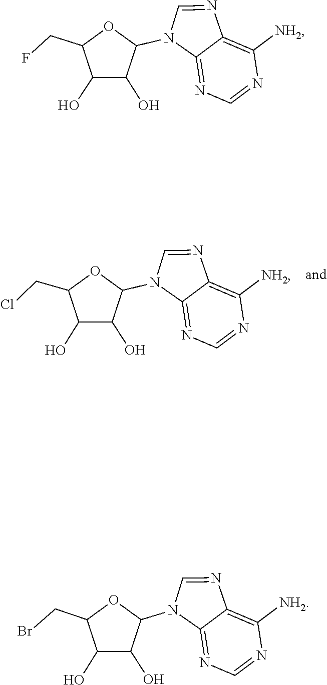 [SMILES: Nc1ncnc2c1ncn2C1OC(CF)C(O)C1O ; Nc1ncnc2c1ncn2C1OC(CCl)C(O)C1O ; Nc1ncnc2c1ncn2C1OC(CBr)C(O)C1O]

23. The cell according to any one of the preceding items, wherein the fluorinase gene is codon-optimised.
24. The cell according to any one of the preceding items, wherein the fluorinase gene is as set forth in SEQ ID NO: 2 or SEQ ID NO: 3, or a homologue thereof having at 70% sequence homology, similarity or identity thereto, such as at least 71%, such as at least 72%, such as at least 73%, such as at least 74%, such as at least 75%, such as at least 76%, such as at least 77%, such as at least 78%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence homology, similarity or identity to SEQ ID NO: 2 or SEQ ID NO: 3.
25. The cell according to any one of the preceding items, wherein the phosphorylase is the phosphorylase as set forth in SEQ ID NO: 11 or SEQ ID NO: 15, or a functional variant thereof having at least 70% sequence homology, similarity or identity thereto, such as at least 71%, such as at least 72%, such as at least 73%, such as at least 74%, such as at least 75%, such as at least 76%, such as at least 77%, such as at least 78%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence homology, similarity or identity to SEQ ID NO: 11 or SEQ ID NO: 15.
26. The cell according to any one of the preceding items, wherein the nucleosidase is the nucleosidase as set forth in SEQ ID NO: 17, or a functional variant thereof having at least 70% sequence homology, similarity or identity thereto, such as at least 71%, such as at least 72%, such as at least 73%, such as at least 74%, such as at least 75%, such as at least 76%, such as at least 77%, such as at least 78%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence homology, similarity or identity to SEQ ID NO: 17.
27. The cell according to any one of the preceding items, wherein the phosphorylase gene is as set forth in SEQ ID NO: 12, SEQ ID NO: 13 or SEQ ID NO: 14, or a homologue thereof having at least 70% sequence homology, similarity or identity thereto, such as at least 71%, such as at least 72%, such as at least 73%, such as at least 74%, such as at least 75%, such as at least 76%, such as at least 77%, such as at least 78%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence homology, similarity or identity to SEQ ID NO: 12, SEQ ID NO: 13 or SEQ ID NO: 14.
28. The cell according to any one of the preceding items, wherein the nucleosidase gene is as set forth in SEQ ID NO: 16, or a homologue thereof having at least 70% sequence homology, similarity or identity thereto, such as at least 71%, such as at least 72%, such as at least 73%, such as at least 74%, such as at least 75%, such as at least 76%, such as at least 77%, such as at least 78%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence homology, similarity or identity to SEQ ID NO: 16.
29. The cell according to any one of the preceding items, wherein the phosphorylase gene, the nucleosidase gene and/or the fluorinase gene is codon-optimised.
30. The cell according to any one of the preceding items, wherein the activator of transcription is a polymerase such as a T7 RNA polymerase and the first promoter is the native promoter of said polymerase such as a T7 promoter.
31. The cell according to any one of the preceding items, wherein the polymerase is a T7 RNA polymerase and the first promoter is a T7 promoter.
32. The cell according to any one of the preceding items, wherein the riboswitch is the fluoride-responsive riboswitch (FRS) from Pseudomonas syringae as set forth in SEQ ID NO: 4, or a homologue thereof having at least 90% sequence homology, similarity or identity to SEQ ID NO: 4, such at least 91%, such as at least 92%, such as at least 93%, such as at least 94%, such as at least 95%, such as at least 96%, such as at least 97%, such as at least 98%, such as at least 99%, such as 100% sequence homology, similarity or identity to SEQ ID NO:4.
33. The cell according to any one of the preceding items, wherein the second nucleic acid is as set forth in SEQ ID NO: 5, SEQ ID NO: 6, SEQ ID NO: 7 or SEQ ID NO: 8, preferably as set forth in SEQ ID NO: 5, SEQ ID NO: 6 or SEQ ID NO: 8, most preferably as set forth in SEQ ID NO: 5 or SEQ ID NO: 6; or wherein the nucleic acid comprises or consists of a sequence having at least 90% sequence homology, similarity or identity to SEQ ID NO: 5, SEQ ID NO: 6, SEQ ID NO: 7 or SEQ ID NO: 8, such as at least 91%, such as at least 92%, such as at least 93%, such as at least 94%, such as at least 95%, such as at least 96%, such as at least 97%, such as at least 98%, such as at least 99%, such as 100% sequence homology, similarity or identity to SEQ ID NO: 5, SEQ ID NO: 6, SEQ ID NO: 7 or SEQ ID NO: 8, preferably to SEQ ID NO: 5, SEQ ID NO: 6 or SEQ ID NO: 8, most preferably to SEQ ID NO: 5 or SEQ ID NO: 6.
34. The cell according to any one of the preceding items, wherein the inducer is a fluoride salt such as NaF or KF.
35. The cell according to any one of the preceding items, further comprising a mutation in at least one fluoride transporter gene encoding a fluoride transporter, said mutation resulting in a partial or total loss of function of said fluoride transporter.
36. The cell according to item 35, wherein the mutation is a deletion.
37. The cell according to any one of items 35 to 36, wherein the cell is a Pseudomonas putida cell and the fluoride transporter gene is the crcB gene as set forth in SEQ ID NO: 9.
38. The cell according to any one of the preceding items, wherein 5′-deoxy-5′-fluoroadenosine is produced with a titer of at least 1 μM, such as at least 1.5 μM, such as at least 2 μM, such as at least 2.5 μM, such as at least 3 μM, such as at least 3.5 μM, such as at least 4 μM, such as at least 4.5 μM, such as at least 5 μM, such as at least 5.5 μM, such as at least 6 μM, such as at least 6.5 μM, such as at least 7 μM, such as at least 7.5 μM, such as at least 8 μM, such as at least 8.5 μM, such as at least 9 μM, such as at least 9.5 μM, such as at least 10 μM, such as at least 15 μM, such as at least 20 μM, such as at least 25 μM, such as at least 30 μM, such as at least 35 μM, such as at least 40 μM, such as at least 45 μM, such as at least 50 μM, such as at least 55 μM, such as at least 60 μM, such as at least 70 μM, such as at least 80 μM, such as at least 90 μM, such as at least 100 μM, or more.
39. An expression system for expression in a cell, comprising:
   a) a first nucleic acid comprising a first promoter and a fluorinase gene encoding a fluorinase (EC 2.5.1.63) capable of catalysing the formation of a C—F bond, said fluorinase gene being under the control of the first promoter;
   b) optionally a second nucleic acid comprising a riboswitch, wherein transcription of the fluorinase gene from the first promoter is induced in the presence of an inducer, wherein said riboswitch is responsive to said inducer,
   wherein the fluorinase is the fluorinase of Methanosaeta sp. PtaU1.Bin055 as set forth in SEQ ID NO: 1 (FlA_{PtaU1}), or a functional variant thereof having at least 70% sequence homology, similarity or identity thereto.
40. The expression system in a cell according to item 39, wherein the second nucleic acid further comprises a second promoter and a gene encoding an activator of transcription, said gene being under the control of the second promoter, wherein transcription of the activator of transcription can be induced by the inducer, wherein the activator of transcription upon expression activates transcription from the first promoter.
41. The expression system in a cell according to any one of items 39 to 40, wherein the first nucleic acid and the second nucleic acid are the same nucleic acid.
42. The expression system in a cell according to any one of items 39 to 41, wherein the first nucleic acid further comprises a phosphorylase gene such as flB1 or deoD, said gene encoding a phosphorylase (EC 2.4.2.28 and/or EC 2.4.2.1) capable of catalysing the phosphorylation of a fluorinated adenosine, said phosphorylase gene being under control of the first promoter.
43. The expression system in a cell according to any one of items 39 to 42, wherein the first nucleic acid further comprises a gene encoding a nucleosidase (EC 3.2.2.9) such as Pfs, said nucleosidase being capable of catalysing the conversion of 5′-FDA to 5′-FDR, said nucleosidase gene being under control of the first promoter.
44. The expression system according to any one of items 39 to 43, wherein the second nucleic acid further comprises a second promoter and a gene encoding an activator of transcription, said gene being under the control of the second promoter, wherein transcription of the activator of transcription can be induced by the inducer, wherein the activator of transcription upon expression activates transcription from the first promoter.
45. The expression system according to any one of the preceding items, wherein the first nucleic acid further comprises a nucleosidase gene such as Pfs, said gene encoding a nucleosidase (EC 3.2.2.9) capable of catalysing the conversion of 5′-FDA to 5′-FDR, said nucleosidase gene being under control of the first promoter.
46. The expression system according to any one of the preceding items, wherein the first nucleic acid further comprises a phosphorylase gene such as flB1 or deoD, said gene encoding a phosphorylase (EC 2.4.2.28 and/or EC 2.4.2.1) capable of catalysing the phosphorylation of a fluorinated adenosine, said phosphorylase gene being under control of the first promoter.
47. The expression system according to any one of items 44 to 45, wherein the second nucleic acid further comprises a second promoter and a gene encoding an activator of transcription, said gene being under the control of the second promoter, wherein transcription of the activator of transcription can be induced by an inducer, wherein the activator of transcription upon expression activates transcription from the first promoter.
48. The expression system of items 44 to 47, wherein the cell is as defined in any one of items 1 to 37.
49. The expression system of any one of items 44 to 48, wherein the first nucleic acid and/or the second nucleic acid are independently comprised in a vector or are integrated in the genome of the cell.
50. A method for in vivo fluorination of a substrate, comprising the steps of:
i) propagating a cell in a medium, said cell comprising:
   a) a first nucleic acid comprising a first promoter and a fluorinase gene encoding a fluorinase (EC 2.5.1.63) capable of catalysing the formation of a C—F bond, said fluorinase gene being under the control of the first promoter;
   b) optionally a second nucleic acid comprising a riboswitch, wherein transcription of the fluorinase gene from the first promoter is induced in the presence of an inducer, wherein said riboswitch is responsive to said inducer;
ii) adding the inducer to the medium and incubating the cell in the presence of inducer and a co-substrate such as a fluoride salt,
   wherein the fluorinase is the fluorinase of Methanosaeta sp. PtaU1.Bin055 as set forth in SEQ ID NO: 1 (FlA_{PtaU1}), or a functional variant thereof having at least 70% sequence homology, similarity or identity thereto,
   whereby transcription of the fluorinase gene is induced,
   thereby inducing fluorination of the substrate to yield a fluorinated compound.
51. The method according to item 51, wherein;
   i. the fluorinated compound is 5′-FDA;
   ii. the cell further comprises a gene encoding a nucleosidase (EC 3.2.2.9) such as Pfs, said nucleosidase being capable of catalysing the conversion of 5′-FDA to 5′-FDR, said gene being under control of the first promoter,
      thereby yielding conversion of 5′-FDA to 5′-FDR.
52. A method for in vivo production of 5′-FDR comprising the steps of:
i) propagating a cell in a medium, said cell comprising:
   a) a first nucleic acid comprising a first promoter and a fluorinase gene encoding a fluorinase (EC 2.5.1.63) capable of catalysing the formation of a C—F bond, and a gene encoding a nucleosidase (EC 3.2.2.9) such as Pfs, said nucleosidase being capable of catalysing the conversion of 5′-FDA to 5′-FDR, said fluorinase gene and said nucleosidase gene being under the control of the first promoter;
   b) optionally a second nucleic acid comprising a riboswitch, wherein transcription of the fluorinase gene from the first promoter is induced in the presence of an inducer, wherein said riboswitch is responsive to said inducer;
ii) adding the inducer to the medium and incubating the cell in the presence of inducer and a co-substrate such as a fluoride salt,
   wherein the fluorinase is the fluorinase of Methanosaeta sp. PtaU1.Bin055 as set forth in SEQ ID NO: 1 (FlA_{PtaU1}), or a functional variant thereof having at least 70% sequence homology, similarity or identity thereto,
   whereby transcription of the fluorinase gene and of the nucleosidase is induced,
   thereby inducing production of 5′-FDA and its conversion to 5′-FDR.
53. The method according to any one of items 50 to 51, wherein the second nucleic acid further comprises a second promoter and a gene encoding an activator of transcription, said gene being under the control of the second promoter, wherein transcription of the activator of transcription can be induced by the inducer, wherein the activator of transcription upon expression activates transcription from the first promoter, thereby inducing transcription of the fluorinase gene.
54. The method according to any one of items 50 to 53, wherein the first nucleic acid and the second nucleic acid are the same nucleic acid.
55. The method according to any one of items 50 to 54, wherein the first nucleic acid further comprises a phosphorylase gene such as flB1 or deoD, said gene encoding a phosphorylase (EC 2.4.2.28 and/or EC 2.4.2.1) capable of catalysing the phosphorylation of a fluorinated substrate such as a fluorinated adenosine, said gene being under control of the first promoter.
56. The method according to any one of items 50 to 54, wherein the second nucleic acid further comprises a second promoter and a gene encoding an activator of transcription, said gene being under the control of the second promoter, wherein transcription of the activator of transcription can be induced by the inducer, wherein the activator of transcription upon expression activates transcription from the first promoter, thereby inducing transcription of the fluorinase gene, the nucleosidase gene and/or the phosphorylase gene.
57. The method according to any one of the preceding items, wherein the cell further comprises a phosphorylase gene such as flB1 or deoD, said gene encoding a phosphorylase (EC 2.4.2.28 and/or EC 2.4.2.1) capable of catalysing the phosphorylation of a fluorinated substrate such as a fluorinated adenosine, said gene being under control of the first promoter, thereby yielding a fluorinated and phosphorylated product.
58. The method according to item 57, wherein the second nucleic acid further comprises a second promoter and a gene encoding an activator of transcription, said gene being under the control of the second promoter, wherein transcription of the activator of transcription can be induced by an inducer, wherein the activator of transcription upon expression activates transcription from the first promoter.
59. The method according to any one of items 50 to 58, wherein the cell is as defined in any one of items 1 to 37, or wherein the cell comprises an expression system as defined in any one of items 39 to 49.
60. The method according to any one of items 50 to 59, wherein the inducer is the co-substrate.
61. The method according to any one of items 50 to 60, wherein the method is for in vivo fluorination of the substrate, and the co-substrate or inducer is a fluoride salt such as NaF or KF.
62. The method according to any one of items 50 to 61, wherein NaF or KF is added at a concentration of at least 0.05 mM, such as at least 0.1 mM, such as at least 0.2 mM, such as at least 0.25 mM, such as at least 0.3 mM, such as at least 0.4 mM, such as at least 0.5 mM, such as at least 0.6 mM, such as at least 0.7 mM, such as at least 0.75 mM, such as at least 0.8 mM, such as at least 0.9 mM, such as at least 1 mM, such as at least 2 mM, such as at least 3 mM, such as at least 4 mM, such as at least 5 mM, such as at least 6 mM, such as at least 7 mM, such as at least 8 mM, such as at least 9 mM, such as at least 10 mM, such as at least 11 mM, such as at least 12 mM, such as at least 13 mM, such as at least 14 mM, such as at least 15 mM, such as at least 20 mM, such as 25 mM or more.
63. The method according to any one of items 50 to 62, wherein the inducer and/or co-substrate is added at the culture onset or in mid-exponential phase.
64. The method according to any one of items 50 to 63, wherein the cell is incubated in the presence of inducer and/or co-substrate for at least 4 hours, such as at least 8 hours, such as at least 12 hours, such as at least 16 hours, such as at least 20 hours, such as at least 24 hours, such as at least 36 hours, such as at least 48 hours, or more.
65. The method according to any one of the preceding items, wherein 5′-deoxy-5′-fluoroadenosine is produced with a titer of at least 1 μM, such as at least 1.5 μM, such as at least 2 μM, such as at least 2.5 μM, such as at least 3 μM, such as at least 3.5 μM, such as at least 4 μM, such as at least 4.5 μM, such as at least 5 μM, such as at least 5.5 μM, such as at least 6 μM, such as at least 6.5 μM, such as at least 7 μM, such as at least 7.5 μM, such as at least 8 μM, such as at least 8.5 μM, such as at least 9 μM, such as at least 9.5 μM, such as at least 10 μM, such as at least 15 μM, such as at least 20 μM, such as at least 25 μM, such as at least 30 μM, such as at least 35 μM, such as at least 40 μM, such as at least 45 μM, such as at least 50 μM, such as at least 55 μM, such as at least 60 μM, such as at least 70 μM, such as at least 80 μM, such as at least 90 μM, such as at least 100 μM, or more.
66. A method for in vitro fluorination of a substrate, comprising the steps of:
   i. providing a fluorinase as set forth in SEQ ID NO: 1 (FlA_{PtaU1}), or a functional variant thereof having at least 70% sequence homology, similarity or identity thereto;
   ii. contacting said fluorinase with the substrate in the presence of fluoride;
   whereby said substrate is fluorinated to yield a fluorinated compound.
67. The method according to item 66, wherein the fluorinase is a functional variant of the fluorinase of Methanosaeta sp. PtaU1.Bin055 as set forth in SEQ ID NO: 1 (FlA_{PtaU1}), having at least 70% sequence homology, similarity or identity thereto, such as at least 71%, such as at least 72%, such as at least 73%, such as at least 74%, such as at least 75%, such as at least 76%, such as at least 77%, such as at least 78%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence homology, similarity or identity to SEQ ID NO: 1.
68. The method according to any one of items 66-67, wherein the substrate is as defined in any one of items 10-15.
69. The method according to any one of items 66-67, wherein the fluorinase comprises or consists of the sequence as set forth in SEQ ID NO: 1, with the exception that at the most 30 residues are mutated.
70. The method according to any one of items 50 to 69, further comprising a step of recovering the fluorinated compound.
71. A fluorinated compound or 5′-FDR obtainable by the method according to any one of items 50 to 70.
72. A composition comprising a fluorinated compound according to item 71.
73. A method for manufacturing a fluorinated compound of interest, said method comprising the steps of:
i) providing a fluorinated compound by the method of any one of items 50 to 70; and
ii) optionally converting said fluorinated compound to the fluorinated compound of interest.
74. The fluorinated compound or the 5′-FDR according to item 71 or the composition according to item 72 for use as a medicament.
75. Use of the fluorinated compound according to item 71 or the composition according to item 72 as a building block or a precursor of polymers.
76. Use of a polypeptide as set forth in SEQ ID NO: 1 or a functional variant thereof having at least 70% sequence homology, similarity or identity thereto for catalysing fluorination of a substrate.
77. The use according to item 76, wherein the polypeptide comprises or consists of the sequence as set forth in SEQ ID NO: 1, with the exception that at the most 30 residues are mutated.
78. The use according to any one of items 76 to 77, wherein fluorination is performed in vitro or in vivo.
79. The use according to any one of items 76 to 78, wherein the substrate is as defined in any one of items 10 to 15.

### Items 2

1. A cell capable of producing a fluorinated compound from a substrate in the presence of fluoride, said cell comprising:
   a) a first nucleic acid comprising a first promoter and a fluorinase gene encoding a fluorinase (EC 2.5.1.63) capable of catalysing the formation of a C—F bond, said fluorinase gene being under the control of the first promoter;
   b) optionally a second nucleic acid comprising a riboswitch,
   wherein transcription of the fluorinase gene from the first promoter is induced in the presence of an inducer, wherein said riboswitch is responsive to said inducer;
   wherein the cell is capable of expressing the fluorinase at least in the presence of said inducer,
   wherein the fluorinase is the fluorinase of Methanosaeta sp. PtaU1.Bin055 as set forth in SEQ ID NO: 1 (FlA_{PtaU1}), or a functional variant thereof having at least 70% sequence homology, similarity or identity thereto.
2. The cell according to item 1, wherein the first nucleic acid and the second nucleic acid are the same nucleic acid or wherein the first nucleic acid and the second nucleic acid are different nucleic acids.
3. The cell according to any one of the preceding items, wherein the second nucleic acid further comprises a second promoter and a gene encoding an activator of transcription, said gene being under the control of the second promoter, wherein transcription of the activator of transcription can be induced by the inducer, wherein the activator of transcription upon expression activates transcription from the first promoter.
4. The cell according to any one of the preceding items, wherein the cell can tolerate toxic compounds, such as fluorinated compounds.
5. The cell according to any one of the preceding items, wherein the cell is a non-pathogenic organism.
6. The cell according to any one of the preceding items, wherein the cell is a mammalian cell, a plant cell, an insect cell, a yeast cell or a bacterial cell.
7. The cell according to any one of the preceding items, wherein the cell is a bacterial cell of the Pseudomonas genus, the Bacillus genus, the Streptomyces genus, the Vibrio genus or the Escherichia genus or a yeast cell of the genus Saccharomyces, Pichia, Yarrowia, Kluyveromyces, Candida, Rhodotorula, Rhodosporidium, Cryptococcus, Trichosporon or Lipomyces.
8. The cell according to any one of the preceding items, wherein the cell is a bacterial cell selected from Pseudomonas putida, Pseudomonas fluorescens, Pseudomonas taiwanensis, Pseudomonas syringae, Pseudomonas stutzeri, Pseudomonas oleovorans, Pseudomonas mendocina, Bacillus subtilis, Bacillus cereus, Bacillus megaterium, Streptomyces albus, Streptomyces venezuelae, Streptomyces coelicolor, Vibrio natriegens and Escherichia coli, or a yeast cell selected from Saccharomyces cerevisiae, Pichia pastoris, Kluyveromyces marxianus, Cryptococcus albidus, Lipomyces lipofer, Lipomyces starkeyi, Rhodosporidium toruloides, Rhodotorula glutinis, Trichosporon pullulans and Yarrowia lipolytica, preferably the cell is Pseudomonas putida KT2440.
9. The cell according to any one of the preceding items, wherein the cell is Pseudomonas putida KT2440.
10. The cell according to any one of the preceding items, wherein the fluorinase is capable of catalysing fluorination of a substrate such as adenosine to obtain a fluorinated compound, such as 5′-deoxy-5′-fluoroadenosine.
11. The cell according to any one of the preceding items, wherein the substrate is selected from the group consisting of S-adenosyl-L-methionine (SAM) or a derivative thereof, such as a methylaza derivative, 5′-chloro-5′-deoxyadenosine, a 2-deoxyadenosine analogue, an L-methionine analogue, a di-cyclic peptide conjugate of 5′-chlorodeoxy-2-ethynyladenosine, a tri-cyclic peptide conjugate of 5′-chlorodeoxy-2-ethynyladenosine, fluoride, and ¹⁸F.
12. The cell according to any one of the preceding items, wherein the substrate is a SAM derivative of formula (I):

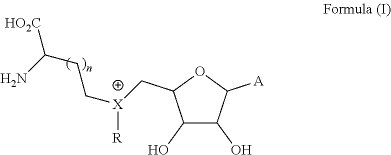

wherein:
n is 0, 1 or 2;
X is selected from the group consisting of: S, Se and NMe;
R is selected from the group consisting of: Me and propargyl;
A is a heterocycle.
13. The cell according to item 12, wherein the SAM derivative of formula (I) is of formula (II):

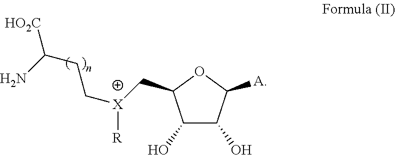

14. The cell according to any one of items 12 to 13, wherein A is selected from the group consisting of

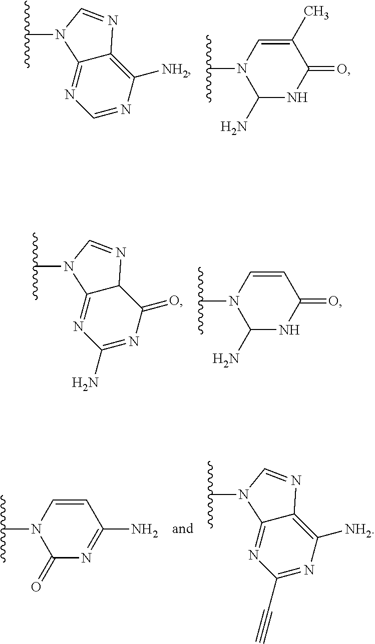

15. The cell according to any one of items 12 to 14, wherein the SAM derivative of formula (I) is of formula (III):

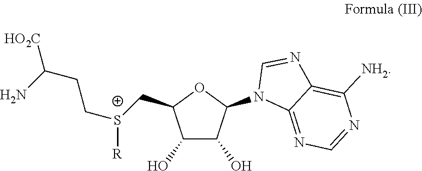

16. The cell according to any one of the preceding items, wherein the fluorinase gene is codon-optimised.
17. The cell according to any one of the preceding items, wherein the fluorinase gene is as set forth in SEQ ID NO: 2 or SEQ ID NO: 3, or a homologue thereof having at least 70% sequence homology, similarity or identity thereto, such as at least 71%, such as at least 72%, such as at least 73%, such as at least 74%, such as at least 75%, such as at least 76%, such as at least 77%, such as at least 78%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence homology, similarity or identity to SEQ ID NO: 2 or SEQ ID NO: 3.
18. The cell according to any one of the preceding items, wherein 5′-deoxy-5′-fluoroadenosine is produced with a titer of at least 1 μM, such as at least 1.5 μM, such as at least 2 μM, such as at least 2.5 μM, such as at least 3 μM, such as at least 3.5 μM, such as at least 4 μM, such as at least 4.5 μM, such as at least 5 μM, such as at least 5.5 μM, such as at least 6 μM, such as at least 6.5 μM, such as at least 7 μM, such as at least 7.5 μM, such as at least 8 μM, such as at least 8.5 μM, such as at least 9 μM, such as at least 9.5 μM, such as at least 10 μM, such as at least 15 μM, such as at least 20 μM, such as at least 25 μM, such as at least 30 μM, such as at least 35 μM, such as at least 40 μM, such as at least 45 μM, such as at least 50 μM, such as at least 55 μM, such as at least 60 μM, such as at least 70 μM, such as at least 80 μM, such as at least 90 μM, such as at least 100 μM, or more.
19. The cell according to any one of the preceding items, wherein the first nucleic acid further comprises a phosphorylase gene such as flB1 or deoD, said gene encoding a phosphorylase (EC 2.4.2.28 and/or EC 2.4.2.1) capable of catalysing the phosphorylation of a fluorinated substrate such as a fluorinated adenosine, said gene being under control of the first promoter, whereby the cell is capable of producing a phosphorylated and fluorinated compound such as 5′-deoxy-5′-fluoro-D-ribose 1-phosphate.
20. The cell according to any one of the preceding items, wherein the phosphorylase gene is codon optimised.
21. The cell according to any one of the preceding items, wherein the gene encoding the phosphorylase is the flB1 gene as set forth in SEQ ID NO: 12 or SEQ ID NO: 13, or the deoD gene as set forth in SEQ ID NO: 14, or a homologue thereof having at least 70% sequence homology, similarity or identity thereto, such as at least 71%, such as at least 72%, such as at least 73%, such as at least 74%, such as at least 75%, such as at least 76%, such as at least 77%, such as at least 78%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence homology, similarity or identity to SEQ ID NO: 12, SEQ ID NO: 13 or SEQ ID NO: 14.
22. The cell according to any one of the preceding items, wherein the first nucleic acid further comprises a gene encoding a nucleosidase (EC 3.2.2.9) such as Pfs, said nucleosidase being capable of catalysing the conversion of 5′-FDA to 5′-FDR, said nucleosidase gene being under control of the first promoter, optionally wherein the gene encoding the nucleosidase is codon-optimised.
23. The cell according to item 22, wherein the nucleosidase gene is the pfs gene as set forth in SEQ ID NO: 16, or a homologue thereof having at least 70% sequence homology, similarity or identity thereto, such as at least 71%, such as at least 72%, such as at least 73%, such as at least 74%, such as at least 75%, such as at least 76%, such as at least 77%, such as at least 78%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence homology, similarity or identity to SEQ ID NO: 16.
24. The cell according to any one of the preceding items, wherein the activator of transcription is a polymerase such as a T7 RNA polymerase and the first promoter is the native promoter of said polymerase such as a T7 promoter.
25. The cell according to any one of the preceding items, wherein the polymerase is a T7 RNA polymerase and the first promoter is a T7 promoter.
26. The cell according to any one of the preceding items, wherein the riboswitch is the fluoride-responsive riboswitch (FRS) from Pseudomonas syringae as set forth in SEQ ID NO: 4, or a homologue thereof having at least 90% sequence homology, similarity or identity to SEQ ID NO: 4, such at least 91%, such as at least 92%, such as at least 93%, such as at least 94%, such as at least 95%, such as at least 96%, such as at least 97%, such as at least 98%, such as at least 99%, such as 100% sequence homology, similarity or identity to SEQ ID NO: 4.
27. The cell according to any one of the preceding items, wherein the second nucleic acid is as set forth in SEQ ID NO: 5, SEQ ID NO: 6, SEQ ID NO: 7 or SEQ ID NO: 8, preferably as set forth in SEQ ID NO: 5, SEQ ID NO: 6 or SEQ ID NO: 8, most preferably as set forth in SEQ ID NO: 5 or SEQ ID NO: 6; or wherein the nucleic acid comprises or consists of a sequence having at least 90% sequence homology, similarity or identity to SEQ ID NO: 5, SEQ ID NO: 6, SEQ ID NO: 7 or SEQ ID NO: 8, such as at least 91%, such as at least 92%, such as at least 93%, such as at least 94%, such as at least 95%, such as at least 96%, such as at least 97%, such as at least 98%, such as at least 99%, such as 100% sequence homology, similarity or identity to SEQ ID NO: 5, SEQ ID NO: 6, SEQ ID NO: 7 or SEQ ID NO: 8, preferably to SEQ ID NO: 5, SEQ ID NO: 6 or SEQ ID NO: 8, most preferably to SEQ ID NO: 5 or SEQ ID NO: 6.
28. The cell according to any one of the preceding items, wherein the inducer is a fluoride salt such as NaF or KF.
29. The cell according to any one of the preceding items, further comprising a mutation in at least one fluoride transporter gene encoding a fluoride transporter, said mutation resulting in a partial or total loss of function of said fluoride transporter.
30. The cell according to item 29, wherein the mutation is a deletion.
31. The cell according to any one of items 29 to 30, wherein the cell is a Pseudomonas putida cell and the fluoride transporter gene is the crcB gene as set forth in SEQ ID NO: 9.
32. An expression system for expression in a cell, comprising:
   a) a first nucleic acid comprising a first promoter and a fluorinase gene encoding a fluorinase (EC 2.5.1.63) capable of catalysing the formation of a C—F bond, said fluorinase gene being under the control of the first promoter;
   b) optionally a second nucleic acid comprising a riboswitch, wherein transcription of the fluorinase gene from the first promoter is induced in the presence of an inducer, wherein said riboswitch is responsive to said inducer, whereby the cell is capable of producing a fluorinated compound such as 5′-fluoro-5′-deoxyadenosine or 5′-deoxy-5′-fluoro-D-ribose 1-phosphate,
      wherein the fluorinase is the fluorinase of Methanosaeta sp. PtaU1.Bin055 as set forth in SEQ ID NO: 1 (FlA_{PtaU1}), or a functional variant thereof having at least 70% sequence homology, similarity or identity thereto.
33. The expression system according to item 32, wherein the second nucleic acid further comprises a second promoter and a gene encoding an activator of transcription, said gene being under the control of the second promoter, wherein transcription of the activator of transcription can be induced by the inducer, wherein the activator of transcription upon expression activates transcription from the first promoter.
34. The expression system according to any one of items 32 to 33, wherein the first nucleic acid and the second nucleic acid are the same nucleic acid or wherein the first nucleic acid and the second nucleic acid are different nucleic acids.
35. The expression system according to any one of items 32 to 34, wherein the first nucleic acid further comprises a gene encoding a phosphorylase such as flB1 or deoD, said phosphorylase (EC 2.4.2.28 and/or EC 2.4.2.1) being capable of catalysing the phosphorylation of a fluorinated adenosine and/or a gene encoding a nucleosidase (EC 3.2.2.9) such as Pfs, said nucleosidase being capable of catalysing the conversion of 5′-FDA to 5′-FDR, said phosphorylase gene and/or said nucleosidase gene being under control of the first promoter.
36. The expression system in a cell according to any one of items 32 to 35, wherein the second nucleic acid further comprises a second promoter and a gene encoding an activator of transcription, said gene being under the control of the second promoter, wherein transcription of the activator of transcription can be induced by the inducer, wherein the activator of transcription upon expression activates transcription from the first promoter.
37. The expression system according to any one of items 32 to 36, wherein the gene encoding the phosphorylase is the flB1 gene as set forth in SEQ ID NO: 12 or SEQ ID NO: 13, or the deoD gene as set forth in SEQ ID NO: 14, or a homologue thereof having at least 70% sequence homology, similarity or identity thereto, such as at least 71%, such as at least 72%, such as at least 73%, such as at least 74%, such as at least 75%, such as at least 76%, such as at least 77%, such as at least 78%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence homology, similarity or identity to SEQ ID NO: 12, SEQ ID NO: 13 or SEQ ID NO: 14.
38. The expression system according to any one of items 32 to 37, wherein the nucleosidase gene is as set forth in SEQ ID NO: 16, or a homologue thereof having at 70% sequence homology, similarity or identity thereto, such as at least 71%, such as at least 72%, such as at least 73%, such as at least 74%, such as at least 75%, such as at least 76%, such as at least 77%, such as at least 78%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence homology, similarity or identity to SEQ ID NO: 16.
39. The expression system of any one of items 32 to 38, wherein the cell is as defined in any one of items 1 to 31.
40. The expression system of any one of items 32 to 39, wherein the first nucleic acid and/or the second nucleic acid are independently comprised in a vector or are integrated in the genome of the cell.
41. A method for in vivo fluorination of a substrate, comprising the steps of:
i) propagating a cell in a medium, said cell comprising:
   a) a first nucleic acid comprising a first promoter and a fluorinase gene encoding a fluorinase (EC 2.5.1.63) capable of catalysing the formation of a C—F bond, said fluorinase gene being under the control of the first promoter;
   b) optionally a second nucleic acid comprising a riboswitch, wherein transcription of the fluorinase gene from the first promoter is induced in the presence of an inducer, wherein said riboswitch is responsive to said inducer;
ii) adding the inducer to the medium and incubating the cell in the presence of inducer and a co-substrate selected from a fluoride salt, wherein optionally the inducer is the co-substrate,
   wherein the fluorinase is the fluorinase of Methanosaeta sp. PtaU1.Bin055 as set forth in SEQ ID NO: 1 (FlA_{PtaU1}), or a functional variant thereof having at least 70% sequence homology, similarity or identity thereto, whereby transcription of the fluorinase gene is induced, thereby inducing fluorination of the substrate to yield a fluorinated compound.
42. The method according to item 41, wherein the second nucleic acid further comprises a second promoter and a gene encoding an activator of transcription, said gene being under the control of the second promoter, wherein transcription of the activator of transcription can be induced by the inducer, wherein the activator of transcription upon expression activates transcription from the first promoter, thereby inducing transcription of the fluorinase gene.
43. The method according to any one of items 41 to 42, wherein the first nucleic acid and the second nucleic acid are the same nucleic acid or wherein the first nucleic acid and the second nucleic acid are different nucleic acids.
44. The method according to any one of items 41 to 43, wherein the first nucleic acid further comprises a gene encoding a phosphorylase such as FlB1 or DeoD, said phosphorylase (EC 2.4.2.28 and/or EC 2.4.2.1) being capable of catalysing the phosphorylation of a fluorinated substrate such as a fluorinated adenosine, said gene being under control of the first promoter.
45. The method according to any one of items 41 to 44, wherein the first nucleic acid further comprises a gene encoding a nucleosidase (EC 3.2.2.9) such as Pfs, said nucleosidase being capable of catalysing the conversion of 5′-FDA to 5′-FDR, said nucleosidase gene being under control of the first promoter.
46. The method for in vivo fluorination according to any one of items 41 to 45, wherein the second nucleic acid further comprises a second promoter and a gene encoding an activator of transcription, said gene being under the control of the second promoter, wherein transcription of the activator of transcription can be induced by the inducer, wherein the activator of transcription upon expression activates transcription from the first promoter, thereby inducing transcription of the fluorinase gene and the phosphorylase gene.
47. The method according to any one of items 41 to 46, wherein the gene encoding the phosphorylase is the flB1 gene as set forth in SEQ ID NO: 12 or SEQ ID NO: 13, or the deoD gene as set forth in SEQ ID NO: 14, or a homologue thereof having at least 70% sequence homology, similarity or identity thereto, such as at least 71%, such as at least 72%, such as at least 73%, such as at least 74%, such as at least 75%, such as at least 76%, such as at least 77%, such as at least 78%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence homology, similarity or identity to SEQ ID NO: 12, SEQ ID NO: 13 or SEQ ID NO: 14.
48. The method according to any one of items 41 to 47, wherein the nucleosidase is the nucleosidase as set forth in SEQ ID NO: 17, or a functional variant thereof having at least 70% sequence homology, similarity or identity thereto, such as at least 71%, such as at least 72%, such as at least 73%, such as at least 74%, such as at least 75%, such as at least 76%, such as at least 77%, such as at least 78%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence homology, similarity or identity to SEQ ID NO: 17.
49. The method according to any one of items 41 to 48, wherein the cell is as defined in any one of items 1 to 31, or wherein the cell comprises an expression system as defined in item 32 to 40.
50. The method according to any one of items 41 to 49, wherein the inducer is the co-substrate.
51. The method according to any one of items 41 to 50, wherein the co-substrate or inducer is a fluoride salt such as NaF or KF.
52. The method according to any one of items 41 to 51, wherein the inducer and/or co-substrate is added at a concentration of at least 0.05 mM, such as at least 0.1 mM, such as at least 0.2 mM, such as at least 0.25 mM, such as at least 0.3 mM, such as at least 0.4 mM, such as at least 0.5 mM, such as at least 0.6 mM, such as at least 0.7 mM, such as at least 0.75 mM, such as at least 0.8 mM, such as at least 0.9 mM, such as at least 1 mM, such as at least 2 mM, such as at least 3 mM, such as at least 4 mM, such as at least 5 mM, such as at least 6 mM, such as at least 7 mM, such as at least 8 mM, such as at least 9 mM, such as at least 10 mM, such as at least 11 mM, such as at least 12 mM, such as at least 13 mM, such as at least 14 mM, such as at least 15 mM, such as at least 20 mM, such as 25 mM or more.
53. The method according to any one of items 41 to 52, wherein NaF or KF is added at a concentration of at least 0.05 mM, such as at least 0.1 mM, such as at least 0.2 mM, such as at least 0.25 mM, such as at least 0.3 mM, such as at least 0.4 mM, such as at least 0.5 mM, such as at least 0.6 mM, such as at least 0.7 mM, such as at least 0.75 mM, such as at least 0.8 mM, such as at least 0.9 mM, such as at least 1 mM, such as at least 2 mM, such as at least 3 mM, such as at least 4 mM, such as at least 5 mM, such as at least 6 mM, such as at least 7 mM, such as at least 8 mM, such as at least 9 mM, such as at least 10 mM, such as at least 11 mM, such as at least 12 mM, such as at least 13 mM, such as at least 14 mM, such as at least 15 mM, such as at least 20 mM, such as 25 mM or more.
54. The method according to any one of items 41 to 53, wherein the inducer and/or co-substrate is added at the culture onset or in mid-exponential phase.
55. The method according to any one of items 41 to 54, wherein the cell is incubated in the presence of inducer and/or co-substrate for at least 4 hours, such as at least 8 hours, such as at least 12 hours, such as at least 16 hours, such as at least 20 hours, such as at least 24 hours, such as at least 36 hours, such as at least 48 hours, or more.
56. The method according to any one of the preceding items, wherein 5′-deoxy-5′-fluoroadenosine is produced with a titer of at least 1 μM, such as at least 1.5 μM, such as at least 2 μM, such as at least 2.5 μM, such as at least 3 μM, such as at least 3.5 μM, such as at least 4 μM, such as at least 4.5 μM, such as at least 5 μM, such as at least 5.5 μM, such as at least 6 μM, such as at least 6.5 μM, such as at least 7 μM, such as at least 7.5 μM, such as at least 8 μM, such as at least 8.5 μM, such as at least 9 μM, such as at least 9.5 μM, such as at least 10 μM, such as at least 15 μM, such as at least 20 μM, such as at least 25 μM, such as at least 30 μM, such as at least 35 μM, such as at least 40 μM, such as at least 45 μM, such as at least 50 μM, such as at least 55 μM, such as at least 60 μM, such as at least 70 μM, such as at least 80 μM, such as at least 90 μM, such as at least 100 μM, or more.
57. The method according to any one of items 41 to 56, further comprising a step of recovering the product.
58. A method for in vitro fluorination of a substrate, comprising the steps of:
   i. providing a fluorinase as set forth in SEQ ID NO: 1 (FlA_{PtaU1}), or a functional variant thereof having at least 70% sequence homology, similarity or identity thereto;
   ii. contacting said fluorinase with the substrate in the presence of fluoride;
   whereby said substrate is fluorinated.
59. The method according to item 58, wherein the fluorinase is a functional variant of the fluorinase of Methanosaeta sp. PtaU1.Bin055 as set forth in SEQ ID NO: 1 (FlA_{PtaU1}), having at least 70% sequence homology, similarity or identity thereto, such as at least 71%, such as at least 72%, such as at least 73%, such as at least 74%, such as at least 75%, such as at least 76%, such as at least 77%, such as at least 78%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence homology, similarity or identity to SEQ ID NO: 1.
60. The method according to any one of items 58 to 59, wherein the substrate is as defined in any one of items 10-15.
61. The method according to any one of items 58 to 60, wherein the fluorinase comprises or consists of the sequence as set forth in SEQ ID NO: 1, with the exception that at the most 30 residues are mutated.
62. A fluorinated compound obtainable by the method according to any one of items 41 to 61.
63. A composition comprising a fluorinated compound according to item 62.
64. A method for manufacturing a fluorinated compound of interest, said method comprising the steps of:
i) providing a fluorinated compound by the method of any one of items 41 to 57; and
ii) optionally converting said fluorinated compound to the fluorinated compound of interest.
65. The fluorinated compound according to item 62 or the composition according to item 63 for use as a medicament.
66. Use of the fluorinated compound according to item 62 or the composition according to item 63 as a building block or a precursor of polymers.
67. Use of a polypeptide as set forth in SEQ ID NO: 1 or a functional variant thereof having at least 70% sequence homology, similarity or identity thereto for catalysing fluorination of a substrate.
68. The use according to item 67, wherein the polypeptide comprises or consists of the sequence as set forth in SEQ ID NO: 1, with the exception that at the most 30 residues are mutated.
69. The use according to any one of items 67 to 68, wherein fluorination is performed in vitro or in vivo.
70. The use according to any one of items 67 to 69, wherein the substrate is as defined in any one of items 10 to 15.

### Items 3

1. A cell capable of producing F-acetaldehyde (FAld) and optionally F-acetate (FAc), F-ethanol (FEtOH) and/or F-acetyl-CoA (FAcCoA) and/or one or more derivatives thereof from a fluorinated compound selected from 5′-fluoro-5′-deoxy-D-ribose 1-phosphate (5′-FDRP) and/or (3R,4S)-5′-fluoro-5′-deoxy-D-ribulose-1-phosphate (5′-FDRuIP), said cell expressing:
   i. an isomerase (EC 5.3.1.23); and
   ii. an aldolase (EC 4.1.2.62);
   whereby said cell is capable of catalysing formation of FAld and optionally FEtOH; and
   iii. optionally, said cell further expressing:
      a. an acetylating acetaldehyde dehydrogenase (EC 1.2.1.10); and/or
      b. a fluoroacetaldehyde dehydrogenase (EC 1.2.1.69), and optionally an acetyl-CoA synthetase (EC 6.2.1.1);
      whereby said cell is capable of catalysing formation of FAcCoA, FEtOH, and/or FAc, and/or one or more derivatives of FAld, FAc, FEtOH, and/or FAcCoA,
optionally said cell further expressing:
   iv. a fluorinase (EC 2.5.1.63), such as a fluorinase selected from the group consisting of FlA_{PtaU1} as set forth in SEQ ID NO: 1 and FlA1_{MA37} as set forth in SEQ ID NO: 30, or a functional variant thereof having at least 70% homology, identity or similarity to SEQ ID NO: 1 or SEQ ID NO: 30, respectively,
   v. a purine nucleoside phosphorylase (PNP) (EC 2.4.2.1 and/or EC 2.4.2.28); and/or
   vi. a nucleosidase (EC 3.2.2.9) and/or a kinase (EC 2.7.1.100),
   whereby said cell is further capable of fluorinating a substrate in the presence of fluoride, thereby producing the fluorinated compound, such as 5′-FDRP.
2. A cell capable of producing F-acetaldehyde (FAld) and optionally F-acetate (FAc), F-ethanol (FEtOH) and/or F-acetyl-CoA (FAcCoA) and/or one or more derivatives thereof from a fluorinated compound selected from 5′-fluoro-5′-deoxy-D-ribose 1-phosphate (5′-FDRP) and/or (3R,4S)-5′-fluoro-5′-deoxy-D-ribulose-1-phosphate (5′-FDRuIP), said cell expressing:
   i. an isomerase (EC 5.3.1.23); and
   ii. an aldolase (EC 4.1.2.62);
   whereby said cell is capable of catalysing formation of FAld and optionally FEtOH; and
   iii. optionally, said cell further expressing:
      a. an acetylating acetaldehyde dehydrogenase (EC 1.2.1.10); and/or
      b. a fluoroacetaldehyde dehydrogenase (EC 1.2.1.69), and/or an acetyl-CoA synthetase (EC 6.2.1.1);
      whereby said cell is capable of catalysing formation of FAcCoA, FEtOH, and/or FAc, and/or one or more derivatives of FAld, FAc, FEtOH, and/or FAcCoA,
   optionally said cell further expressing:
   iv. a fluorinase (EC 2.5.1.63), such as a fluorinase selected from the group consisting of FlA_{PtaU1} as set forth in SEQ ID NO: 1 and FlA1_{MA37} as set forth in SEQ ID NO: 30, or a functional variant thereof having at least 70% homology, identity or similarity to SEQ ID NO: 1 or SEQ ID NO: 30, respectively,
   v. a purine nucleoside phosphorylase (PNP) (EC 2.4.2.1 and/or EC 2.4.2.28); and/or
   vi. a nucleosidase (EC 3.2.2.9) and/or a kinase (EC 2.7.1.100),
   whereby said cell is further capable of fluorinating a substrate in the presence of fluoride, thereby producing the fluorinated compound, such as 5′-FDRP.
3. A cell capable of producing F-acetaldehyde (FAld) and optionally F-acetate (FAc), F-ethanol (FEtOH) and/or F-acetyl-CoA (FAcCoA) and/or one or more derivatives thereof from a fluorinated compound selected from 5′-FDA, 5′-FDR, 5′-FDRP, 5-FDRuIP, FAld, FAc, FEtOH and FAcCoA, said cell expressing:
   i. an isomerase (EC 5.3.1.23); and/or
   ii. an aldolase (EC 4.1.2.62);
   whereby said cell is capable of catalysing formation of FAld and optionally FEtOH; and
   iii. optionally, said cell further expressing:
      a. an acetylating acetaldehyde dehydrogenase (EC 1.2.1.10); and/or
      b. a fluoroacetaldehyde dehydrogenase (EC 1.2.1.69), and optionally an acetyl-CoA synthetase (EC 6.2.1.1);
      whereby said cell is capable of catalysing formation of FAcCoA, FEtOH, and/or FAc, and/or one or more derivatives of FAld, FAc, FEtOH, and/or FAcCoA.
4. A cell capable of producing F-acetaldehyde (FAld) and optionally F-acetate (FAc), F-ethanol (FEtOH) and/or F-acetyl-CoA (FAcCoA) and/or one or more derivatives thereof from a fluorinated compound selected from 5′-FDA, 5′-FDR, 5′-FDRP, 5-FDRuIP, FAld, FAc, FEtOH and FAcCoA, said cell expressing:
   i. an isomerase (EC 5.3.1.23); and/or
   ii. an aldolase (EC 4.1.2.62);
   whereby said cell is capable of catalysing formation of FAld and optionally FEtOH; and
   iii. optionally, said cell further expressing:
      a. an acetylating acetaldehyde dehydrogenase (EC 1.2.1.10); and/or
      b. a fluoroacetaldehyde dehydrogenase (EC 1.2.1.69), and/or an acetyl-CoA synthetase (EC 6.2.1.1);
      whereby said cell is capable of catalysing formation of FAcCoA, FEtOH, and/or FAc, and/or one or more derivatives of FAld, FAc, FEtOH, and/or FAcCoA.
5. The cell according to any one of the preceding items, wherein the cell expresses a fluorinase, and wherein the fluorinase is FlA_{PtaU1}, or a functional variant thereof having at least 70% homology, identity or similarity to SEQ ID NO: 1.
6. The cell according to any one of the preceding items, wherein the fluorinated compound is;
   i. 5′-FDRP, and wherein the isomerase is capable of catalysing the isomerisation of 5′-FDRP, thereby producing an isomer of 5′-FDRP such as 5′-FDRuIP; and/or
   ii. 5′-FDRuIP, and wherein the aldolase is capable of catalysing the conversion of 5′-FDRuIP to FAld.
7. The cell according to any one of the preceding items, wherein the cell expresses an acetylating acetaldehyde dehydrogenase (EC 1.2.1.10), a fluoroacetaldehyde dehydrogenase (EC 1.2.1.69) and/or an acetyl-CoA synthetase (EC 6.2.1.1), and wherein the cell is capable of producing;
   i. FAld, and wherein the acetylating acetaldehyde dehydrogenase (AcAldh, EC 1.2.1.10) is capable of catalysing the conversion of FAld to FAcCoA;
   ii. FAld, and wherein the fluoroacetaldehyde dehydrogenase (F-Aldh, EC 1.2.1.69) is capable of catalysing the conversion of FAld to FAc;
   iii. FAld, and wherein the fluoroacetaldehyde dehydrogenase (EC 1.2.1.69) and the acetylating acetaldehyde dehydrogenase (EC 1.2.1.10) are identical such as an acetaldehyde dehydrogenase (Aldh) capable of catalysing the conversion of FAld to FAc and/or FAcCoA; and/or
   iv. FAc, and wherein the acetyl-CoA synthetase (Acs) is capable of catalysing the conversion of FAc to FAcCoA.
8. The cell according to any one of the preceding items, wherein the cell is capable of producing FAld, and wherein said FEtOH is produced by an alcohol dehydrogenase (ADH, EC 1.1.1.1), said alcohol dehydrogenase being capable of catalysing the conversion of FAld to FEtOH.
9. The cell according to any one of the preceding items, wherein the cell expresses a fluorinase (EC 2.5.1.63), a purine nucleoside phosphorylase (PNP) (EC 2.4.2.1 and/or EC 2.4.2.28), a nucleosidase (EC 3.2.2.9) and/or a kinase (EC 2.7.1.100), and wherein;
   i. the fluorinase is capable of catalysing fluorination of a substrate in the presence of fluoride, whereby the cell is capable of producing a fluorinated compound such as 5′-fluoro-5′-deoxyadenosine (5′-FDA); and/or wherein the PNP is capable of catalysing phosphorylation of a fluorinated compound such as a fluorinated adenosine, preferably 5′-FDA, whereby the cell is capable of producing a phosphorylated and fluorinated compound such as 5′-FDRP;
   ii. the nucleosidase is capable of catalysing the conversion of 5′-fluoro-5′-deoxyadenosine (5′-FDA) to 5′-fluorodeoxyribose (5′-FDR), whereby the cell is capable of producing 5′-FDR; and/or
   iii. the kinase is capable of catalysing the conversion of 5′-fluorodeoxyribose (5′-FDR) to 5′-fluoro-5′-deoxy-D-ribose 1-phosphate (5′-FDRP), whereby the cell is capable of producing 5′-FDRP.
10. The cell according to any one of the preceding items, wherein;
   i. the isomerase is an isomerase native to Bacillus such as B. thuringiensis, or an isomerase native to Streptomyces such as S. cattleya, preferably the isomerase is IsoBt as set forth in SEQ ID NO: 35 or IsoSc as set forth in SEQ ID NO: 37;
   ii. the aldolase is an aldolase native to Bacillus such as B. thuringiensis, or an aldolase native to Streptomyces such as S. coelicolor, preferably the aldolase is AldBt as set forth in SEQ ID NO: 29 or AldSc as set forth in SEQ ID NO: 19;
   iii. the F-Aldh and/or the AcAldh is an F-Aldh and/or an AcAldh native to Pseudomonas such as P. aeruginosa, preferably P. aeruginosa 1984 or P. aeruginosa 4022, or an F-Aldh and/or an AcAldh native to Escherichia such as E. coli, or an F-Aldh and/or an AcAldh native to Moorella thermoacetica such as M. thermoacetica, preferably the F-Aldh and/or an AcAldh is selected from the group consisting of ExaC as set forth in SEQ ID NO: 21, HdhA as set forth in SEQ ID NO: 23, EutE as set forth in SEQ ID NO: 25, and Moth_1776p as set forth in SEQ ID NO: 27;
   iv. the Acs is an Acs native to Bacillus such as B. subtilis, or an Acs native to Streptomyces such as S. coelicolor, or an Acs native to Cupriavidus such as C. necator, or an Acs native to Pseudomonas such as P. putida or P. aeruginosa, preferably the Acs is selected from the group consisting of AcsBt as set forth in SEQ ID NO: 39, AcsCn as set forth in SEQ ID NO: 41, and AcsSc as set forth in SEQ ID NO: 43;
   v. the ADH is an ADH native to Pseudomonas such as P. putida;
   vi. the PNP is a PNP native to Streptomyces such as Streptomyces sp. MA37, S. xinghaeiensis or S. cattleya, or a PNP native to Escherichia such as E. coli, preferably the PNP is FlB1 as set forth in SEQ ID NO: 11 or DeoD as set forth in SEQ ID NO: 15;
   vii. the nucleosidase is a nucleosidase native to Escherichia such as E. coli, preferably the nucleosidase is Pfs as set forth in SEQ ID NO: 17;
   viii. the kinase is a kinase native to Bacillus such as Bacillus thuringiensis, preferably the kinase is KinBt as set forth in SEQ ID NO: 33; and/or
   ix. the fluorinase is a fluorinase native to Methanosaeta such as Methanosaeta sp. PtaU1.Bin055, or a fluorinase native to Streptomyces such as Streptomyces sp. MA37, Streptomyces sp. SAJ15, S. cattleya, S. xinghaiensis, or a fluorinase native to Nocardia such as N. brasiliensis or a fluorinase native to Actinoplanes such as Actinoplanes sp. N902-109, preferably wherein the fluorinase is selected from FlA_{PtaU1} as set forth in SEQ ID NO: 1 and FlA1_{MA37} as set forth in SEQ ID NO: 30, further preferably the fluorinase is FlA_{PtaU1} as set forth in SEQ ID NO: 1,
   or functional variants thereof having at least 70% homology, identity or similarity thereto, such as at least 71%, such as at least 72%, such as at least 73%, such as at least 74%, such as at least 75%, such as at least 76%, such as at least 77%, such as at least 78%, such as at least 79%, such as at least 80%, such as at least 81%, such as at least 82%, such as at least 83%, such as at least 84%, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99%, such as 100% homology, identity or similarity to SEQ ID NO: 35, 37, 29, 19, 21, 23, 25, 27, 39, 41, 43, 11, 15, 17, 33, 1 or 30, respectively.
11. The cell according to any one of the preceding items, wherein the substrate is selected from the group consisting of S-adenosyl-L-methionine (SAM) or a derivative thereof, such as a methylaza derivative, 5′-chloro-5′-deoxyadenosine, a 2-deoxyadenosine analogue, an L-methionine analogue, a di-cyclic peptide conjugate of 5′-chlorodeoxy-2-ethynyladenosine, a tri-cyclic peptide conjugate of 5′-chlorodeoxy-2-ethynyladenosine, fluoride, and ¹⁸F.
12. The cell according to any one of the preceding items, wherein the cell is a bacterial cell, preferably wherein the genus of said bacterial cell is selected from Pseudomonas, Bacillus, Streptomyces, Vibrio and Escherichia, such as a bacterial cell selected from Pseudomonas putida, Pseudomonas fluorescens, Pseudomonas taiwanensis, Pseudomonas syringae, Pseudomonas stutzeri, Pseudomonas oleovorans, Pseudomonas mendocina, Bacillus subtilis, Bacillus cereus, Bacillus megaterium, Streptomyces albus, Streptomyces venezuelae, Streptomyces coelicolor, Vibrio natriegens and Escherichia coli, or wherein the cell is a yeast cell of the genus Saccharomyces, Pichia, Yarrowia, Kluyveromyces, Candida, Rhodotorula, Rhodosporidium, Cryptococcus, Trichosporon or Lipomyces, such as a yeast cell selected from Saccharomyces cerevisiae, Pichia pastoris, Kluyveromyces marxianus, Cryptococcus albidus, Lipomyces lipofer, Lipomyces starkeyi, Rhodosporidium toruloides, Rhodotorula glutinis, Trichosporon pullulans and Yarrowia lipolytica, preferably the cell is Pseudomonas putida KT2440.
13. A method for production of FAld and optionally FAcCoA, FEtOH, and/or FAc and/or one or more derivatives thereof from a fluorinated compound selected from 5′-fluoro-5′-deoxy-D-ribose 1-phosphate (5′-FDRP) and/or (3R,4S)-5′-fluoro-5′-deoxy-D-ribulose-1-phosphate (5′-FDRuIP), said method comprising the steps of:
   i. providing a cell, said cell expressing:
      a. an isomerase (EC 5.3.1.23); and
      b. an aldolase (EC 4.1.2.62);
      whereby said cell is capable of catalysing formation of FAld and optionally FEtOH; and
      c. optionally, said cell further expressing:
         I. an acetylating acetaldehyde dehydrogenase (EC 1.2.1.10); and/or
         II. a fluoroacetaldehyde dehydrogenase (EC 1.2.1.69), and optionally an acetyl-CoA synthetase (EC 6.2.1.1); and
   ii. propagating said cell in a medium, optionally wherein the medium comprises the fluorinated compound or a compound such as a substrate which can be converted to the fluorinated compound by said cell;
   whereby FAld and optionally FAcCoA, FEtOH, and/or FAc and/or one or more derivatives thereof is produced,
   optionally wherein the method is performed in vitro or in vivo.
14. A method for production of FAld and optionally FAcCoA, FEtOH, and/or FAc and/or one or more derivatives thereof from a fluorinated compound selected from 5′-fluoro-5′-deoxy-D-ribose 1-phosphate (5′-FDRP) and/or (3R,4S)-5′-fluoro-5′-deoxy-D-ribulose-1-phosphate (5′-FDRuIP), said method comprising the steps of:
   i. providing a cell, said cell expressing:
      a. an isomerase (EC 5.3.1.23); and/or
      b. an aldolase (EC 4.1.2.62);
      whereby said cell is capable of catalysing formation of FAld and optionally FEtOH; and
      c. optionally, said cell further expressing:
         I. an acetylating acetaldehyde dehydrogenase (EC 1.2.1.10); and/or
         II. a fluoroacetaldehyde dehydrogenase (EC 1.2.1.69), and/or an acetyl-CoA synthetase (EC 6.2.1.1); and
   ii. propagating said cell in a medium, optionally wherein the medium comprises the fluorinated compound or a compound such as a substrate which can be converted to the fluorinated compound by said cell;
   whereby FAld and optionally FAcCoA, FEtOH, and/or FAc and/or one or more derivatives thereof is produced,
   optionally wherein the method is performed in vitro or in vivo.
15. The method according to item 13, wherein the cell is as defined in anyone of items 1 to 12.
16. The method according to any one of items 13 to 15, further comprising a step of recovering the fluorinated compound, and/or further comprising a step of converting the fluorinated compound to a downstream product, said downstream product being fluorinated and/or non-fluorinated.
17. The method according to any one of items 13 to 16, wherein the substrate is as defined in item 11.
18. The method according to any one of items 13 to 17, wherein FAld and optionally FAcCoA, FEtOH, and/or FAc and/or one or more derivatives thereof is produced with a titer of at least 0.01 mM, such as at least 0.02 mM, such as at least 0.05 mM, such as at least 0.1 mM, such as at least 0.2 mM, such as at least 0.3 mM, such as at least 0.4 mM, such as at least 0.5 mM, such as at least 0.7 mM, such as at least 0.8 mM, such as at least 0.9 mM, such as at least 1 mM, such as at least 1.5 mM, such as at least 2 mM, such as at least 2.5 mM, such as at least 3 mM, such as at least 3.5 mM, such as at least 4 mM, such as at least 4.5 mM, such as at least 5 mM, such as at least 5.5 mM, such as at least 6 mM, such as at least 6.5 mM, such as at least 7 mM, such as at least 7.5 mM, such as at least 8 mM, such as at least 8.5 mM, such as at least 9 mM, such as at least 9.5 mM, such as at least 10 mM, such as at least 10.5 mM, such as at least 11 mM, such as at least 13 mM, such as at least 15 mM, such as at least 20 mM, such as at least 25 mM, or more.
19. A method for manufacturing a fluorinated compound of interest, said method comprising the steps of:
   i. providing a fluorinated compound by the method of any one of items 13 to 18; and
   ii. optionally converting said fluorinated compound to the fluorinated compound of interest,
   optionally wherein the fluorinated compound and/or fluorinated compound of interest is selected from FAld, FAc, FEtOH and FAcCoA.

1-101. (canceled)

102. A cell capable of producing F-acetaldehyde (FAld) or one or more derivatives thereof from a fluorinated compound, wherein the fluorinated compound is 5′-fluoro-5′-deoxy-D-ribose 1-phosphate (5′-FDRP), said cell expressing:
i. an isomerase (EC 5.3.1.23); and
ii. an aldolase (EC 4.1.2.62);
whereby said cell is capable of catalysing formation of FAld;
and
iii. a fluorinase (EC 2.5.1.63) selected from FlA_{PtaU1} as set forth in SEQ ID NO: 1 and FlA1_{MA37} as set forth in SEQ ID NO: 30, or a functional variant thereof having at least 70% homology, identity or similarity thereto;
said cell further expressing:
iv. a purine nucleoside phosphorylase (PNP) or phosphorylase (EC 2.4.2.1 and/or EC 2.4.2.28);
or
v. a nucleosidase (EC 3.2.2.9) and a kinase (EC 2.7.1.100);
whereby said cell is capable of fluorinating a substrate in the presence of fluoride,
thereby producing the fluorinated compound.

103. The cell according to claim 102, wherein the cell is further capable of producing F-acetate (FAc), F-ethanol (FEtOH), or F-acetyl-CoA (FAcCoA), said cell further expressing:
a. an acetylating acetaldehyde dehydrogenase (AcAldh, EC 1.2.1.10), whereby said cell is capable of catalysing formation of FAcCoA or one or more derivatives thereof;
b. a fluoroacetaldehyde dehydrogenase (F-Aldh, EC 1.2.1.69), whereby said cell is capable of catalysing formation of FAc or one or more derivatives thereof, or
c. an F-Aldh and an acetyl-CoA synthetase (Acs, EC 6.2.1.1), whereby said cell is capable of catalysing formation of FAcCoA or one or more derivatives thereof.

104. The cell according to claim 102, wherein:
i. the isomerase is an isomerase native to Bacillus, B. thuringiensis, Streptomyces or S. cattleya;
ii. the aldolase is an aldolase native to Bacillus, B. thuringiensis, Streptomyces or S. coelicolor;
iii. the PNP is a PNP native to Streptomyces, Streptomyces sp. MA37, S. xinghaeiensis, S. cattleya, Escherichia, or E. coli;
iv. the nucleosidase is a nucleosidase native to Escherichia or E. coli;
v. the kinase is a kinase native to Bacillus or Bacillus thuringiensis;
vi. the F-Aldh or the AcAldh is an F-Aldh or an AcAldh native to Pseudomonas, P. aeruginosa, P. aeruginosa 1984 or P. aeruginosa 4022, or an fluoroacetaldehyde dehydrogenase or an acetylating acetaldehyde dehydrogenase native to Escherichia or E. coli, or an fluoroacetaldehyde dehydrogenase or the acetylating acetaldehyde dehydrogenase native to Moorella thermoacetica or M. thermoacetica; or
vii. the acetyl-CoA synthetase (Acs) is an Acs native to Bacillus, B. subtilis, Streptomyces, S. coelicolor, Cupriavidus, C. necator, Pseudomonas, P. putida or P. aeruginosa.

105. The cell according to claim 102, wherein:
i. the isomerase is IsoBt as set forth in SEQ ID NO: 35 or IsoSc as set forth in SEQ ID NO: 37, or a functional variant thereof having at least 70% homology, identity or similarity to SEQ ID NO: 35 or SEQ ID NO: 37, respectively;
ii. the aldolase is AldBt as set forth in SEQ ID NO: 29 or AldSc as set forth in SEQ ID NO: 19, or a functional variant thereof having at least 70% homology, identity or similarity to SEQ ID NO: 29 or SEQ ID NO: 19, respectively;
iii. the PNP is FlB1 as set forth in SEQ ID NO: 11 or DeoD as set forth in SEQ ID NO: 15, or a functional variant thereof having at least 70% homology, identity or similarity to SEQ ID NO: 11 or SEQ ID NO: 15, respectively;
iv. the nucleosidase is Pfs as set forth in SEQ ID NO: 17, or a functional variant thereof having at least 80% homology, identity or similarity thereto;
v. the kinase is KinBt as set forth in SEQ ID NO: 33, or a functional variant thereof having at least 70% homology, identity or similarity thereto;
vi. the F-Aldh or the AcAldh is ExaC as set forth in SEQ ID NO: 21, HdhA as set forth in SEQ ID NO: 23, EutE as set forth in SEQ ID NO: 25, or Moth_1776p as set forth in SEQ ID NO: 27, or a functional variant thereof having at least 70% homology, identity or similarity to SEQ ID NO: 21, SEQ ID NO: 23, SEQ ID NO: 25 or SEQ ID NO: 27, respectively; or
vii. the acetyl-CoA synthetase (Acs) is AcsBt as set forth in SEQ ID NO: 39, AcsCn as set forth in SEQ ID NO: 41, or AcsSc as set forth in SEQ ID NO: 43, or a functional variant thereof having at least 70% homology, identity or similarity to SEQ ID NO: 39, SEQ ID NO: 41, or SEQ ID NO: 43, respectively.

106. The cell according to claim 102, wherein the cell is capable of producing FAld and wherein said FEtOH is produced by an alcohol dehydrogenase (ADH, EC 1.1.1.1), said ADH being capable of catalysing the conversion of FAld to FEtOH.

107. The cell according to claim 102, wherein the substrate is S-adenosyl-L-methionine (SAM) or a derivative thereof, 5′-chloro-5′-deoxyadenosine, a 2-deoxyadenosine analogue, an L-methionine analogue, a di-cyclic peptide conjugate of 5′-chlorodeoxy-2-ethynyladenosine, a tri-cyclic peptide conjugate of 5′-chlorodeoxy-2-ethynyladenosine, fluoride, or ¹⁸F.

108. The cell according to claim 102, wherein the cell is a bacterial cell or a Gram-negative bacterial cell.

109. The cell according to claim 108, wherein the cell is a bacterial cell of the Pseudomonas genus, the Bacillus genus, the Streptomyces genus, the Vibrio genus or the Escherichia genus or a yeast cell of the genus Saccharomyces, Pichia, Yarrowia, Kluyveromyces, Candida, Rhodotorula, Rhodosporidium, Cryptococcus, Trichosporon or Lipomyces.

110. The cell according to claim 108, wherein the cell is a bacterial cell of a species selected from Pseudomonas putida, Pseudomonas fluorescens, Pseudomonas taiwanensis, Pseudomonas syringae, Pseudomonas stutzeri, Pseudomonas oleovorans, Pseudomonas mendocina, Bacillus subtilis, Bacillus cereus, Bacillus megaterium, Streptomyces albus, Streptomyces venezuelae, Streptomyces coelicolor, Vibrio natriegens or Escherichia coli, or a yeast cell of the species Saccharomyces cerevisiae, Pichia pastoris, Kluyveromyces marxianus, Cryptococcus albidus, Lipomyces lipofer, Lipomyces starkeyi, Rhodosporidium toruloides, Rhodotorula glutinis, Trichosporon pullulans or Yarrowia lipolytica.

111. The cell according to claim 102, wherein the cell is further modified to reduce endogenous conversion of F-acetaldehyde (FAld) to F-acetate (FAc), wherein the modification of the cell comprises reduction of endogenous aldehyde dehydrogenase activity.

112. A method for production of FAld or one or more derivatives thereof from a fluorinated compound, wherein the fluorinated compound is 5′-fluoro-5′-deoxy-D-ribose 1-phosphate (5′-FDRP), comprising:
i. providing a cell, said cell expressing:
a. an isomerase (EC 5.3.1.23); and
b. an aldolase (EC 4.1.2.62);
whereby said cell is capable of catalysing formation of FAld; and
c. a fluorinase (EC 2.5.1.63) selected from FlA_{PtaU1} as set forth in SEQ ID NO: 1 and FlA1_{MA37} as set forth in SEQ ID NO: 30, or a functional variant thereof having at least 70% homology, identity or similarity thereto;
and further expressing:
d. a purine nucleoside phosphorylase (PNP) or phosphorylase (EC 2.4.2.1 or EC 2.4.2.28); or
e. a nucleosidase (EC 3.2.2.9) and a kinase (EC 2.7.1.100);
whereby said cell is capable of fluorinating a substrate in the presence of fluoride,
thereby producing the fluorinated compound;
and
ii. propagating said cell in a medium;
whereby FAld or one or more derivatives thereof is produced.

113. The method according to claim 112, wherein the method is a method for production of FAld and F-acetate (FAc), F-ethanol (FEtOH), or F-acetyl-CoA (FAcCoA) or one or more derivatives thereof,
wherein said cell further expresses:
a. an acetylating acetaldehyde dehydrogenase (AcAldh, EC 1.2.1.10), whereby said cell is capable of catalysing formation of FAcCoA or one or more derivatives thereof;
b. a fluoroacetaldehyde dehydrogenase (F-Aldh, EC 1.2.1.69), whereby said cell is capable of catalysing formation of FAc or one or more derivatives thereof; or
c. an F-Aldh and an acetyl-CoA synthetase (Acs, EC 6.2.1.1), whereby said cell is capable of catalysing formation of FAcCoA or one or more derivatives thereof.

114. The method according to claim 112, further comprising a step of recovering the FAld, FAc, FEtOH, or FAcCoA or one or more derivatives thereof.

115. The method according to claim 112, further comprising a step of converting the FAld, FAc, FEtOH, FAcCoA or one or more derivatives thereof to a downstream product, said downstream product being fluorinated or non-fluorinated.

116. The method according to claim 112, wherein the medium comprises a substrate which can be converted to the fluorinated compound by said cell, wherein the substrate is S-adenosyl-L-methionine (SAM) or a derivative thereof, 5′-chloro-5′-deoxyadenosine, a 2-deoxyadenosine analogue, an L-methionine analogue, a di-cyclic peptide conjugate of 5′-chlorodeoxy-2-ethynyladenosine, a tri-cyclic peptide conjugate of 5′-chlorodeoxy-2-ethynyladenosine, fluoride, or ¹⁸F.

117. The method according to claim 112, wherein FAld, FAcCoA, FEtOH, or FAc or one or more derivatives thereof is produced with a titer of at least 0.01 mM, or more.

118. A method for manufacturing a fluorinated compound of interest, said method comprising the steps of:
i. providing a fluorinated compound obtained by the method of claim 112; and
ii. converting said fluorinated compound to the fluorinated compound of interest,
wherein the fluorinated compound or fluorinated compound of interest is FAld, FAc, FEtOH or FAcCoA or one or more derivatives thereof.

119. Use of a polypeptide as set forth in SEQ ID NO: 1 or a functional variant thereof having at least 70% homology, identity or similarity to SEQ ID NO: 1 for catalysing fluorination of a substrate.

120. The use according to claim 119, wherein the polypeptide comprises or consists of the sequence as set forth in SEQ ID NO: 1, with the proviso that at the most 30 residues are mutated, or
wherein the substrate is S-adenosyl-L-methionine (SAM) or a derivative thereof, 5′-chloro-5′-deoxyadenosine, a 2-deoxyadenosine analogue, an L-methionine analogue, a di-cyclic peptide conjugate of 5′-chlorodeoxy-2-ethynyladenosine, a tri-cyclic peptide conjugate of 5′-chlorodeoxy-2-ethynyladenosine, fluoride, or ¹⁸F.
